(19) United States
(12) Patent Application Publication
(10) Pub. No.: US 2015/0044232 A1
(43) Pub. Date: Feb. 12, 2015

(54) MARKERS FOR THE RESPONSIVENESS TO ANTI-CD44 ANTIBODIES

(71) Applicant: HOFFMANN-LA ROCHE INC., Little Falls, NJ (US)

(72) Inventors: Fabian Birzele, Benediktbeuern (DE); Michael Cannarile, Muenchen (DE); Friedrich Feuerhake, Hannover (DE); Thomas Fischer, Muenchen (DE); Florian Heil, Germering (DE); Konrad Honold, Penzberg (DE); Adam Nopora, Penzberg (DE); Annette Schmitt-Graeff, Stegen (DE); Edgar Voss, Bichl (DE); Stefan Weigand, Penzberg (DE)

(73) Assignee: HOFFMANN-LA ROCHE INC., Little Falls, NJ (US)

(21) Appl. No.: 14/303,166

(22) Filed: Jun. 12, 2014

(30) Foreign Application Priority Data
Jun. 12, 2013 (EP) 13171610.2

(51) Int. Cl. C12Q 1/68; C07K 16/28

(52) U.S. Cl. CPC C12Q 1/6886; C07K 16/2884; C12Q 2600/158; C12Q 2600/16; C12Q 2600/106
USPC 424/174.1; 435/6.17; 435/6.12; 435/6.11; 506/9

(57) ABSTRACT

The present invention relates to means and methods of determining whether a tumor cell or a cancer cell is responsive to an anti-CD44 antibody or to an antigen binding fragment thereof. The method comprises the determination of the major CD44 isoform in a sample, wherein if the major CD44 isoform is CD44s, the tumor cell or cancer cell is responsive to said anti-CD44 antibody. Also means and methods of treating a cancer patient that has been determined to respond to an anti-CD44 antibody are subject of the present invention.

### CROSS REFERENCE TO RELATED APPLICATIONS

This application claims priority to European Patent Application No. EP 13171610.2, filed Jun. 12, 2013, the disclosure of which is incorporated herein by reference in its entirety.

### SEQUENCE LISTING

The present application contains a Sequence Listing which has been submitted in ASCII format via EFS-Web and is hereby incorporated by reference in its entirety. Said ASCII copy, created on Jun. 9, 2014, is named P5834US_ST25.txt and is 368,006 bytes in size.

### FIELD OF THE INVENTION

The present invention relates to means and methods of determining whether a tumor cell or a cancer cell is responsive to an anti-CD44 antibody or to an antigen binding fragment thereof. The method comprises the determination of the major CD44 isoform in a sample, wherein if the major CD44 isoform is CD44s, the tumor cell or cancer cell is responsive to said anti-CD44 antibody. Also means and methods of treating a cancer patient that has been determined to respond to an anti-CD44 antibody are subject of the present invention.

### BACKGROUND

The cell adhesion molecule CD44 is a transmembrane glycoprotein overexpressed and linked to bad prognosis in a number of tumor entities like breast (Gotte et al. Cancer Res. 2006; Afify et al. Exp. Mol. Pathol. 2009; Louderbough et al. Mol. Cancer. Res. 2011) and hematological malignancies (Liu et al. Cellular Mol. Immunolgy, 2006). CD44 receptor varies in size and function owing to N- and O-glycosylation and alternative splicing, which affects predominantly the extracellular domain of the protein. Despite not being a direct signaling molecule, CD44 is said to be involved in a variety of functions that promote tumor development, progression and metastasis (Zoller et al. Nature Rev. Cancer, 2011) via cellular functions such as cell migration, cell adhesion and metastatic spread (Haynes et al. Immunol. Today, 1989). Anti-CD44 antibodies for use in the treatment of head and neck squamous cell carcinoma are disclosed in WO 2011/095498. WO 2008/036333 discloses that elevated levels of soluble CD44 might be used to identify head and neck squamous cell carcinoma patients responsive to CD44 targeting therapies. WO 2008/036333 does not suggest that the determination of the major isoform of CD44 might be useful. Qian (Cancer Biol & Ther 13, 1276-1283 (2012)) speculates that cells containing CD44s might be sensitive to antibody-induced apoptosis. Yet, Qian does not propose that the determination of the major isoform of CD44 in a tumor or cancer cell, let alone the determination of CD44s as major CD44 isoform, might be of any use in determining responsiveness to an anti-CD44 antibody.

The standard form of CD44 (containing none of the variable exons v2-v10), also called CD44H or CD44s, contains several binding sites for Hyaluronic Acid (HA; Peach et al., J. Cell Biol. 1993), one of the natural ligands of CD44 (Toole et al. Curr. Pharm. Biotechnol., 2008). The most prominent of these binding sites was confirmed by solving the crystal structure of a CD44-HA complex (Banerji et al., Nature Struc Mol Biol 2007).

CD44 isoforms are expressed in hematopoietic and leukemic stem cells (dissertation of I. Taubert, University of Heidelberg, 2011). CD44 isoforms are reported to be overexpressed in certain cancer types (Olsson et al. BMC Cancer, 2011, 11:418; Van Driel et al. Leukemia, 2002; Raso-Barnett, PLOS ONE, 2013, e53883; Banky, Mol. Cancer, 2012, 11:83) and expression of these CD44 variant isoforms was linked to bad prognosis in terms of reduced disease free and overall survival (Mima et al. Cancer Research, 2012; Gvozdenovic et al. J Bone Miner Res., 2012).

Thus, the technical problem underlying the present invention is the provision of reliable means and methods to determine responsiveness of tumor cells, cancer cells and/or patients to an anti-CD44 antibody.

### SUMMARY OF THE INVENTION

Accordingly, the present invention relates to a method of determining whether a tumor cell or a cancer cell is responsive to an anti-CD44 antibody or to an antigen binding fragment thereof, said method comprising determining the major CD44 isoform in a sample of a patient, wherein if said major CD44 isoform is CD44s, said tumor cell or cancer cell is responsive to said anti-CD44 antibody or to an antigen binding fragment thereof.

The present invention also provides for a therapy of patients with an anti-CD44 antibody, wherein (a) tumor cell(s) or (a) cancer cell(s) of a sample of said patient is determined to have CD44s as major CD44 isoform.

The present invention relates to a method of treating a patient, said method comprising selecting a cancer patient, wherein a tumor cell or cancer cell of a sample of said patient is determined to have CD44s as major CD44 isoform and administering to the patient an effective amount of an anti-CD44 antibody or an antigen binding fragment thereof.

The present invention relates to an anti-CD44 antibody or an antigen binding fragment thereof for use in treating a cancer patient, wherein a tumor cell or cancer cell of a sample of said patient has CD44s as major CD44 isoform.

### BRIEF DESCRIPTION OF THE DRAWINGS

FIG. 1. CD44 gene and isoform structure. FIG. 1 shows selected, relevant transcripts of the CD44 gene.

FIG. 2. Dose dependent antitumor efficacy of RG7356. FIG. 2 shows dose dependent antitumor efficacy of RG7356 compared to the anti-human/mouse CD44 specific antibody Im7 in the subcutaneous human hepatocellular carcinoma xenograft model in female SCID/bg mice. Established SK-HEP-1 tumors (87 mm³; n=10 mice per group) were treated every 7 days, 5 times, intraperitoneally with RG7356 or Im7. Compared with vehicle control (dotted line with open circles) RG7356 treatment resulted in a dose-dependent tumor growth inhibition of 71% for 1 mg/kg (solid line) p<0.0001 and 74% for 10 mg/kg (solid line with squares) p<0.0001 compared to Tumor growth inhibition of Im7 with 15% for 1 mg/kg (dashed line with diamonds) p=0.69 and 31% for 10 mg/kg (dashed line with triangles) p=0.08.

Animals were treated once per week starting from day 8 until day 36 (days 8, 15, 22, 29, 36 of the study). Comparisons for all pairs using Tukey-Kramer HSD. Vehicle (20 mM Histidine, 150 mM NaCL, 0.01% Tween 80)

FIG. 3. Mosaic plot visualization of data shown in Table 2.

FIG. 4. CD44 major isoform distribution in TCGA (The Cancer Genome Atlas, http://cancergenome.nih.gov/) primary tumor RNASeq data from 17 tumor indications.

FIG. 5a. Binding of rhCD44s-Fc (recombinant human CD44-Fc fusion protein) to HA (hyaluronic acid)-coating in the presence of excess RG7356 or antibody isotype control. FIG. 5b. Titration of two fixed concentrations of rhCD44-Fc with RG7356.

FIG. 6a. Binding of muCD44s-Fc (recombinant murine CD44-Fc fusion protein) to HA-coating in the presence of excess IM7 or antibody isotype control. FIG. 6b. Binding of muCD44s-Fc to HA-coating in the presence of excess RG7356 or antibody isotype control.

FIG. 7. Effect of RG7356 treatment of adhesion of MDA-MB-231 or PL45 cells to HA (hyaluronic acid)-coated plates. Controls included: without antibody, human anti-Digoxigenin antibody (Dig_IgG1), and antibody buffer control (buffer). Slope obtained without antibody was used for normalization (100%).

FIG. 8. CD44 isoform status and association with HA(hyaluronic acid) levels measured in 22 cell lines.

FIG. 9. CD44 expression is associated with hyaluronic acid in peritrabecular bone marrow.

FIG. 9 shows that CD44 expression is limited to few blast cells; their preferential localization in the HA(hyaluronic acid)-rich peritrabecular bone marrow microenvironment may reflect a role of HA/CD44 interaction

FIG. 10. CD44 expression on AML blast cells is associated with hyaluronic acid in peritrabecular bone marrow. FIG. 10 shows consistent CD44 expression on AML blast cells in HA-rich intertrabecular bone marrow microenvironment, frequently observed in, but not limited to cases with fibrotic changes.

FIG. 11. The expression level of hyaluronic acid synthetases 1-3 correlates with hyaluronic acid levels. FIG. 11A shows the correlation of HA (hyaluronic acid) measures (from Table 4) with expression levels of HAS1-3 (hyaluronic acid synthetases 1-3) (the sum of the respective RNASeq expression values) measured by RNASeq data. FIG. 11B shows the correlation of HA (hyaluronic acid) measures (from Table 4) with expression levels of HAS 1-3 (hyaluronic acid synthetases 1-3) measured by qRT-PCR

FIG. 12. Comparison of ELISA assays to determine the level of hyaluronic acid. FIG. 12 shows a comparison of commercially available ELISA assays to measure HA.

FIG. 13. Equilibration and hydration of 2 hyaluronic acid in bicarbonate buffer. FIG. 13 shows the equilibration and hydration of 2.5 mg/ml rooster comb HA (hyaluronic acid) in bicarbonate buffer. Left to right: 0.5 h, 3 h, 16 h, respectively.

FIG. 14. Binding of rhCD44s-Fc to HA-coating. FIG. 14 shows binding of rhCD44s-Fc to HA-coating.

FIG. 15. RNASeq data details for each cell line. FIG. 15 shows plots showing read information obtained by RNASeq for the CD44 gene on top of the CD44 gene and isoform structure as defined by Ensembl (v60). The Ensembl gene (ENSG) and transcript (ENST) ids are supplemented by “_{—}11” indicating chromosome 11 where the CD44 gene is located in the human genome.

FIG. 16 (A) shows the Detection of Hyaluronic Acid by Affinity Histochemistry, and CD44 immunohistochemistry using the primary antibodies muARH 460 and SP37 in FFPET samples of Human Colorectal cancer. Consistent CD44 positivity (intensity 3+, left image), and generally low, focal HA staining—occasionally single tumor cells with high HA positive cytoplasm (right image). Methods: CD44 (SP37) IHC on Ventana Benchmark XT, HA AHC Manual assay FIG. 16 (B) shows the CSF1R advanced prevalence analysis in various human tumors including CD68/CD163 duplex immunohistochemistry, CSF1R, CSF1, CD 44 and HA. The left image shows a CRC with CD44 score 3 (3+ intensity in >30% of tumor, whereas the right image shows the same CRC case which has low HA in tumor cells and high HA in the surrounding stroma. Methods: CD44 (SP37) IHC on Ventana Benchmark XT, HA AHC Manual assay

FIG. 17 shows the selectivity of RG7356 for human CD44 in a concentration dependent manner. There is no binding to murine CD44 up to 100 ng of mCD44 per lane. The data was generated using anti-CD44-Mab RG7356 (1:500, 5% powdered milk (Roth, T145.3) in TBST, Roche, 12014939001), anti-human Fab-HRP 1: 10.000 (Jackson, 109-035-097) and a Super signal West Femto (Thermo Scientific, #34095), 2 sec.

### DETAILED DESCRIPTION OF THE INVENTION

It was surprisingly shown herein that a tumor or cancer cell having CD44s as major isoform respond to an anti-CD44 antibody cancer therapy. In contrast, a tumor or cancer cell that does not have CD44s as major isoform does not respond (or responds worse) to said therapy. It is demonstrated herein that a threshold of 50%, preferably at least 60% of all CD44 isoform molecules is the lowest level of CD44s isoform molecules in responding models as determined by e.g. RNASeq and quantitative Real-Time PCR (qRT-PCR); see Example 1 and Table 1. Accordingly, the present invention provides for the selection of cancer patients that respond to an anti-CD44 antibody, if the major isoform is CD44s.

In accordance with the present invention, (a) tumor cell(s), (a) cancer cell(s) and/or a patient(s) is determined to be responsive to an anti-CD44 antibody or to an antigen binding fragment thereof, if the major isoform in a sample is CD44s. For example, CD44s is the major CD44 isoform, if CD44s is the predominant CD44 isoform of all CD44 isoform molecules in a given tissue/cell/blood sample. “Predominant” means that the number or amount of CD44s molecules in a given tissue/cell/blood sample is bigger than that of any individual further CD44 isoform. For example, if all CD44s molecules constitute 40% of all CD44 isoform molecules, all CD44v3-v10 molecules constitute 30% of all CD44 isoform molecules, all CD44v2-10 molecules constitute 20% of all CD44 isoform molecules and all CD44v10 molecules constitute the remaining 10% of all CD44 isoform molecules, then the CD44s isoform can be considererd the predominant isoform and hence the major isoform.

CD44s can be considered to be the major CD44 isoform, if CD44s molecules constitute the majority of all CD44 isoform molecules in a given tissue/cell/blood sample. For example, CD44s is the major CD44 isoform, if at least 50%, preferably at least 60% of all CD44 isoform molecules in a given tissue/cell/blood sample are CD44s molecules.

The below explanations and definitions in relation to “tumor cell(s)”, “cancer cell(s)”, “anti-CD44 antibody or antigen binding fragment thereof”, “major CD44 isoform”, “CD44s”, “sample”, “patient”, and the like apply, mutatis mutandis, to the herein provided “method of determining whether a tumor cell or a cancer cell is responsive to an anti-CD44 antibody or to an antigen binding fragment thereof”, “method of treating a patient” and “anti-CD44 antibody or an antigen binding fragment thereof for use in treating a cancer patient”.

The following relates to the “CD44 isoforms”, like the “CD44s isoform”, and methods for determining whether a “CD44 isoform”, like the “CD44s isoform”, is the major isoform.

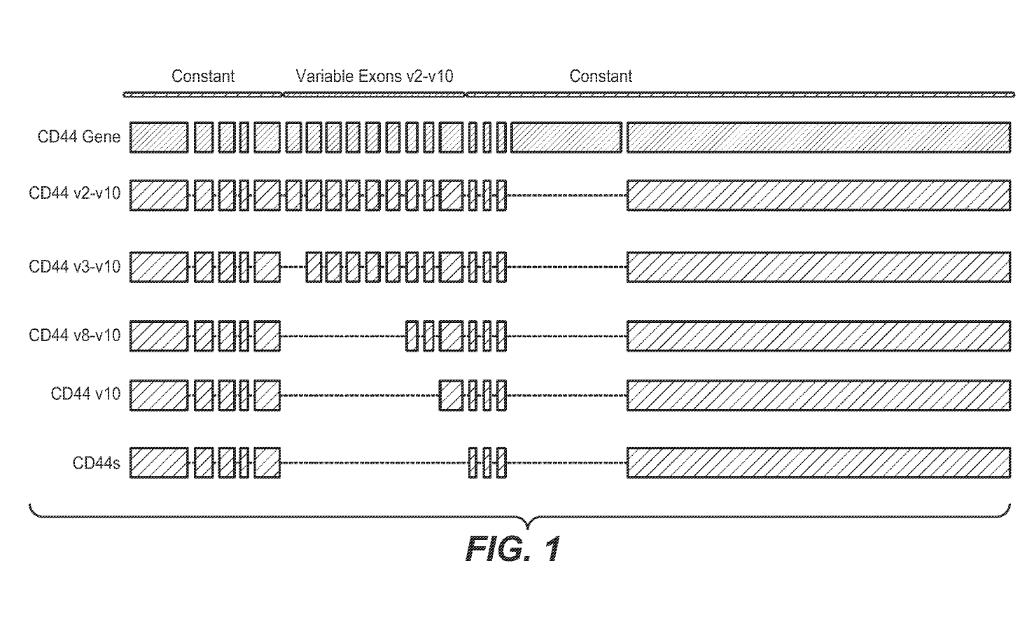

CD44 as used herein refers preferably to a human gene with 19 exons (according to Ensembl v60). The CD44 gene consists of a constant region that is expressed and contained in all known transcripts and a variable region consisting of 9 exons in human (v2-v10) that give rise to a variety of different transcripts. Selected transcripts are shown in FIG. 1.

For the purpose of the present invention, CD44 isoforms can be proteins that consist of/comprise the amino acid sequence encoded by one or more of constant exons C1 to C5 and C6 to C9 and that can optionally comprise the amino acid sequence encoded by one or more of variable exons v2 to v10. For the purpose of the present invention, CD44 isoforms can be proteins that can consist of/comprise the amino acid sequence encoded by all constant exons C1 to C5 and C6 to C9 and that can optionally comprise the amino acid sequence encoded by one or more variable exons v2 to v10. Exemplary nucleotide sequences of constant exons C1 to C9 and variable exons v2 to v10 and amino acid sequences encoded by constant exons C1 to C9 and by variable exons v2 to v10 are shown in SEQ ID NOs: 44 to 79. Exemplary corresponding RNA sequences of constant exons C1 to C9 and variable exons v2 to v10 are shown in SEQ ID NO. 133 to 150.

As mentioned above, (a) tumor cell(s) or (a) cancer cell(s) and/or a patient is responsive to an anti-CD44 antibody or antigen binding fragment thereof, if the major CD44 isoform in a sample is determined to be “CD44s”. “CD44s” is the shortest CD44 isoform. “CD44s” is the standard form of CD44 and is also termed “CD44H”. Accordingly, the terms “CD44s”, “CD44H” “standard form of CD44”, “CD44s isoform” are used interchangeably herein. The term “CD44s isoform” can refer to a protein that is encoded by a transcript variant (or splice variant) of the CD44 gene.

The term “CD44s isoform” as used herein can refer to a protein which is characterized in that it does not contain an amino acid sequence encoded by any of the variable exons v2 to v10 of the CD44 gene. Thus, CD44s isoforms consist of/comprise an amino acid sequence encoded by one or more of constant exons C1 to C5 and C6 to C9, provided that the CD44s isoforms do not contain an amino acid sequence encoded by any of the variable exons v2 to v10. CD44s isoforms can consist of/comprise an amino acid sequence encoded by all constant exons C1 to C5 and C6 to C9, provided that the CD44s isoforms do not contain an amino acid sequence encoded by any of the variable exons v2 to v10. Exemplary human nucleic acid sequences encoding the CD44 isoform CD44s are shown in SEQ ID NOs: 12 and 14. Exemplary corresponding RNA sequences are shown in SEQ ID NOs: 117 and 118. Exemplary human amino acid sequences of the CD44 isoform CD44s are shown in SEQ ID NO: 13 and 15. Also the use of fragments (or variants, like genetic variants) of the sequences shown in SEQ ID NO: 12 and/or 14 and/or SEQ ID NO: 117 and/or 118 and/or shown in SEQ ID NO: 13 and/or 15 is envisaged in the methods of the present invention. Further, the use of sequences which are orthologous or have a certain level of identity to SEQ ID NO: 12 and/or 14, SEQ ID NO: 117 and/or 118 and SEQ ID NO: 13 and/or 15, respectively, is encompassed without deferring from the gist of the present invention. Such fragments and/or orthologous/identical sequences are described herein further below.

(A) tumor cell(s) or (a) cancer cell(s) and/or a patient is not responsive to an anti-CD44 antibody or antigen binding fragment thereof, if the major CD44 isoform is not CD44s. For example, if the major CD44 isoform is a protein that is encoded by a transcript/splice variant of the CD44 gene, wherein said protein contains an amino acid sequence encoded by one or more of the variable exons v2-v10, (a) tumor cell(s) or (a) cancer cell(s) and/or a patient are determined to not respond to an anti-CD44 antibody or antigen binding fragment thereof in accordance with the present invention.

As used herein the term “CD44v” can refer to a protein encoded by any of the transcript variants (or splice variants) of the CD44 gene, whereby the protein contains an amino acid sequence encoded by any one of the variable exons v2 to v10. Thus, CD44v isoforms consist of/comprise an amino acid sequence encoded by one or more of constant exons C1 to C5 and C6 to C9 and an amino acid sequence encoded by one or more of the variable exons v2 to v10. CD44v isoforms can consist of/comprise an amino acid sequence encoded by all constant exons C1 to C5 and C6 to C9 and by one or more of the variable exons v2 to v10. As mentioned, if the major isoform is, for example, a “CD44v”, (the) tumor cell(s) or cancer cell(s) and/or patient(s) is/are not responsive to an anti-CD44 antibody or antigen binding fragment thereof. Exemplary “CD44v” isoforms to be used herein are, inter alia, “CD44 v2-v10”, “CD44 v3-v10”, “CD44 v8-v10”, and “CD44 v10”. The term “CD44 v2-v10” refers to a protein which is encoded by a transcript/splice variant that comprises a RNA sequence corresponding to all variable exons v2 to v10. The term “CD44 v3-v10” refers to a protein which is encoded by a transcript/splice variant that contains solely an RNA sequence corresponding to variable exons v3 to v10 (i.e. it does not contain exon v2). The term “CD44 v8-v10” refers to a protein which is encoded by a transcript/splice variant that contains solely an RNA sequence corresponding to variable exons v8 to v10 (i.e. it does not contain exon v2 to v7). The term “CD44 v10” refers to a protein which is encoded by a transcript/splice variant that contains solely an RNA sequence corresponding to variable exon v10 (i.e. it does not contain exon v2 to v9). Exemplary nucleic acid sequences encoding “CD44 v2-v10”, “CD44 v3-v10”, “CD44 v8-v10”, and “CD44 v10” and exemplary amino acid sequences of “CD44 v2-v10”, “CD44 v3-v10”, “CD44 v8-v10”, and “CD44 v10” are, inter alia, shown in SEQ ID NO: 18/19, 30/31, 34/35 and 42/43, respectively. Exemplary RNA sequence corresponding to such nucleic acid sequences encoding “CD44 v2-v10”, “CD44 v3-v10”, “CD44 v8-v10”, and “CD44 v10” are shown in 120, 126, 128, 132, respectively. The term “RNA sequence corresponding to” as used herein means that the respective RNA sequence is identical to a given DNA sequence as provided herein, with the exception that the tymidine (T) nucleic acid residues are replaced by uracil (U) nucleic acid residues.

In order to determine the “major CD44 isoform” in a sample, like the CD44s isoform, the amount and/or number of all CD44 isoform molecules in a sample can be assessed (or measured or determined) in accordance with the present invention. The terms “assessing”, “measuring” and “determining” and grammatical variants thereof can be used interchangeably herein. The amount and/or number of all CD44 isoform molecules can be assessed (or measured or determined) on a nucleic acid level, like RNA level, in particular mRNA level, or on a protein level. In this context the term “CD44 isoform molecule” can refer to “CD44 isoform RNA molecule” (preferably “CD44 isoform mRNA molecule”) or to “CD44 isoform protein molecule”.

For example, an assessment on a protein level involves determining the amount and/or number of all CD44 isoform protein molecules in a sample.

For example, an assessment on an mRNA level involves determining the amount and/or number of all CD44 isoform mRNA molecules. Thus, the amount and/or number of all transcript variants/splice variants (i.e. CD44 isoform mRNA molecules) of the CD44 gene can be determined in accordance with the herein provided methods. For example, Real Time PCR or Whole Transcriptome Shotgun Sequencing (RNAseq) can be used to determine/assess the amount and/or number of CD44 isoform mRNA molecules.

The term “all CD44 isoform molecules” can refer to essentially all molecules in a sample. A person skilled in the art will appreciate that the determination of all molecules can depend on the technique used. Thus, it is contemplated herein that only a portion of all CD44 isoform molecules in a sample can be determined without deferring from the gist of the present invention. If only a portion of all CD44 isoform molecules is determined, it is envisaged that this portion is representative of the entire population of CD44 isoform molecules in a sample. In other words, if a portion of all CD44 isoform molecules is determined, it is envisaged that the relative percentages of the CD44 isoforms as defined herein represent the percentages of the CD44 isoforms as they are present in the sample. Thus, the “major isoform” can be determined, for example, if at least 50%, preferably at least 60% of all CD44 isoform molecules are molecules of said isoform, even if not all, for example, only a portion of all CD44 isoform molecules is determined.

The term “all CD44 isoform molecules” refers to protein or mRNA molecules of any CD44 isoform, in particular of any CD44v and CD44s isoform as defined herein. Preferably, the term “all CD44 isoform molecules” refers to “all CD44 isoform mRNA molecules”. For the purpose of the present invention, the term “all CD44 isoform molecules” can refer to “all CD44 isoform mRNA molecules” comprising/consisting of an RNA sequence corresponding to one or more (preferably all) of constant exons C1 to C5 and C6 to C9 and that can optionally comprise an RNA sequence corresponding to one or more of variable exons v2 to v10. The term “RNA sequence corresponding to” as used herein means that the respective RNA sequence is identical to a given DNA sequence as provided herein, with the exception that the tymidine (T) nucleic acid residues are replaced by uracil (U) nucleic acid residues.

For the purpose of the present invention, “all CD44 isoform molecules” can refer to “all CD44 isoform protein molecules” that can consist of/comprise the amino acid sequence encoded by one or more of (preferably all) constant exons C1 to C5 and C6 to C9 and that can optionally comprise the amino acid sequence encoded by one or more variable exons v2 to v10. Exemplary nucleotide sequences of constant exons C1 to C9 and variable exons v2 to v10 and amino acid sequences encoded by constant exons C1 to C9 and by variable exons v2 to v10 are shown in SEQ ID NOs: 44 to 79. Exemplary RNA sequences corresponding to such nucleic acid sequences encoding/of exons C1 to C9 and variable exons v2 to v10 are shown in SEQ ID NO:s 133 to 150.

Exemplary nucleic acid sequences encoding CD44v and CD44s isoforms and exemplary amino acid sequences of CD44v and CD44s are, inter alia, shown in SEQ ID NO: 12 to 43. Exemplary RNA sequences corresponding to such nucleic acid sequences encoding CD44v and CD44s isoforms are shown in SEQ ID NOs: 118 to 132. Preferred nucleic acid sequences encoding CD44s isoforms and preferred amino acid sequences of CD44s are, inter alia, shown in SEQ ID NO: 12/13 and 14/15, respectively. Corresponding preferred RNA sequences of CD44s isoforms are shown in SEQ ID NOs: 117 and 118. Preferred nucleic acid sequences encoding CD44v isoforms “CD44 v2-v10”, “CD44 v3-v10”, “CD44 v8-v10”, and “CD44 v10” and preferred amino acid sequences of CD44v isoforms “CD44 v2-v10”, “CD44 v3-v10”, “CD44 v8-v10”, and “CD44 v10” are, inter alia, shown in SEQ ID NO: 18/19, 30/31, 34/35 and 42/43, respectively. Corresponding preferred RNA sequences of CD44v isoforms “CD44 v2-v10”, “CD44 v3-v10”, “CD44 v8-v10”, and “CD44 v10” are showin in SEQ ID NOs: 120, 126, 128 and 132.

If the amount and/or number of a CD44 isoform RNA (preferably a CD44 isoform mRNA) molecule is to be determined in accordance with the present invention, the mRNA molecule can have an RNA sequence corresponding to any of the herein above defined nucleic acid sequences encoding CD44v isoforms or CD44s isoforms, like SEQ ID NO: 12, SEQ ID NO. 14, SEQ ID NO: 18, SEQ ID NO: 30, SEQ ID NO: 34 or SEQ ID NO: 42. The term “RNA sequence corresponding to” as used herein means that the respective RNA sequence is identical to a given DNA sequence as provided herein, with the exception that the tymidine (T) nucleic acid residues are replaced by uracil (U) nucleic acid residues. Thus, the mRNA molecule having an RNA sequence corresponding to any of the herein above defined nucleic acid sequences encoding CD44v isoforms or CD44s isoforms can have an RNA sequences as shown, for example, in SEQ ID NO: 117, SEQ ID NO: 118, SEQ ID NO: 120, SEQ ID NO: 126, SEQ ID NO: 128 or SEQ ID NO: 132

The terms “major CD44 isoform”, “CD44 isoform”, “CD44s”, CD44v″ and the like as used herein refer preferably to an mRNA molecule (or splice variant or transcript variant) corresponding to a nucleic acid sequence encoding a “(major) CD44 isoform” protein, like CD44s isoform protein or CD44v isoform proteins. “CD44 isoform” proteins (like CD44s isoform protein or CD44v isoform proteins) are explained and defined herein above. The term “RNA sequence corresponding to” (or likewise “RNA molecule corresponding to”) as used herein means that the respective RNA sequence (or RNA molecule) is identical to a given DNA sequence as provided herein, with the exception that the tymidine (T) nucleic acid residues are replaced by uracil (U) nucleic acid residues.

In accordance with the above, the term “(major) CD44 isoform” as used herein can refer to an mRNA molecule corresponding to a nucleic acid sequence consisting of/comprising one or more of constant exons C1 to C5 and C6 to C9 and optionally comprising one or more of variable exons v2 to v10. For the purpose of the present invention, the term “(major) CD44 isoform” as used herein can refer to an mRNA molecule corresponding to a nucleic acid sequence consisting of/comprising all constant exons C1 to C5 and C6 to C9 and optionally comprising one or more variable exons v2 to v10. Exemplary nucleotide sequences of constant exons C1 to C9 and variable exons v2 to v10 and amino acid sequences encoded by constant exons C1 to C9 and by variable exons v2 to v10 are shown in SEQ ID NOs: 44 to 79. Exemplary RNA sequences corresponding to nucleotide sequences encoding/of constant exons C1 to C9 and variable exons v2 to v10 are shown in SEQ ID NOs:133 to 150.

The term “CD44s isoform” as used herein can refer to an mRNA molecule corresponding to a nucleic acid sequence which is characterized in that it does not contain any of the variable exons v2 to v10 of the CD44 gene. Thus, CD44s isoforms can consist of/comprise an mRNA molecule corresponding to one or more of constant exons C1 to C5 and C6 to C9, provided that the mRNA molecule does not contain an RNA sequence corresponding to any of the variable exons v2 to v10. CD44s isoforms can consist of/comprise an mRNA molecule corresponding to all of constant exons C1 to C5 and C6 to C9, provided that the mRNA molecule does not contain an RNA sequence corresponding to any of the variable exons v2 to v10.

Exemplary RNA sequences (or RNA molecules) of (a) “CD44s isoform(s)” can be RNA sequences (or RNA molecules) corresponding to human nucleic acid sequences as shown in SEQ ID NOs: 12 and 14. Exemplary RNA sequences (or RNA molecules) corresponding to such human nucleic acid sequences are shown in SEQ ID NOs: 117 and 118, respectively. Also the use of fragments (or variants, like genetic variants) of the sequences shown in SEQ ID NO: 12 and/or 14 (or SEQ ID NOs: 117 and/or 118) is envisaged in the methods of the present invention. Further, the use of sequences which are orthologous or have a certain level of identity to SEQ ID NO: 12 and/or 14 (or SEQ ID NOs: 117 and/or 118) is encompassed without deferring from the gist of the present invention.

As used herein the term “CD44v” can refer to an mRNA molecule corresponding to a nucleic acid sequence containing any one of the variable exons v2 to v10. Thus, CD44v isoforms can consist of/comprise an mRNA molecule corresponding to a nucleic acid sequence containing one or more of constant exons C1 to C5 and C6 to C9 and containing one or more of the variable exons v2 to v10. CD44v isoforms can consist of/comprise an mRNA molecule corresponding to a nucleic acid sequence containing all of constant exons C1 to C5 and C6 to C9 and containing one or more of the variable exons v2 to v10. Exemplary “CD44v” isoforms to be used herein are, inter alia, “CD44 v2-v10”, “CD44 v3-v10”, “CD44 v8-v10”, and “CD44 v10”. In this context, the term “CD44 v2-v10” can refer to an mRNA molecule corresponding to a nucleic acid that comprises all variable exons v2 to v10. The term “CD44 v3-v10” can refer to an mRNA molecule corresponding to a nucleic acid that comprises solely variable exons v3 to v10 (i.e. it does not contain exon v2). The term “CD44 v8-v10” can refer to mRNA molecule corresponding to a nucleic acid that comprises solely an RNA sequence corresponding to variable exons v8 to v10 (i.e. it does not contain exon v2 to v7). The term “CD44 v10” can refer to an mRNA molecule corresponding to a nucleic acid that comprises solely an RNA sequence corresponding to variable exon v10 (i.e. it does not contain exon v2 to v9). Exemplary RNA sequences correspond to nucleic acid sequences encoding “CD44 v2-v10”, “CD44 v3-v10”, “CD44 v8-v10”, and “CD44 v10” as, inter alia, shown in SEQ ID NO: 18, 30, 34 and 42, respectively. Exemplary RNA sequences that correspond to nucleic acid sequences encoding “CD44 v2-v10”, “CD44 v3-v10”, “CD44 v8-v10”, and “CD44 v10” are, inter alia, shown in SEQ ID NO: 120, 126,128, and 132, respectively. Further exemplary RNA sequences correspond to nucleic acid sequences shown in SEQ ID NO: 16, 20, 22, 24, 26, 28, 30, 32, 36, 38 and 40, respectively. Exemplary RNA sequences corresponding to these nucleic acid sequences are shown in SEQ ID NO: 119, 121, 122, 123, 124, 125, 126, 127, 129, 130 and 131, respectively. Also the use of fragments (or variants, like genetic variants) of these sequences is envisaged in the methods of the present invention. Again, the term “RNA sequence corresponding to” as used herein means that the respective RNA sequence is identical to a given DNA sequence as provided herein, with the exception that the tymidine (T) nucleic acid residues are replaced by uracil (U) nucleic acid residues.

In accordance with the above, the methods of the present invention can comprise the use of methods for determination/quantification of the expression level, amount and/or number of CD44s and/or further CD44 isoforms. For example, Real Time PCR or Whole Transcriptome Shotgun Sequencing (RNAseq) can be used to determine the RNA expression level, amount and/or number of RNA molecules. Such methods (like Real Time PCR or Whole Transcriptome Shotgun Sequencing (RNAseq)) can involve the generation or use of cDNA molecules of the CD44 isoforms.

If the gene product to be assessed is an RNA, such as an mRNA (e.g. partially spliced or spliced mRNA), determination can be performed by taking advantage of northern blotting techniques, in situ hybridization, hybridization on microarrays or DNA chips equipped with one or more probes or probe sets specific for mRNA transcripts or PCR techniques, like, quantitative PCR techniques, such as Real time PCR and Whole Transcriptome Shotgun Sequencing (RNAseq). These and other suitable methods for assessing (specific) mRNA are well known in the art and are, for example, described in Sambrook and Russell (2001, loc. cit.). A skilled person is capable of determining the expression level, amount and/or number of the gene product (like RNA, e.g. mRNA), by taking advantage of a correlation, preferably a linear correlation, between the intensity of a detection signal and the amount of the gene product to be determined.

The methods of the present invention can also comprise the use of methods for determination/quantification of the expression level, amount and/or number of CD44s and/or further CD44 isoforms, taking, for example, advantage of techniques like immunoassay, gel- or blot-based methods, IHC, mass spectrometry, flow cytometry, FACS, Western blotting techniques or Elisa. In such a case, the terms “CD44s”, “CD44H”, “standard form of CD44”, and “CD44v”, can refer to an amino acid molecule thereof, such as an protein (like CD44s protein). Quantification of the protein expression level can be performed by taking advantage of well known techniques. Generally, a person skilled in the art is aware of methods for the quantitation of (a) polypeptide(s)/protein(s). Amounts of purified polypeptide in solution can be determined by physical methods, e.g. photometry. Methods of quantifying a particular polypeptide in a mixture may rely on specific binding, e.g. of antibodies. Antibodies to be used for quantification and detection of the expression of CD44 isoforms as described herein are well known. Exemplary, commercially available antibodies are BBA10 (R&D Systems) and 16-0441 (eBioscience) for the analysis of CD44s; VFF-327 (AbD Serotec) for CD44v3, BMS124 (eBioscience) for CD44v3-v10; BMS115 (eBioscience) for CD44v5 and BMs125 (eBioscience) for CD44v6.

In accordance with the above, (a) tumor cell(s), (a) cancer cell(s) and/or a patient(s) is determined to be responsive to an anti-CD44 antibody or to an antigen binding fragment thereof, if the major isoform in a sample is CD44s. For example, CD44s is the major CD44 isoform, if at least 50%, preferably at least 60% of all CD44 isoform molecules in a sample are CD44s molecules.

As mentioned, it was shown herein that a threshold of at least 60% of all CD44 isoform molecules is the lowest major isoform level of CD44s in responding models as determined by RNASeq and quantitative Real-Time PCR (qRT-PCR); see Example 1 and Table 1. For example, the value can be determined either by using results from qRT-PCR or mRNASeq read counts.

The methods of the present invention can, therefore, comprise determining the amount and/or number of all CD44 isoform molecules in the sample. Determining the amount and/or number of all CD44 isoform molecules in a sample can be performed by methods for quantifying (a) gene product(s), like CD44 isoforms as defined and explained herein. Exemplary methods are Real Time PCR and Whole Transcriptome Shotgun Sequencing (RNAseq). If the amount and/or number of all CD44 isoform molecules in the sample has been determined, the portion/ratio of individual CD44 isoforms can be determined. If at least 50%, preferably at least 60% of all CD44 isoforms in the sample are/constitute the CD44s isoform, the CD44s isoform is determined to be the major isoform in accordance with the present invention.

In other words, if less than 50%, preferably less than 60% of all CD44 isoforms in the sample are/constitute the CD44s isoform, the CD44s isoform is not the major isoform. For example, another isoform than CD44s may be the major isoform; e.g. CD44vs-v10, CD44-v3-v10, CD44 v8-v10 or CD44v10 may be the major isoform (i.e. at least 60% of all CD44 isoforms in the sample are/constitute CD44vs-v10, CD44-v3-v10, CD44 v8-v10 or CD44v10, respectively). Alternatively, none of the CD44 isoforms may be or may constitute at least 50%, preferably at least 60% of all CD44 isoforms in a sample. In these cases, the tumor cell(s) or cancer cell(s) and/or patient(s) will likely not respond to an anti-CD44 antibody or to an antigen binding fragment thereof.

In the following exemplary protocols for RNASeq assays and RT-PCR assays are described that can be used in accordance with the present invention.

RNASeq data can be generated in order to analyze the isoform status of samples/tumor cell(s)/cancer cell(s) using commercially available sequencing system like the Illumina HiSeq2000 sequencing system. RNA and sequencing libraries can be prepared according to the manufacturer's protocols (using e.g. the Illumina TruSeq Standard protocol). A minimum number of 30 Mio. paired-end reads of 50 bp length (for each read of a pair) can be generated per sample.

Raw RNASeq data can be aligned to the human transcriptome (Ensembl v60) and genome (hg19) using for example Bowtie2 (Langmead et al. Nature Methods, 2012, 9:357-359). Read data for the CD44 gene locus can be extracted from the alignments and visualized. The major CD44 isoform (expressed) in a sample can be identified as follows. For example, read counts for unique splice junctions that exist for each isoform of CD44 can be used, defining the isoform whose unique splice junction count comprises 60% of all unique junction read counts for any CD44 isoform (e.g. the major isoform is the isoform with the largest number of reads crossing its unique junction). Alternatively, MMSeq (Turro et al. Genome Biology, 2011, 12:R13) can be used. MMSeq is a method dedicated to quantify isoform levels in RNASeq data to quantify the frequency of the different CD44 isoforms defined in Ensembl v60. Langmead (loc. Cit.) and Turro (loc. cit.) are incorporated herein by reference.

Also real-time PCR can be used to determine the major CD44 isoform as follows: The CD44 gene contains 9 constant exons (E1-E9/10) and 9 variable exons (v2-v10). The variable exons are alternatively spliced, resulting in different CD44 splice variants. A set of PCR reactions can be designed to discriminate and quantify the following CD44 isoforms:

CD44-v3 (containing variable exon v3)

CD44-v6 (containing variable exon v6)

CD44-v8 (containing variable exon v8)

CD44-v10 (containing variable exon v10)

CD44-v2-v10 (containing variable exons v2-v10)

CD44-v2-v9 (containing variable exons v2-v9)

CD44-v3-v10 (containing variable exons v3-v10)

CD44-v6-v10 (containing variable exon v6-v10)

CD44-v8-v10 (containing variable exon v8-v10)

CD44s (containing no variable exon)

In order to determine whether CD44s is the major CD44 isoform, it may be sufficient to quantify isoforms CD44s, CD44v2-v10, CD44 v3-v10, CD44v8-v10 and CD44v10, as these isoforms have been found herein to be mainly expressed in tumor/cancer cells. Thus, determining the amount or number of these isoforms in a given sample can suffice to determine the amount/number of all CD44 isoform molecules in accordance with the method of the present invention. Examplary sequences of these isoforms are shown in SEQ ID No. 12/13, 14/15, 18/19, 30/31, 34/35 and 42/43. Corresponding RNA sequences are shown in SEQ ID NO:s 117, 118, 120, 126, 128 and 132, respectively.

Primers and probes can be desigend based on the complete mRNA of the longest version of CD44 at NCBI (Refseq NM_{—}000610) or ENST00000278385 as shown in SEQ ID NO.18. This mRNA comprises 18 exons. The corresponding amino acid sequence is shown in SEQ ID NO. 19.

If, for example, Real Time PCR is used for assessing the amount and/or number of all CD44 isoform molecules, the methods of the present invention can further comprise using primers and probes as explained below.

The PCR assay for CD44-v3 can contain a forward primer spanning the splice junction between exon E5 and exon v3 (bp 1092-1101 and 1231-1240) and a reverse primer spanning the splice junction between exon v3 and exon E6 (bp 1353-1356 and 2245-2259). The probe of the PCR assay for CD44-v3 can bind to by 1264-129

The PCR assay for CD44-v6 can contain a forward primer spanning the splice junction between exon E5 and exon v6 (bp 1087-1101 and 1588-1592) and a reverse primer spanning the splice junction between exon v6 and exon E6 (bp 1713-1716 and 2245-2257). The probe of the PCR assay for CD44-v6 can bind to by 1610-1640.

The PCR assay for CD44-v10 can contain a forward primer spanning the splice junction between exon E5 and exon v10 (bp 1084-1101 and 2041-2043) and a reverse primer binding to exon v10 (bp 2087-2106). The probe of the PCR assay for CD44-v10 can bind to by 2053-2081.

The PCR assay for CD44-v3-10 can contain a forward primer spanning the splice junction between exon E5 and exon v3 (bp 1091-1101 and 1231-1242) and a reverse primer binding to exon v3 (bp 1293-1315). The probe of the PCR assay for CD44-v3-10 can bind to by 1252-1260.

To discriminate the CD44-v3 splice variant from the CD44-v3-10 splice variant, an additional assay can be designed for long CD44 splice variants (CD44-long), containing a forward primer spanning the splice junction between exon v8 and exon v9 (bp 1934-1960) and a reverse primer spanning the splice junction between exon v9 and exon v10 (bp 2024-2046). The probe of this PCR assay can bind to by 1984-2015.

The PCR assay for CD44-v6-10 can contain a forward primer spanning the splice junction between exon E5 and exon v6 (bp 1087-1101 and 1588-1593) and a reverse primer binding to exon v6 (bp 1645-1661). The probe of the PCR assay for CD44-v6-10 can bind to by 1610-1640. To discriminate the CD44-v6 splice variant from the CD44-v6-10 splice variant, the CD44-long assay can be used.

The PCR assay for CD44-v8-10 can contain a forward primer spanning the splice junction between exon E5 and exon v8 (bp 1090-1101 and 1849-1858) and a reverse primer binding to exon v8 (bp 1885-1905). The probe of the PCR assay for CD44-v8-10 can bind to by 1874-1882. To unambiguously identify the CD44-v8-10 splice variant, the CD44-long assay can be used.

The PCR assay for CD44-s can contain a forward primer spanning the splice junction between exon E5 and exon E6 (bp 1085-1101 and 2245-2248) and a reverse primer spanning the splice junction between exon E6 and exon E7 (bp 2305-2322). The probe of the PCR assay for CD44-v6 can bind to by 2266-2287.

The assays can be used to detect CD44v3-10 and CD44v6-10 as well as variants with different compositions regarding exons 4-7 and 7, respectively.

The real-time PCR can be performed as follows:

RNA can be isolated as follows: sample material (e.g. frozen cell pellets) can be isolated using a commercially available kit (e.g. High Pure RNA Isolation Kit (Roche Applied Science, Id. No. 11828665001). RNA can be quantified by UV photospectrometer and analyzed for quality by agarose gel electrophoresis.

cDNA can be synthesized as follows: about 1 μg RNA can be transcribed into cDNA using a commercially available kit (e.g. the Transcriptor First Strand cDNA Kit (Roche Applied Science, Id. No. 04896866001). cDNA can be quantified by UV photospectrometer and diluted in water, PCR-grade, to a concentration of e.g. 5 ng/gl.

cDNA can be quantified as follows: cDNA can be quantified by real-time PCR using e.g. the LightCycler 480 II (Roche Applied Science, Id. No. 05015243001) in relation to two housekeeping genes (IP08 and HPRT). PCR reactions can be set up according to the following scheme:

forward primer (10 μM): 1 μl

reverse primer (10 μM): 1 μl

probe (10 μM): 0.4 μl

LightCycler 480 Probes Master (Roche Applied Science, Id. No. 04707494001): 10 μl

cDNA (5 μg/μl): 5 μl

water, PCR-grade: 2.6 μl

PCR reactions ca be set up in a 384-well multi-well plate (Roche Applied Science, Id. No. 04729749001) and the following PCR program was run on a LightCycler 480 II instrument:

Pre-incubation:

| | |
|---|---|
| 95° C. | 10 minutes |

Amplification (45 cycles):

| | |
|---|---|
| 95° C. | 10 seconds |
| 60° C. | 30 seconds |
| 72° C. | 1 second |

Cooling:

| | |
|---|---|
| 40° C. | 10 minutes |

Data analysis and relative quantification can be done using the LightCycler 480 II software, version LCS480 1.5.0.39.

The following primers and probes can be used to identify and quantify the respective CD44 splice variants/CD44 isoform molecules. Accordingly, the methods of the present invention can further comprise using one or more of the following sets of primers and probes, if, for example, the amount and/or number of CD44 isoform molecules is to be assessed by Real Time PCR:

CD44-v3:
Forward:

(SEQ ID NO: 87)

cctgctaccagtacgtcttc
Reverse:

(SEQ ID NO: 88)

accagagaccaagacacat
Probe:

(SEQ ID NO: 89)

agccaaatgaagaaaatgaagatgaaagagacagac
Cd44-v6:
Forward:

(SEQ ID NO: 90)

gaatccctgctaccatccag
Reverse:

(SEQ ID NO: 91)

gctggagaccaagacac
Probe:

(SEQ ID NO: 92)

aacggaagaaacagctacccagaaggaacag
CD44-v10:
Forward:

(SEQ ID NO: 93)

acagaatccctgctaccaata
Reverse:

(SEQ ID NO: 94)

aggctcaactactttactgg
Probe:

(SEQ ID NO: 95)

tcacaggtggaagaagagacccaaatcat
CD44-v3-10:
Forward:

(SEQ ID NO: 96)

ccctgctaccagtacgtcttcaa
Reverse:

(SEQ ID NO: 97)

gacagacacctcagtttttctgg
Probe (LNA):
cagcaggct
CD44-v6-10:
Forward:

(SEQ ID NO: 98)

gaatccctgctaccatccagg
Reverse:

(SEQ ID NO: 99)

ttggcaacagatggcat
Probe:

(SEQ ID NO: 100)

aacggaagaaacagctacccagaaggaacag
CD44-v8-10:
Forward:

(SEQ ID NO: 101)

tccctgctaccaatatggactc
Reverse:

(SEQ ID NO: 102)

ctgcaaatccaaacacaggtt
Probe (LNA):
gcttcagcc
CD44-s:
Forward:

(SEQ ID NO: 103)

cagaatccctgctaccagaga
Reverse:

(SEQ ID NO: 104)

atggacactcacatggga
Probe:

(SEQ ID NO: 105)

ccagtggggggtcccataccac
CD44-long:
Forward:

(SEQ ID NO: 106)

tctttcaatgacaacgcagcagagtaa
Reverse:

(SEQ ID NO: 107)

tactctgacatcaagcaatagga
Probe:

(SEQ ID NO: 108)

atgaaggcttggaagaagataaagaccatcca

CD44-pan primers and probe can be used to determine the amount or number of all CD44 isoforms (transcript) in a sample:

CD44 Pan (measures any CD44 transcript
irrespective of isoform):
Forward:

(SEQ ID NO: 109)

gaatcagatggacactcaca
Reverse:

(SEQ ID NO: 110)

ttccagaatggctgatcatc
Probe (LNA):
aggtggag

By determining/quantifying the amount or number of all molecules of any CD44 isoform using CD44-pan primers/probe in a sample and by determining/quantifying the amount or number of all molecules of the CD44s isoform using CD44s specific primers/probe (like the ones exemplified above), the ratio of CD44s isoform molecules vs. all CD44 isoform molecules can easily be calculated. Thereby, it can easily be determined whether the CD44s isoform is the major isoform.

The present invention relates to a method of determining whether a tumor cell or a cancer cell is responsive to an anti-CD44 antibody or to an antigen binding fragment thereof, said method comprising determining the major CD44 isoform in a sample of a patient, wherein if said major CD44 isoform is CD44s, said tumor cell or cancer cell is responsive to said anti-CD44 antibody or to an antigen binding fragment thereof, said method further comprising determining the amount and/or number of all CD44 isoform molecules in said sample.

The present invention relates to a method of determining whether a tumor cell or a cancer cell is responsive to an anti-CD44 antibody or to an antigen binding fragment thereof, said method comprising determining the major CD44 isoform in a sample of a patient, wherein if said major CD44 isoform is CD44s, said tumor cell or cancer cell is responsive to said anti-CD44 antibody or to an antigen binding fragment thereof, wherein said CD44s isoform is determined to be the major isoform, if at least 50%, preferably at least 60% of all CD44 isoform molecules in said sample are molecules of said CD44s isoform.

The present invention relates to a method of determining whether a tumor cell or a cancer cell is responsive to an anti-CD44 antibody or to an antigen binding fragment thereof, said method comprising determining the major CD44 isoform in a sample of a patient, wherein if said major CD44 isoform is CD44s, said tumor cell or cancer cell is responsive to said anti-CD44 antibody or to an antigen binding fragment thereof, said method further comprising determining the amount and/or number of all CD44 isoform molecules in said sample, wherein said CD44s isoform is determined to be the major isoform, if at least 50%, preferably at least 60% of all CD44 isoform molecules in said sample are molecules of said CD44s isoform.

The present invention relates to a method of determining whether a tumor cell or a cancer cell is responsive to an anti-CD44 antibody or to an antigen binding fragment thereof, said method comprising determining the major CD44 isoform in a sample of a patient, wherein if said major CD44 isoform is CD44s, said tumor cell or cancer cell is responsive to said anti-CD44 antibody or to an antigen binding fragment thereof, said method further comprising determining the amount and/or number of all CD44 isoform molecules in said sample, wherein the amount and/or number of all CD44 isoform molecules in said sample is assessed by Real Time PCR or Whole Transcriptome Shotgun Sequencing (RNAseq).

The present invention relates to a method of determining whether a tumor cell or a cancer cell is responsive to an anti-CD44 antibody or to an antigen binding fragment thereof, said method comprising determining the major CD44 isoform in a sample of a patient, wherein if said major CD44 isoform is CD44s, said tumor cell or cancer cell is responsive to said anti-CD44 antibody or to an antigen binding fragment thereof, wherein said CD44s isoform is determined to be the major isoform, if at least 50%, preferably at least 60% of all CD44 isoform molecules in said sample are molecules of said CD44s isoform, wherein the amount and/or number of all CD44 isoform molecules in said sample is assessed by Real Time PCR or Whole Transcriptome Shotgun Sequencing (RNAseq).

The present invention relates to a method of determining whether a tumor cell or a cancer cell is responsive to an anti-CD44 antibody or to an antigen binding fragment thereof, said method comprising determining the major CD44 isoform in a sample of a patient, wherein if said major CD44 isoform is CD44s, said tumor cell or cancer cell is responsive to said anti-CD44 antibody or to an antigen binding fragment thereof, said method further comprising determining the amount and/or number of all CD44 isoform molecules in said sample, wherein said CD44s isoform is determined to be the major isoform, if at least 50%, preferably at least 60% of all CD44 isoform molecules in said sample are molecules of said CD44s isoform, wherein the amount and/or number of all CD44 isoform molecules in said sample is assessed by Real Time PCR or Whole Transcriptome Shotgun Sequencing (RNAseq).

It is to be understood that the tumor cell(s) or cancer cell(s) to be evaluated/assessed/scrutinized may be part of a sample (like and preferably a biopsy in case of solid cancers, or preferably a bone marrow sample or a blood sample in case of haematological cancers). Thus, the sample to be used in accordance with the present invention is, in case of solid cancers, primarily and preferably a biopsy. The sample to be used in accordance with the present invention is, in case of haematological cancers, primarily and preferably a bone marrow sample or a blood sample.

In relation to haematological cancers (like AML), the term “tumor cell(s) or cancer cell(s)” can refer to (a) “proliferative diseased cell(s)”. In this context, also the major CD44 isoform of cells other than “proliferative diseased cell(s)” from a given sample (like a bone marrow sample or a blood sample) may be evaluated/assessed/scrutinized without deferring from the gist of this invention.

The sample comprising the at least one “tumor cell(s) or cancer cell(s)” (or, in context of haematological cancers (like AML) “proliferative diseased cell”) can be obtained from a patient. The sample can preferably be a biopsy in case of solid cancers, or preferably a bone marrow sample or a blood sample in case of haematological cancers (like AML).

The present invention relates to a method of determining whether a tumor cell or a cancer cell is responsive to an anti-CD44 antibody or to an antigen binding fragment thereof, said method comprising obtaining a sample from a patient, determining the major CD44 isoform in said sample of said patient, wherein if said major CD44 isoform is CD44s, said tumor cell or cancer cell is responsive to said anti-CD44 antibody or to an antigen binding fragment thereof. All explanations and definitions provided herein apply, mutatis mutandis, in this context.

The method of determining whether a tumor cell or a cancer cell is responsive to an anti-CD44 antibody or to an antigen binding fragment thereof as provided in the present invention can also be a non-invasive method which does not involve interaction with a human or animal body (like taking/obtaining a sample from a patient). All explanations and definitions provided herein apply, mutatis mutandis, in this context.

The present invention relates to an in vitro method of determining whether a tumor cell or a cancer cell is responsive to an anti-CD44 antibody or to an antigen binding fragment thereof, said method comprising determining the major CD44 isoform in a sample of a patient, wherein if said major CD44 isoform is CD44s, said tumor cell or cancer cell is responsive to said anti-CD44 antibody or to an antigen binding fragment thereof. All explanations and definitions provided herein apply, mutatis mutandis, in this context.

The present invention relates to a non-invasive method of determining whether a tumor cell or a cancer cell is responsive to an anti-CD44 antibody or to an antigen binding fragment thereof, said method comprising determining the major CD44 isoform in a sample of a patient, wherein if said major CD44 isoform is CD44s, said tumor cell or cancer cell is responsive to said anti-CD44 antibody or to an antigen binding fragment thereof. All explanations and definitions provided herein apply, mutatis mutandis, in this context.

The present invention relates to an in vitro method of determining whether a tumor cell or a cancer cell is responsive to an anti-CD44 antibody or to an antigen binding fragment thereof, said method comprising determining the major CD44 isoform in a sample, wherein if said major CD44 isoform is CD44s, said tumor cell or cancer cell is responsive to said anti-CD44 antibody or to an antigen binding fragment thereof.

The present invention relates to a non-invasive method of determining whether a tumor cell or a cancer cell is responsive to an anti-CD44 antibody or to an antigen binding fragment thereof, said method comprising determining the major CD44 isoform in a sample, wherein if said major CD44 isoform is CD44s, said tumor cell or cancer cell is responsive to said anti-CD44 antibody or to an antigen binding fragment thereof. All explanations and definitions provided herein apply, mutatis mutandis, in this context.

Typically, patients suffering from a hematological cancer like neoplasia (e.g. leukemia, such as acute myoloid leukemia (AML)) have a pathologically (abnormally) increased cell proliferation of certain cell types, in particular of white blood cells (white blood cells are also termed “leukocytes”). The term “white blood cell(s)” is used interchangeably herein with the term “leukocyte(s)”. Typically, neoplasia (like leukemia, such as acute myoloid leukemia (AML)) is characterized by/associated with a pathological (abnormal) increase of immature white blood cells (immature white blood cells are also termed “blast(s)”). The terms “immature white blood cell”, “immature leukocyte” and “blast” are used interchangeably herein. Thus, the “proliferative diseased cell(s)” can, in accordance with the present invention, be (a) white blood cell(s)/(a) leukocyte, such as (an) “immature white blood cell(s)”/“immature leukocyte(s)”/“blast”(s).

The term “patient” as used herein refers to an individual suffering from cancer, suspected to suffer from cancer, or being prone to suffer from cancer.

The herein provided methods can be useful in a therapeutic setting, for example, if a patient suffers from cancer. In other words, if the cancer has already been diagnosed and anti-cancer therapy is contemplated or has already started, the methods of the present invention can allow stratification of patients which can benefit from therapy with an anti-CD44 antibody or an antigen binding fragment thereof. If the major CD44 isoform is determined to be CD44s, the patient can be eligible for therapy with an anti-CD44 antibody or an antigen binding fragment thereof. For such patients the anti-CD44 antibody or an antigen binding fragment thereof might be the sole anti-cancer therapy or it might be administered as co-therapy (e.g. in combination with conventional therapy). The methods of the present invention may also be useful in neoadjuvant therapy in order to stratify patients which can benefit from a reduction of the tumor volume prior to a surgical intervention or in order to stratify patients which are not amenable to surgical intervention.

The herein provided methods can be useful in a prophylactic setting, for example, if a patient is suspected to suffer from cancer or if a patient is being prone to suffer from cancer.

If a patient is suspected to suffer from cancer, the final histological evaluation of a suspected tumor sample (e.g. after dissection in adjuvant therapy) may not yet be available. In case of haematological cancers, it may not have been finally determined that the patient suffers from said cancer. Yet, it may be desirable to initiate treatment or to decide on a therapy before the patient is finally determined to suffer from said cancer (e.g. before the sample is confirmed to be a tumor sample). Alternatively, the development of a tumor/cancer may be at an early stage, so that it may not yet be possible to diagnose the presence of a tumor by routine methods (like imaging techniques). Yet, the presence of tumor markers and/or of other clinical symptoms may give rise to suspecting the patient to suffer from a cancer.

The term “responsiveness” (and likewise “respond” and grammatical variants thereof) as used herein means that (a) tumor cell or (a) cancer cell and/or a patient as defined above responds to or has an increased likelihood of responding to an anti-CD44 antibody or to an antigen binding fragment thereof.

The “response” may be reflected in a “complete response” or “partial response” of the herein identified individuals/patients or tumor(s) (cell(s))/cancer(s) (cell(s) to an anti-CD44 antibody or to an antigen binding fragment thereof. Such a “complete response” or “partial response” can primarily be seen in tumor cells/cancer cells or patients, wherein the major CD44 isoform is CD44s.

The term “response” can refer to a tumor growth inhibition (which can include “tumor shrinkage”) in the novel patient group. Tumor growth inhibition (also termed herein “TGI”) can refer to a reduced tumor growth of tumors/cancers upon treatment with the anti-CD44 antibody or an antigen binding fragment thereof compared to the tumor growth of a control tumor (e.g. a non-responsive tumor, wherein the major CD44 isoform is not CD44s).

Herein it was shown that animal models showed a response in form of a tumor growth inhibition (TGI). Tumor growth inhibition can be determined as follows: Primary tumor volume (TV) was calculated according to the NCl protocol (TV=(length×width²)/2), where “length” and “width” are long and short diameters of tumor mass in mm (Corbett et al., Beverly A. Teicher (ed.) Humana Press, Totowa, N.J., Chapter 5, Page 75-99 (1997)). Calculation was executed from the staging day until study termination, and values were documented as means +/−SEM or medians and inter-quartile ranges (IQR) defined as differences of the third and first quartile.

For calculation of percentage tumor growth inhibition (TGI) during the treatment period, every treated group was compared with its respective vehicle control. TV day z represents the tumor volume of an individual animal at a defined study day (day z) and TV day x represents the tumor volume of an individual animal at the staging day (day x).

The following formula was applied:

$$TGI [ \% ] = 100 - \frac{median ( {TV ( treated )}_{day z} - {TV ( treated )}_{day x} )}{median ( {TV ( resp . control )}_{day z} - {TV ( resp . control )}_{day x} )} \times 100$$

For example, a “tumor growth inhibition” (TGI) of at least 50% can indicate responsiveness herein. A “tumor growth inhibition” (TGI) of 100% can be seen as “stable disease” (tumor does not grow/tumor volume does not increase). Accordingly, a response is (or responding patient/patient group shows) herein preferably a “stable disease”. A “tumor growth inhibition” (TGI) of more than 100% indicates tumor shrinkage (reduction of tumor volume).

“Tumor shrinkage” can refer to a reduced volume of the tumor upon treatment with the anti-CD44 antibody or an antigen binding fragment thereof compared to the initial volume at the start of (i.e. prior to) the treatment with the anti-CD44 antibody or an antigen binding fragment thereof.

A “partial response” may encompass a (temporary) tumor growth inhibition/tumor shrinkage during the course of the treatment as defined above.

The novel patient group can show a “complete response”. The term “complete response” as used herein can refer to the disappearance of all signs of cancer in response to the anti-CD44 antibody or to an antigen binding fragment thereof. The term “complete response” and the term “complete remission” can be used interchangeably herein. For example, a “complete response” may be reflected in the continued shrinkage of the tumor until the tumor has disappeared. A tumor volume of, for example, 0% compared to the initial tumor volume (100%) at the start of (i.e. prior to) the treatment with the anti-CD44 antibody or an antigen binding fragment thereof, can represent a “complete response”. Thus, the term “responsiveness to an anti-CD44 antibody or to an antigen binding fragment thereof” or “responsive patient/individual to an anti-CD44 antibody or to an antigen binding fragment thereof” as used herein may indicate the “complete response”/“complete responsiveness” or the “partial response”/“partial responsiveness” of the tumor (cells), cancer (cells) or individuals/patients determined to respond in accordance with the present invention.

Tumor (cells)/or cancer (cells)/individuals may show a “stable disease” after treatment with an anti-CD44 antibody or an antigen binding fragment thereof, for example the tumor growth is essentially stopped or halted. In other words, the tumor volume does substantially not change during the course of the treatment. Such tumor cells or cancer cells or individuals (i.e. tumor (cells) or cancer (cells) or individuals having CD44s as major isoform can, therefore, show a “stable disease” phenotype.

In contrast to tumor cells or cancer cells or individuals predicted to respond in accordance with the present invention (tumor (cells) or cancer (cells) or individuals having CD44s as major isoform), tumor cells or cancer cells or individuals that do not have CD44s as major isoform do, for example, not show partial or complete response as defined above. For example, (a) non-responding tumor(s) (cells) or cancer (cells) or individuals do not show a tumor growth inhibition as defined above. These “non-responders” can, for example, show a continued tumor growth (e.g. a tumor growth inhibition of less than 50%, less than 40%, less than 30%, less than 20%, less than 10% or lower (like no tumor growth inhibition) during the course of the treatment with an anti-CD44 antibody or an antigen binding fragment thereof. For example, the non-responsive tumor(s) (cells) or cancer (cells) or individuals as defined above can show a tumor growth/increase in tumor volume upon treatment with an an anti-CD44 antibody or to an antigen binding fragment thereof that is not different to a control (control being e.g. tumor(s) (cells) or cancer (cells) or individual(s) not treated with an anti-CD44 antibody or an antigen binding fragment thereof).

A person skilled in the art is readily in the position to determine tumor growth, tumor growth inhibition or tumor shrinkage. For example, the tumor volume at the start of the treatment (i.e. prior to the treatment) with an anti-CD44 antibody or an antigen binding fragment thereof can be determined taking advantage of e.g. Positron emission tomography (PET) or x-ray radiograph. The tumor growth or tumor shrinkage can be monitored during the course of the treatment by taking advantage of e.g. Positron emission tomography (PET) or x-ray radiograph. Thus, the tumor volume at any given time (e.g. prior to treatment, during treatment and/or at the end of the treatment) can be determined and the relative change (increase or reduction) of the tumor volume can be calculated e.g. in %. Thus, it is easily possible to confirm that a tumor or cancer or a tumor/cancer patient determined to respond in accordance with the invention does indeed respond.

In accordance with the present invention, a response may be reflected in the prevention of the development of a tumor or metastases, for example after resection of a tumor, in the prolongation of time to disease progression, or in the reduction of the size of (a) tumor(s) (as explained above) and/or (a) metastases, for example in neoadjuvant therapy. A responsive patient can, for example, benefit from treatment with an anti-CD44 antibody or to an antigen binding fragment thereof in that, e.g. (a) tumor(s) and/or (a) metastasis(es) which has been resected does not recur within 1 year after termination of the treatment with the anti-CD44 antibody or an antigen binding fragment thereof, or within 2 years, 3 years, 4 years, 5 years, 10 years or, within 15 year after termination of the treatment.

A person skilled in the art appreciates that a positive test for CD44s as major isoform in the novel patient sub-group identified by the herein provided methods, necessarily translates 1:1 into a successful treatment. However, by these methods sub-groups of patients are identified that have a higher chance of response (=show a better response rate) to a treatment with an anti-CD44 antibody or an antigen binding fragment thereof, as compared to the sub-group of patients not showing these positive test results. In other words, a positive result indicates that the individual or patient has a higher chance to respond to treatment with an anti-CD44 antibody or an antigen binding fragment thereof, as compared to a patient having, for example, a “CD44v” transcript/splice variant as major isoform (like “CD44 v2-v10”, “CD44 v3-v10”, “CD44 v8-v10”, and “CD44 v10”).

The term “determining whether a tumor cell or a cancer cell is responsive to an anti-CD44 antibody or to an antigen binding fragment thereof” as used herein refers in particular and preferably to “predicting whether a tumor cell or a cancer cell is responsive to an anti-CD44 antibody or to an antigen binding fragment thereof”. Thus, these terms can be used interchangeably herein. The meaning of the term “determining responsiveness to therapy/drug” (like “determining whether a tumor cell or a cancer cell is responsive to an anti-CD44 antibody or to an antigen binding fragment thereof”) is well known in the art and used accordingly herein. For example, it is well known in the art that predictive factors determine the responsiveness to a treatment (Lonning, Annals of Oncology 18, supp 8 (2007) (doi:10.1093/annonc/mdm260). Thus, predictive factors indicate, for example, which adjuvant therapy is most appropriate (Lonning, loc. cit.). It is therefore contemplated and preferred herein that the determination of the major CD44 isoform is performed prior to (the start of) the treatment with the anti-CD44 antibody or an antigen binding fragment thereof. Accordingly, “predicting whether a tumor cell or a cancer cell is responsive to an anti-CD44 antibody or to an antigen binding fragment thereof” is preferably performed before the first cycle of therapy/treatment with the anti-CD44 antibody or an antigen binding fragment thereof. Thus, the herein provided markers are preferably used as predictive markers.

Yet, it is also envisaged that the herein provided markers can be used as monitoring markers. In other words, the markers can be used to monitor the response to an anti-CD44 antibody or an antigen binding fragment thereof after treatment has started (e.g. during treatment, encompassing treatment breaks). Thus, “determining whether a tumor cell or a cancer cell is responsive to an anti-CD44 antibody or to an antigen binding fragment thereof” as used herein can refer to “monitoring whether a tumor cell or a cancer cell is responsive to an anti-CD44 antibody or to an antigen binding fragment thereof”.

As illustrated below, the present invention provides for the therapy of patient (sub-)groups with an anti-CD44 antibody or to an antigen binding fragment thereof, inter alia, because not all cancer patients for whom said antibody therapy was contemplated in the art do indeed respond to the therapy.

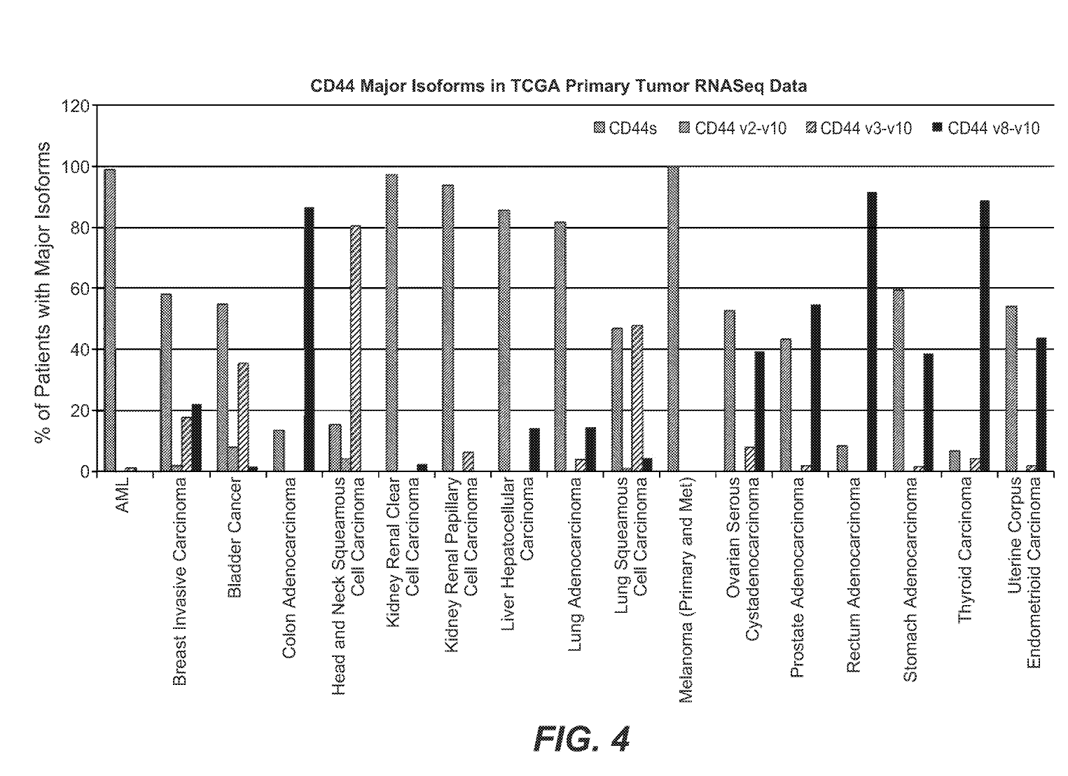

FIG. 4 shows major CD44 isoforms in tumor samples of various cancer entities. In certain cancer entities, only one major CD44 isoform exists (i.e. the same CD44 isoform is determined to be the major isoform in most tumor samples of a given cancer entity). For example, more than 80% or 85% of samples of AML patients, kidney renal clear cell carcinoma patients, kidney renal papillary cell carcinoma patients, liver hepatocellular carcinoma patients, lung adenocarcinoma patients, and melanoma patients are determined to have CD44s as major isoform. Because CD44s as major isoform indicates responsiveness to treatment with an anti-CD44 antibody or to an antigen binding fragment thereof, most of these cancer patients are expected to respond to said antibody therapy. These patients will therefore likely benefit from said therapy.

On the other hand, those patients may highly benefit from the herein provided methods, wherein CD44s is only in few samples determined as major isoform. It may be extremely valuable to stratify those patients that do have CD44s as major isoform (and therefore likely respond to treatment with an anti-CD44 antibody or to an antigen binding fragment thereof) from patients that do not have CD44s as major isoform. For example, less than 60% or 65% of samples of breast invasive carcinoma patients, bladder cancer patients, colon adenocarcinoma patients, head and neck squameous cell carcinoma patients, lung squameous cell carcinoma patients, ovarian serous cystadenocarcinoma patients, prostate adenocarcinoma patients, rectum adenocarcinoma patients, stomach adenocarcinoma patients, thyroid carcinoma patients, and uterine corpus endometrioid carcinoma patients are determined to have CD44s as major isoform. In other words, less than 60% or 65% of the above patients are expected to respond to the herein provided antibody treatment. The remaining at least 40% or at least 35% are expected not to respond/benefit from the treatment. For the remaining at least 40% (or at least 35%) patients other treatment options that therapy with anti-CD44 antibodies might be conceivable.

The herein provided methods for stratifying eligible patients might be particularly advantageous in situations where only few samples are determined to have CD44s as major isoform. For example, from about less than 60% (or less than 65%) to about more than 40% (or more than 45%) of samples of breast invasive carcinoma patients, bladder cancer patients, lung squameous cell carcinoma patients, ovarian serous cystadenocarcinoma patients, prostate adenocarcinoma patients, stomach adenocarcinoma patients, thyroid carcinoma patients, and uterine corpus endometrioid carcinoma patients are determined to have CD44s as major isoform. For example, about less than 20% (or less than 25%) of samples of colon adenocarcinoma patients, head and neck squameous cell carcinoma patients, rectum adenocarcinoma patients, thyroid carcinoma patients are determined to have CD44s as major isoform.

In accordance with the above, the patient to be stratified for treatment with an anti-CD44 antibody or an antigen binding fragment thereof, can be suffering from a hematological or solid cancer, can be suspected of suffering from a hematological or solid cancer, or can be prone to suffering from a hematological or solid cancer. The solid cancer can be metastatic and/or locally advanced. The cancer/tumor can not be amenable to standard treatment (like chemotherapy and/or radiation therapy). Hematological cancer is preferred herein.

In one aspect of the present invention, the hematological cancer is preferably leukemia, like acute myeloid leukemia (AML), chronic myeloid leukemia (CML) or chronic lymphoid leukemia (CLL). Most preferably, said leukemia is acute myeloid leukemia (AML). The hematological cancer can be multiple myeloma (MM) or myelodysplastic syndrome (MDS).

In one aspect of the present invention the solid cancer is head or neck cancer, melanoma, lung adenocarcinoma, renal cancer, kidney cancer, mesothelioma, lung squeamous cell carcinoma, breast cancer, liver hepatocellular carcinoma, glioblastoma, gastric cancer, ovarian cancer, bladder cancer, thymus carcinoma, colorectal carcinoma, or prostate cancer. Colorectal carcinoma includes colorectal adenocarcinoma. Colorectal adenocarcinoma includes two sub-types, colon adenocarcinoma and rectum adenocarcinoma. Accordingly, also colon adenocarcinoma or rectum adenocarcinoma can be treated in accordance with the present invention. Of the solid cancers, head or neck cancer (like head and neck squameous cell carcinoma) is preferred herein

In one aspect of the present invention, the solid cancer can be head and neck squameous cell carcinoma, melanoma, lung adenocarcinoma, kidney renal clear cell carcinoma, kidney renal papillary cell carcinoma, lung squameous cell carcinoma, breast invasive carcinoma, liver hepatocellular carcinoma, stomach adenocarcinoma, ovarian serous cystadenocarcinoma, bladder cancer, prostate adenocarcinoma, colorectal adenocarcinoma, colon adenocarcinoma, rectum adenocarcinoma, thymus carcinoma,thyroid carcinoma, and uterine corpus endometrioid carcinoma. Colorectal adenocarcinoma includes two sub-types, colon adenocarcinoma and rectum adenocarcinoma.

The following relates to anti-CD44 antibodies (or antigen binding fragments thereof) that can be used in accordance with the present invention.

Anti-CD44 antibodies (or antigen binding fragments thereof or anti-CD44 binding molecules derived from antibodies as disclosed herein) to be employed in a medical setting as disclosed herein, i.e. the treatment of a tumorous disease/cancer are antibodies/antigen binding fragments/anti-CD44 binding molecules which are derived or based on antibody molecules as produced by the hybridoma deposited with the ATCC having accession number PTA-4621. These antibodies/anti-CD44 binding molecules also relate to variants and/or derivatives of the antibodies as obtainable from said hybridoma PTA-4621. Such variants comprise, e.g., chimeric and humanized versions of PTA-4621. The invention further encompasses the use of antibodies, antigen binding fragments thereof or anti-CD44 binding molecules derived from antibodies as obtainable from said hybridoma PTA-4621 that compete for binding to an epitope as bound to/interacted with the antibody as obtained from said hybridoma PTA-4621.

The anti-CD44 antibodies (or antigen binding fragments thereof) to be used in accordance with the present invention can bind to a glycosylation and conformation dependent epitope on the constant region of CD44 that involves the amino acid sequence AFDGPITITIV (SEQ ID NO. 86).

The antibodies and fragments to be used in accordance with the means and methods as provided herein are specific for CD44, in particular CD44 as expressed on the surface of a cell of tumor cells that expresses human CD44 on the surface wherein the corresponding cancer/tumor cell comprises as major CD44 isoform CD44s.

In certain aspects the antibodies for use according to the means and methods provided herein are humanized or chimeric versions of the antibody produced by the hybridoma deposited with the ATCC having accession number PTA-4621.

In specific embodiments the antibodies for use according to the methods disclosed herein, or antigen binding fragments thereof, comprise one or more of a heavy chain variable domain (V_{H}) CDR1 having the amino acid sequence of RYWMS (SEQ ID NO:3),

a V_{H} CDR2 having the amino acid sequence of EVNPDSTSINYTPSLKD (SEQ ID NO:4),

a V_{H} CDR3 having the amino acid sequence of PNYYGSRYHYYAMDY (SEQ ID NO:5),

a light chain variable domain (V_{L}) CDR1 having the amino acid sequence of RASQDINNYLN (SEQ ID NO:6),

a V_{L} CDR2 having the amino acid sequence of YTSRLHS (SEQ ID NO:7); and

a V_{L} CDR3 having the amino acid sequence of QQGSTLPFT (SEQ ID NO:8).

The antibodies for use according to the methods disclosed herein, or antigen binding fragments thereof, comprise preferably all of

a heavy chain variable domain (V_{H}) CDR1 having the amino acid sequence of RYWMS (SEQ ID NO:3),

a V_{H} CDR2 having the amino acid sequence of EVNPDSTSINYTPSLKD (SEQ ID NO:4),

a V_{H} CDR3 having the amino acid sequence of PNYYGSRYHYYAMDY (SEQ ID NO:5),

a light chain variable domain (V_{L}) CDR1 having the amino acid sequence of RASQDINNYLN (SEQ ID NO:6),

a V_{L} CDR2 having the amino acid sequence of YTSRLHS (SEQ ID NO:7); and

a V_{L} CDR3 having the amino acid sequence of QQGSTLPFT (SEQ ID NO:8).

The antibody termed “RG7356” (or “R05429083”) as used in the appended examples comprises the above defined CDR sequences as shown in SEQ ID NO: 3 to 8.

The antibodies as to be employed in medical intervention and as determined by the means and methods provided herein may also comprise two or more of, three or more of, four or more of, five or more of, and, in certain aspects, all of a V_{H} CDR1 having the amino acid sequence of SEQ ID NO:3, a V_{H} CDR2 having the amino acid sequence of SEQ ID NO:4, a V_{H} CDR3 having the amino acid sequence of SEQ ID NO:5, a V_{L} CDR1 having the amino acid sequence of SEQ ID NO:6, a V_{L} CDR2 having the amino acid sequence of SEQ ID NO:7 and a V_{L} CDR3 having the amino acid sequence of SEQ ID NO:8.

The anti-CD44 antibody can be a chimeric antibody. The anti-CD44 antibody can comprise a V_{H} domain having the amino acid sequence of SEQ ID NO:1. The anti-CD44 antibody can comprise a V_{L} domain having the amino acid sequence of SEQ ID NO:2. The antibodies/antigen binding fragments/anti CD44 binding molecules to be used in accordance with the means and methods of the invention, i.e. in the cancer/tumor prevention and treatment with anti-CD44 antibody molecules/anti-CD44 antigen binding fragments/anti-CD44 binding molecules on and in cancer/tumor cells that comprise as major CD44 isoform CD44s, may comprise the following variable regions:

a V_{H} domain having the amino acid sequence of

(SEQ ID NO: 1)

EVKLLESGGGLVQPGGSLKLSCATSGFDFSRYWMSWVRQAPGKGLEWIGE
VNPDSTSINYTPSLKDQFIISRDNAKNTLDLQMSKVSSEDTALYYCTRPN
YYGSRYHYYAMDYWGQGTSVTVSS

and/or a V_{L} domain having the amino acid sequence of

(SEQ ID NO: 2)

DIQMTQTTSSLSVSLGDRVTINCRASQDINNYLNWYQQKPDGTVKLLIYY
TSRLHSGVPSRFSGSGSGTDFSLTISNLEKEDVATYFCQQGSTLPFTFGS
GTKLEIK.

In certain aspects the invention encompasses the use of an anti-CD44 antibody or antigen binding fragment comprising a V_{H} domain having the amino acid sequence of SEQ ID NO:1 and a V_{L} domain having the amino acid sequence of SEQ ID NO:2.

The invention further encompasses the use of medical use of a humanized anti-CD44 antibody, a humanized antigen binding fragments thereof or a humanized anti-CD44 binding molecule for treating or preventing cancer in patients that have tumor or cancer cells that comprise as major CD44 isoform CD44s. The anti-CD44 antibody can be a humanized antibody.

In some aspects, such humanized anti-CD44 antibody or humanized antigen binding fragment/anti-CD44 binding molecules may comprise

a V_{H} domain having the amino acid sequence of

(SEQ ID NO: 9)

EVQLVESGGGLVQPGGSLRLSCAASGFDFSRYWMSWVRQAPGKGLVWVGE
VNPDSTSINYTPSLKDRFTISRDNAKNTLYLQMNSLRAEDTAVYYCTRPN
YYGSRYHYYAMDYWGQGTLVTVSS
or

(SEQ ID NO: 10)

EVQLVESGGGLVQPGGSLRLSCATSGFDFSRYWMSWVRQAPGKGLVWIGE
VNPDSTSINYTPSLKDQFTISRDNAKNTLYLQMNSLRAEDTAVYYCTRPN
YYGSRYHYYAMDYWGQGTLVTVSS

and/or a V_{L} domain having the sequence of

(SEQ ID NO: 11)

DIQMTQSPSSLSASVGDRVTITCRASQDINNYLNWYQQKPGKAPKLLIYY
TSRLHSGVPSRFSGSGSGTDFTFTISSLQPEDIATYYCQQGSTLPFTFGQ
GTKLEIK.

Preferably, the humanized anti-CD44 antibody or humanized antigen binding fragment/anti-CD44 binding molecules to be used herein comprise

a V_{H} domain having the amino acid sequence of

(SEQ ID NO: 10)

EVQLVESGGGLVQPGGSLRLSCATSGFDFSRYWMSWVRQAPGKGLVWIGE
VNPDSTSINYTPSLKDQFTISRDNAKNTLYLQMNSLRAEDTAVYYCTRPN
YYGSRYHYYAMDYWGQGTLVTVSS

and a V_{L} domain having the sequence of

(SEQ ID NO: 11)

DIQMTQSPSSLSASVGDRVTITCRASQDINNYLNWYQQKPGKAPKLLIYY
TSRLHSGVPSRFSGSGSGTDFTFTISSLQPEDIATYYCQQGSTLPFTFGQ
GTKLEIK.

The antibody termed “RG7356” (or “R05429083”) as used in the appended examples comprises the above defined V_{H} domain and V_{L} domain as shown in SEQ ID NO: 10 and 11.

In a particular aspect, the invention provides for the medical use of a humanized anti-CD44 antibody or antigen binding fragment thereof in preventing disease or treating disease in a subject suffering from cancer or a tumor, wherein the corresponding cancer/tumor cells comprise as major CD44 isoform CD44s. As disclosed herein and as surprisingly found, herein a tumor cell or cancer cell that comprises CD44s as major isoform is responsive to anti-CD44 antibodies as disclosed herein. The humanized anti-CD44 antibody can comprise a V_{H} domain having the amino acid sequence of SEQ ID NO:9 or SEQ ID NO: 10. The humanized anti-CD44 antibody can comprise a V_{L} domain having the amino acid sequence of SEQ ID NO:11. A humanized anti-CD44 antibody to be employed in context of this invention may comprises a V_{H} domain having the amino acid sequence of SEQ ID NO: 9 and a V_{L} domain having the amino acid sequence of SEQ ID NO: 11, or wherein said anti-CD44 antibody comprises a V_{H} domain having the amino acid sequence of SEQ ID NO:10 and a V_{L} domain having the amino acid sequence of SEQ ID NO: 11. In particular aspects the subject/patient/individual is a human. The invention also provides for the use of a humanized anti-CD44 antibody or a humanized antigen binding fragment for the prevention or treatment or diagnosis of a cancer/tumor disease wherein the cancer/tumor cells comprise CD44s as major isoform, wherein said humanized anti-CD44 antibody comprises a V_{H} domain having the amino acid sequence of SEQ ID NO:9 and a V_{L} domain having the amino acid sequence of SEQ ID NO:11, or wherein said anti-CD44 antibody comprises a V_{H} domain having the amino acid sequence of SEQ ID NO:10 and a V_{L} domain having the amino acid sequence of SEQ ID NO:11.

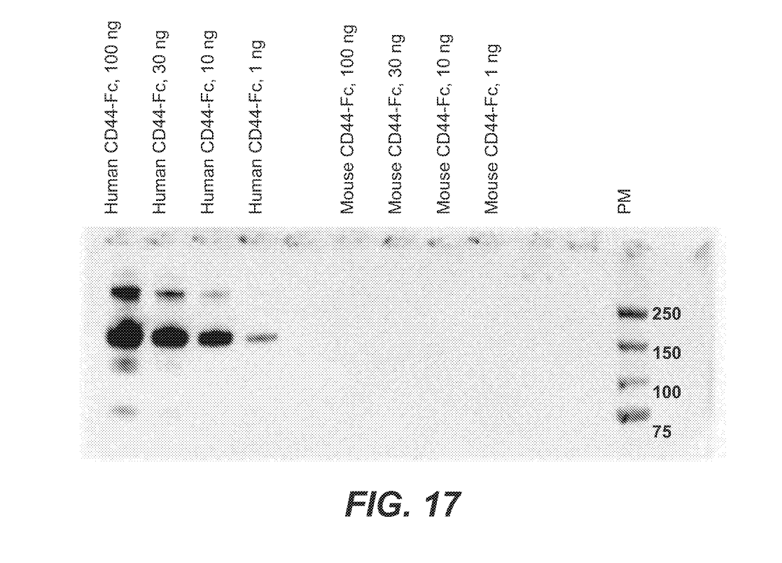

In one aspect, the anti-CD44 antibody specifically binds to human CD44 isoforms, i.e. the anti-CD44 antibody does not bind to non-human CD44 isoforms. For example, the anti-CD44 antibody does not bind to murine CD44 isoforms, which is proven by Western Blot analysis as shown in FIG. 17.

In one aspect, the anti-CD44 antibody may compete for binding with the anti-CD44 antibody produced by the hybridoma deposited with the ATCC with Accession number PTA-4621.

In one aspect, the anti-CD44 antibody interferes with the interaction of CD44 and hyaluronic acid. The interference of the anti-CD44 antibody to be used herein with the interaction of CD44 and hyaluronic acid may be determined/assayed by ELISA-Assays and/or ACEA-Assays.

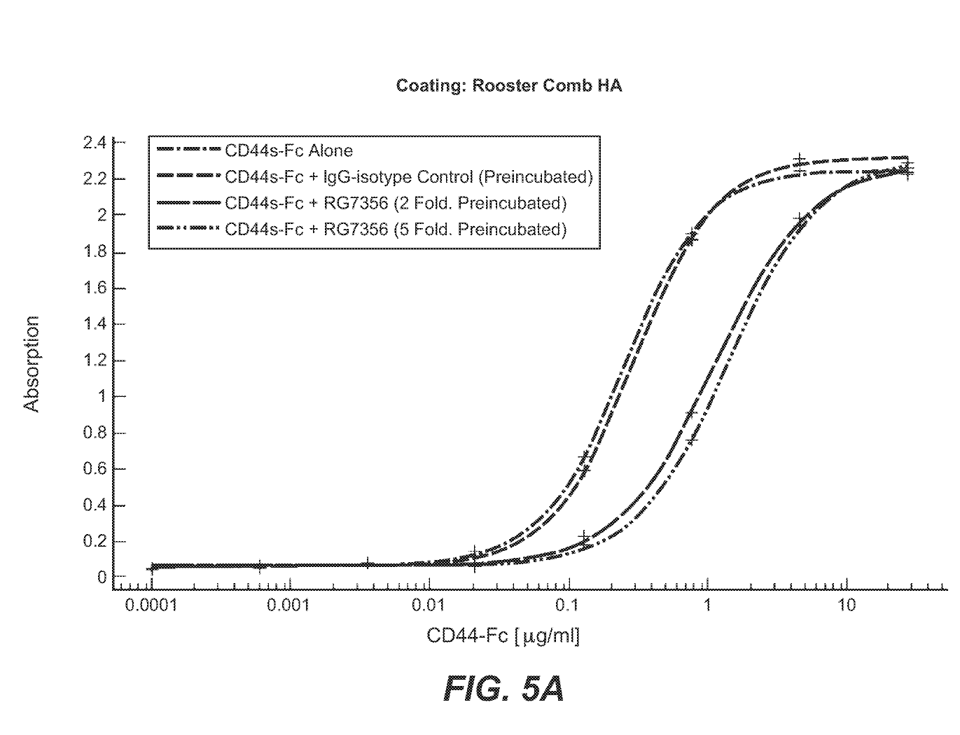

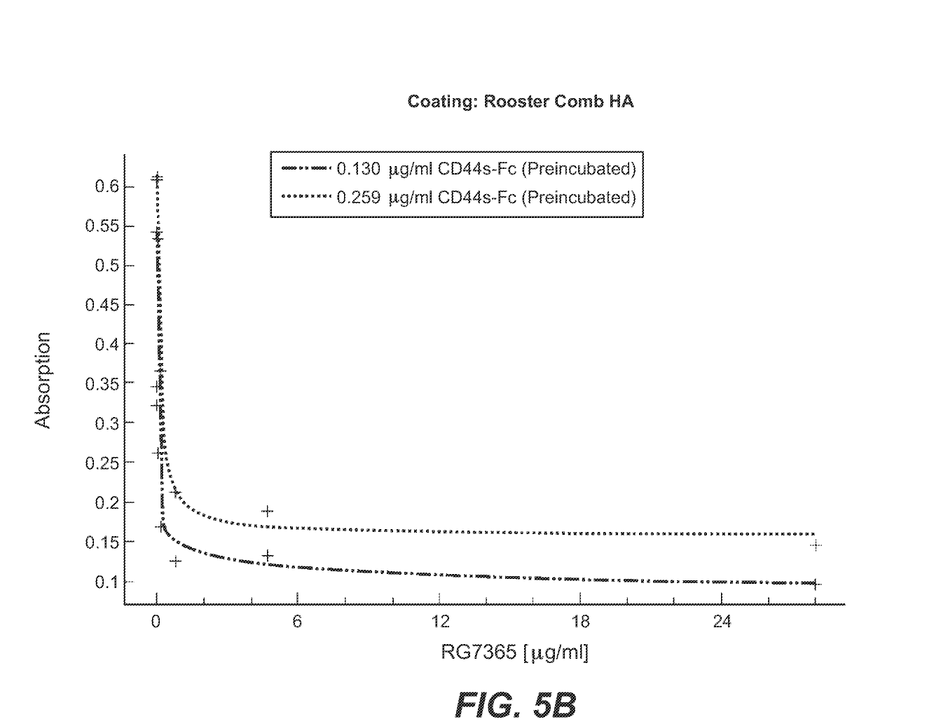

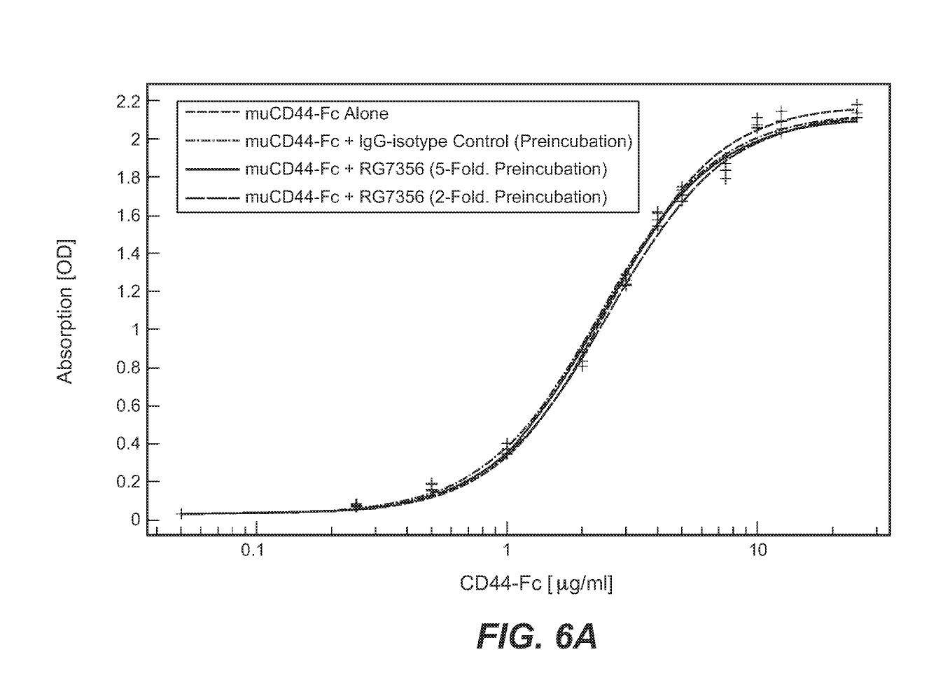

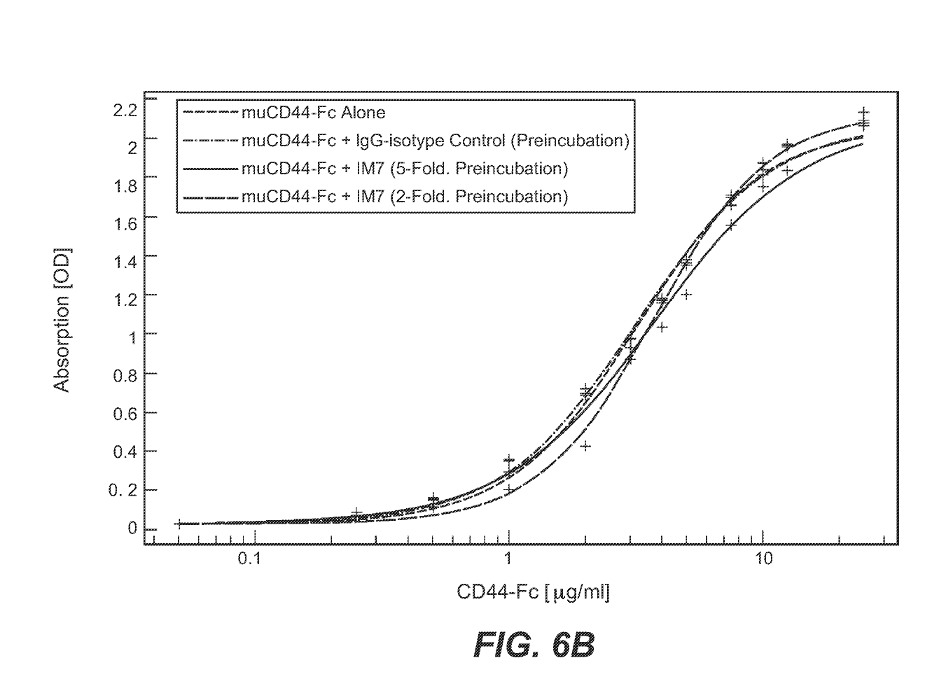

This is exemplified by the results shown in FIGS. 5a and 5b that show clear interference of RG7356 with binding of human CD44 to Hyaluronic Acid (HA) coated plates. IM7, in contrast to its strong binding affinity for murine CD44 does not interfere with the binding of murine CD44 to HA-coated plates (FIG. 6a). Since RG7356 does not recognize murine CD44, there is no interference with the mCD44/HA-interaction (FIG. 6b). This is in line with other results (Zheng et al., J. Cell Biol. 1995, 485-495) suggesting that IM7 does not compete with HA for CD44 binding.

The herein provided methods can further comprise determining the level of hyaluronic acid and/or the level of one or more hyaluronic acid synthetases in order to confirm/validate the determination of a responsive tumor cell/cancer cell or patient. For example, the level of one or more of hyaluronic acid synthetase 1, hyaluronic acid synthetase 2 and/or hyaluronic acid synthetase 3 can be determined. Exemplary nucleic acid sequences and amino acid sequences of hyaluronic acid synthetases are shown in SEQ ID NO.s 80 to 85, respectively.

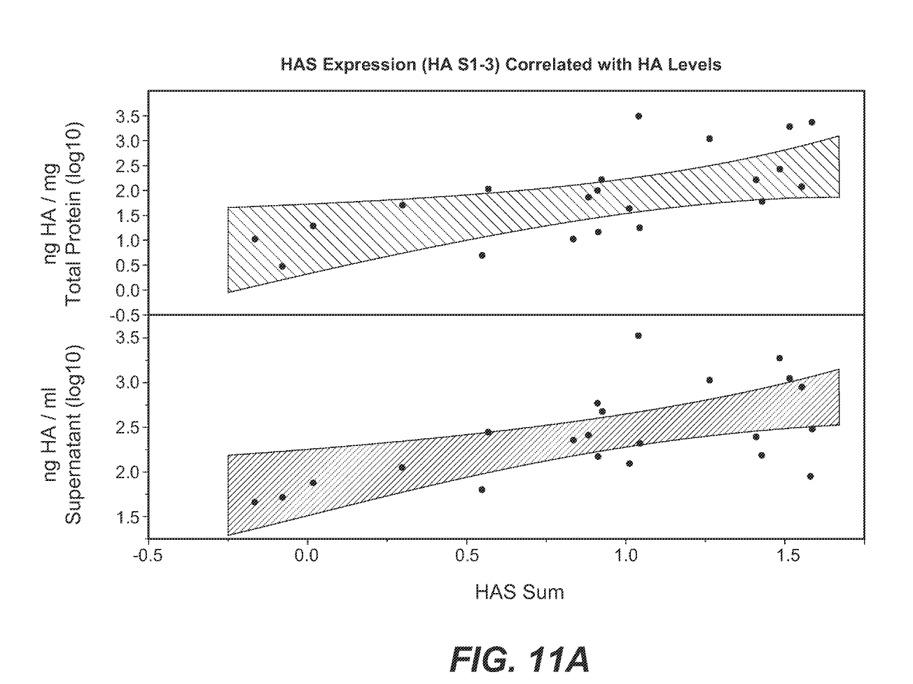

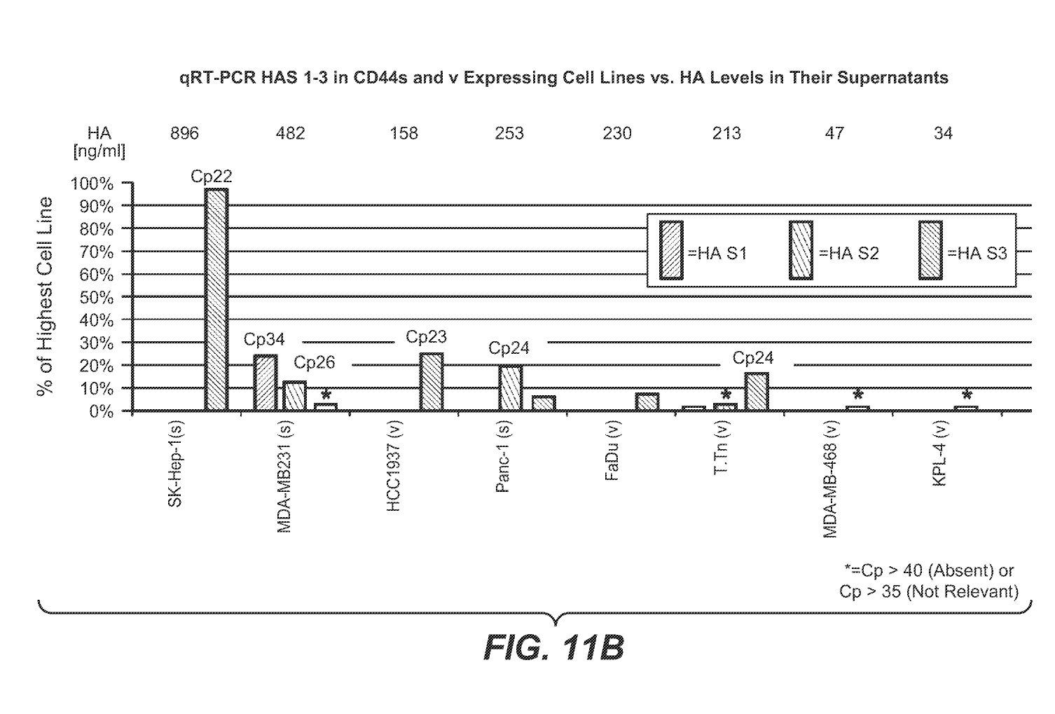

It is shown herein that the level of hyaluronic acid and likewise of hyaluronic acid synthetases is increased if the cancer cell(s)/tumor cell(s) are determined to have CD44s as major isoform; see FIGS. 11A and 11B. Furthermore, the level of hyaluronic acid and of hyaluronic acid synthetases correlate.

If the level of hyaluronic acid is increased in comparison with a control, said tumor cell or cancer cell is responsive to said anti-CD44 antibody. In addition, or in the alternative, an increased level of one or more hyaluronic acid synthetases in comparison with a control can indicate responsiveness of (a) tumor cell(s) or (a) cancer cell(s) to the anti-CD44 antibody. For example, the level of hyaluronic acid and/or said level of one or more hyaluronic acid synthetases can be at least 2.5-fold, preferably at least 5-fold increased in comparison to the control.

The level of hyaluronic acid can, for example, be assessed/determined taking advantage of ELISA assays. For example, in a pre-clinically setting (tumor lysates needed) HA levels of at least 150 ng/ml can indicate responsiveness to an anti-CD44 antibody.

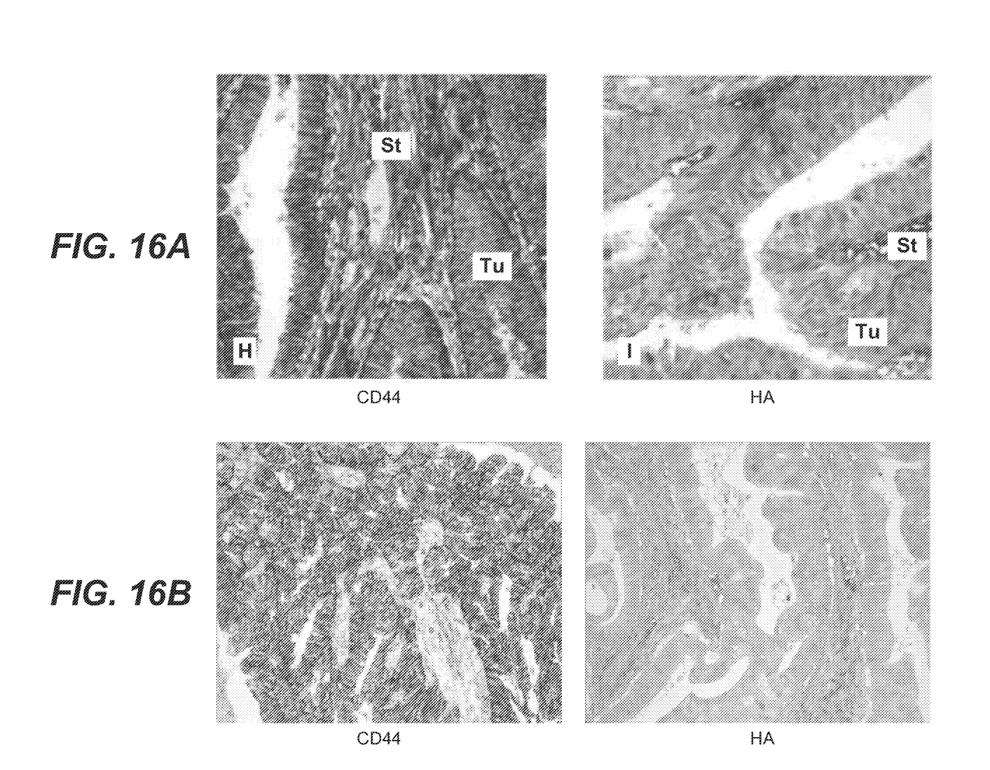

Further, hyaluronic acid affinity histochemistry assays can be used to distinguish semi-quantitatively between HA low, medium and high levels. Tumors showing a medium or high hyaluronic acid (HA) level in the tumor or tumor stroma are considered responsive to therapy with an anti-CD44 antibody (or an antigen-binding fragment thereof). FIG. 16 shows representative pictures of CD44 positive tumors that do not express HA in the tumor and rather low HA in the tumor stroma.

If a tumor cell(s)/cancer cell(s)/sample shows expression of at least one of the three HA synthetases (HAS1-3), the tumor cell(s)/cancer cell(s)/sample is considered to be positive for hyaluronic acid (and vice versa).

The level of the one or more hyaluronic acid synthetases can be the expression level, for example, the mRNA expression level. Methods for assessing/quantification of the mRNA expression level have been described herein above in detail. Examplary methods to be used are in situ hybridization, micro-arrays, or RealTime PCR (such as quantitative Real Time PCR).

The expression level can be the protein expression level. Non-limiting methods for assessing/determining the protein expression level are immunoassay, gel- or blot-based methods, IHC, mass spectrometry, flow cytometry, and FACS.

As used in context of the methods of the present invention, a non-limiting example of a “control” is preferably a “non-responder” control, for example a sample/cell/tissue obtained from one or more healthy subjects or one or more patients that suffer from a haematological cancer or solid cancer and are known to be not responsive to an anti-CD44 antibody. Another example for a “non-responder” control is a cell line/sample/cell/tissue that shows no response to an anti-CD44 antibody in an ex-vivo test. Another non-limiting example of a “control” is an “internal standard”, for example purified or synthetically produced proteins and/or peptides or a mixture thereof, where the amount of each protein/peptide is gauged by using the “non-responder” control described above.

The control may also be a sample/cell/tissue obtained from the individual or patient suspected of suffering from the haematological cancer/neoplasia provided that the sample/cell/tissue does not contain proliferative diseased cells as defined herein. In a further alternative, the “control” may be a sample/cell/tissue obtained from an individual or patient suffering from the haematological cancer/neoplasia (like leukemia, such as AML), that has been obtained prior to the development or diagnosis of said haematological cancer/neoplasia.

The following relates to exemplary nucleic acid sequences e.g. of hyaluronic acid synthetases to be used in accordance with the present invention. Exemplary nucleic acid sequences of hyaluronic acid synthetases are shown herein in SEQ ID NO. 80, 82 and 84. Corresponding RNA sequences are shown in SEQ ID NOs 151, 152 and 153, respectively. Fragments thereof or further nucleic acid sequence of hyaluronic acid synthetases to be used herein can be identified by the skilled person using methods known in the art, e.g. by using hybridization assays or by using alignments, either manually or by using computer programs such as those mentioned herein below in connection with the definition of the term “hybridization” and degrees of homology. For example, the nucleic acid sequence encoding for orthologs of hyaluronic acid synthetases is at least 40% homologous to the nucleic acid sequence as shown in SEQ ID NO. 80, 82 and 84. More preferably, the nucleic acid sequence encoding for orthologs of hyaluronic acid synthetases is at least 45%, 50%, 55%, 60%, 65%, 70%, 75%, 80%, 85%, 90%, 91%, 92%, 93%, 94%, 95%, 96%, 97% or 98% homologous to the nucleic acid sequence as shown in SEQ ID NO. 80, 82 and 84, wherein the higher values are preferred. Most preferably, the nucleic acid sequence encoding for orthologs of hyaluronic acid synthetases is at least 99% homologous to the nucleic acid sequence as shown in SEQ ID NO. 80, 82 and 84. The term “orthologous” or “orthologous gene” as used herein refers to genes in different species that are similar to each other because they originated from a common ancestor.

Hybridization assays for the characterization of orthologs of known nucleic acid sequences are well known in the art; see e.g. Sambrook, Russell “Molecular Cloning, A Laboratory Manual”, Cold Spring Harbor Laboratory, N.Y. (2001); Ausubel, “Current Protocols in Molecular Biology”, Green Publishing Associates and Wiley Interscience, N.Y. (1989). The term “hybridization” or “hybridizes” as used herein may relate to hybridizations under stringent or non-stringent conditions. If not further specified, the conditions are preferably non-stringent. Said hybridization conditions may be established according to conventional protocols described, e.g., in Sambrook (2001) loc. cit.; Ausubel (1989) loc. cit., or Higgins and Hames (Eds.) “Nucleic acid hybridization, a practical approach” IRL Press Oxford, Washington D.C., (1985). The setting of conditions is well within the skill of the artisan and can be determined according to protocols described in the art. Thus, the detection of only specifically hybridizing sequences will usually require stringent hybridization and washing conditions such as, for example, the highly stringent hybridization conditions of 0.1×SSC, 0.1% SDS at 65° C. or 2×SSC, 60° C., 0.1% SDS. Low stringent hybridization conditions for the detection of homologous or not exactly complementary sequences may, for example, be set at 6×SSC, 1% SDS at 65° C. As is well known, the length of the probe and the composition of the nucleic acid to be determined constitute further parameters of the hybridization conditions.

In accordance with the present invention, the terms “homology” or “percent homology” or “identical” or “percent identity” or “percentage identity” or “sequence identity” in the context of two or more nucleic acid sequences refers to two or more sequences or subsequences that are the same, or that have a specified percentage of nucleotides that are the same (preferably at least 40% identity, more preferably at least 45%, 50%, 55%, 60%, 65%, 70%, 75%, 80%, 85%, 90%, 91%, 92%, 93%, 94%, 95%, 96%, 97% or 98% identity, most preferably at least 99% identity), when compared and aligned for maximum correspondence over a window of comparison, or over a designated region as measured using a sequence comparison algorithm as known in the art, or by manual alignment and visual inspection. Sequences having, for example, 75% to 90% or greater sequence identity may be considered to be substantially identical. Such a definition also applies to the complement of a test sequence. Preferably the described identity exists over a region that is at least about 15 to 25 nucleotides in length, more preferably, over a region that is at least about 50 to 100 nucleotides in length and most preferably, over a region that is at least about 800 to 1200 nucleotides in length (or over the entire length of the sequence e.g. shown in SEQ ID NO. 80, 82 or 84). Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art.

Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul, (1997) Nucl. Acids Res. 25:3389-3402; Altschul (1993) J. Mol. Evol. 36:290-300; Altschul (1990) J. Mol. Biol. 215:403-410). The BLASTN program for nucleic acid sequences uses as defaults a word length (W) of 11, an expectation (E) of 10, M=5, N=4, and a comparison of both strands. The BLOSUM62 scoring matrix (Henikoff (1989) PNAS 89:10915) uses alignments (B) of 50, expectation (E) of 10, M=5, N=4, and a comparison of both strands. In order to determine whether an nucleotide residue in a nucleic acid sequence corresponds to a certain position in the nucleotide sequence of e.g. SEQ ID NO:11, 13 or 15, or e.g. 80, 82 and 84, the skilled person can use means and methods well-known in the art, e.g., alignments, either manually or by using computer programs such as those mentioned herein. For example, BLAST 2.0, which stands for Basic Local Alignment Search Tool BLAST (Altschul (1997), loc. cit.; Altschul (1993), loc. cit.; Altschul (1990), loc. cit.), can be used to search for local sequence alignments. BLAST, as discussed above, produces alignments of nucleotide sequences to determine sequence similarity. Because of the local nature of the alignments, BLAST is especially useful in determining exact matches or in identifying similar sequences. The fundamental unit of BLAST algorithm output is the High-scoring Segment Pair (HSP). An HSP consists of two sequence fragments of arbitrary but equal lengths whose alignment is locally maximal and for which the alignment score meets or exceeds a threshold or cut-off score set by the user. The BLAST approach is to look for HSPs between a query sequence and a database sequence, to evaluate the statistical significance of any matches found, and to report only those matches which satisfy the user-selected threshold of significance. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the program output. Analogous computer techniques using BLAST (Altschul (1997), loc. cit.; Altschul (1993), loc. cit.; Altschul (1990), loc. cit.) are used to search for identical or related molecules in nucleotide databases such as GenBank or EMBL. This analysis is much faster than multiple membrane-based hybridizations. In addition, the sensitivity of the computer search can be modified to determine whether any particular match is categorized as exact or similar. The basis of the search is the product score which is defined as:

$$\frac{\% sequence identity \times \% maximum BLAST score}{100}$$

and it takes into account both the degree of similarity between two sequences and the length of the sequence match. For example, with a product score of 40, the match will be exact within a 1-2% error; and at 70, the match will be exact. Similar molecules are usually identified by selecting those which show product scores between 15 and 40, although lower scores may identify related molecules. Another example for a program capable of generating sequence alignments is the CLUSTALW computer program (Thompson (1994) Nucl. Acids Res. 2:4673-4680) or FASTDB (Brutlag (1990) Comp. App. Biosci. 6:237-245), as known in the art.

The meaning of the term “homologous” and “homology”, respectively, particularly with respect to two amino acid sequences to be compared, is also known in the art. These terms are used herein accordingly. For example, the term “homology”/“homologous” is used herein in context of a polypeptide which has a homology, that is to say a sequence identity, of at least 60%, preferably of at least 70%, more preferably of at least 80%, even more preferably of at least 90% and particularly preferred of at least 95%, especially preferred of at least 98% and even more preferred of at least 99% to the, preferably entire, amino acid sequence of another polypeptide. Herein, “another polypeptide” is a polypeptide encoded by a hyaluronic acid synthetases gene, for example a polypeptide as shown in SEQ ID NO. 81, 83 or 85. Methods for sequence comparison, particularly amino acid sequence comparison, and hence, determination of homology are well known in the art. For example, the degree of homology can be determined conventionally using known computer programs such as the DNASTAR program with the ClustalW analysis. This program can be obtained from DNASTAR, Inc., 1228 South Park Street, Madison, Wis. 53715 or from DNASTAR, Ltd., Abacus House, West Ealing, London W13 OAS UK (support@dnastar.com) and is accessible at the server of the EMBL outstation.

When using the Clustal analysis method to determine whether a particular sequence is, for instance, 90% identical to a reference sequence default settings may be used or the settings are preferably as follows: Matrix: blosum 30; Open gap penalty: 10.0; Extend gap penalty: 0.05; Delay divergent: 40; Gap separation distance: 8 for comparisons of amino acid sequences. For nucleotide sequence comparisons, the Extend gap penalty is preferably set to 5.0.

The above methods can also be used to identify homologous/orthologous sequences of CD44 isoforms described herein and to be used/assessed in accordance with the present invention, for example, the CD44s isoform. Thus, the definitions and explanations provided herein above in relation to homologous/orthologous sequences or sequences with a certain level of identity apply, mutatis mutandis, to CD44 isoforms as defined herein, in particular to CD44s isoforms. Exemplary human nucleic acid sequences encoding the CD44 isoform CD44s are shown in SEQ ID NOs: 12 and 14. Exemplary corresponding RNA sequences are shown in SEQ ID NOs: 117 and 118. Exemplary human amino acid sequences of the CD44 isoform CD44s are shown in SEQ ID NO: 13 and 15.

The following exemplary primers can be used in the quantification of hyaluronic acid synthetase 1 to 3 in accordance with the present invention e.g. by taking advantage of real time PCR:

HAS1:
forward (1072-1090)

(SEQ ID NO: 111)

tgctcagcatgggttatgc
reverse(1115-1134)

(SEQ ID NO: 112)

agggcgtctctgagtagcag
HAS2 (ENST00000303924, NM_005328.2)
Amplikon 86 bp,
primer fwd:

(SEQ ID NO: 113)

ctccgggaccacacagac;
primer rev:

(SEQ ID NO: 114)

tcaggatacatagaaacctctcaca
HAS3 transcript variant 1 (ENST00000306560,
NM 005329.2) Amplikon 111 bp,
primer fwd:

(SEQ ID NO: 115)

cgattcggtggactacatcc;
primer rev:

(SEQ ID NO: 116)

ccgactccccctacttgg

PCR methods for use in the quantification of gene products have been described herein above in detail in relation to CD44 isoforms. These PCR methods can likewise be used in the quantification of hyaluronic acid synthetases (HAS). All corresponding explanations and definitions apply, mutatis mutandis, in this context, with the exception that the primer pairs above are to be used in the quantification of the respective hyaluronic acid synthetase(s).

The following primers and probes were designed to identify and quantify the respective CD44 splice variants:

CD44-v3:
Forward:

(SEQ ID NO: 87)

cctgctaccagtacgtcttc
Reverse:

(SEQ ID NO: 88)

accagagaccaagacacat
Probe:

(SEQ ID NO: 89)

agccaaatgaagaaaatgaagatgaaagagacagac
Cd44-v6:
Forward:

(SEQ ID NO: 90)

gaatccctgctaccatccag
Reverse:

(SEQ ID NO: 91)

gctggagaccaagacac
Probe:

(SEQ ID NO: 92)

aacggaagaaacagctacccagaaggaacag
CD44-v10:
Forward:

(SEQ ID NO: 93)

acagaatccctgctaccaata
Reverse:

(SEQ ID NO: 94)

aggctcaactactttactgg
Probe:

(SEQ ID NO: 95)

tcacaggtggaagaagagacccaaatcat
CD44-v3-10:
Forward:

(SEQ ID NO: 96)

ccctgctaccagtacgtcttcaa
Reverse:

(SEQ ID NO: 97)

gacagacacctcagtttttctgg
Probe (LNA):
cagcaggct
CD44-v6-10:
Forward:

(SEQ ID NO: 98)

gaatccctgctaccatccagg
Reverse:

(SEQ ID NO: 99)

ttggcaacagatggcat
Probe:

(SEQ ID NO: 100)

aacggaagaaacagctacccagaaggaacag
CD44-v8-10:
Forward:

(SEQ ID NO: 101)

tccctgctaccaatatggactc
Reverse:

(SEQ ID NO: 102)

ctgcaaatccaaacacaggtt
Probe (LNA):
gcttcagcc
CD44-s:
Forward:

(SEQ ID NO: 103)

cagaatccctgctaccagaga
Reverse:

(SEQ ID NO: 104)

atggacactcacatggga
Probe:

(SEQ ID NO: 105)

ccagtggggggtcccataccac
CD44-long:
Forward:

(SEQ ID NO: 106)

tctttcaatgacaacgcagcagagtaa
Reverse:

(SEQ ID NO: 107)

tactctgacatcaagcaatagga
Probe:

(SEQ ID NO: 108)

atgaaggcttggaagaagataaagaccatcca
CD44 Pan (measures any CD44 transcript
irrespective of isoform):
Forward:

(SEQ ID NO: 109)

gaatcagatggacactcaca
Reverse:

(SEQ ID NO: 110)

ttccagaatggctgatcatc
Probe (LNA):
aggtggag

The herein above provided methods can comprise a step of administering an anti-CD44 antibody to the patient who is determined to have the CD44s isoform as major isoform. Corresponding pharmaceutical compositions and the like to be used in that context are described herein further below.

The following relates to the therapy of patients/patient group with an anti-CD44 antibody or an antigen binding fragment thereof. These patients have CD44s as major CD44 isoform and therefore respond to treatment with the an anti-CD44 antibody or an antigen binding fragment thereof, as explained herein above in detail. Again, the explanations and definitions provided herein in relation to “tumor cell(s)”, “cancer cell(s)”, “anti-CD44 antibody or antigen binding fragment thereof”, “major CD44 isoform”, “CD44s”, “sample”, “patient”, and the like apply, mutatis mutandis, to the herein below provided “method of treating a patient” and “anti-CD44 antibody or an antigen binding fragment thereof for use in treating a cancer patient”.

The present invention relates to a method of treating a patient, said method comprising selecting a cancer patient, wherein a tumor cell or cancer cell of a sample of said patient is determined to have CD44s as major CD44 isoform and administering to the patient an effective amount of an anti-CD44 antibody or an antigen binding fragment thereof.

The present invention relates to an anti-CD44 antibody or an antigen binding fragment thereof for use in treating a cancer patient, wherein a tumor cell or cancer cell of a sample of said patient has CD44s as major CD44 isoform.

The present invention relates to the use of an anti-CD44 antibody or an antigen binding fragment thereof for the preparation of a pharmaceutical composition for the treatment of a cancer patient, wherein a tumor cell or cancer cell of a sample of said patient has CD44s as major CD44 isoform.

The following relates to the anti-CD44 antibody (or the antigen-binding fragment thereof) to be used in medical intervention of the cancer patients. Reference is made to the more detailed description of the anti-CD44 antibody (or the antigen-binding fragment thereof) herein above which applies in this context, mutatis mutandis.

The anti-CD44 antibody can be a chimeric version of the monoclonal antibody produced by the hybridoma deposited with the ATCC with accession number PTA-4621.

The anti-CD44 antibody can be a humanized version of the monoclonal antibody produced by the hybridoma deposited with the ATCC with accession number PTA-4621.

The anti-CD44 antibody can comprise one or more of a V_{H} CDR1 having the amino acid sequence of SEQ ID NO:3, a V_{H} CDR2 having the amino acid sequence of SEQ ID NO:4, a V_{H} CDR3 having the amino acid sequence of SEQ ID NO:5, a V_{L} CDR1 having the amino acid sequence of SEQ ID NO:6, a V_{L} CDR2 having the amino acid sequence of SEQ ID NO:7 and a V_{L} CDR3 having the amino acid sequence of SEQ ID NO:8. The anti-CD44 antibody can comprise all of a V_{H} CDR1 having the amino acid sequence of SEQ ID NO:3, a V_{H} CDR2 having the amino acid sequence of SEQ ID NO:4, a V_{H} CDR3 having the amino acid sequence of SEQ ID NO:5, a V_{L} CDR1 having the amino acid sequence of SEQ ID NO:6, a V_{L} CDR2 having the amino acid sequence of SEQ ID NO:7 and a V_{L} CDR3 having the amino acid sequence of SEQ ID NO:8.

The anti-CD44 antibody can be a chimeric antibody. The anti-CD44 antibody can comprise a V_{H} domain having the amino acid sequence of SEQ ID NO:1. The anti-CD44 antibody can comprise a V_{L} domain having the amino acid sequence of SEQ ID NO:2. The anti-CD44 antibody can comprise a V_{H} domain having the amino acid sequence of SEQ ID NO:1 and a V_{L} domain having the amino acid sequence of SEQ ID NO:2.

The anti-CD44 antibody can be a humanized antibody. The humanized anti-CD44 antibody can comprise a V_{H} domain having the amino acid sequence of SEQ ID NO:9 or SEQ ID NO: 10. The humanized anti-CD44 antibody can comprise a V_{L} domain having the amino acid sequence of SEQ ID NO:11. The humanized anti-CD44 antibody can comprise a V_{H} domain having the amino acid sequence of SEQ ID NO:9 and a V_{L} domain having the amino acid sequence of SEQ ID NO:11. The humanized anti-CD44 antibody can comprise a V_{H} domain having the amino acid sequence of SEQ ID NO: 10 and a V_{L} domain having the amino acid sequence of SEQ ID NO:11.

The anti-CD44 antibody can compete for binding with the anti-CD44 antibody produced by the hybridoma deposited with the ATCC with Accession number PTA-4621.

The anti-CD44 antibody can interfere with the interaction of CD44 and hyaluronic acid.

The following relates to the determination of a CD44s as major isoform in a sample of the patient. In accordance with the above, a cancer patient is to be selected for therapy with an anti-CD44 antibody (or an antigen-binding fragment thereof) (i.e. the patient is eligible for said therapy), if the CD44s isoform is determined to be the major isoform. Reference is made to the more detailed description of the determination of the CD44s isoform above which applies in this context, mutatis mutandis.

A patient can be selected if the CD44s isoform is determined to be the major isoform. The CD44s isoform is determined to be the major isoform if at least 60% of all CD44 isoform molecules in a sample are molecules of said CD44s isoform. It was shown herein that a threshold of at least 60% of all CD44 isoform molecules is the lowest major isoform level of CD44s in responding models as determined by RNASeq and quantitative Real-_Time PCR (qRT-PCR); see Example 1 and Table 1 The value can be determined either by using results from qRT-PCR or mRNASeq read counts.

Exemplary nucleic acid sequences of the CD44 isoform CD44s are shown in SEQ ID NO: 12 and 14. Exemplary amino acid sequences of the CD44 isoform CD44s are shown in SEQ ID NO: 13 and 15. Corresponding RNA sequences of the CD44 isoform CD44s are shown in SEQ ID NO: 117 and 118.

The amount and/or number of all CD44 isoform molecules in said sample can be assessed by Real Time PCR or Whole Transcriptome Shotgun Sequencing (RNAseq).

The patient may suffer from a hematological or solid cancer, may be suspected of suffering from a hematological or solid cancer, or may be prone to suffering from a hematological or solid cancer. The cancer to be treated with the anti-CD44 antibody (or an antigen-binding fragment thereof) can be (and is preferably) characterized by/associated with the presence of the CD44s isoform. The cancer to be treated with the anti-CD44 antibody (or an antigen-binding fragment thereof) can be (and is preferably) suspected of being characterized by/associated with the presence of the CD44s isoform. Exemplary cancers are described below.

In one aspect of the present invention, the hematological cancer is acute myeloid leukemia (AML), chronic myeloid leukemia (CML), chronic lymphoid leukemia (CLL), multiple myeloma (MM), or myelodysplastic syndrome (MDS). The hematological cancer can be acute myeloid leukemia (AML).

In one aspect of the present invention the solid cancer is head or neck cancer, melanoma, lung adenocarcinoma, renal cancer, kidney cancer, mesothelioma, lung squeamous cell carcinoma, breast cancer, liver hepatocellular carcinoma, glioblastoma, gastric cancer, ovarian cancer, bladder cancer, thymus carcinoma, colorectal carcinoma or prostate cancer. Colorectal carcinoma includes colorectal adenocarcinoma. Colorectal adenocarcinoma includes two sub-types, colon adenocarcinoma and rectum adenocarcinoma. Accordingly, also colon adenocarcinoma or rectum adenocarcinoma can be treated in accordance with the present invention. Of the solid cancers, head or neck cancer (like head and neck squameous cell carcinoma) is preferred herein.

In one aspect of the present invention, the solid cancer can be head and neck squameous cell carcinoma, melanoma, lung adenocarcinoma, kidney renal clear cell carcinoma, kidney renal papillary cell carcinoma, lung squameous cell carcinoma, breast invasive carcinoma, liver hepatocellular carcinoma, stomach adenocarcinoma, ovarian serous cystadenocarcinoma, bladder cancer, prostate adenocarcinoma, colorectal adenocarcinoma, colon adenocarcinoma, rectum adenocarcinoma, thymus carcinoma, thyroid carcinoma, and uterine corpus endometrioid carcinoma. Colorectal adenocarcinoma includes two sub-types, colon adenocarcinoma and rectum adenocarcinoma. Accordingly, also colon adenocarcinoma or rectum adenocarcinoma can be treated in accordance with the present invention.

The tumor cell or cancer cell of a sample of said patient can be determined to have (or a tumor cell or cancer cell of a sample of said patient has) an increased level of hyaluronic acid and/or an increased level of one or more hyaluronic acid synthetases in comparison with a control.

The level of hyaluronic acid and/or said level of one or more hyaluronic acid synthetases is at least 2.5-fold, preferably at least 5-fold increased in comparison to the control. The level of said one or more hyaluronic acid synthetases can be the expression level, for example, the mRNA expression level or the protein expression level. The mRNA expression level can be assessed by in situ hybridization, micro-arrays, or RealTime PCR. The protein expression level can assessed by immunoassay, gel- or blot-based methods, IHC, mass spectrometry, flow cytometry, or FACS.

Anti-CD44 antibodies or antigen-binding fragments thereof that are useful in the practice of the present invention can be used as a composition for treating cancer. The terms “treatment”, “treating” and the like are used herein to generally mean obtaining a desired pharmacological and/or physiological effect. The effect may be prophylactic in terms of completely or partially preventing cancer or symptom thereof and/or may be therapeutic in terms of partially or completely curing cancer and/or adverse effect attributed to the cancer. The term “treatment” as used herein covers any treatment of cancer in a subject and includes: (a) preventing a cancer from occurring in a subject which may be predisposed to the disease; (b) inhibiting the cancer, i.e. arresting its development; or (c) relieving the cancer, i.e. causing regression of the disease. A “patient” or “subject” or “individual” for the purposes of the present invention includes both humans and other animals, particularly mammals. Thus, the methods are applicable to both human therapy and veterinary applications. Most preferably, the “patient” or “subject” or “individual” is human.

The compositions comprising anti-CD44 antibodies or antigen-binding fragments thereof for use in the herein provided therapeutic methods have low-toxicity and can be administered as they are in the form of liquid preparations, or as pharmaceutical compositions of suitable preparations to human or mammals parenterally (e.g., intravascularly, intraperitoneally, subcutaneously, etc.). Antibodies useful in the practice of the present invention may be administered by themselves, or may be administered as appropriate compositions. A composition used for such administration may contain a pharmacologically acceptable carrier with the antibody or its salt, a diluent or excipient. Such a composition is provided in the form of pharmaceutical preparations suitable for parenteral administration.

Examples of the composition for parenteral administration are injectable preparations, suppositories, etc. The injectable preparations may include dosage forms such as intravenous, subcutaneous, intracutaneous and intramuscular injections, drip infusions, intraarticular injections, etc. A preferred administration route is intravenous administration. These injectable preparations may be prepared by methods publicly known. For example, the injectable preparations may be prepared by dissolving, suspending or emulsifying the antibody of the present invention or its salt in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant (e.g., polysorbate 80, HCO-50 (polyoxyethylene (50 mols) adduct of hydrogenated castor oil)), etc. As the oily medium, there are employed, e.g., sesame oil, soybean oil, etc., which may be used in combination with a solubilizing agent such as benzyl benzoate, benzyl alcohol, etc. The injection thus prepared is usually filled in an appropriate ampoule. The suppository used for rectal administration may be prepared by blending the antibody of the present invention or its salt with conventional bases for suppositories.

Advantageously, the compositions for parenteral use described above are prepared into pharmaceutical preparations with a unit dose suited to fit a dose of the active ingredients. Such unit dose preparations include, for example, tablets, pills, capsules, injections (ampoules), suppositories, etc.

The dose of the aforesaid prophylactic/therapeutic agent comprising the antibody according to the methods of the present invention may vary depending upon subject to be administered, target disease, conditions, route of administration, etc. For example, when used for the purpose of treating/preventing cancer in an adult, it is advantageous to administer the antibody of the present invention intravenously in a dose of about 0.0001 mg/kg to 200 mg/kg or 0.0001 mg/kg to 100 mg/kg of the patient's body weight. In other aspects, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention or fragments thereof may be reduced by enhancing uptake and tissue penetration of the antibodies by modifications such as, for example, lipidation. In other aspects, the antibodies of the invention are used in combination with other therapeutic compositions and the dosage administered to a subject are lower than when said antibodies are used as a single agent therapy.

The dosage amounts and frequencies of administration provided herein are encompassed by the terms therapeutically effective and prophylactically effective. The dosage and frequency further will typically vary according to factors specific for each patient depending on the specific therapeutic or prophylactic agents administered, the severity, the route of administration, as well as age, body weight, response, and the past medical history of the patient. Suitable regimens can be selected by one skilled in the art by considering such factors and by following, for example, dosages reported in the literature and recommended in the Physician's Desk Reference (56.sup.th ed., 2002). The daily dosage of the antibody, fragment or composition of the invention can be administered as a single bolus dose or divided into multiple doses to be delivered over a 24 hour period. Alternatively, the total daily dosage may be administered over an extended period of time via, e.g., an infusion, such that the total dosage is administered over 12 hours, 6 hours, 4 hours, 2 hours, 1.5 hours, 1.0 hours. 45 minutes, 30 minutes, 25 minutes, 20 minutes, 15 minutes, 10 minutes, 9 minutes, 8 minutes, 7 minutes, 6 minutes, 5 minutes, 4 minutes, 3 minutes, 2 minutes or 1 minute. When the condition is especially severe, the dose may be increased according to the condition.

The antibody for use according to the methods of the present invention may be administered as it stands or in the form of an appropriate composition. The composition used for the administration may contain a pharmacologically acceptable carrier with the aforesaid antibody or its salts, a diluent or excipient. Such a composition is provided in the form of pharmaceutical preparations suitable for parenteral administration (e.g., intravascular injection, subcutaneous injection, etc.). Each composition described above may further contain other active ingredients. Furthermore, the antibody of the present invention may be used in combination with other drugs, for example, alkylating agents (e.g., cyclophosphamide, ifosfamide, etc.), metabolic antagonists (e.g., methotrexate, 5-fluorouracil, etc.), anti-tumor antibiotics (e.g., mitomycin, adriamycin, etc.), plant-derived anti-tumor agents (e.g., vincristine, vindesine, Taxol, etc.), cisplatin, carboplatin, etoposide, irinotecan, etc. The antibody of the present invention and the drugs described above may be administered simultaneously or at staggered times to the patient. Methods of administering a humanized antibody of the invention include, but are not limited to, parenteral administration (e.g., intradermal, intramuscular, intraperitoneal, intravenous and subcutaneous), epidural, and mucosal (e.g., intranasal and oral routes). In a specific embodiment, the antibodies of the invention are administered intramuscularly, intravenously, or subcutaneously. The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent.

The chemotherapeutic agent/other antibody regimens utilized include any regimen believed to be optimally suitable for the treatment of the patient's condition. Different malignancies can require use of specific anti-tumor antibodies and specific chemotherapeutic agents, which will be determined on a patient to patient basis. In a preferred embodiment of the invention, chemotherapy is administered concurrently with or, more preferably, subsequent to antibody therapy. It should be emphasized, however, that the present invention is not limited to any particular method or route of administration.

As described above, it may not be necessary to stratify certain cancer patients, because the majority of these patients has CD44s as major isoform, as demonstrated in the appended examples and shown in FIG. 4. For example, more than 80% of samples of AML patients, kidney renal clear cell carcinoma patients, melanoma patients, kidney renal papillary cell carcinoma patients, liver hepatocellular carcinoma patients, and lung adenocarcinoma patients have CD44s as major isoform. More than about 90% of samples of AML patients, kidney renal clear cell carcinoma patients and melanoma patients have CD44s as major isoform.

The present invention relates to a method of treating a patient suffering from, suspected to suffer from or being prone to suffer from a hematological cancer, kidney renal clear cell carcinoma or melanoma, comprising administering to the patient an effective amount of an anti-CD44 antibody or an antigen-binding fragment thereof. Preferably, the patient is a human.

The present invention relates to an anti-CD44 antibody or an antigen-binding fragment thereof for use in treating a hematological cancer, kidney renal clear cell carcinoma or melanoma.

In one aspect of the present invention, the hematological cancer is chronic myeloid leukemia (CML), chronic lymphoid leukemia (CLL), multiple myeloma (MM), or myelodysplastic syndrome (MDS).

In one aspect of the present invention, the solid cancer is kidney renal clear cell carcinoma, melanoma, kidney renal papillary cell carcinoma, liver hepatocellular carcinoma, or lung adenocarcinoma.

The anti-CD44 antibody (or an antigen-binding fragment thereof) and pharmaceutical compositions comprising the antibody have been described herein above in great detail. These explanations apply, mutatis mutandis, in this context.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of” Thus, the terms “comprising”/“including”/“having” mean that any further component (or likewise features, integers, steps and the like) can be present.

The term “consisting of” means that no further component (or likewise features, integers, steps and the like) can be present.

The term “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

Thus, the term “consisting essentially of” means that specific further components (or likewise features, integers, steps and the like) can be present, namely those not materially affecting the essential characteristics of the composition, device or method. In other words, the term “consisting essentially of” (which can be interchangeably used herein with the term “comprising substantially”), allows the presence of other components in the composition, device or method in addition to the mandatory components (or likewise features, integers, steps and the like), provided that the essential characteristics of the device or method are not materially affected by the presence of other components.

The term “method” refers to manners, means, techniques and procedures for accomplishing a given task including, but not limited to, those manners, means, techniques and procedures either known to, or readily developed from known manners, means, techniques and procedures by practitioners of the chemical, biological and biophysical arts.

As used herein the term “about” may refer to ±10%.

The present invention relates to the following items:
1. A method of determining whether a tumor cell or a cancer cell is responsive to an anti-CD44 antibody or to an antigen binding fragment thereof, said method comprising determining the major CD44 isoform in a sample of a patient, wherein if said major CD44 isoform is CD44s, said tumor cell or cancer cell is responsive to said anti-CD44 antibody or to an antigen binding fragment thereof
2. The method of item 1, wherein said anti-CD44 antibody is a chimeric version of the monoclonal antibody produced by the hybridoma deposited with the ATCC with accession number PTA-4621.
3. The method of item 1, wherein said anti-CD44 antibody is a humanized version of the monoclonal antibody produced by the hybridoma deposited with the ATCC with accession number PTA-4621.
4. The method of any one of items 1 to 3, wherein said anti-CD44 antibody comprises one or more of a V_{H} CDR1 having the amino acid sequence of SEQ ID NO:3, a V_{H} CDR2 having the amino acid sequence of SEQ ID NO:4, a V_{H} CDR3 having the amino acid sequence of SEQ ID NO:5, a V_{L} CDR1 having the amino acid sequence of SEQ ID NO:6, a V_{L} CDR2 having the amino acid sequence of SEQ ID NO:7 and a V_{L} CDR3 having the amino acid sequence of SEQ ID NO:8.
5. The method of item 1 or 4, wherein said anti-CD44 antibody is a chimeric antibody.
6. The method of any one of items 1, 2, 4 and 5, wherein said anti-CD44 antibody comprises a V_{H} domain having the amino acid sequence of SEQ ID NO:1.
7. The method of any one of items 1, 2, and 4 to 6, wherein said anti-CD44 antibody comprises a V_{L} domain having the amino acid sequence of SEQ ID NO:2.
8. The method of item 1 or 4, wherein said anti-CD44 antibody is a humanized antibody.
9. The method of item 8, wherein said humanized anti-CD44 antibody comprises a V_{H} domain having the amino acid sequence of SEQ ID NO:9 or SEQ ID NO: 10.
10. The method of item 8 or 9, wherein said humanized anti-CD44 antibody comprises a V_{L} domain having the amino acid sequence of SEQ ID NO:11.
11. The method of any one of items 1 to 10, wherein said anti-CD44 antibody competes for binding with the anti-CD44 antibody produced by the hybridoma deposited with the ATCC with Accession number PTA-4621.
12. The method of any one of items 1 to 11, wherein said anti-CD44 antibody interferes with the interaction of CD44 and hyaluronic acid.
13. The method of any one of items 1 to 12, further comprising determining the amount and/or number of all CD44 isoform molecules in said sample.
14. The method of any one of items 1 to 13, wherein a CD44 isoform is determined to be the major isoform, if at least 60% of all CD44 isoform molecules in said sample are molecules of said CD44 isoform.
15. The method of item 13 or 14, wherein the amount and/or number of all CD44 isoform molecules in said sample is assessed by Real Time PCR or Whole Transcriptome Shotgun Sequencing (RNAseq).
16. The method of any one of items 1 to 15, wherein the nucleic acid sequence encoding said CD44 isoform CD44s is shown in SEQ ID NO: 12 or 14.
17. The method of any one of items 1 to 15, wherein the amino acid sequence of said CD44 isoform CD44s is shown in SEQ ID NO: 13 or 15.
18. The method of any one of items 1 to 16, wherein said patient is suffering from a hematological or solid cancer, is suspected of suffering from a hematological or solid cancer, or is prone to suffering from a hematological or solid cancer.
19. The method of item 18, wherein said hematological cancer is selected from the group consisting of acute myeloid leukemia (AML), chronic myeloid leukemia (CML), chronic lymphoid leukemia (CLL), multiple myeloma (MM), and myelodysplastic syndrome (MDS), and wherein said solid cancer is selected from the group consisting of melanoma, lung adenocarcinoma, renal cancer, kidney cancer, mesothelioma, lung squeamous cell carcinoma, breast cancer, liver hepatocellular carcinoma, glioblastoma, gastric cancer, ovarian cancer, bladder cancer, prostate cancer, thymus carcinoma, colorectal carcinoma, head and neck cancer.
20. The method of any one of items 1 to 19, further comprising determining the level of hyaluronic acid and/or the level of one or more hyaluronic acid synthetases 1 to 3.
21. The method of item 20, wherein if the level of hyaluronic acid is increased in comparison with a control and/or if the level of one or more hyaluronic acid synthetases is increased in comparison with a control, said tumor cell or cancer cell is responsive to said anti-CD44 antibody.
22. The method of item 21, wherein said level of hyaluronic acid and/or said level of one or more hyaluronic acid synthetases is at least 2.5-fold, preferably at least 5-fold increased in comparison to the control.
23. The method of any one of items 20 to 22, wherein said level of said one or more hyaluronic acid synthetases is the expression level.
24. The method of item 23, wherein said expression level is the mRNA expression level.
25. The method of item 24, wherein the mRNA expression level is assessed by in situ hybridization, micro-arrays, or RealTime PCR.
26. The method of item 23, wherein said expression level is the protein expression level.
27. The method of item 26, wherein said protein expression level is assessed by immunoassay, gel- or blot-based methods, IHC, mass spectrometry, flow cytometry, or FACS.
28. The method of any one of items 1 to 27, wherein an anti-CD44 antibody is to be administered to the patient who is determined to have the CD44s isoform as major isoform.
29. A method of treating a patient, said method comprising selecting a cancer patient, wherein a tumor cell or cancer cell of a sample of said patient is determined to have CD44s as major CD44 isoform and administering to the patient an effective amount of an anti-CD44 antibody or an antigen binding fragment thereof.
30. An anti-CD44 antibody or an antigen binding fragment thereof for use in treating a cancer patient, wherein a tumor cell or cancer cell of a sample of said patient has CD44s as major CD44 isoform.
31. The method of item 29, or the anti-CD44 antibody for use in treating a cancer patient of item 30, wherein said anti-CD44 antibody is a chimeric version of the monoclonal antibody produced by the hybridoma deposited with the ATCC with accession number PTA-4621.
32. The method of item 29, or the anti-CD44 antibody for use in treating a cancer patient of item 30, wherein said anti-CD44 antibody is a humanized version of the monoclonal antibody produced by the hybridoma deposited with the ATCC with accession number PTA-4621.
33. The method of any one of items 29, 31 and 33; or the anti-CD44 antibody for use in treating a cancer patient of any one of items 30 to 32, wherein said anti-CD44 antibody comprises one or more of a V_{H} CDR1 having the amino acid sequence of SEQ ID NO:3, a V_{H} CDR2 having the amino acid sequence of SEQ ID NO:4, a V_{H} CDR3 having the amino acid sequence of SEQ ID NO:5, a V_{L} CDR1 having the amino acid sequence of SEQ ID NO:6, a V_{L} CDR2 having the amino acid sequence of SEQ ID NO:7 and a V_{L} CDR3 having the amino acid sequence of SEQ ID NO:8.
34. The method of item 29 or 33; or the anti-CD44 antibody for use in treating a cancer patient of items 30 or 33, wherein said anti-CD44 antibody is a chimeric antibody.
35. The method of any one of items 29, 31, 33 and 34; or the anti-CD44 antibody for use in treating a cancer patient of any one of items 30, 31, 33 and 34, wherein said anti-CD44 antibody comprises a V_{H} domain having the amino acid sequence of SEQ ID NO:1.
36. The method of any one of items 29, 31, and 33 to 35; or the anti-CD44 antibody for use in treating a cancer patient of any one of items 30, 31, and 33 to 35, wherein said anti-CD44 antibody comprises a V_{L} domain having the amino acid sequence of SEQ ID NO:2.
37. The method of item 29 or 33; or the anti-CD44 antibody for use in treating a cancer patient of item 30 or 33, wherein said anti-CD44 antibody is a humanized antibody.
38. The method of item 37, or the anti-CD44 antibody for use in treating a cancer patient of item 37, wherein said humanized anti-CD44 antibody comprises a V_{H} domain having the amino acid sequence of SEQ ID NO:9 or SEQ ID NO: 10.
39. The method of items 37 or 38; or the anti-CD44 antibody for use in treating a cancer patient of items 37 or 38, wherein said humanized anti-CD44 antibody comprises a V_{L} domain having the amino acid sequence of SEQ ID NO:11.
40. The method of any one of items 29, and 31 to 39; or the anti-CD44 antibody for use in treating a cancer patient of any one of items 30 to 39, wherein said anti-CD44 antibody competes for binding with the anti-CD44 antibody produced by the hybridoma deposited with the ATCC with Accession number PTA-4621.
41. The method of any one of items 29, and 31 to 40; or the anti-CD44 antibody for use in treating a cancer patient of any one of items 30 to 40, wherein said anti-CD44 antibody interferes with the interaction of CD44 and hyaluronic acid.
42. The method of any one of items 29, and 31 to 41; or the anti-CD44 antibody for use in treating a cancer patient of any one of items 30 to 41, wherein the CD44s isoform is determined to be the major isoform, if at least 60% of all CD44 isoform molecules in said sample are molecules of said CD44s isoform.
43. The method of item 42; or the anti-CD44 antibody for use in treating a cancer patient of item 42, wherein the amount and/or number of all CD44 isoform molecules in said sample is assessed by Real Time PCR or Whole Transcriptome Shotgun Sequencing (RNAseq).
44. The method of any one of items 29, and 31 to 43; or the anti-CD44 antibody for use in treating a cancer patient of any one of items 30 to 43, wherein the nucleic acid sequence encoding said CD44 isoform CD44s is shown in SEQ ID NO: 12 or 14.
45. The method of any one of items 29, and 31 to 43; or the anti-CD44 antibody for use in treating a cancer patient of any one of items 30 to 43, wherein the amino acid sequence of said CD44 isoform CD44s is shown in SEQ ID NO: 13 or 15.
46. The method of any one of items 29, and 31 to 45; or the anti-CD44 antibody for use in treating a cancer patient of any one of items 30 to 45, wherein said patient is suffering from a hematological or solid cancer, is suspected of suffering from a hematological or solid cancer, or is prone to suffering from a hematological or solid cancer.
47. The method of item 46; or the anti-CD44 antibody for use in treating a cancer patient of item 46, wherein said hematological cancer is selected from the group consisting of acute myeloid leukemia (AML), chronic myeloid leukemia (CML), chronic lymphoid leukemia (CLL), multiple myeloma (MM), and myelodysplastic syndrome (MDS), and wherein said solid cancer is selected from the group consisting of melanoma, lung adenocarcinoma, renal cancer, kidney cancer, mesothelioma, lung squeamous cell carcinoma, breast cancer, liver hepatocellular carcinoma, glioblastoma, gastric cancer, ovarian cancer, bladder cancer, prostate cancer, thymus carcinoma, colorectal carcinoma, head and neck cancer.
48. The method of any one of items 29, and 31 to 47; or the anti-CD44 antibody for use in treating a cancer patient of any one of items 30 to 47, wherein a tumor cell or cancer cell of a sample of said patient is determined to have (or wherein a tumor cell or cancer cell of a sample of said patient has) an increased level of hyaluronic acid and/or an increased level of one or more hyaluronic acid synthetases in comparison with a control.
49. The method of items 48; or the anti-CD44 antibody for use in treating a cancer patient of item 48, wherein said level of hyaluronic acid and/or said level of one or more hyaluronic acid synthetases is at least 2.5-fold, preferably at least 5-fold increased in comparison to the control.

50. The method of item 48 or 49; or the anti-CD44 antibody for use in treating a cancer patient of item 48 or 49, wherein said level of said one or more hyaluronic acid synthetases is the expression level.
51. The method of item 50; or the anti-CD44 antibody for use in treating a cancer patient of item 50, wherein said expression level is the mRNA expression level.
52. The method of item 51; or the anti-CD44 antibody for use in treating a cancer patient of item 51, wherein the mRNA expression level is assessed by in situ hybridization, micro-arrays, or RealTime PCR.
53. The method of item 50; or the anti-CD44 antibody for use in treating a cancer patient of item 50, wherein said expression level is the protein expression level.
54. The method of item 53; or the anti-CD44 antibody for use in treating a cancer patient of item 53, wherein said protein expression level is assessed by immunoassay, gel- or blot-based methods, IHC, mass spectrometry, flow cytometry, or FACS.
55. A method of treating a patient suffering from, suspected to suffer from or being prone to suffer from a hematological cancer, kidney renal clear cell carcinoma or melanoma, comprising administering to the patient an effective amount of an anti-CD44 antibody.
56. An anti-CD44 antibody for use in treating a hematological cancer, kidney renal clear cell carcinoma or melanoma.
57. The method of item 55, or the anti-CD44 antibody for use in treating a hematological cancer of item 56, wherein said hematological cancer is selected from the group consisting of chronic myeloid leukemia (CML), chronic lymphoid leukemia (CLL), multiple myeloma (MM), and myelodysplastic syndrome (MDS).

Furthermore, the present invention relates to the following items:
1. A method of determining whether a tumor cell or a cancer cell is responsive to an anti-CD44 antibody or to an antigen binding fragment thereof, said method comprising determining the major CD44 isoform in a sample of a patient, wherein if said major CD44 isoform is CD44s, said tumor cell or cancer cell is responsive to said anti-CD44 antibody or to an antigen binding fragment thereof
2. The method of item 1, wherein said anti-CD44 antibody comprises one or more of a V_{H} CDR1 having the amino acid sequence of SEQ ID NO:3, a V_{H} CDR2 having the amino acid sequence of SEQ ID NO:4, a V_{H} CDR3 having the amino acid sequence of SEQ ID NO:5, a V_{L} CDR1 having the amino acid sequence of SEQ ID NO:6, a V_{L} CDR2 having the amino acid sequence of SEQ ID NO:7 and a V_{L} CDR3 having the amino acid sequence of SEQ ID NO:8.
3. The method of item 1 or 2, wherein said antibody is a humanized anti-CD44 antibody that comprises a V_{H} domain having the amino acid sequence of SEQ ID NO:9 or SEQ ID NO: 10 and wherein said humanized anti-CD44 antibody comprises a V_{L} domain having the amino acid sequence of SEQ ID NO:11.
4. The method of any one of items 1 to 3, further comprising determining the amount and/or number of all CD44 isoform molecules in said sample.
5. The method of any one of items 1 to 4, wherein a CD44 isoform is determined to be the major isoform, if at least 60% of all CD44 isoform molecules in said sample are molecules of said CD44 isoform.
6. The method of item 4 or 5, wherein the amount and/or number of all CD44 isoform molecules in said sample is assessed by Real Time PCR or Whole Transcriptome Shotgun Sequencing (RNAseq).
7. The method of any one of items 1 to 6, wherein the nucleic acid sequence of said CD44 isoform CD44s is shown in SEQ ID NO: 12 or 14 and/or wherein the amino acid sequence of said CD44 isoform CD44s is shown in SEQ ID NO: 13 or 15.
8. The method of any one of items 1 to 7, wherein said patient is suffering from a hematological or solid cancer, is suspected of suffering from a hematological or solid cancer, or is prone to suffering from a hematological or solid cancer.
9. The method of item 8, wherein said hematological cancer is selected from the group consisting of acute myeloid leukemia (AML), chronic myeloid leukemia (CML), chronic lymphoid leukemia (CLL), multiple myeloma (MM), and myelodysplastic syndrome (MDS), and wherein said solid cancer is selected from the group consisting of melanoma, lung adenocarcinoma, renal cancer, kidney cancer, mesothelioma, lung squeamous cell carcinoma, breast cancer, liver hepatocellular carcinoma, glioblastoma, gastric cancer, ovarian cancer, bladder cancer, prostate cancer, thymus carcinoma, colorectal carcinoma, head and neck cancer.

10. An anti-CD44 antibody or an antigen binding fragment thereof for use in treating a cancer patient, wherein a tumor cell or cancer cell of a sample of said patient has CD44s as major CD44 isoform.
11. The anti-CD44 antibody for use in treating a cancer patient of item 10, wherein said anti-CD44 antibody comprises one or more of a V_{H} CDR1 having the amino acid sequence of SEQ ID NO:3, a V_{H} CDR2 having the amino acid sequence of SEQ ID NO:4, a V_{H} CDR3 having the amino acid sequence of SEQ ID NO:5, a V_{L} CDR1 having the amino acid sequence of SEQ ID NO:6, a V_{L} CDR2 having the amino acid sequence of SEQ ID NO:7 and a V_{L} CDR3 having the amino acid sequence of SEQ ID NO:8.
12. The anti-CD44 antibody for use in treating a cancer patient of item 10 or 11, wherein said anti-CD44 antibody is a humanized anti-CD44 antibody that comprises a V_{H} domain having the amino acid sequence of SEQ ID NO:9 or SEQ ID NO: 10 and wherein said humanized anti-CD44 antibody comprises a V_{L} domain having the amino acid sequence of SEQ ID NO:11.
13. The anti-CD44 antibody for use in treating a cancer patient of any one of items 10 to 12, wherein the CD44s isoform is determined to be the major isoform, if at least 60% of all CD44 isoform molecules in said sample are molecules of said CD44s isoform.
14. The anti-CD44 antibody for use in treating a cancer patient of item 13, wherein the amount and/or number of all CD44 isoform molecules in said sample is assessed by Real Time PCR or Whole Transcriptome Shotgun Sequencing (RNAseq).
15. The anti-CD44 antibody for use in treating a cancer patient of any one of items 10 to 14, wherein the nucleic acid sequence of said CD44 isoform CD44s is encoded by SEQ ID NO: 12 or 14 and/or wherein the amino acid sequence of said CD44 isoform CD44s is shown in SEQ ID NO: 13 or 15.
16. The anti-CD44 antibody for use in treating a cancer patient of any one of items 10 to 15, wherein said patient is suffering from a hematological or solid cancer, is suspected of suffering from a hematological or solid cancer, or is prone to suffering from a hematological or solid cancer.
17. The anti-CD44 antibody for use in treating a cancer patient of item 16, wherein said hematological cancer is selected from the group consisting of acute myeloid leukemia (AML), chronic myeloid leukemia (CML), chronic lymphoid leukemia (CLL), multiple myeloma (MM), and myelodysplastic syndrome (MDS), and wherein said solid cancer is selected from the group consisting of melanoma, lung adenocarcinoma, renal cancer, kidney cancer, mesothelioma, lung squeamous cell carcinoma, breast cancer, liver hepatocellular carcinoma, glioblastoma, gastric cancer, ovarian cancer, bladder cancer, prostate cancer, thymus carcinoma, colorectal carcinoma, head and neck cancer.
18. An anti-CD44 antibody for use in treating a hematological cancer, kidney renal clear cell carcinoma or melanoma, wherein said hematological cancer is selected from the group consisting of chronic myeloid leukemia (CML), chronic lymphoid leukemia (CLL), multiple myeloma (MM), and myelodysplastic syndrome (MDS).

It is envisaged that any of the above items (and combinations thereof) can be combined with any of the items/aspects provided herein in accordance with the present invention.

The present invention is further described by reference to the following illustrative non-limiting figures and examples. Unless otherwise indicated, established methods of recombinant gene technology were used as described, for example, in Sambrook, Russell “Molecular Cloning, A Laboratory Manual”, Cold Spring Harbor Laboratory, N.Y. (2001)) which is incorporated herein by reference in its entirety.

### EXAMPLES

### Example 1

### RG7356 treated Xenograft Models and CD44 Isoform Status

### Material and Methods

If not explained otherwise, the material and methods which are described in the following have been used in the examples.

### Mice and Cell Lines

Immunodeficient SCID/bg mice were purchased from Charles River (Sulzfeld, Germany). Animals used in experiments were between 6 and 7 weeks of age. All experiments were conducted in accordance with local governmental regulations (Regierung von Oberbayern) and Roche internal guidelines. SK-HEP-1 cell line was obtained from American Type Culture Collection (ATCC). SK-HEP-1 were grown in RPMI1640 supplemented with 2 mM L-Glutamine and 20% heat-inactivated FBS. All media and supplements were purchased from PAN Biotech (Aidenbach, Germany). In vitro passage number 3 were used for inoculation. Mice were inoculated s.c. in the flank with 5×106 SK-HEP-1 cells.

### RNASeq Data Generation

In order to analyze the isoform status of each of the 39 cancer cell lines we used RNASeq data generated using the Illumina HiSeq2000 sequencing system. RNA and sequencing libraries were prepared according to the manufacturer's protocols (using the Illumina TruSeq Standard protocol) and an average number of 30 Mio. paired-end reads of 50 bp length (for each read of a pair) were generated per sample.

### RNASeq Data Analysis

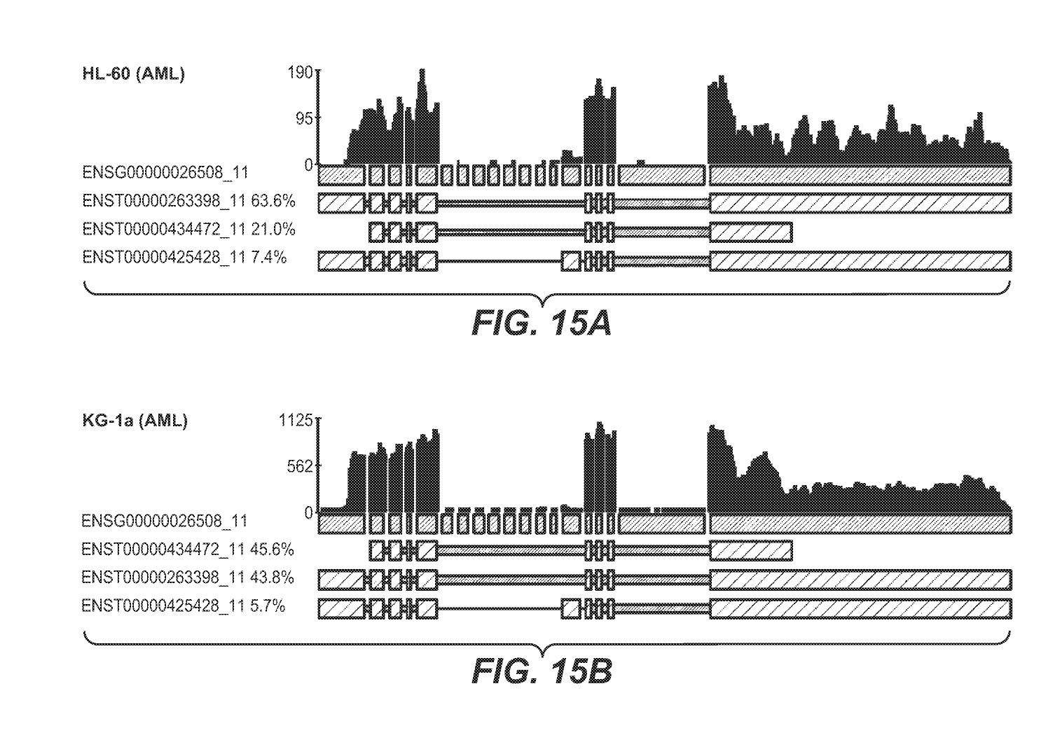

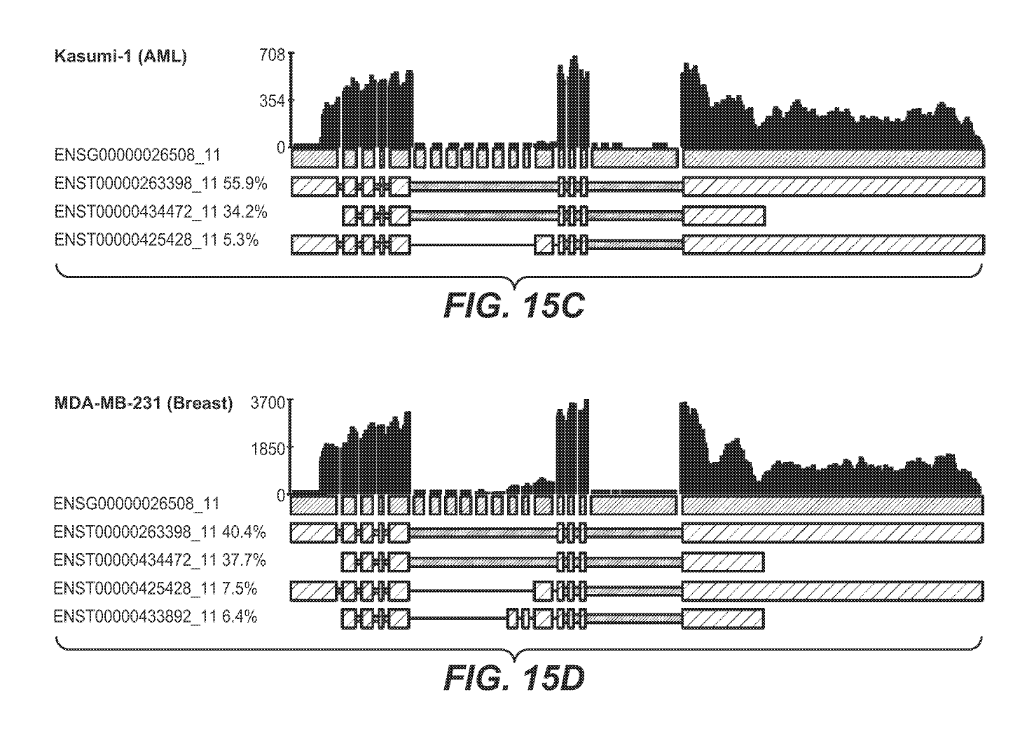

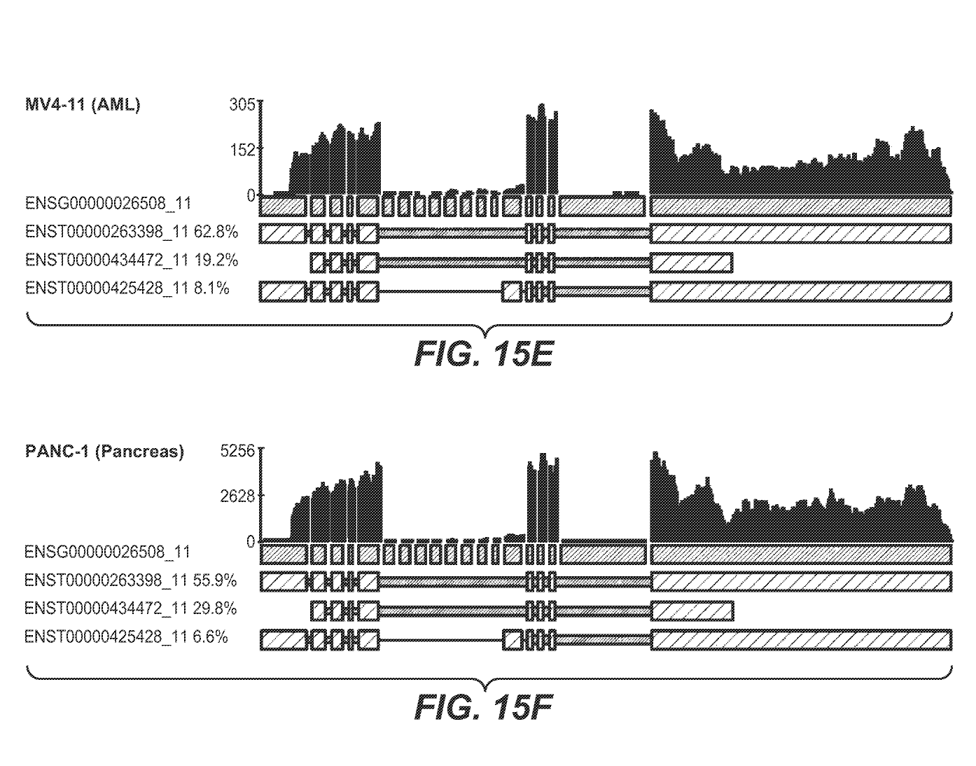

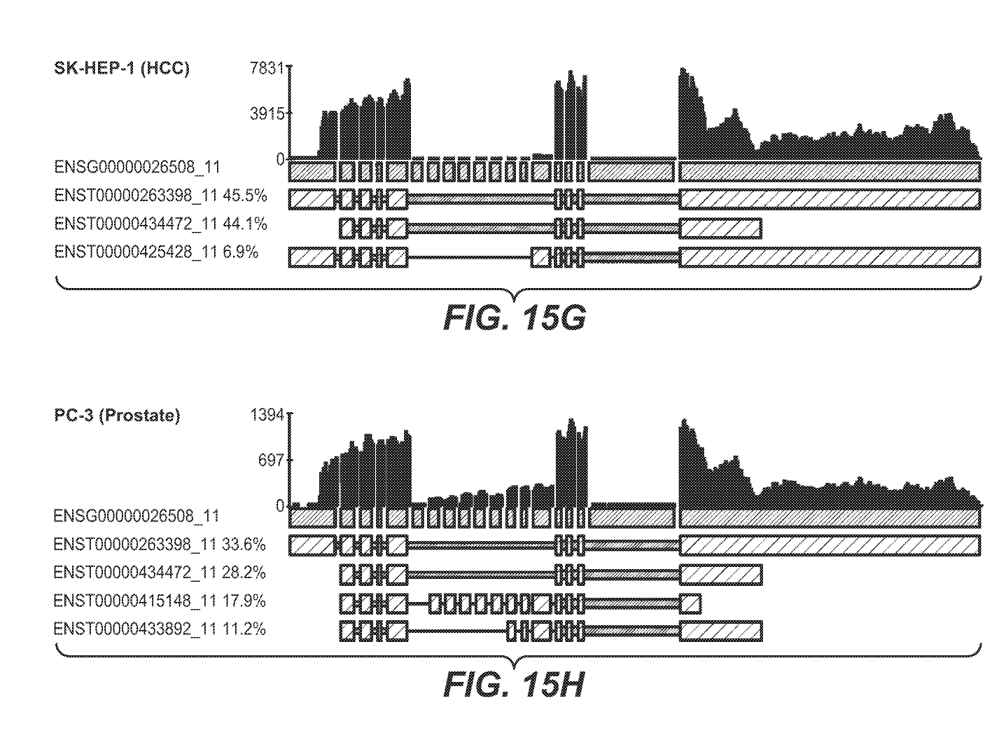

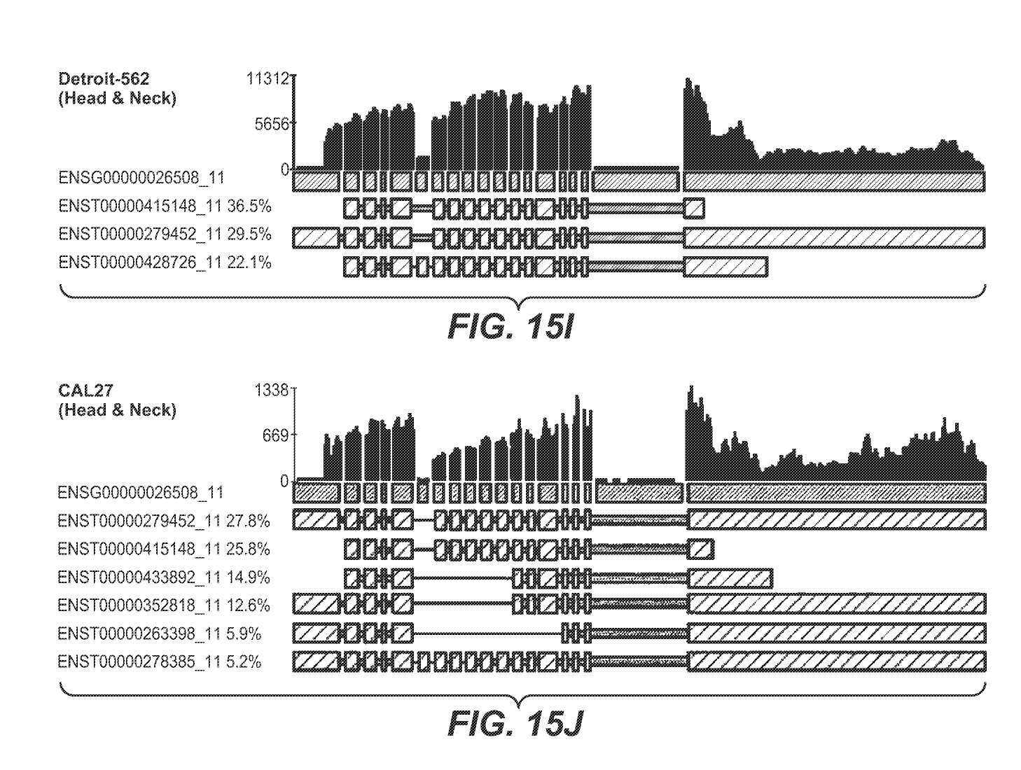

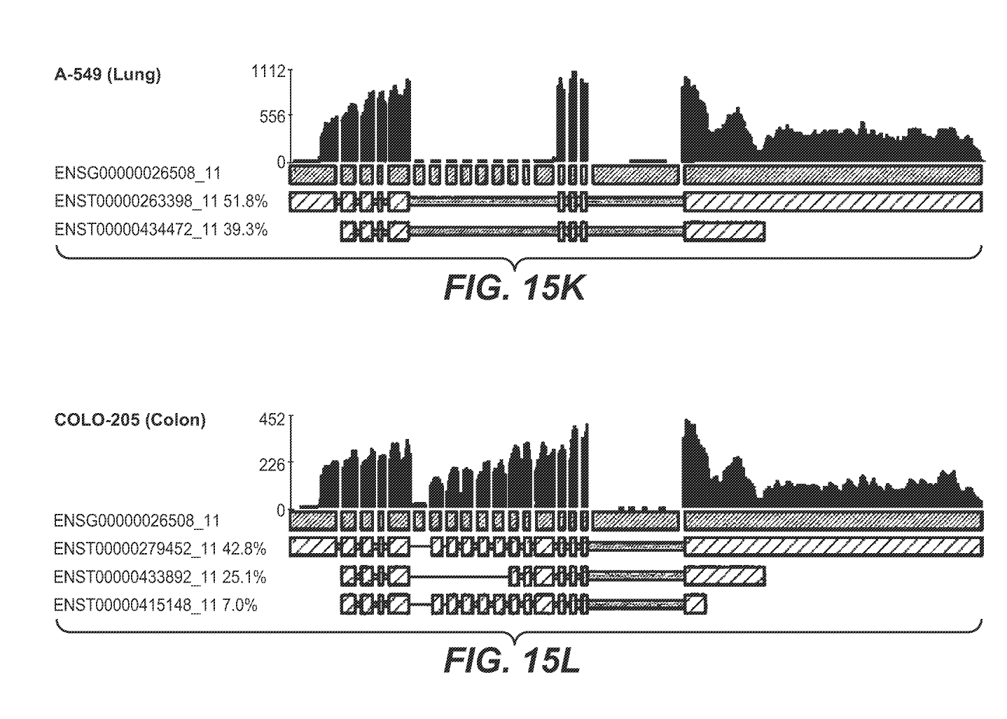

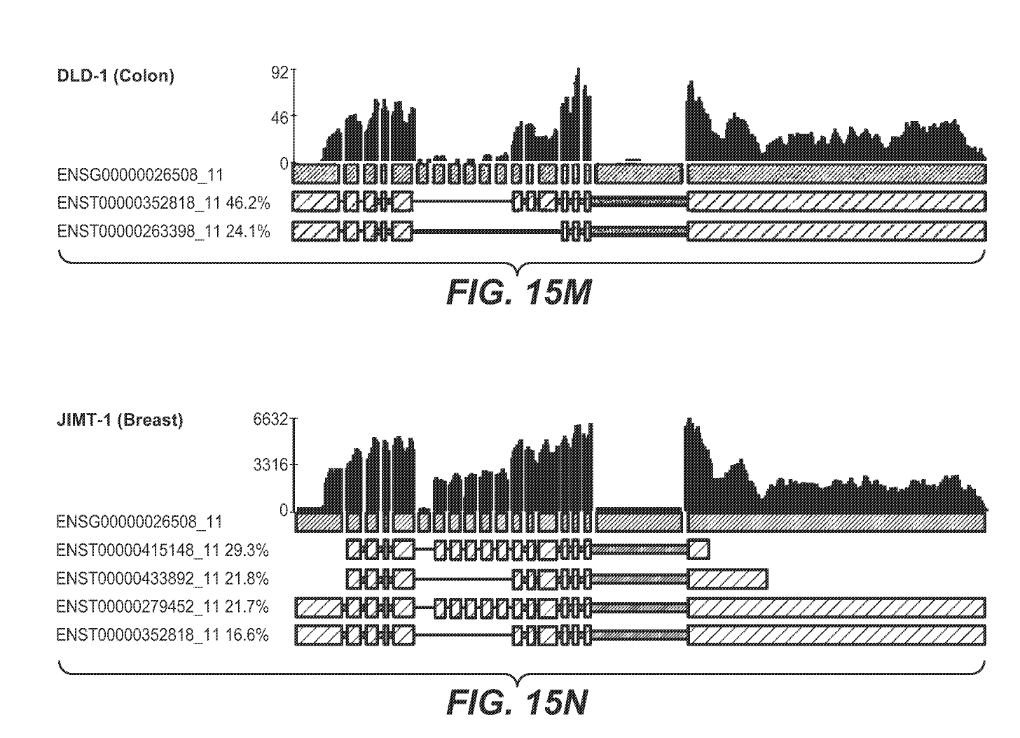

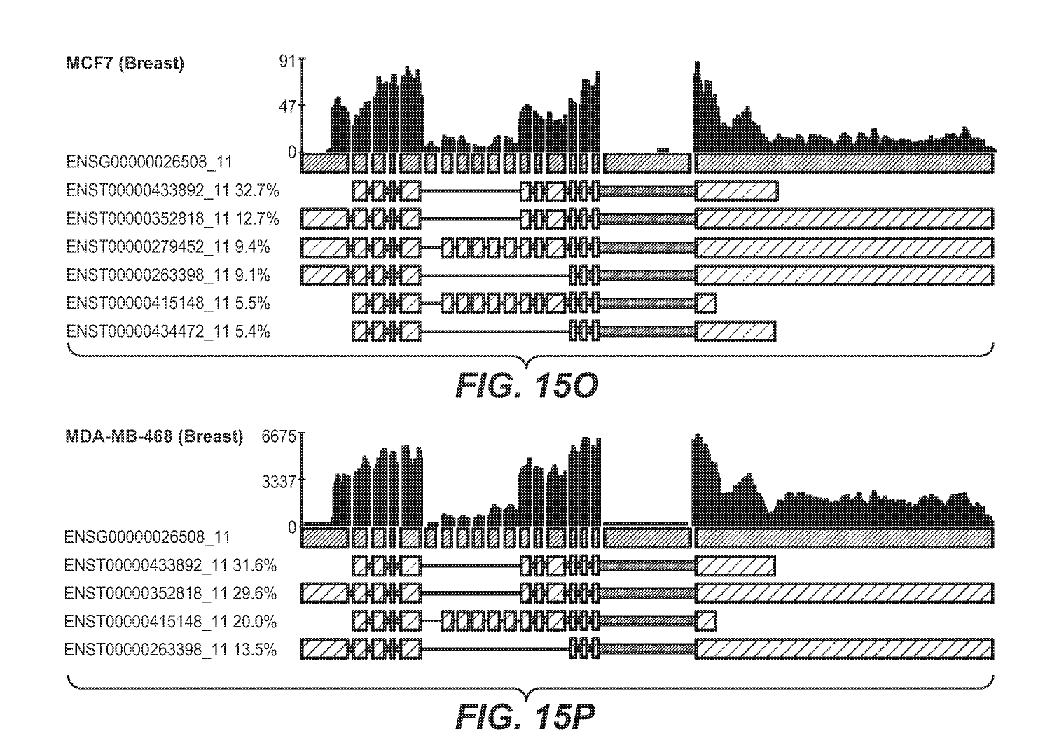

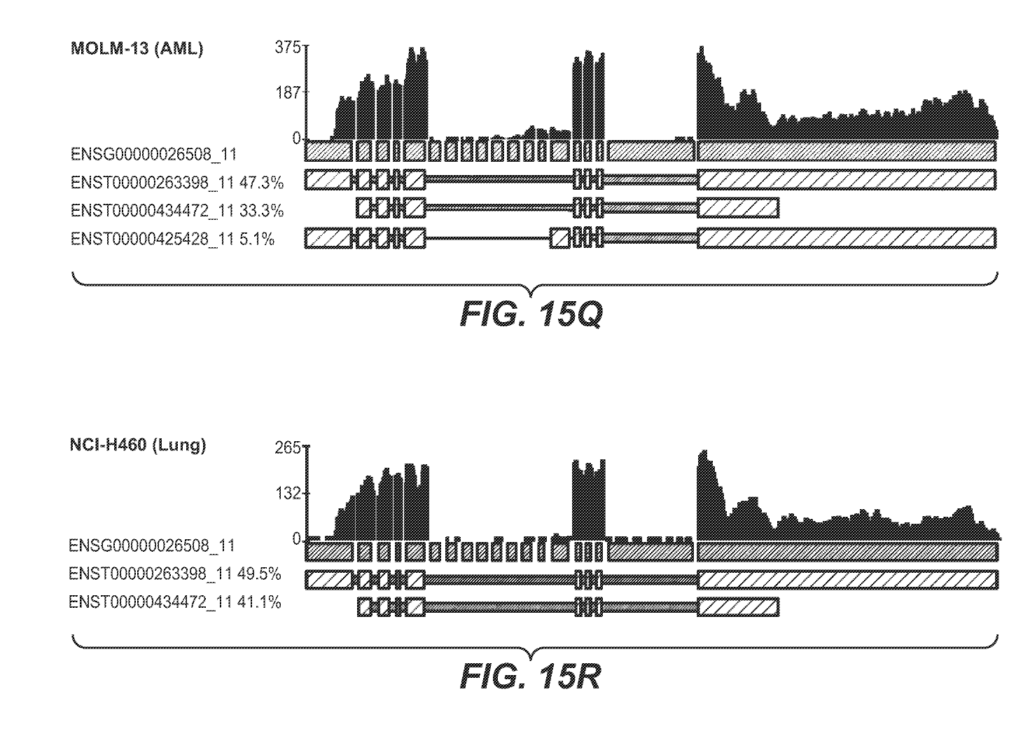

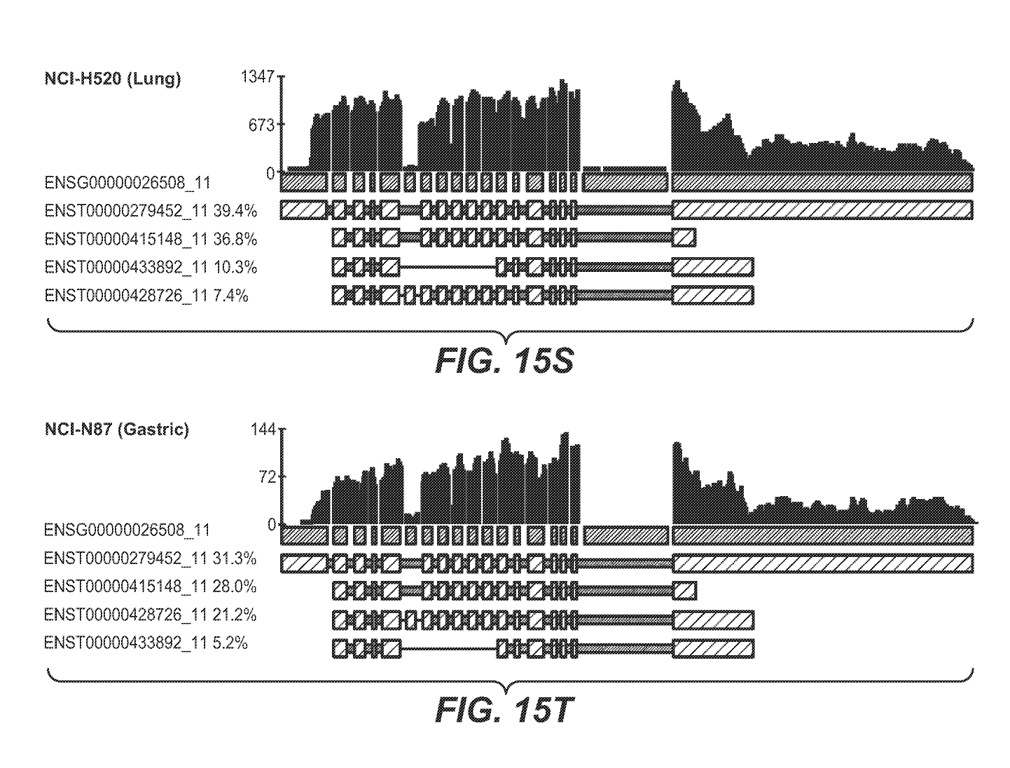

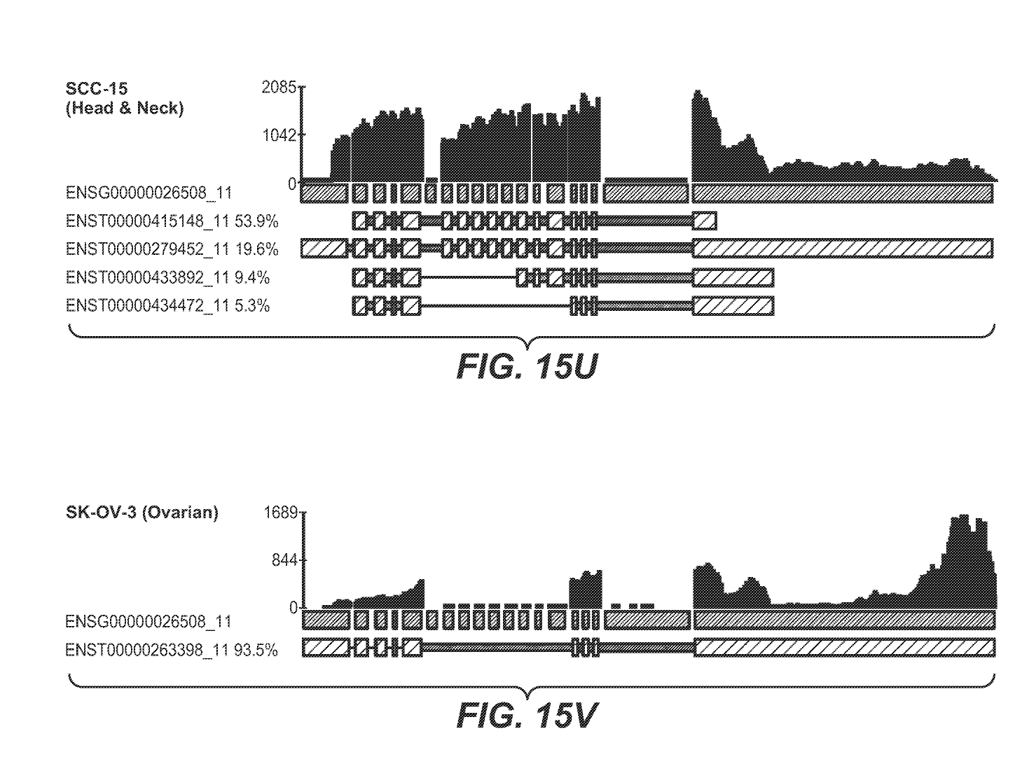

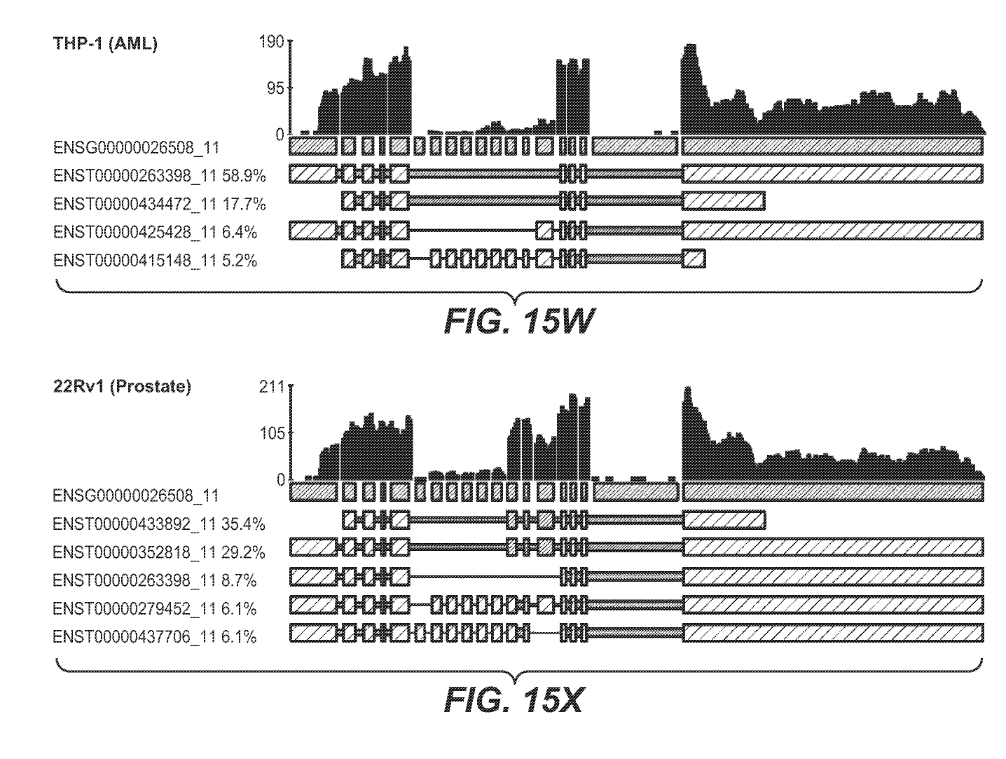

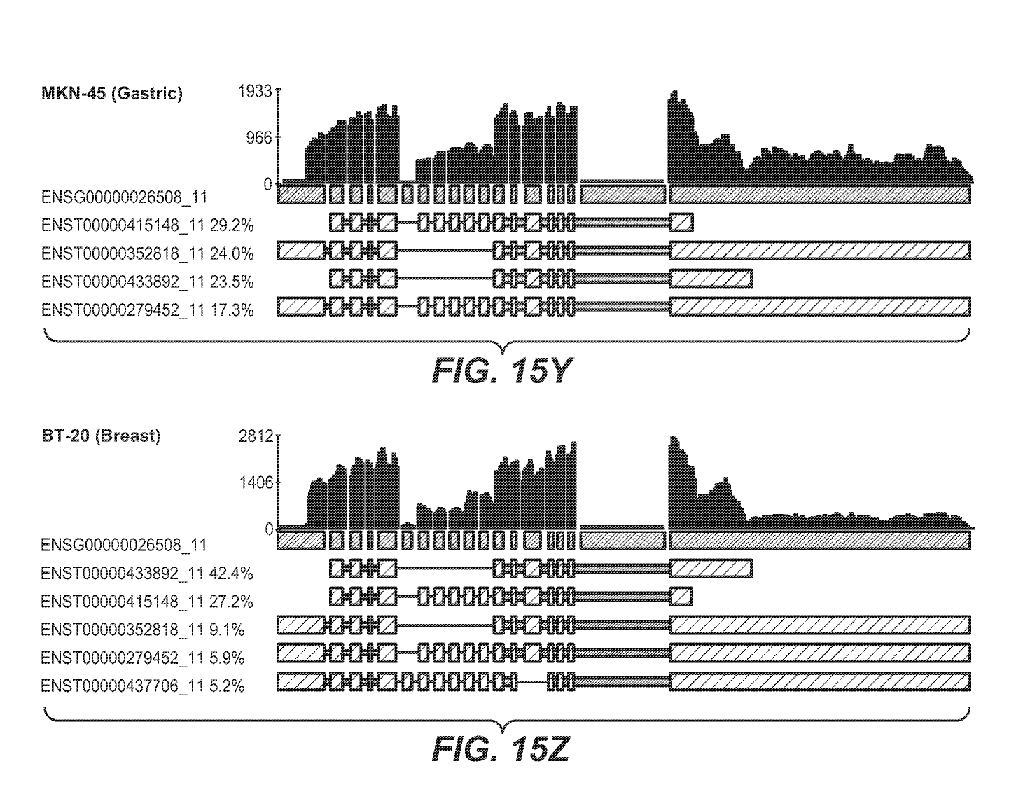

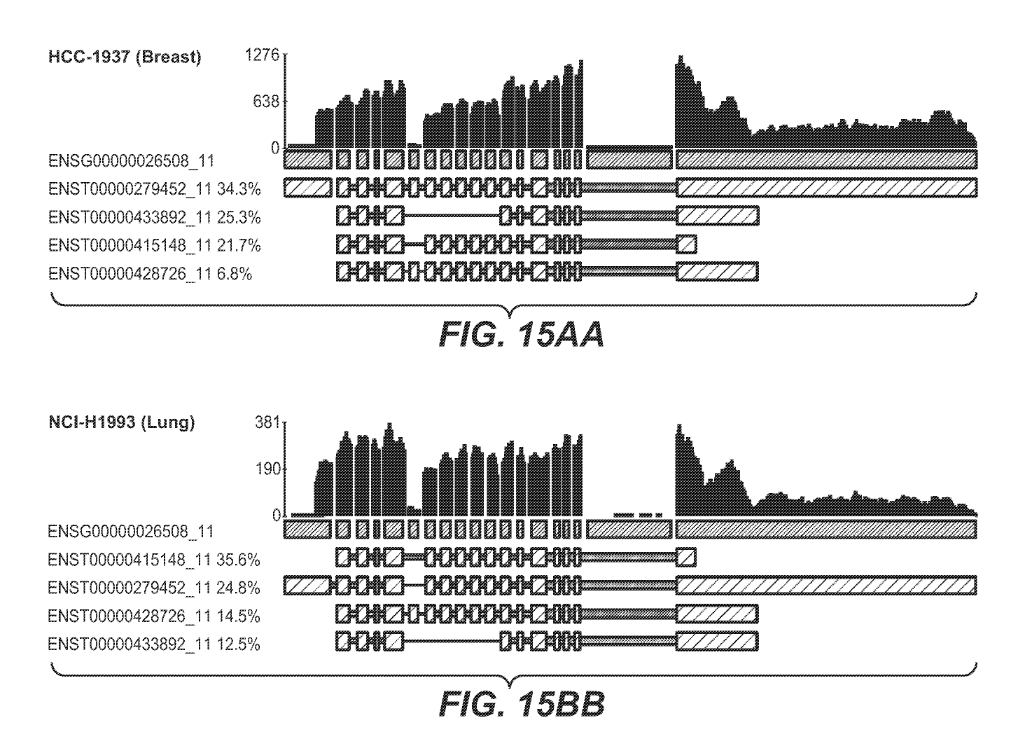

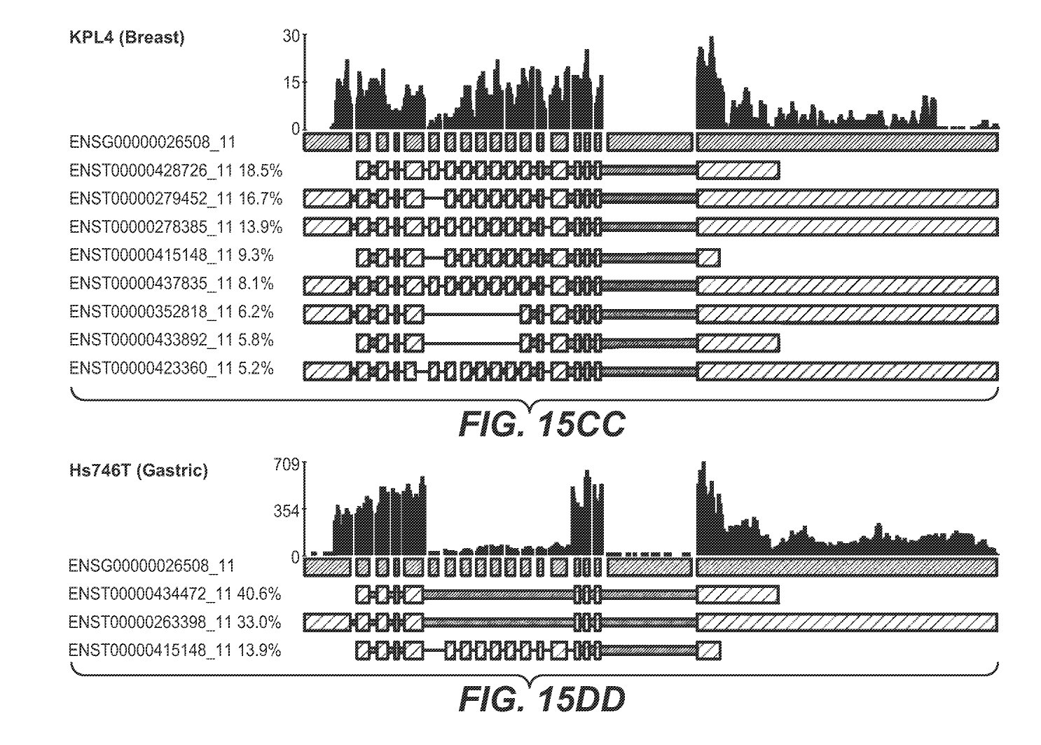

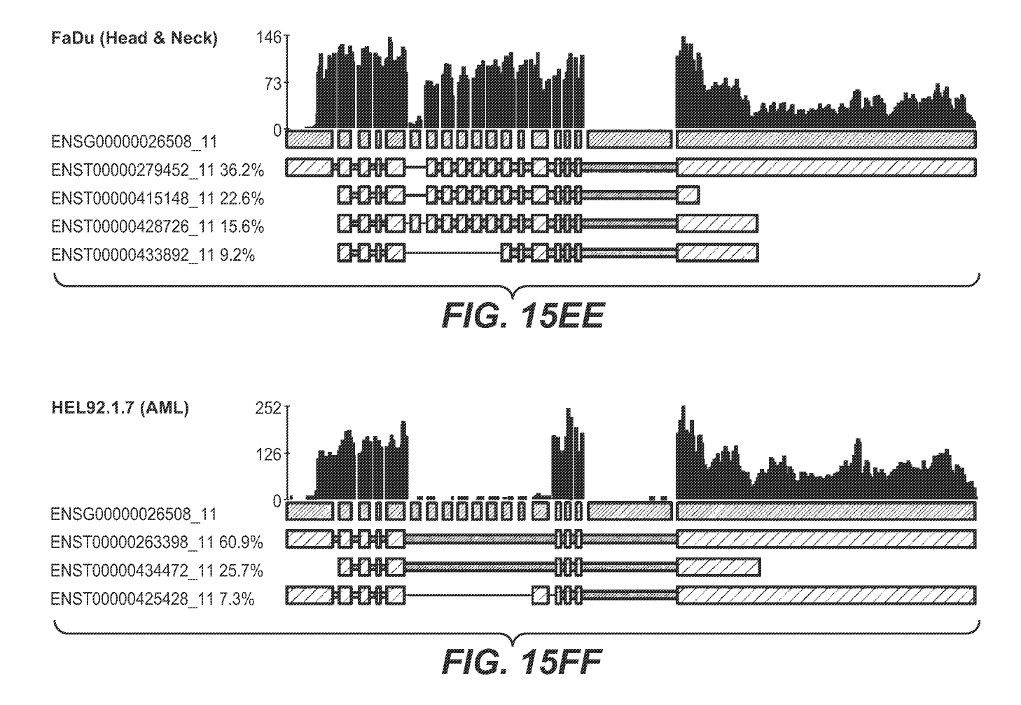

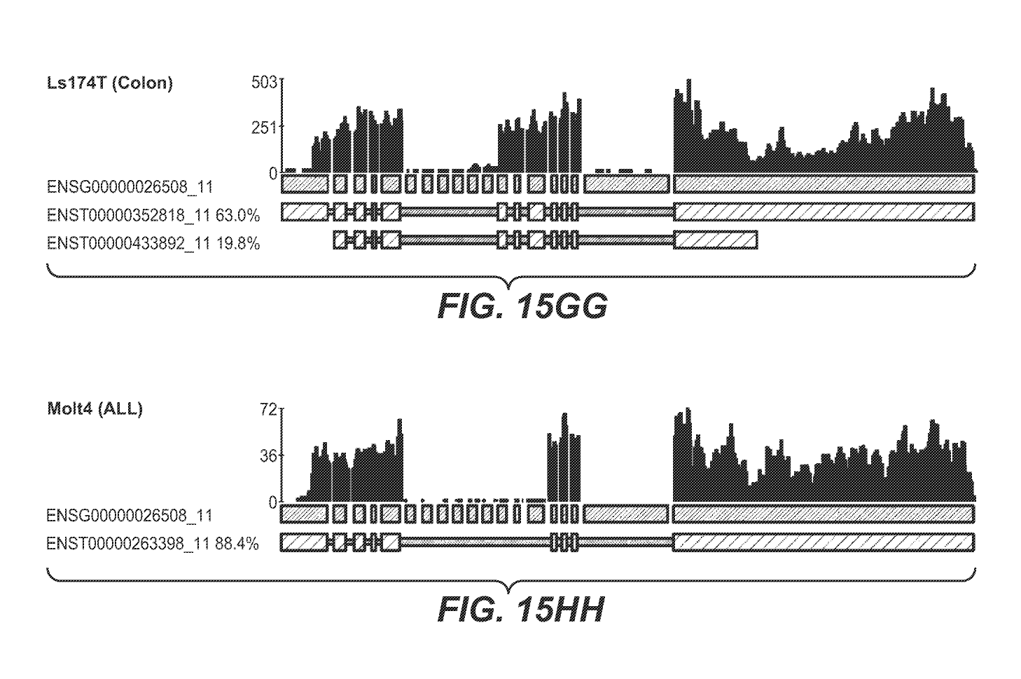

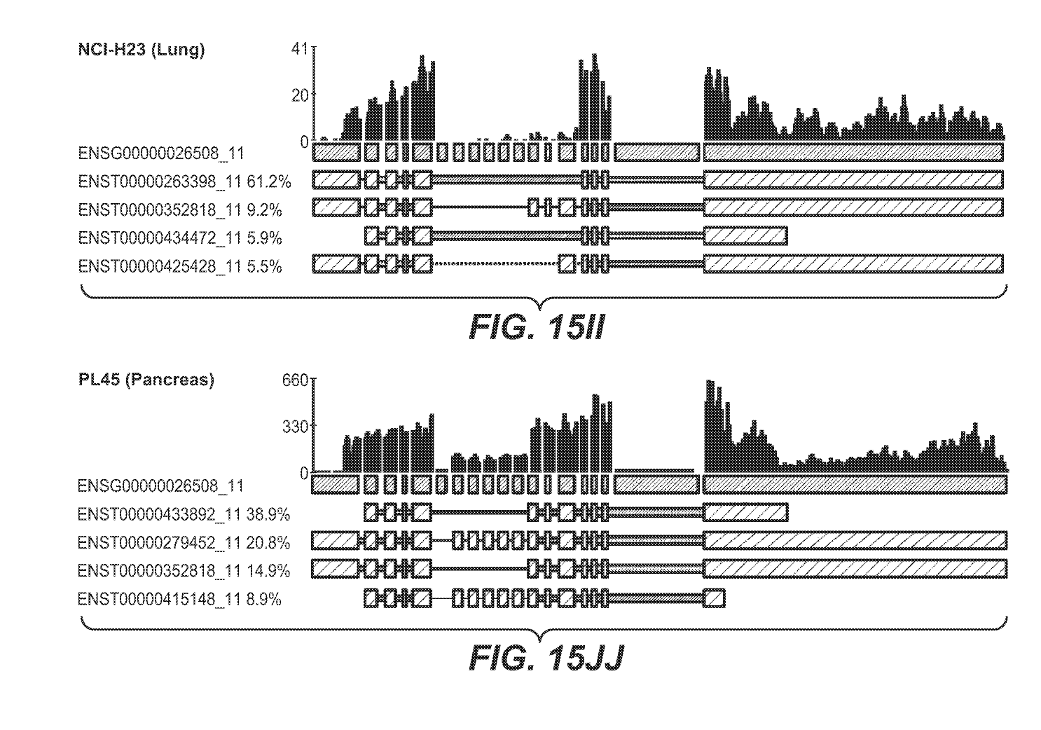

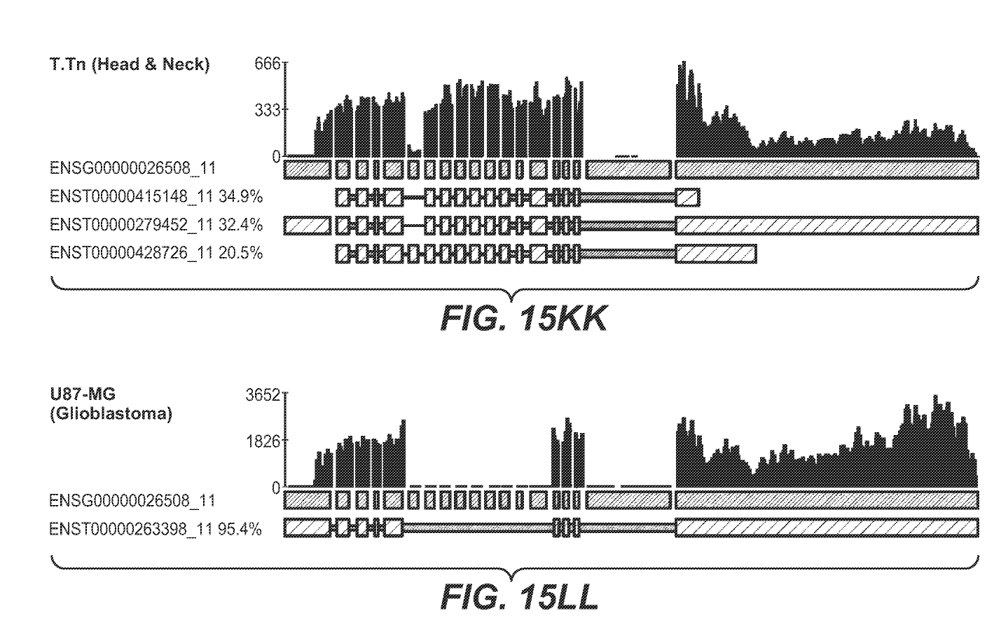

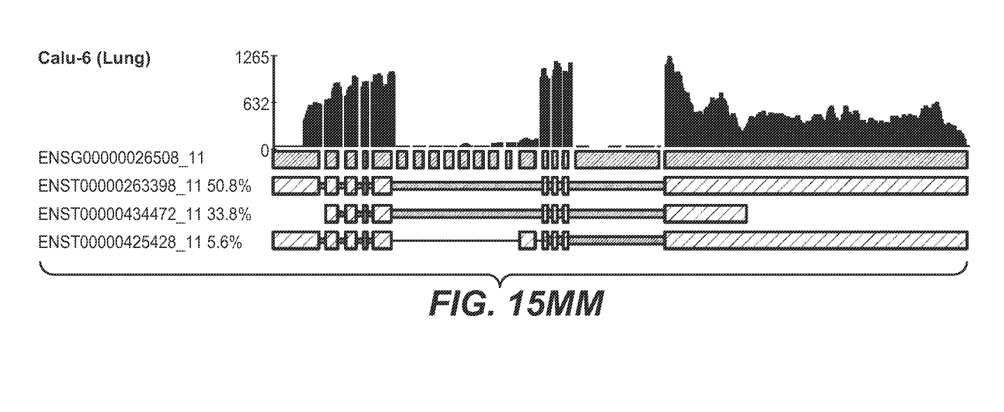

Raw RNASeq data was aligned to the human transcriptome (Ensembl v60) and genome (hgl9) using Bowtie2 (Langmead et al. Nature Methods, 2012, 9:357-359). Read data for the CD44 gene locus was extracted from the alignments and visualized as shown in FIG. 15 for every cell line. In FIG. 15, the first row of squares displays in all exons of the CD44 gene. On top of the CD44 exons, read coverage (the number of reads found for a specific position of the gene) is shown in the form of gray bars. Legend on the left hand side indicates what the maximal read coverage height of the bars means in terms of number of reads located on top of a certain position. Below the first row of squares indicating the gene structure, known transcripts of the CD44 gene are shown (displaying the exon composition of the specific transcript). Lines between exons indicate splice junctions. Thickness of black bars is an indication for the coverage of the junction by reads, i.e. how many reads cross a splice junction (the thicker a bar, the more reads span the junction). This visualization allows identification of the major isoform by eye. In addition, percentage values shown beside the transcript indicate the predicted percentage of an isoform in the complete population of CD44 transcripts expressed in the cell. For prediction we use MMSeq as described in the examples. For simplicity, isoforms with an MMSeq predicted frequency smaller than 5% are not shown in the plots.

The transcripts are identified by their official Ensembl IDs (Release v60). The mapping between Ensembl transcript IDs and the isoform names used above are as follows: CD44s: ENST00000263398_{—}11 and ENST00000434472_{—}11, CD44v2-v10: ENST00000278385_{—}11, CD44v3-v10: ENST00000279452_{—}11, CD44v8-v10: ENST00000352818_{—}11 and ENST00000433892_{—}11, CD44v10: ENST00000425428_{—}11. There are two isoforms representing CD44s that vary mostly in the non-coding region (ENST00000434472 has a truncated version of exon 19 lacking several non-coding nucleotides). MMSeq can not distinguish between the two variants such that the percentage of CD44s in the population of isoforms has to be measured by the sum of the two percentage values given for ENST00000263398_{—}11 and ENST00000434472_{—}11.

We used two approaches to identify the CD44 major isoform expressed in a sample. First, we used read counts for unique splice junctions that exist for each isoform of CD44, defining the isoform whose unique splice junction had the highest read coverage as major isoform. Second, we used MMSeq (Turro et al. Genome Biology, 2011, 12:R13) a method dedicated to quantify isoform levels in RNASeq data to quantify the frequency of the different CD44 isoforms defined in Ensembl v60. Both approaches resulted in the same CD44 major isoforms defined for each sample analyzed.

### TCGA Data Analysis

In order to determine CD44 isoform prevalence in a variety of 17 different tumor indications we used publicly available RNASeq data published by “The cancer genome atlas” (TCGA) consortium (The Cancer Genome Atlas Research Network, Nature, 2008, 455:1061-1068). Processed RNASeq data (TCGA data level 3) was downloaded from the TCGA Data portal in October 2012. In order to determine isoform levels we used junction coverage of the unique splice junctions used by CD44s, CD44v2-10, CD44v3-10 and CD44v8-10 and determined the major isoform as the isoform with the largest number of reads crossing its unique junction.

### Identification and Quantification of CD44 Splice Variants by Real-Time PCR

The CD44 gene contains 9 constant exons (E1-E9/10) and 9 variable exons (v2-v10). The variable exons are alternatively spliced, resulting in different CD44 splice variants. A set of PCR reactions was designed to discriminate and quantify the following CD44 isoforms:

CD44-v3 (containing variable exon v3)

CD44-v6 (containing variable exon v6)

CD44-v10 (containing variable exon v10)

CD44-v3-10 (containing variable exons v3-v10)

CD44-v6-10 (containing variable exon v6-v10)

CD44-v8-10 (containing variable exon v8-v10)

CD44s (containing no variable exon)

Design of primers and probes was based on the complete mRNA of the longest version of CD44 at NCBI (Refseq NM_{—}000610). This mRNA consists of 18 exons.

The PCR assay for CD44-v3 contained a forward primer spanning the splice junction between exon E5 and exon v3 (bp 1092-1101 and 1231-1240) and a reverse primer spanning the splice junction between exon v3 and exon E6 (bp 1353-1356 and 2245-2259). The probe of the PCR assay for CD44-v3 binds to by 1264-1299.

The PCR assay for CD44-v6 contained a forward primer spanning the splice junction between exon E5 and exon v6 (bp 1087-1101 and 1588-1592) and a reverse primer spanning the splice junction between exon v6 and exon E6 (bp 1713-1716 and 2245-2257). The probe of the PCR assay for CD44-v6 binds to by 1610-1640.

The PCR assay for CD44-v10 contained a forward primer spanning the splice junction between exon E5 and exon v10 (bp 1084-1101 and 2041-2043) and a reverse primer binding to exon v10 (bp 2087-2106). The probe of the PCR assay for CD44-v10 binds to by 2053-2081.

The PCR assay for CD44-v3-10 contained a forward primer spanning the splice junction between exon E5 and exon v3 (bp 1091-1101 and 1231-1242) and a reverse primer binding to exon v3 (bp 1293-1315). The probe of the PCR assay for CD44-v3-10 binds to by 1252-1260. To discriminate the CD44-v3 splice variant from the CD44-v3-10 splice variant, an additional assay was designed for long CD44 splice variants (CD44-long), containing a forward primer spanning the splice junction between exon v8 and exon v9 (bp 1934-1960) and a reverse primer spanning the splice junction between exon v9 and exon v10 (bp 2024-2046). The probe of this PCR assay binds to by 1984-2015.

The PCR assay for CD44-v6-10 contained a forward primer spanning the splice junction between exon E5 and exon v6 (bp 1087-1101 and 1588-1593) and a reverse primer binding to exon v6 (bp 1645-1661). The probe of the PCR assay for CD44-v6-10 binds to by 1610-1640. To discriminate the CD44-v6 splice variant from the CD44-v6-10 splice variant, the CD44-long assay was used.

The PCR assay for CD44-v8-10 contained a forward primer spanning the splice junction between exon E5 and exon v8 (bp 1090-1101 and 1849-1858) and a reverse primer binding to exon v8 (bp 1885-1905). The probe of the PCR assay for CD44-v8-10 binds to by 1874-1882. To unambiguously identify the CD44-v8-10 splice variant, the CD44-long assay was used.

The PCR assay for CD44-s contained a forward primer spanning the splice junction between exon E5 and exon E6 (bp 1085-1101 and 2245-2248) and a reverse primer spanning the splice junction between exon E6 and exon E7 (bp 2305-2322). The probe of the PCR assay for CD44-v6 binds to by 2266-2287.

The exact composition of CD44v3-10 and CD44v6-10 could not be specified unambiguously using this PCR approach, but quantification of these splice variants is nevertheless meaningfull. The assays will detect CD44v3-10 and CD44v6-10 as well as variants with different compositions regarding exons 4-7 and 7, respectively.

### Experimental Setup:

### RNA Isolation:

Sample material: frozen cell pellets

Isolation protocol: High Pure RNA Isolation Kit (Roche Applied Science, Id. No. 11828665001) RNA was quantified by UV photospectrometer and analyzed for quality by agarose gel electrophoresis

cDNA Synthesis:

1 μg RNA was transcribed into cDNA using the Transcriptor First Strand cDNA Kit (Roche Applied Science, Id. No. 04896866001). cDNA was quantified by UV photospectrometer and diluted in water, PCR-grade, to a concentration of 5 ng/μl.

cDNA Quantification:

cDNA was quantified by real-time PCR using the LightCycler 480 II (Roche Applied Science, Id. No. 05015243001) in relation to two housekeeping genes (IPO8 and HPRT). PCR reactions were set up according to the following scheme:

forward primer (10 μM): 1 μl

reverse primer (10 μM): 1 μl

probe (10 μM): 0.4 μl

LightCycler 480 Probes Master (Roche Applied Science, Id. No. 04707494001): 10 μl

cDNA (5 μg/μl): 5 μl

water, PCR-grade: 2.6 μl

PCR reactions were set up in a 384-well multi-well plate (Roche Applied Science, Id. No. 04729749001) and the following PCR program was run on a LightCycler 480 II instrument:

Pre-incubation:

| | | |
|---|---|---|
| 95° C. | 10 | minutes |

Amplification (45 cycles):

| | | |
|---|---|---|
| 95° C. | 10 | seconds |
| 60° C. | 30 | seconds |
| 72° C. | 1 | second |

Cooling:

| | | |
|---|---|---|
| 40° C. | 10 | minutes |

Data analysis and relative quantification was done using the LightCycler 480 II software, version LCS480 1.5.0.39.

The following primers and probes were designed to identify and quantify the respective CD44 splice variants:

CD44-v3:
Forward:

(SEQ ID NO: 87)

cctgctaccagtacgtcttc
Reverse:

(SEQ ID NO: 88)

accagagaccaagacacat
Probe:

(SEQ ID NO: 89)

agccaaatgaagaaaatgaagatgaaagagacagac
Cd44-v6:
Forward:

(SEQ ID NO: 90)

gaatccctgctaccatccag
Reverse:

(SEQ ID NO: 91)

gctggagaccaagacac
Probe:

(SEQ ID NO: 92)

aacggaagaaacagctacccagaaggaacag
CD44-v10:
Forward:

(SEQ ID NO: 93)

acagaatccctgctaccaata
Reverse:

(SEQ ID NO: 94)

aggctcaactactttactgg
Probe:

(SEQ ID NO: 95)

tcacaggtggaagaagagacccaaatcat
CD44-v3-10:
Forward:

(SEQ ID NO: 96)

ccctgctaccagtacgtcttcaa
Reverse:

(SEQ ID NO: 97)

gacagacacctcagtttttctgg
Probe (LNA):
cagcaggct
CD44-v6-10:
Forward:

(SEQ ID NO: 98)

gaatccctgctaccatccagg
Reverse:

(SEQ ID NO: 99)

ttggcaacagatggcat
Probe:

(SEQ ID NO: 100)

aacggaagaaacagctacccagaaggaacag
CD44-v8-10:
Forward:

(SEQ ID NO: 101)

tccctgctaccaatatggactc
Reverse:

(SEQ ID NO: 102)

ctgcaaatccaaacacaggtt
Probe (LNA):
gcttcagcc
CD44-s:
Forward:

(SEQ ID NO: 103)

cagaatccctgctaccagaga
Reverse:

(SEQ ID NO: 104)

atggacactcacatggga
Probe:

(SEQ ID NO: 105)

ccagtggggggtcccataccac
CD44-long:
Forward:

(SEQ ID NO: 106)

tctttcaatgacaacgcagcagagtaa
Reverse:

(SEQ ID NO: 107)

tactctgacatcaagcaatagga
Probe:

(SEQ ID NO: 108)

atgaaggcttggaagaagataaagaccatcca
CD44 Pan (measures any CD44 transcript
irrespective of isoform):
Forward:

(SEQ ID NO: 109)

gaatcagatggacactcaca
Reverse:

(SEQ ID NO: 110)

ttccagaatggctgatcatc
Probe (LNA):
aggtggag

### Preparation of Cell Lysates and Supernatants

### Cell Culture Conditions:

A T75 cell culture flask was seeded with 1 million cells in RPMI 1640 and grown at 37° C. under 5% CO2 and 95% relative humidity for 3 days. The supernatant was collected, centrifuged, separated from the solid residue, frozen and stored at −80° C. Cells were washed with PBS and lysed with 1 m 1 lysis buffer. The total protein concentration of the lysates was analyzed by BCA-assay (Thermo #23223 and #23224). The obtained lysates were split into aliquots and stored at −80° C.

### Cell Culture Medium:

RPMI 1640, with stab. L-glutamine and 2.0 g/l NaHCO₃; (PAN #P04-18500) was used as standard for most cell lines. DMEM (Gibco #41966-029) was used for BT-20, Cal 27, Hs746T, Panc-1, PL-45 and T.Tn. Calu-6, FaDu, Ls174T and U87MG were grown in EMEM (Gibco #P04-00509). In all cases100 IU/ml penicillin, 100 μg/ml streptomycin (Penicillin G, sodium salt, streptomycin sulfate (Roche Applied Science #11074440001, for 20 ml (500×), lyophilisate, sterile) and 10% FCS (Gibco #10500-64) was added.

### Cell Lysis Buffer:

50 mM Tris buffer, pH 8.0 (Roth #4855.1)

150 mM sodium chloride (Merck #1.06404.1000)

1 mM EGTA (Calbiochem)

1% Triton-X-100 (Roche #789704)

10% glycerol (Gibco #15708-019)

10 μg/ml leupeptine (Sigma #L8511)

10 μg/ml aprotinine (Sigma #L8511)

1 mM PMSF (Sigma #P7602)

0.4 mM ortho-vanadate (Sigma #S6508)

### Cell Lines:

A549, Calu6, FaDu, Huh7, MDA-MB 231, MDA-MB 468, NCI H1993, Panc-1, were obtained from ATCC; Colo205, MCF7, PC-3, SK-Hep-1, SK-OV3 were obtained from NCl; Cal 27, HCC1937, MKN45 and PL45 from DSMZ; Ls174T from ECACC. T.Tn from Arius.

### HA-Measurement: Comparison of Available ELISA-Formats

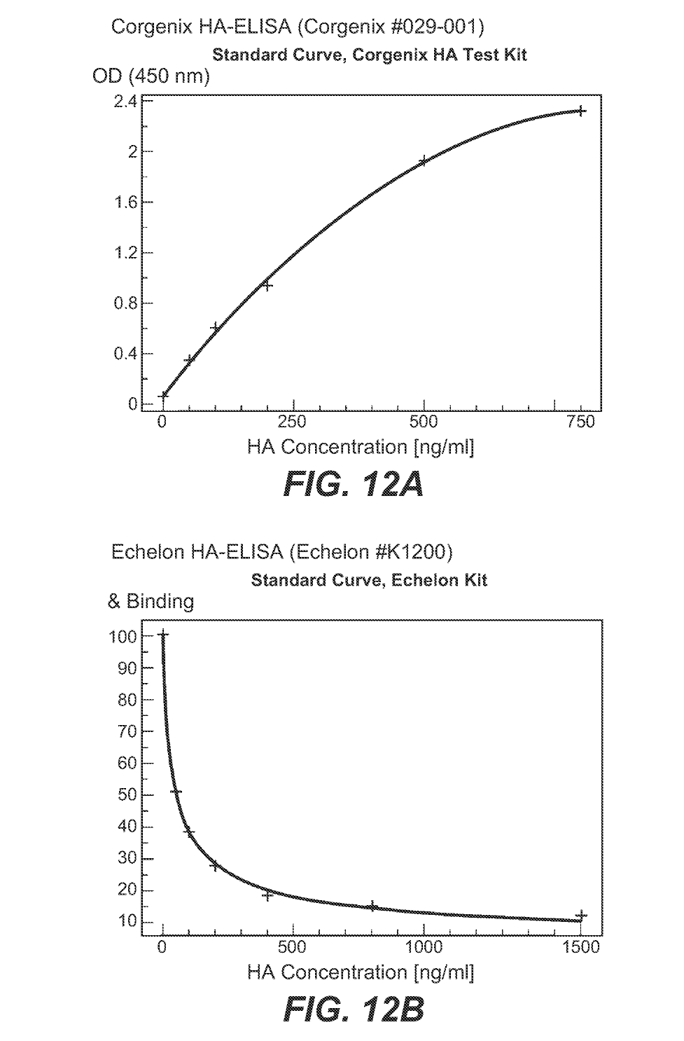

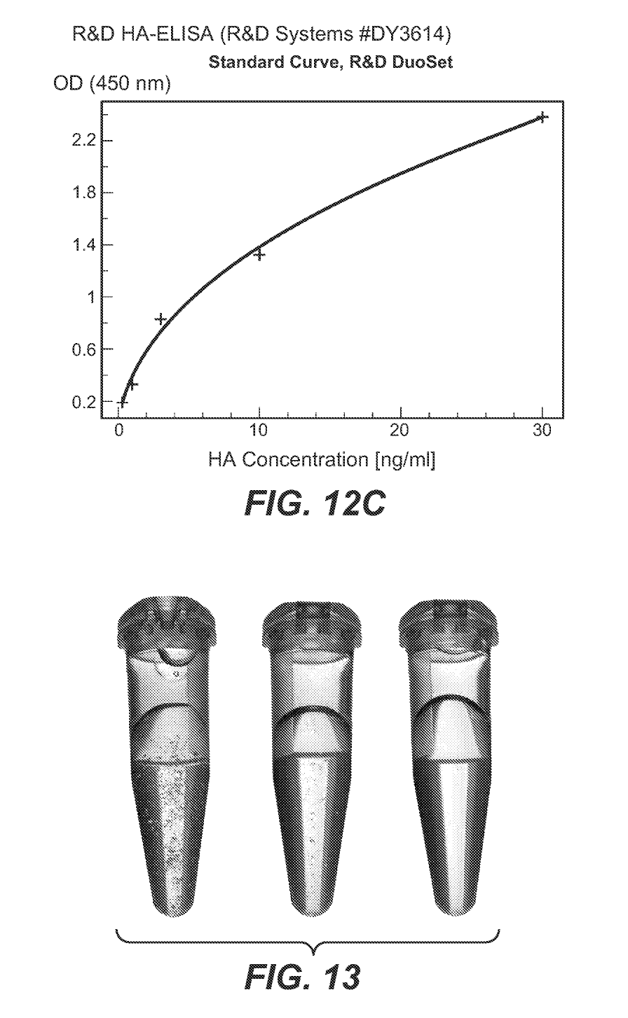

Three commercially available HA ELISA assays were compared based on their calibration curve and the values that were obtained for HA of different molecular weight from 5-1000 kD. Corgenix HA-ELISA was obtained from Corgenix UK, product number #029-001, Echelon HA-ELISA from Echelon Bioscience, product number K-1200, and R&D HA Duoset obtained from R&D Systems, product number DY3614. Hyaluronic acid of different molecular weight was from R&D Systems. HA-probes containing defined concentration were measured according the manuals supplied by the manufacturers of the assays. The results are shown in FIG. 12.

Our results show that the Corgenix ELISA has the broadest detection range which is very comfortable for the different concentrations of HA that were expected in cell cultures. Although there is a high deviation for low molecular weight HA, results are accurate for high molecular weight HA>1000 kD which is expected to be produced in cell cultures.

The R&D Systems Duoset is the most sensitive assay format and therefore most suitable for measuring low concentrations of HA like the background in the culture medium.

The Echelon ELISA (the only one with a competitive detection principle) shows very little dependency from the molecular weight of HA. The very steep calibration curve does not allow a comfortable measurement of many samples with unknown concentrations.

The following relates to data shown in FIG. 12 A-C.

The data underlying FIG. 12 A is shown in the following table:

12 A

| | | | sample | |
|---|---|---|---|---|
| | R&D cat. | | concentration | measured HA |
| | No. | MW [kDa] | [ng/ml] | concentration [ng/ml] |
| HA (ultra low Molecular | GLR003 | 4.7 | 500 | 3 |
| Weight) | | | | |
| HA (low Molecular | GLR001 | 17-35 | | 73 |
| Weight) | | | | |
| HA (medium Molecular | GLR004 | 132 | | 377 |
| Weight) | | | | |
| HA (high Molecular | GLR002 | 980 | | 561 |
| Weight) | | | | |

| | | | sample concentration | | measured HA |
|---|---|---|---|---|---|
| | R&D cat. No. | MW [kDa] | [ng/ml] | mean | concentration [ng/ml] |
| HA (ultra low Molecular Weight) | GLR003 | 4.7 | 500 | 0.0963 | 3 |
| HA (low Molecular Weight) | GLR001 | 17-35 | | 0.4391 | 73 |
| HA (medium Molecular Weight) | GLR004 | 132 | | 1.5929 | 377 |
| HA (high Molecular Weight) | GLR002 | 980 | | 2.0351 | 561 |

The data underlying FIG. 12 B is shown in the following table:

12 B

| | | | sample | |
|---|---|---|---|---|
| | R&D cat. | MW | concentration | measured HA |
| | No. | [kDa] | [ng/ml] | concentration [ng/ml] |
| HA (ultra low Molecular | GLR003 | 4.7 | 1000 | 902 |
| Weight) | | | | |
| HA (low Molecular | GLR001 | 17-35 | | 1016 |
| Weight) | | | | |
| HA (medium Molecular | GLR004 | 132 | | 1021 |
| Weight) | | | | |
| HA (high Molecular | GLR002 | 980 | | 953 |
| Weight) | | | | |

| | | | sample | | | measured HA |
|---|---|---|---|---|---|---|
| | R&D | | concentration | | | concentration |
| | cat. No. | MW [kDa] | [ng/ml] | mean | &Binding | [ng/ml] |
| HA (ultra low Molecular Weight) | GLR003 | 4.7 | 1000 | 0.5122 | 14.6 | 902.0 |
| HA (low Molecular Weight) | GLR001 | 17-35 | | 0.4983 | 13.9 | 1016.0 |
| HA (medium Molecular Weight) | GLR004 | 132 | | 0.4978 | 13.9 | 1021.0 |
| HA (high Molecular Weight) | GLR002 | 980 | | 0.5056 | 14.3 | 953.0 |

The data underlying FIG. 12 C is shown in the following table:

12 C

| | | | sample | |
|---|---|---|---|---|
| | R&D cat. | | concentration | measured HA |
| | No. | MW [kDa] | [ng/ml] | concentration [ng/ml] |
| HA (ultra low Molecular | GLR003 | 4.7 | 30 | 0.1 |
| Weight) | | | | |
| HA (low Molecular | GLR001 | 17-35 | | 2.6 |
| Weight) | | | | |
| HA (medium Molecular | GLR004 | 132 | | 29.3 |
| Weight) | | | | |
| HA (high Molecular | GLR002 | 980 | | 34.8 |
| Weight) | | | | |

| | | | sample concentration | | measured HA |
|---|---|---|---|---|---|
| | R&D cat. No. | MW [kDa] | [ng/ml] | mean | concentration [ng/ml] |
| HA (ultra low Molecular Weight) | GLR003 | 4.7 | 30 | 0.0878 | 0.1 |
| HA (low Molecular Weight) | GLR001 | 17-35 | | 0.7332 | 2.6 |
| HA (medium Molecular Weight) | GLR004 | 132 | | 2.3405 | 29.3 |
| HA (high Molecular Weight) | GLR002 | 980 | | 2.6280 | 34.8 |

### HA-Analysis of Cell Lysates and Supernatants

Lysates and supernatants were analyzed according to the manuals supplied by the manufacturers of the assays (Table 4). In general the Corgenix ELISA was used to measure cell lysates and supernatants. Very low concentrations were validated by a second measurement with the R&D Duoset. HA-background of the used cell culture medium was also measured using the R&D Duoset. The following HA-background concentrations (ng/ml) were measured: RPMI 38.5, EMEM 31.5 and DMEM 35.9.

TABLE 4
HA-levels in cell lysates and supernatants

| | | | | ng | ng | |
|---|---|---|---|---|---|---|
| | | Total | ng HA/ | HA/mg | HA/ml | CD44 |
| Cell line | | Protein | ml | total | super- | Isoform |
| name | Tissue | [mg/ml] | Lysate | protein | natant | Status |
| A549 | Lung | 5.48 | 565 | 103 | 595 | S |
| BT-20 | Breast | 1.45 | 74 | 51 | 110 | V |
| Cal 27 | Head & | 1.85 | 202 | 109 | 282 | S |
| | Neck | | | | | |
| Calu-6 | Lung | 4.9 | 9806 | 2003 | 1140 | S |
| Colo205 | Colon | 3.77 | 169 | 45 | 125 | V |
| FaDu | Head & | 1.12 | 13 | 11 | 230 | V |
| | Neck | | | | | |
| HCC1937 | Breast | 6.94 | 435 | 63 | 158 | V |
| Hs746T | Gastric | 4.16 | 13323 | 3203 | 3438 | S |
| Ls174T | Colon | 6.59 | 32 | 5 | 66 | V |
| MCF-7 | Breast | 6.67 | 19 | 3 | 53 | V |
| MDA- | Breast | 3.27 | 550 | 168 | 482 | S |
| MB231 | | | | | | |
| MDA- | Breast | 1.65 | 18 | 11 | 47 | V |
| MB468 | | | | | | |
| MKN-45 | Gastric | 4.4 | 11 | 3 | 91 | V |
| NCI | Lung | 8.63 | >10000 | >1160 | 1094 | V |
| H1993 | | | | | | |
| NCI-H23 | Lung | 2.58 | 193 | 75 | 261 | S |
| Panc-1 | Pancreas | 1.71 | 304 | 178 | 253 | S |
| PC-3 | Prostate | 1.83 | 520 | 284 | 1940 | S |
| PL-45 | Pancreas | 1.43 | 28 | 20 | 76 | V |
| SK-Hep-1 | HCC | 1.47 | 185 | 126 | 896 | S |
| SK-OV3 | Ovarian | 0.98 | 15 | 15 | 152 | S |
| T.Tn | Head & | 1.3 | 23 | 18 | 213 | V |
| | Neck | | | | | |
| U87MG | Glioblastoma | 2.27 | 5403 | 2380 | 308 | S |

Interference of RG7356 with CD44s-HA-Binding

### Binding of CD44s to Coated HA

The predominant form of CD44, also called CD44s (CD44 standard or historically CD44H), contains several binding sites for Hyaluronic Acid (HA). The most prominent of them was confirmed by solving the crystal structure of a CD44-HA complex (Banerji et al. 2007 (loc. cit.)).

The binding of CD44 derived fusion proteins to surfaces coated with HA is a useful method to study this receptor-ligand interaction. The binding studies of a CD44-Fc fusion protein to polystyrene surfaces coated with HA was first studied by Peach et al., 1993 (loc. cit.). The described assay binding of CD44 mutants to a Hyaluronic Acid coating is a suitable model system to study a potential interference of CD44-antibodies with this receptor ligand interaction.

Nevertheless the binding affinity shown by the work of the Stamenkovic group is too low, mainly because of inappropriate equilibration of lyophilized HA-material.

An adequate equilibration of dehydrated Hyaluronic Acid in aqueous systems is crucial for obtaining high affinity binding to rhCD44-Fc. The kinetics of HA hydration are shown in FIG. 13. After three hours the system is still biphasic, containing species different hydration status (no bubbles).

To achieve a suitable HA solution of 2.5 mg/ml, 30 mg HA (Hyaluronic Acid sodium salt from rooster comb, Sigma #H5388) was allowed to equilibrate in 12 ml of 50 mM bicarbonate buffer pH 9.6 (1.59 g Na2CO3, 2.93 g NaHCO3/l) under shaking at 37° C. for 12 h. The resulting solution is clear and monophasic (FIG. 13). ELISA plates (F96 Maxisorb-Immuno plate, Nunc #442404) were incubated with 100 μl HA-solution per well for 12 h at 4° C., washed 4× with 300 μl of washing buffer(PBS containing 0.05% Tween20, pH 7.4) and used immediately. A freshly prepared HA coated plate was incubated in each well with 200 μl of blocking buffer (PBS containing 3% BSA) for 2 h at 37° C. The wells were washed four times with 300 μl blocking buffer and 50 μl of CD44-Fc (R&D Systems #3660-CD) solution added (two wells containing the same concentration of CD44-Fc) and the plate shaken at r.t. for 1 h. The concentration range of CD44-Fc was set from 100 μg/ml to 1 ng/ml. After four washings with 300 μl washing buffer 100 μl of the goat F(ab′)2 fragment against human IgG-Fc (Jackson #109-036-098) was added (dilution 1:5000 in PBS containing 1% BSA) and shaken at r.t. for 1 h. After four washings with 300 μl wash buffer 50 μl of TMB substrate (OptEIA, BD Bioscience #555214) were added and allowed to develop for 15 min. at r.t. under shaking. The color development was stopped by addition of 50 μl 2N sulfuric acid and the absorption measured at 450 nm versus 620 nm.

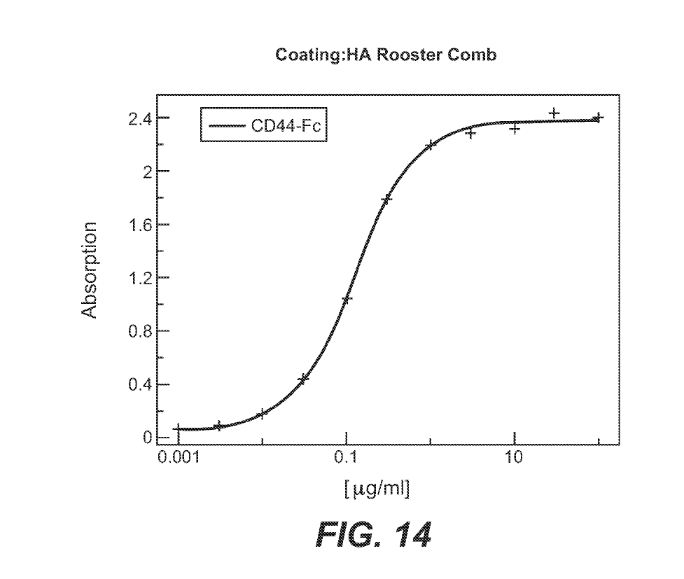

The binding of rhCD44-Fc to wells that were pre-coated with HA from rooster comb is shown in FIG. 14. The apparent EC50 for the binding of under these conditions is ˜0.2 μg/ml.

Interference of RG7356 with Binding of rhCD44s-Fc to HA-Coated Plates

A freshly prepared HA coated plate was incubated in each well with 200 μl of blocking buffer for 2 h at 37° C. The wells were washed four times with 300 μl blocking buffer and 50 μl of CD44-Fc solution containing the desired concentration of either RG7356 or isotype control (muIgG1-kappa isotype control, Sigma #M9269) added (two wells containing the same concentration). The 50 μl of CD44-Fc were prepared from 25 μl CD44-Fc solution and 25 μl RG7356 solution or 25 μl isotype control solution. The combined solutions (50 ml) were allowed to equilibrate for 1 h at 37° C. before adding to the HA-coated wells.

The following concentrations were used:

CD44 alone: 8 different concentrations from 0.000 μg/ml to 28 μg/ml. CD44+ isotype (5-fold excess): CD44: 8 concentrations from 0.0002 μg/ml to 56 μg/ml; Isotype: 8 concentrations from 0.00 μg/ml to 280 μg/ml. CD44+RG7356 (2-fold excess): CD44: 8 concentrations from 0.0002 μg/ml to 56 μg/ml;

RG7356: 8 concentrations from 0.0004 μg/ml to 112 μg/ml. CD44+RG7356 (5-fold excess): CD44: 8 concentrations from 0.0002 μg/ml to 56 μg/ml;

RG7356: 8 concentrations from 0.00 μg/ml to 280 μg/ml.

CD44, titration with RG7356:
1) CD44: 0.26 μg/ml, RG7356: 8 concentrations from 0.0002 μg/ml to 56 μg/ml.
2) CD44: 0.54 μg/ml, RG7356: 8 concentrations from 0.0002 μg/ml to 56 μg/ml.

After addition of the pre-incubated combined CD44-Fc-solutions, the plate was incubated at room temperature for 1 h and washed four times with 300 μl wash buffer. 50 μl of TMB substrate were added and allowed to develop for 15 min. at r.t. under shaking. The color development was stopped by addition of 50 μl stop solution (2N H2SO4) and the absorption measured at 450 nm versus 620 nm.

### Cell Adhesion Assay

Surface attachment of cells was determined using an xCELLigence System/RTCA MP Instrument (Roche). The system monitors cellular events in real time without the incorporation of labels by measuring electrical impedance across inter-digitated micro-electrodes integrated on the bottom of tissue culture E-Plates. Components of this instrument included:

| | |
|---|---|
| RTCA Analyzer | Roche Order # 05228972001 |
| RTCA MP Station | Roche Order # 05331625001 |
| RTCA Control Unit | Roche Order # 05454417001 |
| 96-well E_plates | Roche Order # 05232376001 |

### Technology

The presence of the cells on top of the electrodes will affect the local ionic environment at the electrode/solution interface, leading to an increase in the electrode impedance. The more cells are attached on the electrodes, the larger the increases in electrode impedance. In addition, the impedance depends on the quality of the cell interaction with the electrodes. For example, increased cell adhesion or spreading will lead to a larger change in electrode impedance. Thus, electrode impedance, which is displayed as cell index (CI) values, can be used to monitor cell viability, number, morphology, and adhesion degree in a number of cell-based assays. Data shown in this report include CI values, or change of CI as a function of time (dCI/dt), which is termed “slope”.

### Preparation of HA-Coated 96-Well E-Plate

HA was dissolved in sterile DPBS (4 mg/ml) at 37° C. and kept overnight at this temperature. HA-containing buffer (100 μl/well) was added to each test well and plates were incubated for 2 hours at 37° C. After removal of the HA-buffer wells were washed threefold with PBS.

### Cell Culture

Cells were grown to 80% confluency in media required for growth of selected cell lines using T75 or T175 cell culture flasks. For cell adhesion assays cell culture medium was removed by aspiration and cells were washed with 10 ml DPBS w/o Ca++ and Mg++. 1 ml Trypsin was added and flasks were incubated at 37° C. until cells detached. Cells were transferred to centrifugation tubes, counted, and centrifuged at 250-500×g for 5 minutes. Supernatant was removed and cells were re-suspended in complete media (1,4×105 cells/well).

### Adhesion Assay

The appropriate volume of cell culture medium containing antibodies, control antibodies, or control buffers, was added to cells and the suspension was mixed gently using a 10 ml pipette. Tubes were incubated for two hours at 37° C. to allow antibody binding to cells. Background signal of xCELLigence E-plates was measured in presence of 100 μl cell culture medium 10 minutes before the incubation time ended. To start measurement cell culture medium was removed and 100 μl/well cell suspension was added into each well. xCELLigence signals were measured for 2 hours, fluids were remove carefully with a multi-channel pipette, and 100 μl DPBS was added to wells very slowly. Plates were placed in the xCELLigence System instrument and cell index was determined (“wash step 1” signal). Remove DPBS and add fresh DPBS, put E-plate into instrument and determine cell index (“wash step 2” signal). “wash step 3” signal was determined after repeating the wash step. Finally, DPBS was removed and 100 μl DMEM was added followed by continuous signal monitoring. Effect of test compounds on adherence of cancer cells to HA-coated plates is expressed as change in CI values during initial observation period (approx. 60 minutes), indicated as “slope”. Slope obtained without antibody was used for normalization (100%).

### CD44 Immunohistochemistry Protocol (Monoclonal Rabbit IgG Antibody SP37, Ventana, Cat-No. 790-4537):

This protocol describes an automated procedure for in-situ detection of CD44 by immunohistochemistry (IHC) on the Ventana automated slide stainer BenchMark ULTRA and is approved for formalin fixed paraffin embedded AML and normal skin. The Ventana CD44 clone

SP37 is a rabbit monoclonal antibody directed against a conserved region of the CD44 protein (amino acids 153-171). This antibody exhibits a membranous staining pattern and may be used to detect the CD44 standard and variant proteins in a variety of neoplastic tissues. The intended use is the comparison of the number and distribution of CD44 positive cells identified based on immunohistochemical detection in human Multi tumor tissue (MT), AML, n-skin and normal tissue. A monoclonal rabbit IgG antibody from Cell Signaling (Cat-No#39005) is used as isotype control. Anti-CD44 (SP37) Rabbit Monoclonal Primary Antibody is optimized for use with the Ventana Medical Systems ultraView Universal DAB detection kit procedure (P/N 760-500) on Ventana Medical Systems automated BenchMark ULTRA stainer utilizing NexES version 11.8 software.

Briefly, the procedure is as follows: After baking and de-paraffinization of the sample, pre-treatment (CC2 ultra) for 44 minutes is required. Afterwards, Anti-CD44 (SP37) is applied and incubated for 16 minutes at 36° C. which is followed by an ultrawash step. Finally one drop of Hematoxylin II (counterstain) is added for 4 minutes followed by one drop of Bluing reagent for 4 minutes (post counterstain). The slides need to be washed, dehydrated and coverslipped.

Hyaluronic Assay (HA) Affinity Histochemistry Protocol (AHC): Biotinylated Hyaluronic Acid Binding Protein (HABP, Calbiochem, Merck, Cat-No. 385911): This protocol describes a manual procedure for in situ detection of hyaluronic acid by chromogenic staining and is approved for formalin fixed paraffin embedded human Breast Cancer (BC)—Colorectal Cancer—(CRC), Hepatocellular Carcinoma—(HCC), AML, normal skin and xenograft tissue. HABP binds specifically and strongly to hyaluronic acid (>2000 M.W.) and is composed of two binding polypeptides, which are derived from N-terminal regions of the hyaluronic acid binding proteoglycan and linkage proteins. As this protocol is a manual assay, staining patterns may be variable depending on pre-analytical conditions such as fixation and embedding. Minor changes in protocol procedures, particularly incubation times and temperatures, are likely to impact staining intensity. The intended use is the detection of the distribution of hyaluronic acid in Breast Cancer (BC)—Colorectal Cancer—(CRC), Hepatocellular Carcinoma (HCC) of human origin, AML, normal skin and xenograft tissue. Particularly in comparative studies (e.g., comparing intensities of HA signals before and on treatment in the pre-clinical or clinical setting, or comparing staining intensity across different malignant tumors), the use of controls such as human epidermis is indispensable to control assay sensitivity. It is recommended to perform staining of samples that are to be directly compared to each other in the same batch. As the capacity of handling slides in manual protocols is limited, it is recommended to determine the optimal batch size of an experiment prior to measuring study samples. HABP needs to be freshly prepared for each run. Use of new batches of HABP require concordance testing.

Briefly, the procedure is as follows: The procedure for the HA AHC needs to be split over two days. On day one, the tissue slides are de-paraffinized and re-hydrated using xylene and ethanol according to standard procedure, (ensure to change these liquids frequently). Afterwards, it needs to be proceeded with the antigen retrieval step. Here, a water bath is heated to ˜60° C. and the tissue sections are transferred by using a suitable staining jar filled with the antigen retrieval buffer into the water bath. The tissue sections are incubated with the antigen retrieval buffer in the water bath for 16 h.

On day two, it is important to allow smooth cooling down of the slides for at least 30 minutes before continuing with a washing step and blocking with 1% BSA for 30 minutes. After removal of the blocking solution, the tissue sections are incubated with the HABP in a humidified chamber for 1 h at room temperature. After a washing step, the tissue sections are incubated with the secondary reagent Vectastain ABC-AP Kit (Vector, Cat-No AK-5000) in a humidified chamber for another 30 minutes at room temperature. The subsequent chromogenic development step with New Fuchsin Red (Sigma, Cat-No 72200) has to take place for 30 minutes in darkness at room temperature. After an additional washing and rinse step, counterstaining with Mayer's Hemalaun for 30 seconds follows. After additional wash and rinse steps, a dehydration procedure is, followed by coverslipping.

Slide Analysis and Scoring of the Staining Results:

The immunohistochemical staining of the tumors was analyzed semi-quantitatively based on handwritten lists. The following histopathological and immunohistological categories were recorded for CD44 and HA:

(1) The overall percentage of blast cells in the bone marrow biopsy
(2) The estimated percentage of CD44 positive blasts in categories of negative, <10%, 11-50%, 50-80% and 80-100%
(3) The staining intensity in leukemic blasts in categories of 0-1+=very weak or minimal, 1+=weak or slight, 2+=moderate 3+=strong staining intensity
(4) The HA staining intensity peritrabecular (0-3+; close to bone trabecula)
(5) The HA staining intensity intertrabecular (0-3+; between trabecula, within bone marrow cavity)

### Results

In total, 39 mouse xenograft models for 39 different cancer cell lines from different indications for which RNASeq data is available were assessed in order to quantify CD44 isoform expression. Out of those 39 models, 10 show statistically significant tumor growth inhibition (TGI >50%) when treated with the Roche Anti-CD44 antibody (RG7356) while 29 of the models show no or no statistically significant TGI rates smaller than 50%. All available models together with response information and availability of RNASeq and RT-PCR data are shown in Table 1.

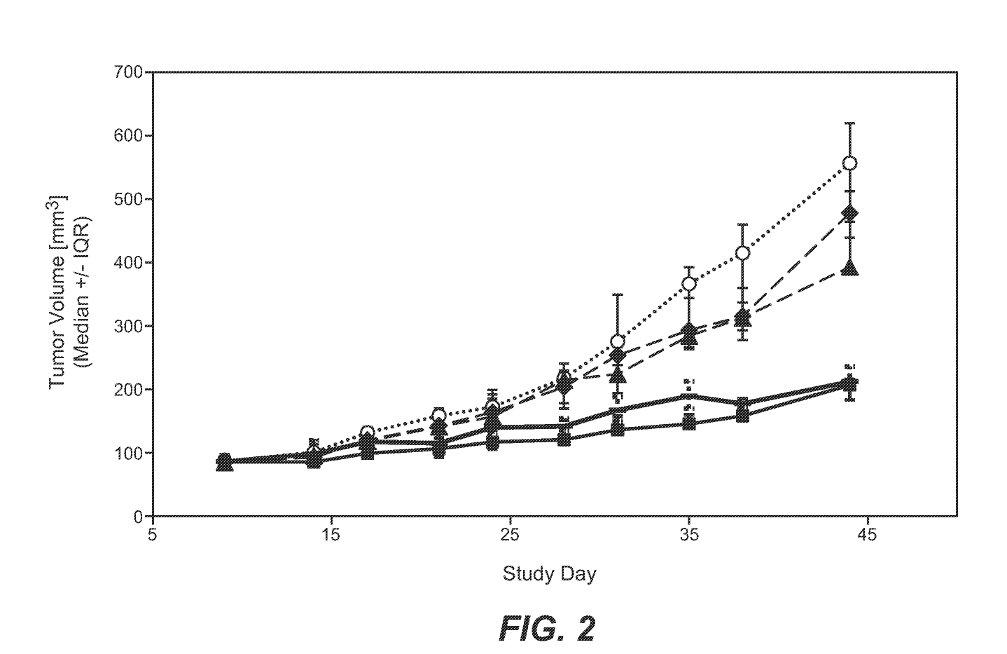

IM7 is a commercially available anti-CD44 antibody that binds to the standard part of IM7 does not induce any significant tumor growth inhibition in a xenograft model that is responsive to RG7356 (FIG. 2): SK-HEP-1 tumor bearing mice treated 5 times weekly with either 1 or 10 mg/kg RG7356 showed highly significant TGI, that was reproducible in several additional experiments, while the effects of IM7 were only minor and non-significant (Tukey-Kramer HSD). Compared with vehicle control (gray line) RG7356 treatment resulted in a dose-dependent tumor growth inhibition of 71% for 1 mg/kg (solid line) p<0.0001 and 74% for 10 mg/kg (solid line with squares) p<0.0001 compared to Tumor growth inhibition of IM7 with 15% for 1 mg/kg (dashed line with diamonds) p=0.69 and 31% for 10 mg/kg (dashed line with triangles) p=0.08.

Moreover, in the IM7 treated groups we observed some transient sedation of the animals following each i.v. administration of the compound which was not observed in the RG7356 treated groups.

Splice variants are not routinely detected and quantified with technologies like Affymetrix or Illumina chip platforms (e.g. HG-U133 Plus 2.0 or HumanHT-12), Western Blot or standard IHC assays (only for selected splice variants where antibodies have been developed). Using RNASeq data was successfully used herein to determine the baseline CD44 isoform composition of all 39 cancer cell lines which were used in the Xenograft models. Detailed images for RNASeq read distribution on all exons and splice junctions of CD44 are shown in FIG. 15. Just like most genes, many cell lines express the CD44 gene as a mixture of different isoforms. For the following analysis, the focus was on the major CD44 isoform (the isoform with the highest frequency among all transcripts that was identified as described in the material and methods section. Further, the CD44s variant that does not contain any of the variable exons and the group of all CD44v variants that contain combinations of variable exons v2-v10 were separately assessed. As major isoforms only CD44s, CD44v3-10 and CD44v8-10 were found to be expressed in the cell lines. The CD44 major isoform status for each cell line is also annotated in Table 1:

TABLE 1
Provides an overview on all currently available Xenograft models, their response to
treatment, availability of experimental data (RNASeq and RT-PCR) as well as major isoform status
as determined by RNASeq. We also show the estimated percentage of CD44s isoform expression
compared to all other CD44v variants as computed on RNASeq Data by MMSeq and (where
available) for RT-PCR. Please note that RT-PCR data and RNASeq data was not generated on
exactly the same samples.

| | | | | | | | CD44s (% |
|---|---|---|---|---|---|---|---|
| | | | | | | | expressed) |
| Tumor | Cell | | | RNA- | RT- | CD44 Major | RNASeq/ |
| Type | Line | Result | Report number | Seq | PCR | Isoform Status | PCR |
| Breast | MDA- | 91- | RDR1033582 | Yes | Yes | S | 78.1/88.4 |
| | MB-231 | 130% | RDR1035833 | | | | |
| | | TGI | RDR1040987 | | | | |
| | | | RDR1040989 | | | | |
| | KPL4 | no | RDR1038914 | Yes | No | V (v3-10) | <5 |
| | | effect | | | | | |
| | MCF7 | no | RDR1038997 | Yes | No | V (v8-10) | 5.4 |
| | | effect | | | | | |
| | MDA- | no | RDR1041407 | Yes | Yes | V (v8-10) | 13.5/18.54 |
| | MB-468 | effect | | | | | |
| | HCC1937 | 15% | RDR1038915 | Yes | No | V (v3-10) | <5 |
| | | TGI | RDR1040253 | | | | |
| | | | RDR1040732 | | | | |
| | JIMT-1 | no | RDR1040990 | Yes | No | V (v3-10) | <5 |
| | | effect | | | | | |
| | BT-20 | 22% | — | Yes | No | V (v8-10) | <5 |
| | | TGI | | | | | |
| HCC | SK-Hep-1 | 30-83% | RDR1035958 | Yes | Yes | S | 89.6/93.1 |
| | | TGI | RDR1037990 | | | | |
| | | | RDR1041071 | | | | |
| | | | RDR1041106 | | | | |
| AML | KG1a | up to | RDR1040973 | Yes | Yes | S | 89.4/92.3 |
| | | 102% | RDR1040974 | | | | |
| | | TGI | RDR1040975 | | | | |
| | HL60 | 81% | RDR1040971 | Yes | Yes | S | 84.6/93.8 |
| | | TGI | | | | | |
| | MV-4- | 94% | RDR1040978 | Yes | No | S | 82.6 |
| | 11 | TGI | RDR1040979 | | | | |
| | | | RDR1040980 | | | | |
| | Kasumi-1 | 92% | RDR1040985 | Yes | No | S | 90.1 |
| | | TGI | | | | | |
| | THP1 | 40% | RDR1040984 | Yes | No | S | 76.6 |
| | | TGI | | | | | |
| | HEL | no | RDR1040986 | Yes | No | S | 86.6 |
| | 92.1.7 | effect | | | | | |
| | MOLM- | 30% | — | Yes | No | S | 80.6 |
| | 13 | TGI | | | | | |
| ALL | MOLT4 | no | RDR1040976 | Yes | Yes | S | 88.4/94.3 |
| | | effect | | | | | |
| Gastric | NCI- | no | RDR1041000 | Yes | No | V (v3-10) | <5 |
| | N87 | effect | | | | | |
| | Hs746T | no | RDR1041001 | Yes | Yes | S | 73.6/95.2 |
| | | effect | | | | | |
| Head& | FaDu | no | RDR1040991 | Yes | Yes | V (v3-10) | <5/<5 |
| Neck | | effect | | | | | |
| | SCC15 | 45% | RDR1040992 | Yes | No | V (v3-10) | 5.3 |
| | | TGI | | | | | |
| | T.tn | 42% | RDR1041666 | Yes | Yes | V (v3-10) | <5/20.6 |
| | | TGI | | | | | |
| | Detroit- | 52% | RDR1040993 | Yes | No | V (v3-10) | <5 |
| | 562 | TGI | | | | | |
| | CAI27 | 100% | RDR1041663 | Yes | No | V (v3-10) | 5.9 |
| Prostate | PC3 | 57% | RDR1041675 | Yes | Yes | S | 61.8/77.8 |
| | 22RV1 | no | RDR1040999 | Yes | No | V (v8-10) | 8.7 |
| | | effect | | | | | |
| Pancreas | PL45 | no | RDR1041106 | Yes | Yes | V (v8-10) | <5/<5 |
| | | effect | RDR1040732 | | | | |
| | Panc-1 | 52% | RDR1041106 | Yes | No | S | 85.7 |
| | | TGI | | | | | |
| Lung | A549 | 15% | RDR1039481 | Yes | Yes | S | 91.1/96.9 |
| | | TGI | | | | | |
| | NCI- | no | RDR1041676 | Yes | No | V (v3-10) | <5 |
| | H520 | effect | | | | | |
| | NCI- | no | RDR1041667 | Yes | Yes | S | 90.6/96.9 |
| | H460 | effect | | | | | |
| | NCI- | no | — | Yes | No | V (v3-10) | <5 |
| | H1993 | effect | | | | | |
| | NCI- | no | — | Yes | No | S | 67.1 |
| | H23 | effect | | | | | |
| | Calu-6 | 17% | — | Yes | No | S | 84.6 |
| Ovarian | SKOV-3 | 38% | RDR1040996 | Yes | No | S | 93.5 |
| | | TGI | | | | | |
| Colon | LS-174T | no | RDR1041398 | Yes | Yes | V (v8-10) | <5/6.8 |
| | | effect | | | | | |
| | Colo205 | no | RDR1040995 | Yes | No | V (v3-10) | <5 |
| | | effect | | | | | |
| | DLD-1 | no | RDR1040994 | Yes | No | V (v8-10) | 24.1 |
| | | effect | | | | | |
| Gastric | MKN45 | No | — | Yes | No | V (v3-10) | <5 |
| Cancer | | effect | | | | | |
| Glio- | U87-MG | 28% | — | Yes | No | S | 95.4 |
| blastoma | | TGI | | | | | |

### Example 2

### CD44 Isoform Status is Predictive for Response to Treatment with RG7356

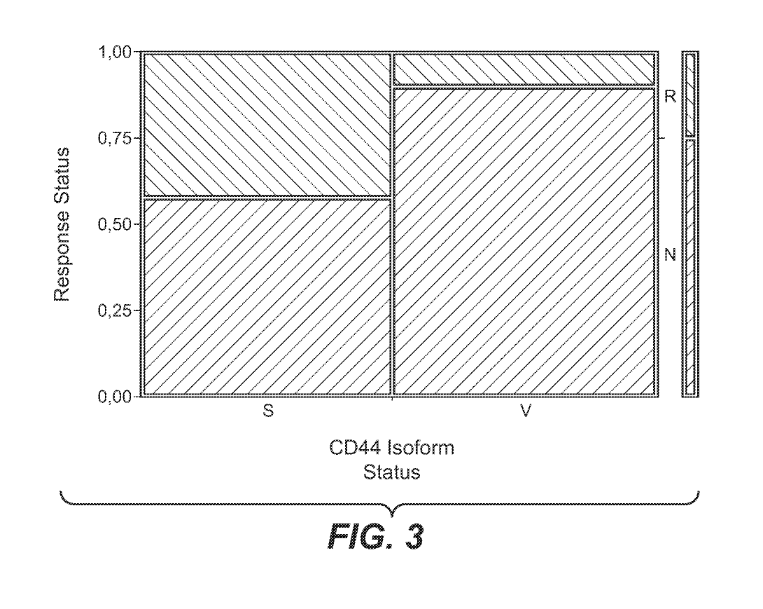

Using the data shown in Table 1 for 39 Xenograft models and the major isoforms of CD44 identified by RNASeq, the relevance of CD44 isoform status for response to treatment was determined. The data showing by response status (R=responder, N=non-responder) in correspondence to isoform status (S=CD44s is major isoform, V=any CD44v variant is major isoform) is shown in Table 2 and FIG. 3.

Given this data, it was computed if CD44s or CD44v are overrepresented in responding or non-responding models. Using the Chi² test it was shown that distribution of isoforms (S and V) is significantly different between responding and non-responding models at a significance level of 0.0186. Surprisingly CD44s variants are overrepresented in the responding group while CD44v variants are under-represented. This data suggests that CD44s is a necessary prerequisite for response to treatment with RG7356. As determined by RNASeq and confirmed by RT-PCR (see Table 1 and 3) expression of CD44s of >60% of the expressed isoform species is found in all CD44s expressing, responding models.

TABLE 2
Distribution of the 39 Xenograft models in Responder (R) and
Non-Responder (N) models as well as CD44s (S) or CD44v (V, any
V variant) expressing models.

| Count | | | |
|---|---|---|---|
| Total % | N | R | |
| S | 11 | 8 | 19 |
| | 28.21 | 20.51 | 48.72 |
| V | 18 | 2 | 20 |
| | 46.15 | 5.13 | 51.28 |
| | 29 | 10 | 39 |
| | 74.36 | 25.64 | |

### Example 3

### Measuring CD44 Isoform Status in the Clinic

In patient samples and the clinical situation RNASeq is likely not the method of choice due to time (turnaround time until results are available) and also cost considerations. The method to be used in the clinic, which is already applied in many approved assays, is RT-PCR. As shown in Table 1, a total of 18 cell lines (for 3 cell lines, namely QG56, BxPC3, HT29 RNASeq and qRT-PCR but no xenograft response data were generated) were assessed for which RNASeq and CD44 RT-PCR data exist. It is of note that measurements were not taken on exactly the same RNA samples. In 94% of the cases (all except for one cell line), major isoform calls made by RNASeq and by qRT-PCR agree. In the single exception, LS174T, the prediction for CD44s vs. CD44v is correct (PCR identifies a CD44v variant as major isoform) but RNASeq identifies CD44v8-10 as major isoform while RT-PCR assigns the highest expression level to CD44v10 (Table 3). This shows that CD44 isoform status can be reliably identified using a technology which is approved for use on clinical patient samples and results in the same CD44s vs. CD44v calls that we obtain with RNASeq.

TABLE 3
Isoform expression levels measured by RT-PCR (columns 2-5) and corresponding major
isoform calls made by RNASeq.

| | | | | | | | RNASeq |
|---|---|---|---|---|---|---|---|
| | | | | | | RNASeq | (Isoform |
| | Tissue | s | v10 | v3-10 | v8-10 | (Major) | Mixture) |
| PC3 | Prostate | 1.467 | 0.2026 | 2.09E−02 | 0.193 | equal | v3-10 and v8-10 |
| | | | | | | | expressed, no |
| | | | | | | | indication for |
| | | | | | | | v10 |
| FaDu | Head and | 3.27E−02 | 0.2221 | 0.4284 | 0.2418 | equal | v8-10 |
| | Neck | | | | | | expressed, v2- |
| | | | | | | | 10 expressed |
| MDA- | Breast | 7.832 | 0.5913 | 0.1693 | 0.262 | equal | v8-10 and v10 |
| MB-231 | | | | | | | expressed |
| MDA- | Breast | 3.886 | 5.26 | 2.496 | 9.31 | equal | s, v3-10 |
| MB-468 | | | | | | | expressed, no |
| | | | | | | | indication for |
| | | | | | | | v10 |
| HT29 | Colon | 3.41E−02 | 0.5062 | 0.1401 | 0.6034 | equal | v3-10, v10 |
| | | | | | | | expressed |
| T.tn | Head and | 0.4447 | 0.2213 | 1.18 | 0.3065 | equal | v2-10 expressed |
| | Neck | | | | | | |
| MOLT4 | ALL | 0.3096 | 7.86E−03 | 2.77E−03 | 7.95E−03 | equal | no other |
| | | | | | | | isoforms |
| KG1 | AML | 8.452 | 0.2856 | 8.15E−02 | 0.3372 | equal | v10 expressed, |
| | | | | | | | some evidence |
| | | | | | | | for v8-10 |
| PL45 | Pancreas | 0.3506 | 3.082 | 1.821 | 3.276 | equal | V3-10 |
| | | | | | | | expressed, v10 |
| | | | | | | | only very low |
| HL60 | AML | 0.8437 | 1.83E−02 | 9.93E−03 | 2.75E−02 | equal | v10 expressed |
| SK-Hep1 | Liver | 18.71 | 0.9449 | 0.1748 | 0.2632 | equal | v10 expressed |
| NCI- | Lung | 1.803 | 3.00E−02 | 7.21E−03 | 2.06E−02 | equal | no other |
| H460M2 | | | | | | | isoforms |
| A549 | Lung | 1.28 | 2.00E−02 | 6.85E−03 | 1.36E−02 | equal | no other |
| | | | | | | | isoforms |
| QG56 | Lung | 0.3999 | 0.958 | 3.029 | 1.472 | equal | v8-10 |
| | | | | | | | expressed, no |
| | | | | | | | indication for |
| | | | | | | | v10 |
| LS174T | Colon | 0.3343 | 2.556 | 0.144 | 1.892 | different | V8-10 is major |
| | | | | | | | isoform in |
| | | | | | | | RNAseq. Very |
| | | | | | | | low expression |
| | | | | | | | of v10 |
| HCC- | Breast | 0.2578 | 1.757 | 1.795 | 1.219 | equal | v8-10 |
| 1937 | | | | | | | expressed, no |
| | | | | | | | indication for |
| | | | | | | | v10 |
| HS_746T | Gastric | 4.842 | 0.1532 | 2.33E−02 | 6.23E−02 | equal | v3-10 expressed |
| BxPC3 | Pancreas | 0.3057 | 0.6721 | 2.901 | 0.6974 | equal | v8-10 |
| | | | | | | | expressed, no |
| | | | | | | | indication for |
| | | | | | | | v10 |

### Example 4

### Different Tumor Indications have Different Expression Patterns of CD44 Isoforms

Publicly available RNASeq data from 17 different tumor indications was used that was published by The Cancer Genome Atlas consortium. TCGA data was processed as described in the material and methods section in order to generate prevalence data for all CD44 isoforms in 17 tumor indications. The results of this analysis show that CD44s, CD44v2-10, CD44v3-10 and CD44v8-10 are expressed as major isoforms and their distribution is shown in FIG. 4. The analysis indicates that CD44 major isoform distribution is very different across tumor indications. Following the results shown above that CD44s seems to be a necessary prerequisite for response, different tumor indications are differently well suited for treatment with RG7356. Some of the indications, like Breast or Colorectal cancer would require patient selection in order to identify the subpopulation of patients expressing CD44s as major isoform. Other tumor indications like AML consist of CD44s expressing tumors only and can thus be chosen for treatment with RG7356 without any additional patient selection.

### Example 5

### CD44 Isoform Status and Hyaluronic Acid (HA)

Hyaluronic Acid (HA) is known to be the major ligand of CD44 and RG7356 interferes with binding of HA to CD44 (FIG. 5). The results clearly show that RG7356 interferes with the binding HA to CD44s-Fc whereas an appropriate antibody isotype control does not have any influence (FIG. 5) This is further confirmed by titration of the binding of CD44s-Fc to HA using two fixed concentrations of with an increasing excess of RG7356 (FIG. 6).

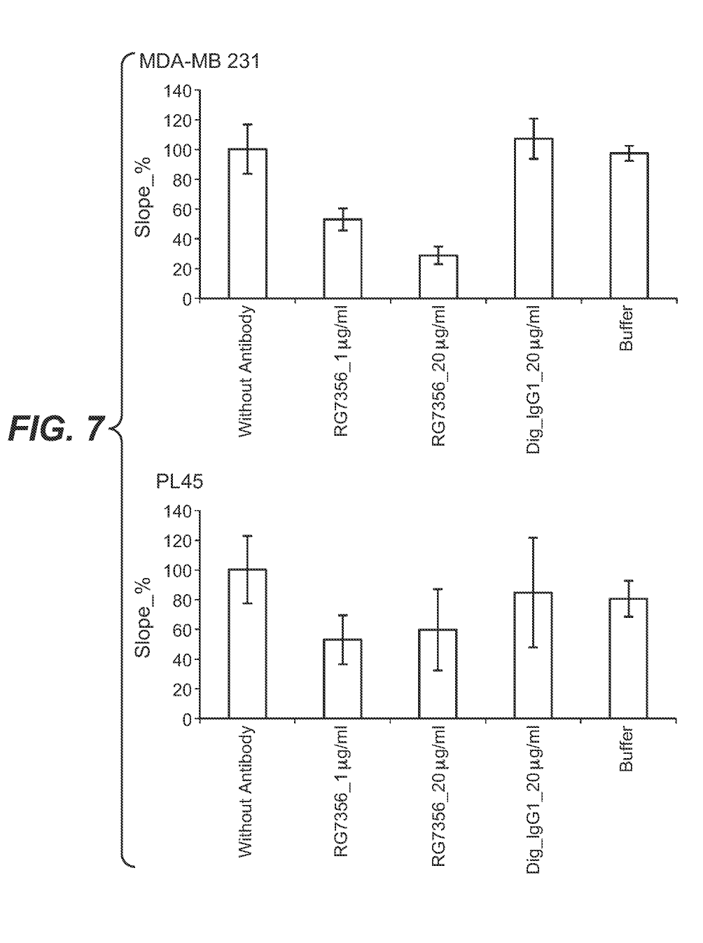

Furthermore, when testing the ability of RG7356 to influence the adherence of CD44 expressing cell lines to HA-coated plates, it was surprisingly found that the adherence of the CD44s expressing cell line MDA-MB-231 is significantly reduced under RG7356 treatment. In contrary, a dose-dependent and statistically significant effect of RG7356 on adherence of CD44v expressing cell line PL45 was not observed (FIG. 7).

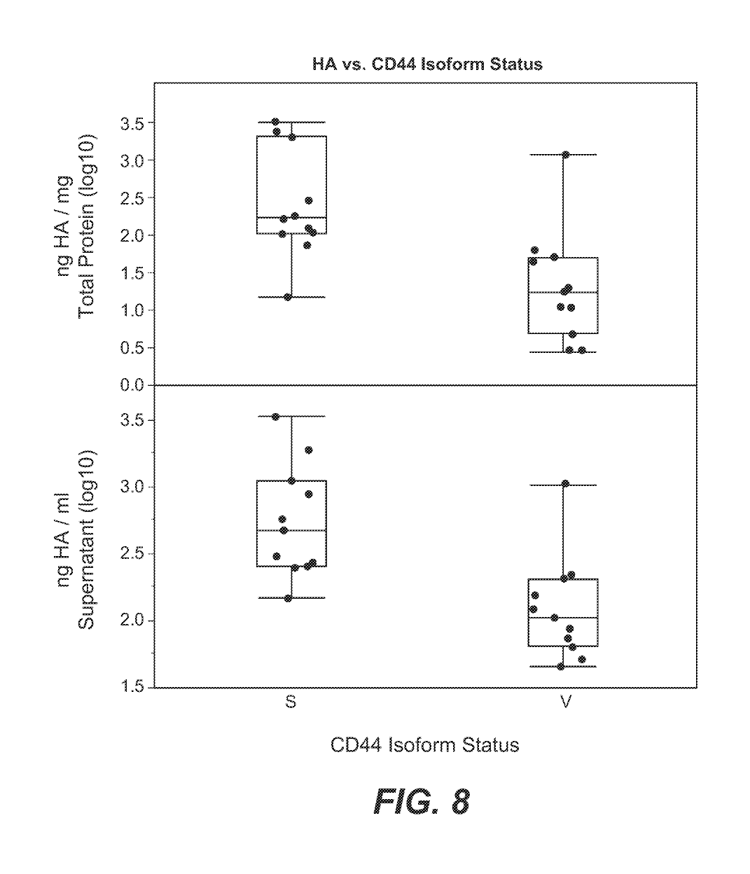

Based on these findings, it was further investigated if HA levels are associated with expression of specific CD44 isoforms. As shown in FIG. 8, it was surprisingly found that HA levels (as measured as ng HA/mg total protein) are higher in cell lines expressing CD44s compared to cell lines expressing CD44v. This difference is statistically significant as measured by a Wilcoxon 1-way test (Chi² approximation) with a p-value of 0.0018. Likewise, when measuring HA in cell line supernatants, cell lines expressing CD44s compared to cell lines expressing CD44v show statistically significant higher HA levels (Wilcoxon 1-way test (Chi² approximation) with a p-value of 0.0012).

Thus, surprisingly HA levels are higher in CD44s-expressing cell lines (responder) compared to non-responder, CD44v expressing cell lines.

### Example 6

### CD44 and Hyaluronic Acid (HA) Expression in AML

When analyzing bone marrow biopsies from AML patients for CD44 and HA expression, we found that 131 of 131 samples examined are CD44 positive. The 131 cases evaluated are composed of 117 primary AML and 14 non-primary AML. The non-primary AML were either MDS (myelodysplastic syndrome)-associated (12 cases), or associated with previous chemotherapy for other malignancies (2 cases).

The percentage of blasts in the biopsies ranged from 25% to 100%, with a majority of cases >50%. Note that the blast content is dependent on the AML subtype, and may per definition be low, for example in cases that evolved out of myelodysplastic syndromes, or in certain AML subtypes with a tendency toward maturation, or prominent fibrosis.

Consistently, CD44 staining was observed at least in a subset of blast cells in all samples. In roughly 50% of AML cases, the percentage of CD44 positive blasts was in a range from 80-100%, and in these cases, with very few exceptions, the intensity of the IHC staining signal was 2+-3+. About 20-25% of cases showed CD44 expression in 50-80% of blast cells at variable intensity. Low percentage of CD44 positive blasts was seen in less than 25% of the cases. The intensity of IHC signals was variable in this subset. The staining for CD44 was not specific for blast cells; for example, CD44 positive non-malignant cells included activated T-lymphocytes, as confirmed by additional lineage markers (CD3, CD20, etc.) performed in single cases on consecutive sections.

The staining for HA showed variable intensities in the bone marrow microenvironment, ranging from very faint signals in normal bone marrow, to strong staining in fibrotic bone marrow samples. Variable patterns were observed, including patchy HA detection in intertrabecular spaces, and cases in which the predominant pattern was a fine lining, covering trabecular bone structures, sometimes only faint and largely limited to the peritrabecular area. Blast cells themselves did not show any evidence of HA production.

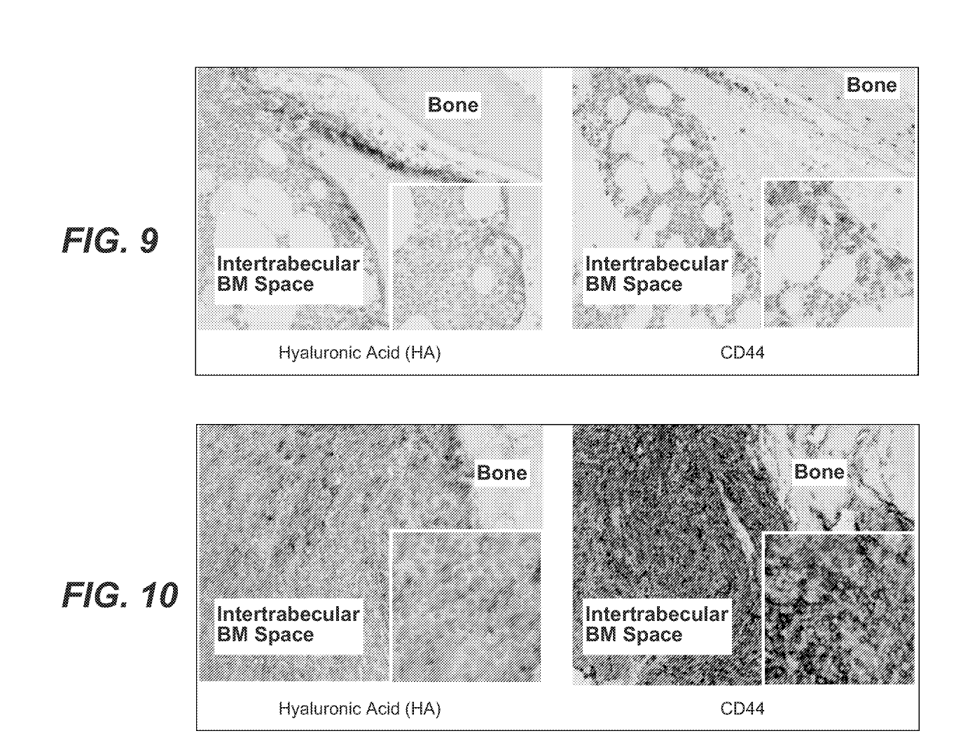

Thus, CD44 and HA seem to be co-localized in AML patient bone marrow, with two characteristic patterns of CD44 expression and HA staining that seem to reflect the role of HA/CD44 interaction (FIG. 9, FIG. 10):

These data further support the RNASeq data obtained for AML and provide additional data that RG7356 treatment should be beneficial for AML patients due to the fact that a) CD44 is overexpressed, b) CD44 expression observed is mainly due to expression of CD44s and c) CD44 and HA co-localize in the bone marrow niche that is causal for AML development, maintenance and resistance to standard therapy.

### Example 7

### Alternative Ways of Measuring HA

HA is synthetized by three protein hyaluronan synthetases 1-3 (HAS 1-3). Expression of HAS mRNA (as measured by RNASeq) significantly correlates with HA levels (Total protein and supernatant) with Pearson correlation coefficients of 0.52 (p-Value 0.01) and 0.60 (p-Value 0.002), respectively; see FIG. 11A.

Likewise, when comparing HA levels as measured by ELISA (from Table 4) with expression levels of HAS 1-3 as measured by qRT-PCR, FIG. 11B shows that HA-rich tumor cell lines have at least one of the three hyaluronan synthetases upregulated (qRT-PCR data). Contrary, cell lines with low or no expression of at least one HA synthetase do not show significant HA levels in their supernatant. Thus, elevated HAS-expression correlates with elevated elvels of HA.

### Example 8

### Clinical Relevance of CD44 Isoform Expression Pattern as Response Prediction Marker for RG7356 Treatment

### Methods: Patient Data (Phase 1 Clinical Trial) and RNASeq of Patient Samples

Data were taken from a recently completed multicenter, open-label dose-escalation Phase I clinical study of RG7356 at 6 study sites in France, the Netherlands and the USA (clinicaltrials.gov identifier NCT01358903). The study was conducted in accordance with the Declaration of Helsinki, current International Conference on Harmonisation of Technical Requirements for Registration of Pharmaceuticals for Human Use (ICH) guidelines and all applicable regulatory and ethical requirements. All subjects provided written informed consent before study-related procedures were performed. Patients with metastatic and/or locally advanced, CD44-expressing, malignant solid tumors who were not amenable to standard treatment were enrolled and received RG7356 on a q2w (every two weeks) or qw (every week) schedule as an intravenous infusion. Tumor response was evaluated according to RECIST (version 1.1) criteria up to 28 days prior to the first drug administration and every 6 weeks ±7 days while a patient was on study.

### Results

To confirm CD44s as response prediction marker in the clinical setting, pre-treatment tumor biopsies of patients from a recently completed phase 1 dose-escalation clinical trial (clinicaltrials.gov identifier NCT01358903) were tested for their predominant CD44 isoform expression via RNASeq and compared to the respective clinical activity of RG7356 observed in those advanced cancer patients.

A tumor biopsy was taken from each patient before enrollment into the study to assess CD44 protein expression by immunohistochemistry (Ventana SP37 Rabbit Monoclonal Primary Antibody, Cat-No 790-4537). Collection of biopsies was guided by ultrasound or computer tomography using an 18 Gauge needle to provide cores of at least 20 mm in length. Total RNA was prepared from fresh frozen tumor tissue of at least 6-8 sections of 10 μm thickness. In cases of insufficient material, the entire biopsy was used.

RNA isolation was performed using RNeasy Minikit adding QlAshredder and RNase-free DNase set (Qiagen, Germany). Kit procedures were performed according to manufacturer's protocol. Each sample was centrifuged at 16.000 g for 2 minutes, aliquoted and analyzed via NanoDrop. RNA quality was checked using Agilent Bioanalyzer. Library preparation from cDNA was performed using the Illumina TruSeq Stranded Total RNA Kit using 0.1-1 μg or total RNA. The generated libraries were applied for RNA Sequencing using a Illumina HiSeq2000 instrument generating on average 50 million reads of 50 bp paired-end reads per sample. RNASeq data was analyzed as described above.

Based on clinical response data and CD44 isoform measurements (determined using RNASeq) it was demonstrated that all patients that showed a reduction in tumor size measured as “best percent”-tumor change on target lesions express CD44s as major isoform. Those patients come from different tumor indications (colorectal and thymus carcinoma). No patient expressing CD44v as major isoform shows signs for clinical response as measured in a reduction of tumor size.

The present invention refers to the following nucleotide and amino acid sequences:

The sequences provided herein are available in the NCBI database or in the ENSEMBL databaswe and can be retrieved from world wide web at ncbi.nlm.nih.gov/sites/entrez?db=gene; Theses sequences also relate to annotated and modified sequences. The present invention also provides techniques and methods wherein homologous sequences, fragments, and variants of the concise sequences provided herein are used. Preferably, such “variants” are genetic variants.

SEQ ID No. 12:

Nucleotide sequence encoding homo sapiens CD44 isoform CD44s, ENSEMBL transcript id:

ENST00000263398.

SEQ ID No. 13:

Amino acid sequence of homo sapiens CD44 isoform CD44s, ENST00000263398.

SEQ ID No. 14:

Nucleotide sequence encoding homo sapiens CD44 isoform CD44s; >ENST00000434472_{—}11; CD44s with shortened UTR

SEQ ID No. 15:

Amino acid sequence of homo sapiens CD44 isoform CD44s; >ENST00000434472_{—}11; CD44s with shortened UTR

SEQ ID No. 16:

Nucleotide sequence encoding homo sapiens CD44 isoform CD44v2-v9; >ENST00000437706|CD44v2-v9

SEQ ID No. 17:

Amino acid sequence of homo sapiens CD44 isoform CD44v2-v9; >ENST00000437706|CD44v2-v9

SEQ ID No. 18:

Nucleotide sequence encoding homo sapiens CD44 isoform CD44v2-10, >ENST00000278385

SEQ ID No. 19:

Amino acid sequence of homo sapiens CD44 isoform CD44v2-10, >ENST00000278385

SEQ ID No. 20:

Nucleotide sequence encoding homo sapiens CD44 isoform CD44v2-10; >ENST00000449691|CD44v2-v10 skipping exon v6

SEQ ID No. 21:

Amino acid sequence of homo sapiens CD44 isoform CD44v2-10; >ENST00000449691|CD44v2-v10 skipping exon v6

SEQ ID No. 22:

Nucleotide sequence encoding homo sapiens CD44 isoform CD44v2-10; >ENST00000437835|CD44v2-v10 shortened exon v3

SEQ ID No. 23:

Amino acid sequence of homo sapiens CD44 isoform CD44v2-10; >ENST00000437835|CD44v2-v10 shortened exon v3

SEQ ID No. 24:

Nucleotide sequence encoding homo sapiens CD44 isoform CD44v2-v10; >ENST00000433354|CD44v2-v10 skipping exon v9

SEQ ID No. 25:

Amino acid sequence of homo sapiens CD44 isoform CD44v2-v10; >ENST00000433354|CD44v2-v10 skipping exon v9

SEQ ID No. 26:

Nucleotide sequence encoding homo sapiens CD44 isoform CD44v2-v10; >ENST00000428726|CD44v2-v10 shortened UTR

SEQ ID No. 27:

Amino acid sequence of homo sapiens CD44 isoform CD44v2-v10; >ENST00000428726|CD44v2-v10 shortened UTR

SEQ ID No. 28:

Nucleotide sequence encoding homo sapiens CD44 isoform CD44v2-v10; >ENST00000423360_{—}11 |CD44v2-v10 shortened constitutive exon right before v2

SEQ ID No. 29:

Amino acid sequence encoding homo sapiens CD44 isoform CD44v2-v10; >ENST000004233601CD44v2-v10 shortened constitutive exon right before v2

SEQ ID No. 30:

Nucleotide sequence encoding homo sapiens CD44 isoform CD44v3-10, >ENST00000279452

SEQ ID No. 31:

Amino acid sequence of homo sapiens CD44 isoform CD44v3-10, >ENST00000279452

SEQ ID No. 32:

Nucleotide sequence encoding homo sapiens CD44 isoform CD44v3-10; >ENST00000415148|CD44v3-v10 shortened UTR

SEQ ID No. 33:

Amino acid sequence of homo sapiens CD44 isoform CD44v3-10; >ENST00000415148|CD44v3-v10 shortened UTR

SEQ ID No. 34:

Nucleotide sequence encoding homo sapiens CD44 isoform CD44v8-10>ENST00000352818

SEQ ID No. 35:

Amino acid sequence of homo sapiens CD44 isoform CD44v8-10, >ENST00000352818

SEQ ID No. 36:

Nucleotide sequence encoding homo sapiens CD44 isoform CD44v8-v10 (shortened UTR); >ENST00000433892|CD44v8-v10 (shortened UTR)

SEQ ID No. 37:

Amino acid sequence of homo sapiens CD44 isoform CD44v8-v10 (shortened UTR); >ENST00000433892|CD44v8-v10 (shortened UTR)

SEQ ID No. 38:

Nucleotide sequence encoding homo sapiens CD44 isoform CD44v6; >not annotated in Ensembl|CD44v6

SEQ ID No. 39:

Amino acid sequence of homo sapiens CD44 isoform CD44v6; >not annotated in Ensembl|CD44v6

SEQ ID No. 40:

Nucleotide sequence encoding homo sapiens CD44 isoform CD44v8, >ENST00000360158|CD44v8

SEQ ID No. 41:

Amino acid sequence of homo sapiens CD44 isoform CD44v8; >ENST00000360158|CD44v8

SEQ ID No. 42:

Nucleotide sequence encoding homo sapiens CD44 isoform CD44v10, >ENST00000425428

SEQ ID No. 43:

Amino acid sequence of homo sapiens CD44 isoform CD44v10, >ENST00000425428

SEQ ID No. 44:

Nucleotide sequence of exon Cl of homo sapiens CD44

SEQ ID No. 45:

Amino acid sequence encoded by exon Cl of homo sapiens CD44

SEQ ID No. 46:

Nucleotide sequence of exon C2 of homo sapiens CD44

SEQ ID No. 47:

Amino acid sequence encoded by exon C2 of homo sapiens CD44

SEQ ID No. 48:

Nucleotide sequence of exon C3 of homo sapiens CD44

SEQ ID No. 49: Amino acid sequence encoded by exon C3 of homo sapiens CD44

SEQ ID No. 50:

Nucleotide sequence of exon C4 of homo sapiens CD44

SEQ ID No. 51:

Amino acid sequence encoded by exon C4 of homo sapiens CD44

SEQ ID No. 52:

Nucleotide sequence of exon C5 of homo sapiens CD44

SEQ ID No. 53:

Amino acid sequence encoded by exon C5 of homo sapiens CD44

SEQ ID No. 54:

Nucleotide sequence of exon V2 of homo sapiens CD44

SEQ ID No. 55:

Amino acid sequence encoded by exon V2 of homo sapiens CD44

SEQ ID No. 56:

Nucleotide sequence of exon V3 of homo sapiens CD44

SEQ ID No. 57:

Amino acid sequence encoded by exon V3 of homo sapiens CD44

SEQ ID No. 58:

Nucleotide sequence of exon V4 of homo sapiens CD44

SEQ ID No. 59:

Amino acid sequence encoded by exon V4 of homo sapiens CD44

SEQ ID No. 60:

Nucleotide sequence of exon V5 of homo sapiens CD44

SEQ ID No. 61:

Amino acid sequence encoded by exon V5 of homo sapiens CD44

SEQ ID No. 62:

Nucleotide sequence of exon V6 of homo sapiens CD44

SEQ ID No. 63:

Amino acid sequence encoded by exon V6 of homo sapiens CD44

SEQ ID No. 64:

Nucleotide sequence of exon V7 of homo sapiens CD44

SEQ ID No. 65:

Amino acid sequence encoded by exon V7 of homo sapiens CD44

SEQ ID No. 66:

Nucleotide sequence of exon V8 of homo sapiens CD44

SEQ ID No. 67:

Amino acid sequence encoded by exon V8 of homo sapiens CD44

SEQ ID No. 68:

Nucleotide sequence of exon V9 of homo sapiens CD44

SEQ ID No. 69:

Amino acid sequence encoded by exon V9 of homo sapiens CD44

SEQ ID No. 70:

Nucleotide sequence of exon V10 of homo sapiens CD44

SEQ ID No. 71:

Amino acid sequence encoded by exon V10 of homo sapiens CD44

SEQ ID No. 72:

Nucleotide sequence of exon C6 of homo sapiens CD44

SEQ ID No. 73:

Amino acid sequence encoded by exon C6 of homo sapiens CD44

SEQ ID No. 74:

Nucleotide sequence of exon C7 of homo sapiens CD44

SEQ ID No. 75:

Amino acid sequence encoded by exon C7 of homo sapiens CD44

SEQ ID No. 76:

Nucleotide sequence of exon C8 of homo sapiens CD44

SEQ ID No. 77:

Amino acid sequence encoded by exon C8 of homo sapiens CD44

SEQ ID No. 78:

Nucleotide sequence of exon C9 of homo sapiens CD44 (for this exon longer and shorter versions of the UTR part are known):

SEQ ID No. 79:

Amino acid sequence encoded by exon C9 of homo sapiens CD44

SEQ ID No. 80:

Nucleotide sequence encoding hyaluronic acid synthetase 1 (HAS1), >ENST00000222115

SEQ ID No. 81:

Amino acid sequence of hyaluronic acid synthetase 1 (HAS1)>ENST00000222115

SEQ ID No. 82:

Nucleotide sequence encoding hyaluronic acid synthetase 2 (HAS2), >ENST00000303924,

SEQ ID No. 83:

Amino acid sequence of hyaluronic acid synthetase 2 (HAS2), >ENST00000303924,

SEQ ID No. 84:

Nucleotide sequence encoding hyaluronic acid synthetase 3 (HAS3), >ENST00000306560

SEQ ID No. 85:

Amino acid sequence of hyaluronic acid synthetase 3 (HAS3)>ENST00000306560

SEQ ID No. 86:

Amino acid sequence of epitope on the constant region of CD44

The following relates to RNA sequences corresponding to given DNA sequence as provided herein above. As described herein above, the term “RNA sequences corresponding to a DNA sequence” as used herein means that the respective RNA sequence is identical to a given DNA sequence as provided herein, with the exception that the tymidine (T) nucleic acid residues are replaced by uracil (U) nucleic acid residues.

SEQ ID No. 117:

RNA sequence encoding homo sapiens CD44 isoform CD44s corresponding to the DNA sequence as shown in SEQ ID NO. 12; ENSEMBL transcript id: ENST00000263398.

SEQ ID No. 118:

RNA sequence encoding homo sapiens CD44 isoform CD44s corresponding to the DNA sequence as shown in SEQ ID NO. 14; >ENST00000434472_{—}11; CD44s with shortened UTR

SEQ ID No. 119:

RNA sequence encoding homo sapiens CD44 isoform CD44v2-v9 corresponding to the DNA sequence as shown in SEQ ID NO. 16; >ENST00000437706|CD44v2-v9

SEQ ID No. 120:

RNA sequence encoding homo sapiens CD44 isoform CD44v2-10 corresponding to the DNA sequence as shown in SEQ ID NO. 18, >ENST00000278385

SEQ ID No. 121:

RNA sequence encoding homo sapiens CD44 isoform CD44v2-10 corresponding to the DNA sequence as shown in SEQ ID NO. 20; >ENST00000449691|CD44v2-v10 skipping exon v6

SEQ ID No. 122:

RNA sequence encoding homo sapiens CD44 isoform CD44v2-10 corresponding to the DNA sequence as shown in SEQ ID NO. 22; >ENST00000437835|CD44v2-v10 shortened exon v3

SEQ ID No. 123:

RNA sequence encoding homo sapiens CD44 isoform CD44v2-v10 corresponding to the DNA sequence as shown in SEQ ID NO. 24; >ENST00000433354|CD44v2-v10 skipping exon v9

SEQ ID No. 124:

RNA sequence encoding homo sapiens CD44 isoform CD44v2-v10 corresponding to the DNA sequence as shown in SEQ ID NO. 26; >ENST00000428726|CD44v2-v10 shortened UTR

SEQ ID No. 125:

RNA sequence encoding homo sapiens CD44 isoform CD44v2-yl0corresponding to the DNA sequence as shown in SEQ ID NO. 28; >ENST00000423360_{—}11 |CD44v2-v10 shortened constitutive exon right before v2

SEQ ID No. 126:

RNA sequence encoding homo sapiens CD44 isoform CD44v3-10 corresponding to the DNA sequence as shown in SEQ ID NO. 30; >ENST00000279452

SEQ ID No. 127:

RNA sequence encoding homo sapiens CD44 isoform CD44v3-10corresponding to the DNA sequence as shown in SEQ ID NO. 32; >ENST00000415148|CD44v3-v10 shortened UTR

SEQ ID No. 128:

RNA sequence encoding homo sapiens CD44 isoform CD44v8-10 corresponding to the DNA sequence as shown in SEQ ID No. 34; >ENST00000352818

SEQ ID No. 129:

RNA sequence encoding homo sapiens CD44 isoform CD44v8-v10 (shortened UTR) corresponding to the DNA sequence as shown in SEQ ID No. 36; >ENST00000433892|CD44v8-v10 (shortened UTR)

SEQ ID No. 130:

RNA sequence encoding homo sapiens CD44 isoform CD44v6 corresponding to the DNA sequence as shown in SEQ ID No. 38

SEQ ID No. 131:

RNA sequence encoding homo sapiens CD44 isoform CD44v8 corresponding to the DNA sequence as shown in SEQ ID No. 40, >ENST00000360158|CD44v8

SEQ ID No. 132:

RNA sequence encoding homo sapiens CD44 isoform CD44v10 corresponding to the DNA sequence as shown in SEQ ID No. 42, >ENST00000425428

SEQ ID No. 133:

RNA sequence of exon Cl of homo sapiens CD44 corresponding to the DNA sequence as shown in SEQ ID No. 44:

SEQ ID No. 134:

RNA sequence of exon C2 of homo sapiens CD44 corresponding to the DNA sequence as shown in SEQ ID No. 46:

SEQ ID No. 135:

RNA sequence of exon C3 of homo sapiens CD44 corresponding to the DNA sequence as shown in SEQ ID No. 48:

SEQ ID No. 136:

RNA sequence of exon C4 of homo sapiens CD44 corresponding to the DNA sequence as shown in SEQ ID No. 50:

SEQ ID No. 137:

RNA sequence of exon C5 of homo sapiens CD44 corresponding to the DNA sequence as shown in SEQ ID No. 52

SEQ ID No. 138:

RNA sequence of exon V2 of homo sapiens CD44 corresponding to the DNA sequence as shown in SEQ ID No. 54:

SEQ ID No. 139:

RNA sequence exon V3 of homo sapiens CD44 corresponding to the DNA sequence as shown in SEQ ID No. 56:

SEQ ID No. 140:

RNA sequence of exon V4 of homo sapiens CD44 corresponding to the DNA sequence as shown in SEQ ID No. 58:

SEQ ID No. 141:

RNA sequence of exon V5 of homo sapiens CD44 corresponding to the DNA sequence as shown in SEQ ID No. 60:

SEQ ID No. 142:

RNA sequence of exon V6 of homo sapiens CD44 corresponding to the DNA sequence as shown in SEQ ID No. 62:

SEQ ID No. 143:

RNA sequence of exon V7 of homo sapiens CD44 corresponding to the DNA sequence as shown in SEQ ID No. 64:

SEQ ID No. 144:

RNA sequence of exon V8 of homo sapiens CD44 corresponding to the DNA sequence as shown in SEQ ID No. 66:

SEQ ID No. 145:

RNA sequence of exon V9 of homo sapiens CD44 corresponding to the DNA sequence as shown in SEQ ID No. 68:

SEQ ID No. 146:

RNA sequence of exon V10 of homo sapiens CD44 corresponding to the DNA sequence as shown in SEQ ID No. 70:

SEQ ID No. 147:

RNA sequence of exon C6 of homo sapiens CD44 corresponding to the DNA sequence as shown in SEQ ID No. 72:

SEQ ID No. 148:

RNA sequence of exon C7 of homo sapiens CD44 corresponding to the DNA sequence as shown in SEQ ID No. 74:

SEQ ID No. 149:

RNA sequence of exon C8 of homo sapiens CD44 corresponding to the DNA sequence as shown in SEQ ID No. 76:

SEQ ID No. 150:

RNA sequence of exon C9 of homo sapiens CD44 (for this exon longer and shorter versions of the UTR part are known):corresponding to the DNA sequence as shown in SEQ ID No. 78:

SEQ ID No. 151:

RNA sequence encoding hyaluronic acid synthetase 1 (HAS1), corresponding to the DNA sequence as shown in SEQ ID No. 80; >ENST00000222115

SEQ ID No. 152:

RNA sequence encoding hyaluronic acid synthetase 2 (HAS2) corresponding to the DNA sequence as shown in SEQ ID No. 82; >ENST00000303924,

SEQ ID No. 153:

RNA sequence encoding hyaluronic acid synthetase 3 (HAS3) corresponding to the DNA sequence as shown in SEQ ID NO.84; >ENST00000306560

All references cited herein are fully incorporated by reference. Having now fully described the invention, it will be understood by a person skilled in the art that the invention may be practiced within a wide and equivalent range of conditions, parameters and the like, without affecting the spirit or scope of the invention or any embodiment thereof.

We claim:

1. A method of determining whether a tumor cell or a cancer cell is responsive to an anti-CD44 antibody or to an antigen binding fragment thereof, said method comprising determining the major CD44 isoform in a sample of a patient, wherein if said major CD44 isoform is CD44s, said tumor cell or cancer cell is responsive to said anti-CD44 antibody or to an antigen binding fragment thereof.

2. The method of claim 1, wherein said anti-CD44 antibody is a chimeric version of the monoclonal antibody produced by the hybridoma deposited with the ATCC with accession number PTA-4621.

3. The method of claim 1, wherein said anti-CD44 antibody is a humanized version of the monoclonal antibody produced by the hybridoma deposited with the ATCC with accession number PTA-4621.

4. The method of claim 1, wherein said anti-CD44 antibody comprises one or more of a V_{H} CDR1 having the amino acid sequence of SEQ ID NO:3, a V_{H} CDR2 having the amino acid sequence of SEQ ID NO:4, a V_{H} CDR3 having the amino acid sequence of SEQ ID NO:5, a V_{L} CDR1 having the amino acid sequence of SEQ ID NO:6, a V_{L} CDR2 having the amino acid sequence of SEQ ID NO:7 and a V_{L} CDR3 having the amino acid sequence of SEQ ID NO:8.

5. The method of claim 4, wherein said anti-CD44 antibody is a chimeric antibody.

6. The method of claim 5, wherein said anti-CD44 antibody comprises a V_{H} domain having the amino acid sequence of SEQ ID NO:1.

7. The method of claim 5, wherein said anti-CD44 antibody comprises a V_{L} domain having the amino acid sequence of SEQ ID NO:2.

8. The method of claim 4, wherein said anti-CD44 antibody is a humanized antibody.

9. The method of claim 8, wherein said humanized anti-CD44 antibody comprises a V_{H} domain having the amino acid sequence of SEQ ID NO:9 or SEQ ID NO: 10.

10. The method of claim 8, wherein said humanized anti-CD44 antibody comprises a V_{L} domain having the amino acid sequence of SEQ ID NO:11.

11. The method of claim 1, wherein said anti-CD44 antibody competes for binding with the anti-CD44 antibody produced by the hybridoma deposited with the ATCC with Accession number PTA-4621.

12. The method of claim 1, wherein said anti-CD44 antibody interferes with the interaction of CD44 and hyaluronic acid.

13. The method of claim 1, further comprising determining the amount and/or number of all CD44 isoform molecules in said sample.

14. The method of claim 1, wherein a CD44 isoform is determined to be the major isoform, if at least 60% of all CD44 isoform molecules in said sample are molecules of said CD44 isoform.

15. The method of claim 13, wherein the amount and/or number of all CD44 isoform molecules in said sample is assessed by Real Time PCR or Whole Transcriptome Shotgun Sequencing (RNAseq).

16. The method of claim 1, wherein the nucleic acid sequence encoding said CD44 isoform CD44s is shown in SEQ ID NO: 12 or 14.

17. The method of claim 1, wherein the amino acid sequence of said CD44 isoform CD44s is shown in SEQ ID NO: 13 or 15.

18. The method of claim 1, wherein said patient is suffering from a hematological or solid cancer, is suspected of suffering from a hematological or solid cancer, or is prone to suffering from a hematological or solid cancer.

19. The method of claim 18, wherein said hematological cancer is selected from the group consisting of acute myeloid leukemia (AML), chronic myeloid leukemia (CML), chronic lymphoid leukemia (CLL), multiple myeloma (MM), and myelodysplastic syndrome (MDS), and wherein said solid cancer is selected from the group consisting of melanoma, lung adenocarcinoma, renal cancer, kidney cancer, mesothelioma, lung squeamous cell carcinoma, breast cancer, liver hepatocellular carcinoma, glioblastoma, gastric cancer, ovarian cancer, bladder cancer, prostate cancer, and head and neck cancer.

20. The method of claim 1, further comprising determining the level of hyaluronic acid and/or the level of one or more hyaluronic acid synthetases 1 to 3.

21. The method of claim 20, wherein if the level of hyaluronic acid is increased in comparison with a control and/or if the level of one or more hyaluronic acid synthetases is increased in comparison with a control, said tumor cell or cancer cell is responsive to said anti-CD44 antibody.

22. The method of claim 21, wherein said level of hyaluronic acid and/or said level of one or more hyaluronic acid synthetases is at least 2.5-fold, preferably at least 5-fold increased in comparison to the control.

23. The method of claim 20, wherein said level of said one or more hyaluronic acid synthetases is the expression level.

24. The method of claim 23, wherein said expression level is the mRNA expression level.

25. The method of claim 24, wherein the mRNA expression level is assessed by in situ hybridization, micro-arrays, or RealTime PCR.

26. The method of claim 23, wherein said expression level is the protein expression level.

27. The method of claim 26, wherein said protein expression level is assessed by immunoassay, gel- or blot-based methods, IHC, mass spectrometry, flow cytometry, or FACS.

28. The method of claim 1, further comprising the step of administering to the patient the anti-CD44 antibody if the patient is determined to have the CD44s isoform as the major isoform.

29. A method of treating a patient, said method comprising selecting a cancer patient, wherein a tumor cell or cancer cell of a sample of said patient is determined to have CD44s as major CD44 isoform and administering to the patient an effective amount of an anti-CD44 antibody or an antigen binding fragment thereof.


SEQUENCE LISTING (153 sequences)

SEQ ID NO: 1 - protein, 124 aa, Artificial Sequence
  VH domain
EVKLLESGGGLVQPGGSLKLSCATSGFDFSRYWMSWVRQAPGKGLEWIGEVNPDSTSINYTPSLKDQFIISRDNAKNTLDLQMSKVSSEDTALYYCTRPNYYGSRYHYYAMDYWGQGTSVTVSS

SEQ ID NO: 2 - protein, 107 aa, Artificial Sequence
  VL domain
DIQMTQTTSSLSVSLGDRVTINCRASQDINNYLNWYQQKPDGTVKLLIYYTSRLHSGVPSRFSGSGSGTDFSLTISNLEKEDVATYFCQQGSTLPFTFGSGTKLEIK

SEQ ID NO: 3 - protein, 5 aa, Artificial Sequence
  VH CDR1
RYWMS

SEQ ID NO: 4 - protein, 17 aa, Artificial Sequence
  VH CDR2
EVNPDSTSINYTPSLKD

SEQ ID NO: 5 - protein, 15 aa, Artificial Sequence
  VH CDR3
PNYYGSRYHYYAMDY

SEQ ID NO: 6 - protein, 11 aa, Artificial Sequence
  VL CDR1
RASQDINNYLN

SEQ ID NO: 7 - protein, 7 aa, Artificial Sequence
  VL CDR2
YTSRLHS

SEQ ID NO: 8 - protein, 9 aa, Artificial Sequence
  VL CDR3
QQGSTLPFT

SEQ ID NO: 9 - protein, 124 aa, Artificial Sequence
  VH domain
EVQLVESGGGLVQPGGSLRLSCAASGFDFSRYWMSWVRQAPGKGLVWVGEVNPDSTSINYTPSLKDRFTISRDNAKNTLYLQMNSLRAEDTAVYYCTRPNYYGSRYHYYAMDYWGQGTLVTVSS

SEQ ID NO: 10 - protein, 124 aa, Artificial Sequence
  VH domain
EVQLVESGGGLVQPGGSLRLSCATSGFDFSRYWMSWVRQAPGKGLVWIGEVNPDSTSINYTPSLKDQFTISRDNAKNTLYLQMNSLRAEDTAVYYCTRPNYYGSRYHYYAMDYWGQGTLVTVSS

SEQ ID NO: 11 - protein, 107 aa, Artificial Sequence
  VL domain
DIQMTQSPSSLSASVGDRVTITCRASQDINNYLNWYQQKPGKAPKLLIYYTSRLHSGVPSRFSGSGSGTDFTFTISSLQPEDIATYYCQQGSTLPFTFGQGTKLEIK

SEQ ID NO: 12 - DNA, 4586 bp, Homo sapiens
  source 1..4586: /mol_type=“unassigned DNA” /note=“Nucleotide sequence encoding homo sapiens CD44 isoform CD44s” /organism=“Homo sapiens”
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccagagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaac

SEQ ID NO: 13 - protein, 361 aa, Homo sapiens
  Amino acid sequence of homo sapiens CD44 isoform CD44s
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATRDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 14 - DNA, 1713 bp, Homo sapiens
  source 1..1713: /mol_type=“unassigned DNA” /note=“Nucleotide sequence encoding homo sapiens CD44 isoform CD44s” /organism=“Homo sapiens”
atttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccagagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttca

SEQ ID NO: 15 - protein, 338 aa, Homo sapiens
  Amino acid sequence of homo sapiens CD44 isoform CD44s
LNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATRDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 16 - DNA, 5525 bp, Homo sapiens
  source 1..5525: /mol_type=“unassigned DNA” /note=“Nucleotide sequence encoding homo sapiens CD44 isoform CD44v2-v9” /organism=“Homo sapiens”
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccactttgatgagcactagtgctacagcaactgagacagcaaccaagaggcaagaaacctgggattggttttcatggttgtttctaccatcagagtcaaagaatcatcttcacacaacaacacaaatggctggtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcagagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaac

SEQ ID NO: 17 - protein, 674 aa, Homo sapiens
  Amino acid sequence of homo sapiens CD44 isoform CD44v2-v9
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSRDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 18 - DNA, 5729 bp, Homo sapiens
  source 1..5729: /mol_type=“unassigned DNA” /note=“Nucleotide sequence encoding homo sapiens CD44 isoform CD44v2-10” /organism=“Homo sapiens”
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccactttgatgagcactagtgctacagcaactgagacagcaaccaagaggcaagaaacctgggattggttttcatggttgtttctaccatcagagtcaaagaatcatcttcacacaacaacacaaatggctggtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaac

SEQ ID NO: 19 - protein, 742 aa, Homo sapiens
  Amino acid sequence of homo sapiens CD44 isoform CD44v2-10
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 20 - DNA, 5600 bp, Homo sapiens
  source 1..5600: /mol_type=“unassigned DNA” /note=“Nucleotide sequence encoding homo sapiens CD44 isoform CD44v2-10” /organism=“Homo sapiens”
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccactttgatgagcactagtgctacagcaactgagacagcaaccaagaggcaagaaacctgggattggttttcatggttgtttctaccatcagagtcaaagaatcatcttcacacaacaacacaaatggctggtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaacagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaac

SEQ ID NO: 21 - protein, 699 aa, Homo sapiens
  Amino acid sequence of homo sapiens CD44 isoform CD44v2-10
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTTASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 22 - DNA, 5705 bp, Homo sapiens
  source 1..5705: /mol_type=“unassigned DNA” /note=“Nucleotide sequence encoding homo sapiens CD44 isoform CD44v2-10” /organism=“Homo sapiens”
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccactttgatgagcactagtgctacagcaactgagacagcaaccaagaggcaagaaacctgggattggttttcatggttgtttctaccatcagagtcaaagaatcatcttcacacaacaacacaaatggctgcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaac

SEQ ID NO: 23 - protein, 734 aa, Homo sapiens
  Amino acid sequence of homo sapiens CD44 isoform CD44v2-10
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 24 - DNA, 5639 bp, Homo sapiens
  source 1..5639: /mol_type=“unassigned DNA” /note=“Nucleotide sequence encoding homo sapiens CD44 isoform CD44v2-v10” /organism=“Homo sapiens”
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccactttgatgagcactagtgctacagcaactgagacagcaaccaagaggcaagaaacctgggattggttttcatggttgtttctaccatcagagtcaaagaatcatcttcacacaacaacacaaatggctggtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaac

SEQ ID NO: 25 - protein, 712 aa, Homo sapiens
  Amino acid sequence of homo sapiens CD44 isoform CD44v2-v10
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTHRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 26 - DNA, 2856 bp, Homo sapiens
  source 1..2856: /mol_type=“unassigned DNA” /note=“Nucleotide sequence encoding homo sapiens CD44 isoform CD44v2-v10” /organism=“Homo sapiens”
atttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccactttgatgagcactagtgctacagcaactgagacagcaaccaagaggcaagaaacctgggattggttttcatggttgtttctaccatcagagtcaaagaatcatcttcacacaacaacacaaatggctggtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttca

SEQ ID NO: 27 - protein, 719 aa, Homo sapiens
  Amino acid sequence of homo sapiens CD44 isoform CD44v2-v10
LNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 28 - DNA, 5636 bp, Homo sapiens
  source 1..5636: /mol_type=“unassigned DNA” /note=“Nucleotide sequence encoding homo sapiens CD44 isoform CD44v2-v10” /organism=“Homo sapiens”
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggagctttgatgagcactagtgctacagcaactgagacagcaaccaagaggcaagaaacctgggattggttttcatggttgtttctaccatcagagtcaaagaatcatcttcacacaacaacacaaatggctggtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaac

SEQ ID NO: 29 - protein, 711 aa, Homo sapiens
  Amino acid sequence encoding homo sapiens CD44 isoform CD44v2-v10
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGALMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 30 - DNA, 5600 bp, Homo sapiens
  source 1..5600: /mol_type=“unassigned DNA” /note=“Nucleotide sequence encoding homo sapiens CD44 isoform CD44v3-10” /organism=“Homo sapiens”
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccagtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaac

SEQ ID NO: 31 - protein, 699 aa, Homo sapiens
  Amino acid sequence of homo sapiens CD44 isoform CD44v3-10
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATSTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 32 - DNA, 2069 bp, Homo sapiens
  source 1..2069: /mol_type=“unassigned DNA” /note=“Nucleotide sequence encoding homo sapiens CD44 isoform CD44v3-10” /organism=“Homo sapiens”
atttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccagtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttg

SEQ ID NO: 33 - protein, 676 aa, Homo sapiens
  Amino acid sequence of homo sapiens CD44 isoform CD44v3-10
LNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATSTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 34 - DNA, 4982 bp, Homo sapiens
  source 1..4982: /mol_type=“unassigned DNA” /note=“Nucleotide sequence encoding homo sapiens CD44 isoform CD44v8-10 ” /organism=“Homo sapiens”
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccaatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaac

SEQ ID NO: 35 - protein, 493 aa, Homo sapiens
  Amino acid sequence of homo sapiens CD44 isoform CD44v8-10
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATNMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 36 - DNA, 2103 bp, Homo sapiens
  source 1..2103: /mol_type=“unassigned DNA” /note=“Nucleotide sequence encoding homo sapiens CD44 isoform CD44v8-v10 ” /organism=“Homo sapiens”
atttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccaatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcga

SEQ ID NO: 37 - protein, 470 aa, Homo sapiens
  Amino acid sequence of homo sapiens CD44 isoform CD44v8-v10
LNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATNMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 38 - DNA, 4718 bp, Homo sapiens
  source 1..4718: /mol_type=“unassigned DNA” /note=“Nucleotide sequence encoding homo sapiens CD44 isoform CD44v6” /organism=“Homo sapiens”
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccacagcctcagctcataccagccatccaatgcaaggaaggacaacaccaagcccagaggacagttcctggactgatttcttcaacccaatctcacaccccatgggacgaggtcatcaagcaggaagaaggatgggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaac

SEQ ID NO: 39 - protein, 405 aa, Homo sapiens
  Amino acid sequence of homo sapiens CD44 isoform CD44v6
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATAASAHTSHPMQGRTTPSPEDSSWTDFFNPISHPMGRGHQAGRRMRDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 40 - DNA, 4688 bp, Homo sapiens
  source 1..4688: /mol_type=“unassigned DNA” /note=“Nucleotide sequence encoding homo sapiens CD44 isoform CD44v8” /organism=“Homo sapiens”
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccaatatggactccagtcatagtataacgcttcagcctactgcaaatccaaacacaggtttggtggaagatttggacaggacaggacctctttcaatgacaacgcgagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaac

SEQ ID NO: 41 - protein, 395 aa, Homo sapiens
  Amino acid sequence of homo sapiens CD44 isoform CD44v8
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATNMDSSHSITLQPTANPNTGLVEDLDRTGPLSMTTRDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 42 - DNA, 4790 bp, Homo sapiens
  source 1..4790: /mol_type=“unassigned DNA” /note=“Nucleotide sequence encoding homo sapiens CD44 isoform CD44v10” /organism=“Homo sapiens”
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaac

SEQ ID NO: 43 - protein, 429 aa, Homo sapiens
  Amino acid sequence of homo sapiens CD44 isoform CD44v10
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 44 - DNA, 501 bp, Homo sapiens
  source 1..501: /mol_type=“unassigned DNA” /note=“Nucleotide sequence encoding exon C1 of homo sapiens CD44” /organism=“Homo sapiens”
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcg

SEQ ID NO: 45 - protein, 22 aa, Homo sapiens
  Amino acid sequence of exon C1 of homo sapiens CD44
MDKFWWHAAWGLCLVPLSLAQI

SEQ ID NO: 46 - DNA, 166 bp, Homo sapiens
  source 1..166: /mol_type=“unassigned DNA” /note=“Nucleotide sequence encoding exon C2 of homo sapiens CD44 ” /organism=“Homo sapiens”
atttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcag

SEQ ID NO: 47 - protein, 55 aa, Homo sapiens
  Amino acid sequence of exon C2 of homo sapiens CD44
DLNITCRFAGVFHVEKNGRYSISRTEAADLCKAFNSTLPTMAQMEKALSIGFETC

SEQ ID NO: 48 - DNA, 134 bp, Homo sapiens
  source 1..134: /mol_type=“unassigned DNA” /note=“Nucleotide sequence encoding exon C3 of homo sapiens CD44” /organism=“Homo sapiens”
gtatgggttcatagaagggcacgtggtgattccccggatccaccccaactccatctgtgcagcaaacaacacaggggtgtacatcctcacatccaacacctcccagtatgacacatattgcttcaatgcttcag

SEQ ID NO: 49 - protein, 45 aa, Homo sapiens
  Amino acid sequence of exon C3 of homo sapiens CD44
RYGFIEGHVVIPRIHPNSICAANNTGVYILTSNTSQYDTYCFNAS

SEQ ID NO: 50 - DNA, 69 bp, Homo sapiens
  source 1..69: /mol_type=“unassigned DNA” /note=“Nucleotide sequence encoding exon C4 of homo sapiens CD44 ” /organism=“Homo sapiens”
ctccacctgaagaagattgtacatcagtcacagacctgcccaatgcctttgatggaccaattaccataa

SEQ ID NO: 51 - protein, 23 aa, Homo sapiens
  Amino acid sequence of exon C4 of homo sapiens CD44
APPEEDCTSVTDLPNAFDGPITI

SEQ ID NO: 52 - DNA, 231 bp, Homo sapiens
  source 1..231: /mol_type=“unassigned DNA” /note=“Nucleotide sequence encoding exon C5 of homo sapiens CD44” /organism=“Homo sapiens”
ctattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctacca

SEQ ID NO: 53 - protein, 77 aa, Homo sapiens
  Amino acid sequence of exon C5 of homo sapiens CD44
TIVNRDGTRYVQKGEYRTNPEDIYPSNPTDDDVSSGSSSERSSTSGGYIFYTFSTVHPIPDEDSPWITDSTDRIPAT

SEQ ID NO: 54 - DNA, 129 bp, Homo sapiens
  source 1..129: /mol_type=“unassigned DNA” /note=“Nucleotide sequence encoding exon V2 of homo sapiens CD44 ” /organism=“Homo sapiens”
ctttgatgagcactagtgctacagcaactgagacagcaaccaagaggcaagaaacctgggattggttttcatggttgtttctaccatcagagtcaaagaatcatcttcacacaacaacacaaatggctg

SEQ ID NO: 55 - protein, 43 aa, Homo sapiens
  Amino acid sequence of exon V2 of homo sapiens CD44
TLMSTSATATETATKRQETWDWFSWLFLPSESKNHLHTTTQMA

SEQ ID NO: 56 - DNA, 126 bp, Homo sapiens
  source 1..126: /mol_type=“unassigned DNA” /note=“Nucleotide sequence encoding exon V3 of homo sapiens CD44” /organism=“Homo sapiens”
gtacgtcttcaaataccatctcagcaggctgggagccaaatgaagaaaatgaagatgaaagagacagacacctcagtttttctggatcaggcattgatgatgatgaagattttatctccagcacca

SEQ ID NO: 57 - protein, 42 aa, Homo sapiens
  Amino acid sequence of exon V3 of homo sapiens CD44
GTSSNTISAGWEPNEENEDERDRHLSFSGSGIDDDEDFISST

SEQ ID NO: 58 - DNA, 114 bp, Homo sapiens
  source 1..114: /mol_type=“unassigned DNA” /note=“Nucleotide sequence encoding exon V4 of homo sapiens CD44” /organism=“Homo sapiens”
tttcaaccacaccacgggcttttgaccacacaaaacagaaccaggactggacccagtggaacccaagccattcaaatccggaagtgctacttcagacaaccacaaggatgactg

SEQ ID NO: 59 - protein, 38 aa, Homo sapiens
  Amino acid sequence of exon V4 of homo sapiens CD44
ISTTPRAFDHTKQNQDWTQWNPSHSNPEVLLQTTTRMT

SEQ ID NO: 60 - DNA, 117 bp, Homo sapiens
  source 1..117: /mol_type=“unassigned DNA” /note=“Nucleotide sequence encoding exon V5 of homo sapiens CD44” /organism=“Homo sapiens”
atgtagacagaaatggcaccactgcttatgaaggaaactggaacccagaagcacaccctcccctcattcaccatgagcatcatgaggaagaagagaccccacattctacaagcacaa

SEQ ID NO: 61 - protein, 39 aa, Homo sapiens
  Amino acid sequence of exon V5 of homo sapiens CD44
DVDRNGTTAYEGNWNPEAHPPLIHHEHHEEEETPHSTST

SEQ ID NO: 62 - DNA, 129 bp, Homo sapiens
  source 1..129: /mol_type=“unassigned DNA” /note=“Nucleotide sequence encoding exon V6 of homo sapiens CD44 ” /organism=“Homo sapiens”
tccaggcaactcctagtagtacaacggaagaaacagctacccagaaggaacagtggtttggcaacagatggcatgagggatatcgccaaacacccaaagaagactcccattcgacaacagggacagctg

SEQ ID NO: 63 - protein, 43 aa, Homo sapiens
  Amino acid sequence of exon V6 of homo sapiens CD44
IQATPSSTTEETATQKEQWFGNRWHEGYRQTPKEDSHSTTGTA

SEQ ID NO: 64 - DNA, 132 bp, Homo sapiens
  source 1..132: /mol_type=“unassigned DNA” /note=“Nucleotide sequence encoding exon V7 of homo sapiens CD44 ” /organism=“Homo sapiens”
cagcctcagctcataccagccatccaatgcaaggaaggacaacaccaagcccagaggacagttcctggactgatttcttcaacccaatctcacaccccatgggacgaggtcatcaagcaggaagaaggatgg

SEQ ID NO: 65 - protein, 44 aa, Homo sapiens
  Amino acid sequence of exon V7 of homo sapiens CD44
AASAHTSHPMQGRTTPSPEDSSWTDFFNPISHPMGRGHQAGRRM

SEQ ID NO: 66 - DNA, 102 bp, Homo sapiens
  source 1..102: /mol_type=“unassigned DNA” /note=“Nucleotide sequence encoding exon V8 of homo sapiens CD44” /organism=“Homo sapiens”
atatggactccagtcatagtataacgcttcagcctactgcaaatccaaacacaggtttggtggaagatttggacaggacaggacctctttcaatgacaacgc

SEQ ID NO: 67 - protein, 34 aa, Homo sapiens
  Amino acid sequence of exon V8 of homo sapiens CD44
DMDSSHSITLQPTANPNTGLVEDLDRTGPLSMTT

SEQ ID NO: 68 - DNA, 90 bp, Homo sapiens
  source 1..90: /mol_type=“unassigned DNA” /note=“Nucleotide sequence encoding exon V9 of homo sapiens CD44 ” /organism=“Homo sapiens”
agcagagtaattctcagagcttctctacatcacatgaaggcttggaagaagataaagaccatccaacaacttctactctgacatcaagca

SEQ ID NO: 69 - protein, 30 aa, Homo sapiens
  Amino acid sequence of exon V9 of homo sapiens CD44
QQSNSQSFSTSHEGLEEDKDHPTTSTLTSS

SEQ ID NO: 70 - DNA, 204 bp, Homo sapiens
  source 1..204: /mol_type=“unassigned DNA” /note=“Nucleotide sequence encoding exon V10 of homo sapiens CD44 ” /organism=“Homo sapiens”
ataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcag

SEQ ID NO: 71 - protein, 68 aa, Homo sapiens
  Amino acid sequence of exon V10 of homo sapiens CD44
NRNDVTGGRRDPNHSEGSTTLLEGYTSHYPHTKESRTFIPVTSAKTGSFGVTAVTVGDSNSNVNRSLS

SEQ ID NO: 72 - DNA, 63 bp, Homo sapiens
  source 1..63: /mol_type=“unassigned DNA” /note=“Nucleotide sequence encoding exon C6 of homo sapiens CD44 ” /organism=“Homo sapiens”
gagaccaagacacattccaccccagtggggggtcccataccactcatggatctgaatcagatg

SEQ ID NO: 73 - protein, 21 aa, Homo sapiens
  Amino acid sequence of exon C6 of homo sapiens CD44
GDQDTFHPSGGSHTTHGSESD

SEQ ID NO: 74 - DNA, 72 bp, Homo sapiens
  source 1..72: /mol_type=“unassigned DNA” /note=“Nucleotide sequence encoding exon C7 of homo sapiens CD44 ” /organism=“Homo sapiens”
gacactcacatgggagtcaagaaggtggagcaaacacaacctctggtcctataaggacaccccaaattccag

SEQ ID NO: 75 - protein, 24 aa, Homo sapiens
  Amino acid sequence of exon C7 of homo sapiens CD44
GHSHGSQEGGANTTSGPIRTPQIP

SEQ ID NO: 76 - DNA, 79 bp, Homo sapiens
  source 1..79: /mol_type=“unassigned DNA” /note=“Nucleotide sequence encoding exon C8 of homo sapiens CD44 ” /organism=“Homo sapiens”
aatggctgatcatcttggcatccctcttggccttggctttgattcttgcagtttgcattgcagtcaacagtcgaagaag

SEQ ID NO: 77 - protein, 26 aa, Homo sapiens
  Amino acid sequence of exon C8 of homo sapiens CD44
EWLIILASLLALALILAVCIAVNSRR

SEQ ID NO: 78 - DNA, 3271 bp, Homo sapiens
  source 1..3271: /mol_type=“unassigned DNA” /note=“Nucleotide sequence encoding exon C9 of homo sapiens CD44” /organism=“Homo sapiens”
gtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaac

SEQ ID NO: 79 - protein, 68 aa, Homo sapiens
  Amino acid sequence of exon C9 of homo sapiens CD44
RCGQKKKLVINSGNGAVEDRKPSGLNGEASKSQEMVHLVNKESSETPDQFMTADETRNLQNVDMKIGV

SEQ ID NO: 80 - DNA, 2087 bp, Homo sapiens
  source 1..2087: /mol_type=“unassigned DNA” /note=“Nucleotide sequence encoding hyaluronic acid synthetase 1 (HAS1)” /organism=“Homo sapiens”
cggagagaagagagagcccggccagacccactgcgatgagacagcaggacgcgcccaagcccactcctgcagcctgccgctgctccggcctggcccggagggtgctgaccatcgccttcgccctgctcatcctgggcctcatgacctgggcctacgccgccggggtgccgctggcctccgatcgctacggcctcctggccttcggcctctacggggccttcctttcagcgcacctggtggcgcagagcctcttcgcgtacctggagcaccggcgggtggcggcggcggcgcgggggccgctggatgcagccaccgcgcgcagtgtggcgctgaccatctccgcctaccaggaggaccccgcgtacctgcgccagtgcctggcgtccgcccgcgccctgctgtacccgcgcgcgcggctgcgcgtcctcatggtggtggatggcaaccgcgccgaggacctctacatggtcgacatgttccgcgaggtcttcgctgacgaggaccccgccacgtacgtgtgggacggcaactaccaccagccctgggaacccgcggcggcgggcgcggtgggcgccggagcctatcgggaggtggaggcggaggatcctgggcggctggcagtggaggcgctggtgaggactcgcaggtgcgtgtgcgtggcgcagcgctggggcggcaagcgcgaggtcatgtacacagccttcaaggcgctcggagattcggtggactacgtgcaggtctgtgactcggacacaaggttggaccccatggcactgctggagctcgtgcgggtactggacgaggacccccgggtaggggctgttggtggggacgtgcggatccttaaccctctggactcctgggtcagcttcctaagcagcctgcgatactgggtagccttcaatgtggagcgggcttgtcagagctacttccactgtgtatcctgcatcagcggtcctctaggcctatataggaataacctcttgcagcagtttcttgaggcctggtacaaccagaagttcctgggtacccactgtacttttggggatgaccggcacctcaccaaccgcatgctcagcatgggttatgctaccaagtacacctccaggtcccgctgctactcagagacgccctcgtccttcctgcggtggctgagccagcagacacgctggtccaagtcgtacttccgtgagtggctgtacaacgcgctctggtggcaccggcaccatgcgtggatgacctacgaggcggtggtctccggcctgttccccttcttcgtggcggccactgtgctgcgtctgttctacgcgggccgcccttgggcgctgctgtgggtgctgctgtgcgtgcagggcgtggcactggccaaggcggccttcgcggcctggctgcggggctgcctgcgcatggtgcttctgtcgctctacgcgcccctctacatgtgtggcctcctgcctgccaagttcctggcgctagtcaccatgaaccagagtggctggggcacctcgggccggcggaagctggccgctaactacgtccctctgctgcccctggcgctctgggcgctgctgctgcttgggggcctggtccgcagcgtagcacacgaggccagggccgactggagcggcccttcccgcgcagccgaggcctaccacttggccgcgggggccggcgcctacgtgggctactgggtggccatgttgacgctgtactgggtgggcgtgcggaggctttgccggcggcggaccgggggctaccgcgtccaggtgtgagtccagccacgcggatgccgcctcaagggtcttcaggggaggccagaggagagctgctgggccccgagccacgaacttgctgggtggttctctgggcctcagtttccctcctctgcaaaacgagggggtcagcccaagattcttcagtctggactatattgggactgggacttctgggtctccagggagggtatttattggtcagggtgtgggatctgaggagtggagggaaagggtcctgctttctcctcgttcttatttaatctccatttctactgtgtgatcaggatgtaataaagaattttatttattttc

SEQ ID NO: 81 - protein, 578 aa, Homo sapiens
  Amino acid sequence of hyaluronic acid synthetase 1 (HAS1)
MRQQDAPKPTPAACRCSGLARRVLTIAFALLILGLMTWAYAAGVPLASDRYGLLAFGLYGAFLSAHLVAQSLFAYLEHRRVAAAARGPLDAATARSVALTISAYQEDPAYLRQCLASARALLYPRARLRVLMVVDGNRAEDLYMVDMFREVFADEDPATYVWDGNYHQPWEPAAAGAVGAGAYREVEAEDPGRLAVEALVRTRRCVCVAQRWGGKREVMYTAFKALGDSVDYVQVCDSDTRLDPMALLELVRVLDEDPRVGAVGGDVRILNPLDSWVSFLSSLRYWVAFNVERACQSYFHCVSCISGPLGLYRNNLLQQFLEAWYNQKFLGTHCTFGDDRHLTNRMLSMGYATKYTSRSRCYSETPSSFLRWLSQQTRWSKSYFREWLYNALWWHRHHAWMTYEAVVSGLFPFFVAATVLRLFYAGRPWALLWVLLCVQGVALAKAAFAAWLRGCLRMVLLSLYAPLYMCGLLPAKFLALVTMNQSGWGTSGRRKLAANYVPLLPLALWALLLLGGLVRSVAHEARADWSGPSRAAEAYHLAAGAGAYVGYWVAMLTLYWVGVRRLCRRRTGGYRVQV

SEQ ID NO: 82 - DNA, 4190 bp, Homo sapiens
  source 1..4190: /mol_type=“unassigned DNA” /note=“Nucleotide sequence encoding hyaluronic acid synthetase 2 (HAS2)” /organism=“Homo sapiens”
gaagtcaagacgtctggaaagaattacccagtcctggcttcgagcagcccattgaaccagagacttgaaacagccccagccaaagacttttctcccaattctgcgcttcctgggttctgctgagtcttccacaggctttttttttttttttttttttttttaagacgaaaaagagattttctgttatcgggggcagaaagactgaagcccaaaaaaaaaaaaaaaaaaaaaagaaaagaaaagaaaaaagaaaagttaatttatttttaaagcataatttttttaagaattagactgaagtgcaacggaaacataaagagaatattagtgaaattattttttaaagtggggaagaatcaaacatttaagactcccctatcctttttaaatgttgtttttaaatttcttattttttttggccggtcgtctcaaattcatctgatctcttattacctcaattttggaaactgcccgccaccgaccctccgggaccacacagacaggctgaggacgactttatgaccaagagctgaacaagatgcattgtgagaggtttctatgtatcctgagaataattggaaccacactctttggagtctctctcctccttggaatcacagctgcttatattgttggctaccagtttatccaaacggataattactatttctcttttggactgtatggtgcctttttggcatcacacctcatcatccaaagcctgtttgcctttttggagcaccgaaaaatgaaaaaatccctagaaacccccataaagttgaacaaaacagttgccctttgcatcgctgcctatcaagaagatccagactacttaaggaaatgtttgcaatctgtgaaaaggctaacctaccctgggattaaagttgtcatggtcatagatgggaactcagaagatgacctttacatgatggacatcttcagtgaagtcatgggcagagacaaatcagccacttatatctggaagaacaacttccacgaaaagggtcccggtgagacagatgagtcacataaagaaagctcgcaacacgtaacgcaattggtcttgtccaacaaaagtatctgcatcatgcaaaaatggggtggaaaaagagaagtcatgtacacagccttcagagcactgggacgaagtgtggattatgtacaggtttgtgattcagacactatgcttgacccagcctcatctgtggagatggtaaaagttttagaagaagatcccatggttggaggtgttgggggagatgtccagattttaaacaagtacgattcctggatctcattcctcagcagtgtaagatattggatggcttttaatatagaaagggcctgtcagtcttattttgggtgtgttcagtgcattagtggacctctgggaatgtacagaaactccttgttgcatgagtttgtggaagattggtacaatcaagaatttatgggcaaccaatgtagctttggtgatgacaggcatctcacgaaccgggtgctgagcctgggctatgcaacaaaatacacagctcgatctaagtgccttactgaaacacctatagaatatctcagatggctaaaccagcagacccgttggagcaagtcctacttccgagaatggctgtacaatgcaatgtggtttcacaaacatcacttgtggatgacctacgaagcgattatcactggattctttcctttctttctcattgccacagtaatccagctcttctaccggggtaaaatttggaacattctcctcttcttgttaactgtccagctagtaggtctcataaaatcatcttttgccagctgccttagaggaaatatcgtcatggtcttcatgtctctctactcagtgttatacatgtcgagtttacttcccgccaagatgtttgcaattgcaacaataaacaaagctgggtggggcacatcaggaaggaaaaccattgttgttaatttcataggactcattccagtatcagtttggtttacaatcctcctgggtggtgtgattttcaccatttataaggagtctaaaaggccattttcagaatccaaacagacagttctaattgttggaacgttgctctatgcatgctattgggtcatgcttttgacgctgtatgtagttctcatcaataagtgtggcaggcggaagaagggacaacaatatgacatggtgcttgatgtatgatcttccatgttttgacgtttgcagtcacacacaacaccttagttcctctaggggctgtacagtattgtggcatcagataatgccaccaaaggagacatatcactgctgctgggacttgaacaaagacatttatatgggtttattttcattctgccaaagtaaaacaatacatcaacaagaagaaactcagatttaacctgttatttctatgaaaatgggatgaattctttgtttatgcactttttccttactgtgcatccgcctgaaagtgttttgccctatatacctcactagccatgctttatgtgggttatcatggaagaaaaggattttggaaactcaaggaaaagttctttcaacctatacaacctaacttatggactgttttgatagatgataattttttttttttaggaaggattttctttttaactttaccaaatgaaatgccaaaggaagttttaaaggccgttggctgtgctgtattttgatataattgtactgtgtttttaaattttgtatgccaatcttaaagacaaattttgcatattctctattttacttttctgccaaaataaacctgttcttccttttttaaaataaaataagttcttaaaaaatttatacttaaaaaatcctgcccaaaatgtgaagcttggttgactgatgttcatgatagaaagaataaaatgtttctctctctctaccttttaaaattgaatagtttatttctgtgaaagaagtatttaaactttcaatattttaactttttgtttttatttcttttagaaaaggccaatatacctatcacactttggaagtaaaaatacacactttcgtgtgtacctaaaaaaaaaatcgttgaaaatcaaggccaaaggtagtgcaattttttcattaagatttaaaaaaaagggaatgatagtctttgaaagaaaacagtaggcatccagcactggacaaaacatgggtatcaaagatgaataatctttggagattctggcagtgttttcccagaacaagtcaagtggaaagtggagaaattatctgtataattttggacacatacaatgcagtttatcaaaggttttgttctgtggcctgaatttactgggtcctacctacacattgaacatgttttgcctgtcttttttttttttttcaacttgccagttcactttacatgttagtataatgtttacacgggtgagttggataaattataaaacatataaattcaaaattggcagatagaatcaccaagtatctatcctcttttactttcaaatgaggaattttgtttttctgaattacacagatcatcacttcctatttcctgttctggacctgtataaaaatgtctacacagtagaagtgacatcaaggtttaataagtatatcaatgattggcacatataaaaattgttgaaccacatactctgaacttggctaatttagttactgcaaggcctccattatccagttttattttttacacgattgaccttgccttgtagctggtgctgtgtagacctgtgttgaaaacacaatcggaatatatgaataattgaataaacagcattatggtgaggcagagacacatggagaagtgttaaaaaaaaaatgggcttcctgcctttctgcctcttttttatgcagtcatctatgttacatctatcctgcctaagaaaaagctgcacatcctaccttcagagtacaaaaaggtacatctgagactcaagactctcactgattggagagcttgtggaaaacaaaacacaccatgccaataaatgagatgaaaacttgagtttgcctttttaactatttatgttctaagttaagctttgataacattcaaatgtcaaattctctcattcttataaaaagttgaattaattgcctgtatttattttagcaattattcaatgtatttccagtataggatgtatagtataattaattttttgtaaataaaatatttttgataaga

SEQ ID NO: 83 - protein, 552 aa, Homo sapiens
  Amino acid sequence of hyaluronic acid synthetase 2 (HAS2)
MHCERFLCILRIIGTTLFGVSLLLGITAAYIVGYQFIQTDNYYFSFGLYGAFLASHLIIQSLFAFLEHRKMKKSLETPIKLNKTVALCIAAYQEDPDYLRKCLQSVKRLTYPGIKVVMVIDGNSEDDLYMMDIFSEVMGRDKSATYIWKNNFHEKGPGETDESHKESSQHVTQLVLSNKSICIMQKWGGKREVMYTAFRALGRSVDYVQVCDSDTMLDPASSVEMVKVLEEDPMVGGVGGDVQILNKYDSWISFLSSVRYWMAFNIERACQSYFGCVQCISGPLGMYRNSLLHEFVEDWYNQEFMGNQCSFGDDRHLTNRVLSLGYATKYTARSKCLTETPIEYLRWLNQQTRWSKSYFREWLYNAMWFHKHHLWMTYEAIITGFFPFFLIATVIQLFYRGKIWNILLFLLTVQLVGLIKSSFASCLRGNIVMVFMSLYSVLYMSSLLPAKMFAIATINKAGWGTSGRKTIVVNFIGLIPVSVWFTILLGGVIFTIYKESKRPFSESKQTVLIVGTLLYACYWVMLLTLYVVLINKCGRRKKGQQYDMVLDV

SEQ ID NO: 84 - DNA, 4220 bp, Homo sapiens
  source 1..4220: /mol_type=“unassigned DNA” /note=“Nucleotide sequence encoding hyaluronic acid synthetase 3 (HAS3)” /organism=“Homo sapiens”
gagtgcgttcgcggctgctttgacctggtgggcgccgcctccggcactgcaccgaggcggggcgccagcgcccaggttgctgggctggccttggcgcccccttcccctacccagagcgcaggcaggcaggggggtcccgccgcgccccttcagcatatgccggtgcagctgacgacagccctgcgtgtggtgggcaccagcctgtttgccctggcagtgctgggtggcatcctggcagcctatgtgacgggctaccagttcatccacacggaaaagcactacctgtccttcggcctgtacggcgccatcctgggcctgcacctgctcattcagagcctttttgccttcctggagcaccggcgcatgcgacgtgccggccaggccctgaagctgccctccccgcggcggggctcggtggcactgtgcattgccgcataccaggaggaccctgactacttgcgcaagtgcctgcgctcggcccagcgcatctccttccctgacctcaaggtggtcatggtggtggatggcaaccgccaggaggacgcctacatgctggacatcttccacgaggtgctgggcggcaccgagcaggccggcttctttgtgtggcgcagcaacttccatgaggcaggcgagggtgagacggaggccagcctgcaggagggcatggaccgtgtgcgggatgtggtgcgggccagcaccttctcgtgcatcatgcagaagtggggaggcaagcgcgaggtcatgtacacggccttcaaggccctcggcgattcggtggactacatccaggtgtgcgactctgacactgtgctggatccagcctgcaccatcgagatgcttcgagtcctggaggaggatccccaagtagggggagtcgggggagatgtccagatcctcaacaagtacgactcatggatttccttcctgagcagcgtgcggtactggatggccttcaacgtggagcgggcctgccagtcctactttggctgtgtgcagtgtattagtgggcccttgggcatgtaccgcaacagcctcctccagcagttcctggaggactggtaccatcagaagttcctaggcagcaagtgcagcttcggggatgaccggcacctcaccaaccgagtcctgagccttggctaccgaactaagtataccgcgcgctccaagtgcctcacagagacccccactaagtacctccggtggctcaaccagcaaacccgctggagcaagtcttacttccgggagtggctctacaactctctgtggttccataagcaccacctctggatgacctacgagtcagtggtcacgggtttcttccccttcttcctcattgccacggttatacagcttttctaccggggccgcatctggaacattctcctcttcctgctgacggtgcagctggtgggcattatcaaggccacctacgcctgcttccttcggggcaatgcagagatgatcttcatgtccctctactccctcctctatatgtccagccttctgccggccaagatctttgccattgctaccatcaacaaatctggctggggcacctctggccgaaaaaccattgtggtgaacttcattggcctcattcctgtgtccatctgggtggcagttctcctgggagggctggcctacacagcttattgccaggacctgttcagtgagacagagctagccttccttgtctctggggctatactgtatggctgctactgggtggccctcctcatgctatatctggccatcatcgcccggcgatgtgggaagaagccggagcagtacagcttggcttttgctgaggtgtgacatggcccccaagcagagcgggtaaagtgcaatgggtaagggagggaaggggaatggaagagaaaagacagggtgggagggaggagggagtgctgtgttttagtctcttaatggtccaaaggacaaatctaaaatgcaaagaacggtgatgtagtatggcctgacagctctgtttagaggaggcaacactgatcccccagatgcagggctgcaggggattctgtgttttcagactgcctgtctgcttgcatctgcacataggcagtagcctcctcctgggctccagagggcactcagaagttgtgctaaaccaagttaagtcccattcagtggcaacttgtgataggtacctgagtgacggcaacctgcggaaggaggttctcccagcccatctgaacacaaccagaggtggcaggagaatttctactgagcgagctgggccggttagtgtatgtcacccccaccccacccctaagtagtcatcaatgcaataagattgcgcctgagatacaaggcccagaagcctgatctttgggcatcagaaaacagggtccaggaatggtgctttatgtgagataccccactccacatcaacattccagggatgagccaaaccagcagggagttagcactgaactgcttttaaaagtgcacattaaaaaggaaagtttgccaggaggaacaaagagattgtggtggtgctaaaggaggccataagctacacagaggccttgggtgttccacctggaaactgctcagacgtctagatgggttcttagcttgtctgtgatctctgctggggagataaaaagattaagccccaacatgttcagaaaagaagtgaagtcttgggtattttaacctgtatactcttgaattcctctcaaattcagctctgatctgaggctaagacacactccccacttcactttcttcaaagccacattttttgaggtatcactgcagtcacctcttctaccctcatcatcataggtaaggttttcaaggtggcaattggggcggagccccggctcttatagaagcttcagcaggaggcaagcgtgttctcagcacatatgggaactatgaggagcctctgatcaaattggctacaatcttggagctgcttggacggattccttggcagccgggttagcatgtgtgactttcaggctactgttcttgacaatcatctccaatggaaagcttttcagtgttcccaaagtgaactctcaaatccaaaatggttatctttgagaccatccattctcctcagtggcttctccagggaattcttacagccaagttgtgacagtcactgcatttgcctgcttctttccagaaaccaaactaggagatgaaactggttcctacatcctaaggttcttgctttctctctcatgcctcctgaggctgtttttggctgttttccctctgctgcttttggggaatgaggggaagccattttccagtgacttgcaatccaggctgttctcagcgttttgagtttaaaacctgggatcctgactaagcctttgacttaagggttgcttgcttgccctccaaatgtcctttctcagaggggccagctaacccgtgcagaaccagcactaaggtggacagcagacaagagggcaagcctctagtgtaccaagtgcttcctacaaagacgcaaggtgtgctctgagccacagatgggcaaaccctggtgctttccttcatctcccacgaactcaagggttttccaggtgtagctaacagttgccacatcacacagacctcgagtttctggtaagactgctggttgacatcagacccaacccatgaaggctggaaggcagcaggcatttgctaaggcagctgatccaggcaatcgttctgctggccaagaagttaaactattttgagcattagaatggaggaaatccggtcagccaagtgcagagttcagacttcgctaagggcttgtttttcttcagcatttacttgaagattaatgtaggatgacaggctctcctggctgtcctaccatcagctctgccttgcactgtggtcgtcaactttcctcaaatcaaaaacaggcaggtacaggtagtgggctcacaacgtttgacctcgactggtttttctaagttattttgtacatttttcagcagcaaaaccaaactgggtcttcagctttatccccgtttcttgcaagggaagagcctttatacaattggacgcattttggtttttcctcattgagaattcaaatcctcttttgtattgtttctacaataatttgtaaacatatttatttttacctgcttttttttttttttttaattttcaggtcaagttttttatactgcacttatttgtcaaaataaagattctcacatatgccggttatctcgaaa

SEQ ID NO: 85 - protein, 553 aa, Homo sapiens
  Amino acid sequence of hyaluronic acid synthetase 3 (HAS3)
MPVQLTTALRVVGTSLFALAVLGGILAAYVTGYQFIHTEKHYLSFGLYGAILGLHLLIQSLFAFLEHRRMRRAGQALKLPSPRRGSVALCIAAYQEDPDYLRKCLRSAQRISFPDLKVVMVVDGNRQEDAYMLDIFHEVLGGTEQAGFFVWRSNFHEAGEGETEASLQEGMDRVRDVVRASTFSCIMQKWGGKREVMYTAFKALGDSVDYIQVCDSDTVLDPACTIEMLRVLEEDPQVGGVGGDVQILNKYDSWISFLSSVRYWMAFNVERACQSYFGCVQCISGPLGMYRNSLLQQFLEDWYHQKFLGSKCSFGDDRHLTNRVLSLGYRTKYTARSKCLTETPTKYLRWLNQQTRWSKSYFREWLYNSLWFHKHHLWMTYESVVTGFFPFFLIATVIQLFYRGRIWNILLFLLTVQLVGIIKATYACFLRGNAEMIFMSLYSLLYMSSLLPAKIFAIATINKSGWGTSGRKTIVVNFIGLIPVSIWVAVLLGGLAYTAYCQDLFSETELAFLVSGAILYGCYWVALLMLYLAIIARRCGKKPEQYSLAFAEV

SEQ ID NO: 86 - protein, 11 aa, Homo sapiens
  Amino acid sequence of epitope on the constant region of CD44
AFDGPITITIV

SEQ ID NO: 87 - DNA, 20 bp, Artificial Sequence
  source 1..20: /mol_type=“unassigned DNA” /note=“forward primer CD44-v3” /organism=“Artificial Sequence”
cctgctaccagtacgtcttc

SEQ ID NO: 88 - DNA, 19 bp, Artificial Sequence
  source 1..19: /mol_type=“unassigned DNA” /note=“reverse primer CD44-v3” /organism=“Artificial Sequence”
accagagaccaagacacat

SEQ ID NO: 89 - DNA, 36 bp, Artificial Sequence
  source 1..36: /mol_type=“unassigned DNA” /note=“probe CD44-v3” /organism=“Artificial Sequence”
agccaaatgaagaaaatgaagatgaaagagacagac

SEQ ID NO: 90 - DNA, 20 bp, Artificial Sequence
  source 1..20: /mol_type=“unassigned DNA” /note=“primer CD44-v6” /organism=“Artificial Sequence”
gaatccctgctaccatccag

SEQ ID NO: 91 - DNA, 17 bp, Artificial Sequence
  source 1..17: /mol_type=“unassigned DNA” /note=“primer CD44-v6” /organism=“Artificial Sequence”
gctggagaccaagacac

SEQ ID NO: 92 - DNA, 31 bp, Artificial Sequence
  source 1..31: /mol_type=“unassigned DNA” /note=“probe CD44-v6” /organism=“Artificial Sequence”
aacggaagaaacagctacccagaaggaacag

SEQ ID NO: 93 - DNA, 21 bp, Artificial Sequence
  source 1..21: /mol_type=“unassigned DNA” /note=“forward primer CD44-v10” /organism=“Artificial Sequence”
acagaatccctgctaccaata

SEQ ID NO: 94 - DNA, 20 bp, Artificial Sequence
  source 1..20: /mol_type=“unassigned DNA” /note=“reverse primer CD44-v10” /organism=“Artificial Sequence”
aggctcaactactttactgg

SEQ ID NO: 95 - DNA, 29 bp, Artificial Sequence
  source 1..29: /mol_type=“unassigned DNA” /note=“probe CD44-v10” /organism=“Artificial Sequence”
tcacaggtggaagaagagacccaaatcat

SEQ ID NO: 96 - DNA, 23 bp, Artificial Sequence
  source 1..23: /mol_type=“unassigned DNA” /note=“forward primer CD44-v3-10” /organism=“Artificial Sequence”
ccctgctaccagtacgtcttcaa

SEQ ID NO: 97 - DNA, 23 bp, Artificial Sequence
  source 1..23: /mol_type=“unassigned DNA” /note=“reverse primer CD44-v3-10” /organism=“Artificial Sequence”
gacagacacctcagtttttctgg

SEQ ID NO: 98 - DNA, 21 bp, Artificial Sequence
  source 1..21: /mol_type=“unassigned DNA” /note=“forward primer CD44-v6-10” /organism=“Artificial Sequence”
gaatccctgctaccatccagg

SEQ ID NO: 99 - DNA, 17 bp, Artificial Sequence
  source 1..17: /mol_type=“unassigned DNA” /note=“reverse primer CD44-v6-10” /organism=“Artificial Sequence”
ttggcaacagatggcat

SEQ ID NO: 100 - DNA, 31 bp, Artificial Sequence; identical to SEQ ID NO: 92
  source 1..31: /mol_type=“unassigned DNA” /note=“probe CD44-v6-10” /organism=“Artificial Sequence”

SEQ ID NO: 101 - DNA, 22 bp, Artificial Sequence
  source 1..22: /mol_type=“unassigned DNA” /note=“forward primer CD44-v8-10” /organism=“Artificial Sequence”
tccctgctaccaatatggactc

SEQ ID NO: 102 - DNA, 21 bp, Artificial Sequence
  source 1..21: /mol_type=“unassigned DNA” /note=“reverse primer CD44-v8-10” /organism=“Artificial Sequence”
ctgcaaatccaaacacaggtt

SEQ ID NO: 103 - DNA, 21 bp, Artificial Sequence
  source 1..21: /mol_type=“unassigned DNA” /note=“forward primer CD44-s” /organism=“Artificial Sequence”
cagaatccctgctaccagaga

SEQ ID NO: 104 - DNA, 18 bp, Artificial Sequence
  source 1..18: /mol_type=“unassigned DNA” /note=“reverse primer CD44-s” /organism=“Artificial Sequence”
atggacactcacatggga

SEQ ID NO: 105 - DNA, 22 bp, Artificial Sequence
  source 1..22: /mol_type=“unassigned DNA” /note=“probe CD44-s” /organism=“Artificial Sequence”
ccagtggggggtcccataccac

SEQ ID NO: 106 - DNA, 27 bp, Artificial Sequence
  source 1..27: /mol_type=“unassigned DNA” /note=“forward primer CD44-long” /organism=“Artificial Sequence”
tctttcaatgacaacgcagcagagtaa

SEQ ID NO: 107 - DNA, 23 bp, Artificial Sequence
  source 1..23: /mol_type=“unassigned DNA” /note=“reverse primer CD44-long” /organism=“Artificial Sequence”
tactctgacatcaagcaatagga

SEQ ID NO: 108 - DNA, 32 bp, Artificial Sequence
  source 1..32: /mol_type=“unassigned DNA” /note=“probe CD44-long” /organism=“Artificial Sequence”
atgaaggcttggaagaagataaagaccatcca

SEQ ID NO: 109 - DNA, 20 bp, Artificial Sequence
  source 1..20: /mol_type=“unassigned DNA” /note=“forward primer CD44-pan” /organism=“Artificial Sequence”
gaatcagatggacactcaca

SEQ ID NO: 110 - DNA, 20 bp, Artificial Sequence
  source 1..20: /mol_type=“unassigned DNA” /note=“reverse primer CD44-pan” /organism=“Artificial Sequence”
ttccagaatggctgatcatc

SEQ ID NO: 111 - DNA, 19 bp, Artificial Sequence
  source 1..19: /mol_type=“unassigned DNA” /note=“forward primer HAS1” /organism=“Artificial Sequence”
tgctcagcatgggttatgc

SEQ ID NO: 112 - DNA, 20 bp, Artificial Sequence
  source 1..20: /mol_type=“unassigned DNA” /note=“reverse primer HAS1” /organism=“Artificial Sequence”
agggcgtctctgagtagcag

SEQ ID NO: 113 - DNA, 18 bp, Artificial Sequence
  source 1..18: /mol_type=“unassigned DNA” /note=“forward primer HAS2” /organism=“Artificial Sequence”
ctccgggaccacacagac

SEQ ID NO: 114 - DNA, 25 bp, Artificial Sequence
  source 1..25: /mol_type=“unassigned DNA” /note=“reverse primer HAS2” /organism=“Artificial Sequence”
tcaggatacatagaaacctctcaca

SEQ ID NO: 115 - DNA, 20 bp, Artificial Sequence
  source 1..20: /mol_type=“unassigned DNA” /note=“forward primer HAS3 transcript variant 1” /organism=“Artificial Sequence”
cgattcggtggactacatcc

SEQ ID NO: 116 - DNA, 18 bp, Artificial Sequence
  source 1..18: /mol_type=“unassigned DNA” /note=“reverse primer HAS3 transcript variant 1” /organism=“Artificial Sequence”
ccgactccccctacttgg

SEQ ID NO: 117 - RNA, 4586 nt, Homo sapiens
  source 1..4586: /mol_type=“unassigned RNA” /note=“Nucleotide sequence encoding homo sapiens CD44 isoform CD44s” /organism=“Homo sapiens”
gagaagaaagccagugcgucucugggcgcaggggccaguggggcucggaggcacaggcaccccgcgacacuccagguuccccgacccacgucccuggcagccccgauuauuuacagccucagcagagcacggggcgggggcagaggggcccgcccgggagggcugcuacuucuuaaaaccucugcgggcugcuuagucacagccccccuugcuuggguguguccuucgcucgcucccucccuccgucuuaggucacuguuuucaaccucgaauaaaaacugcagccaacuuccgaggcagccucauugcccagcggaccccagccucugccagguucgguccgccauccucgucccguccuccgccggccccugccccgcgcccagggauccuccagcuccuuucgcccgcgcccuccguucgcuccggacaccauggacaaguuuugguggcacgcagccuggggacucugccucgugccgcugagccuggcgcagaucgauuugaauauaaccugccgcuuugcagguguauuccacguggagaaaaauggucgcuacagcaucucucggacggaggccgcugaccucugcaaggcuuucaauagcaccuugcccacaauggcccagauggagaaagcucugagcaucggauuugagaccugcagguauggguucauagaagggcacguggugauuccccggauccaccccaacuccaucugugcagcaaacaacacagggguguacauccucacauccaacaccucccaguaugacacauauugcuucaaugcuucagcuccaccugaagaagauuguacaucagucacagaccugcccaaugccuuugauggaccaauuaccauaacuauuguuaaccgugauggcacccgcuauguccagaaaggagaauacagaacgaauccugaagacaucuaccccagcaacccuacugaugaugacgugagcagcggcuccuccagugaaaggagcagcacuucaggagguuacaucuuuuacaccuuuucuacuguacaccccaucccagacgaagacagucccuggaucaccgacagcacagacagaaucccugcuaccagagaccaagacacauuccaccccaguggggggucccauaccacucauggaucugaaucagauggacacucacaugggagucaagaagguggagcaaacacaaccucugguccuauaaggacaccccaaauuccagaauggcugaucaucuuggcaucccucuuggccuuggcuuugauucuugcaguuugcauugcagucaacagucgaagaaggugugggcagaagaaaaagcuagugaucaacaguggcaauggagcuguggaggacagaaagccaaguggacucaacggagaggccagcaagucucaggaaauggugcauuuggugaacaaggagucgucagaaacuccagaccaguuuaugacagcugaugagacaaggaaccugcagaauguggacaugaagauugggguguaacaccuacaccauuaucuuggaaagaaacaaccguuggaaacauaaccauuacagggagcugggacacuuaacagaugcaaugugcuacugauuguuucauugcgaaucuuuuuuagcauaaaauuuucuacucuuuuuguuuuuuguguuuuguucuuuaaagucagguccaauuuguaaaaacagcauugcuuucugaaauuagggcccaauuaauaaucagcaagaauuugaucguuccaguucccacuuggaggccuuucaucccucgggugugcuauggauggcuucuaacaaaaacuacacauauguauuccugaucgccaaccuuucccccaccagcuaaggacauuucccaggguuaauagggccuggucccugggaggaaauuugaauggguccauuuugcccuuccauagccuaaucccugggcauugcuuuccacugagguuggggguugggguguacuaguuacacaucuucaacagacccccucuagaaauuuuucagaugcuucugggagacacccaaagggugaagcuauuuaucuguaguaaacuauuuaucuguguuuuugaaauauuaaacccuggaucaguccuuugaucaguauaauuuuuuaaaguuacuuugucagaggcacaaaaggguuuaaacugauucauaauaaauaucuguacuucuucgaucuucaccuuuugugcugugauucuucaguuucuaaaccagcacugucugggucccuacaauguaucaggaagagcugagaaugguaaggagacucuucuaagucuucaucucagagacccugaguucccacucagacccacucagccaaaucucauggaagaccaaggagggcagcacuguuuuuguuuuuuguuuuuuguuuuuuuuuuuugacacuguccaaagguuuuccauccuguccuggaaucagaguuggaagcugaggagcuucagccucuuuuaugguuuaauggccaccuguucucuccugugaaaggcuuugcaaagucacauuaaguuugcaugaccuguuaucccuggggcccuauuucauagaggcuggcccuauuagugauuuccaaaaacaauauggaagugccuuuugaugucuuacaauaagagaagaagccaauggaaaugaaagagauuggcaaaggggaaggaugaugccauguagauccuguuugacauuuuuauggcuguauuuguaaacuuaaacacaccagugucuguucuugaugcaguugcuauuuaggaugaguuaagugccuggggagucccucaaaagguuaaagggauucccaucauuggaaucuuaucaccagauaggcaaguuuaugaccaaacaagagaguacuggcuuuauccucuaaccucauauuuucucccacuuggcaaguccuuuguggcauuuauucaucagucaggguguccgauugguccuagaacuuccaaaggcugcuugucauagaagccauugcaucuauaaagcaacggcuccuguuaaaugguaucuccuuucugaggcuccuacuaaaagucauuuguuaccuaaacuuaugugcuuaacaggcaaugcuucucagaccacaaagcagaaagaagaagaaaagcuccugacuaaaucagggcugggcuuagacagaguugaucuguagaauaucuuuaaaggagagaugucaacuuucugcacuauucccagccucugcuccucccugucuacccucuccccucccucucucccuccacuucaccccacaaucuugaaaaacuuccuuucucuucugugaacaucauuggccagauccauuuucaguggucuggauuucuuuuuauuuucuuuucaacuugaaagaaacuggacauuaggccacuauguguuguuacugccacuaguguucaagugccucuuguuuucccagagauuuccugggucugccagaggcccagacaggcucacucaagcucuuuaacugaaaagcaacaagccacuccaggacaagguucaaaaugguuacaacagccucuaccugucgccccagggagaaagggguagugauacaagucucauagccagagaugguuuuccacuccuucuagauauucccaaaaagaggcugagacaggagguuauuuucaauuuuauuuuggaauuaaauacuuuuuucccuuuauuacuguuguagucccucacuuggauauaccucuguuuucacgauagaaauaagggaggucuagagcuucuauuccuuggccauugucaacggagagcuggccaagucuucacaaacccuugcaacauugccugaaguuuauggaauaagauguauucucacucccuugaucucaagggcguaacucuggaagcacagcuugacuacacgucauuuuuaccaaugauuuucaggugaccugggcuaagucauuuaaacugggucuuuauaaaaguaaaaggccaacauuuaauuauuuugcaaagcaaccuaagagcuaaagauguaauuuuucuugcaauuguaaaucuuuugugucuccugaagacuucccuuaaaauuagcucugagugaaaaaucaaaagagacaaaagacaucuucgaauccauauuucaagccugguagaauuggcuuuucuagcagaaccuuuccaaaaguuuuauauugagauucauaacaacaccaagaauugauuuuguagccaacauucauucaauacuguuauaucagaggaguaggagagaggaaacauuugacuuaucuggaaaagcaaaauguacuuaagaauaagaauaacaugguccauucaccuuuauguuauagauaugucuuuguguaaaucauuuguuuugaguuuucaaagaauagcccauuguucauucuugugcuguacaaugaccacuguuauuguuacuuugacuuuucagagcacacccuuccucugguuuuuguauauuuauugauggaucaauaauaaugaggaaagcaugauauguauauugcugaguugaaagcacuuauuggaaaauauuaaaaggcuaacauuaaaagacuaaaggaaac

SEQ ID NO: 118 - RNA, 1713 nt, Homo sapiens
  source 1..1713: /mol_type=“unassigned RNA” /note=“Nucleotide sequence encoding homo sapiens CD44 isoform CD44s” /organism=“Homo sapiens”
auuugaauauaaccugccgcuuugcagguguauuccacguggagaaaaauggucgcuacagcaucucucggacggaggccgcugaccucugcaaggcuuucaauagcaccuugcccacaauggcccagauggagaaagcucugagcaucggauuugagaccugcagguauggguucauagaagggcacguggugauuccccggauccaccccaacuccaucugugcagcaaacaacacagggguguacauccucacauccaacaccucccaguaugacacauauugcuucaaugcuucagcuccaccugaagaagauuguacaucagucacagaccugcccaaugccuuugauggaccaauuaccauaacuauuguuaaccgugauggcacccgcuauguccagaaaggagaauacagaacgaauccugaagacaucuaccccagcaacccuacugaugaugacgugagcagcggcuccuccagugaaaggagcagcacuucaggagguuacaucuuuuacaccuuuucuacuguacaccccaucccagacgaagacagucccuggaucaccgacagcacagacagaaucccugcuaccagagaccaagacacauuccaccccaguggggggucccauaccacucauggaucugaaucagauggacacucacaugggagucaagaagguggagcaaacacaaccucugguccuauaaggacaccccaaauuccagaauggcugaucaucuuggcaucccucuuggccuuggcuuugauucuugcaguuugcauugcagucaacagucgaagaaggugugggcagaagaaaaagcuagugaucaacaguggcaauggagcuguggaggacagaaagccaaguggacucaacggagaggccagcaagucucaggaaauggugcauuuggugaacaaggagucgucagaaacuccagaccaguuuaugacagcugaugagacaaggaaccugcagaauguggacaugaagauugggguguaacaccuacaccauuaucuuggaaagaaacaaccguuggaaacauaaccauuacagggagcugggacacuuaacagaugcaaugugcuacugauuguuucauugcgaaucuuuuuuagcauaaaauuuucuacucuuuuuguuuuuuguguuuuguucuuuaaagucagguccaauuuguaaaaacagcauugcuuucugaaauuagggcccaauuaauaaucagcaagaauuugaucguuccaguucccacuuggaggccuuucaucccucgggugugcuauggauggcuucuaacaaaaacuacacauauguauuccugaucgccaaccuuucccccaccagcuaaggacauuucccaggguuaauagggccuggucccugggaggaaauuugaauggguccauuuugcccuuccauagccuaaucccugggcauugcuuuccacugagguuggggguugggguguacuaguuacacaucuucaacagacccccucuagaaauuuuucagaugcuucugggagacacccaaagggugaagcuauuuaucuguaguaaacuauuuaucuguguuuuugaaauauuaaacccuggaucaguccuuugaucaguauaauuuuuuaaaguuacuuugucagaggcacaaaaggguuuaaacugauucauaauaaauaucuguacuucuucgaucuuca

SEQ ID NO: 119 - RNA, 5525 nt, Homo sapiens
  source 1..5525: /mol_type=“unassigned RNA” /note=“Nucleotide sequence encoding homo sapiens CD44 isoform CD44v2-v9” /organism=“Homo sapiens”
gagaagaaagccagugcgucucugggcgcaggggccaguggggcucggaggcacaggcaccccgcgacacuccagguuccccgacccacgucccuggcagccccgauuauuuacagccucagcagagcacggggcgggggcagaggggcccgcccgggagggcugcuacuucuuaaaaccucugcgggcugcuuagucacagccccccuugcuuggguguguccuucgcucgcucccucccuccgucuuaggucacuguuuucaaccucgaauaaaaacugcagccaacuuccgaggcagccucauugcccagcggaccccagccucugccagguucgguccgccauccucgucccguccuccgccggccccugccccgcgcccagggauccuccagcuccuuucgcccgcgcccuccguucgcuccggacaccauggacaaguuuugguggcacgcagccuggggacucugccucgugccgcugagccuggcgcagaucgauuugaauauaaccugccgcuuugcagguguauuccacguggagaaaaauggucgcuacagcaucucucggacggaggccgcugaccucugcaaggcuuucaauagcaccuugcccacaauggcccagauggagaaagcucugagcaucggauuugagaccugcagguauggguucauagaagggcacguggugauuccccggauccaccccaacuccaucugugcagcaaacaacacagggguguacauccucacauccaacaccucccaguaugacacauauugcuucaaugcuucagcuccaccugaagaagauuguacaucagucacagaccugcccaaugccuuugauggaccaauuaccauaacuauuguuaaccgugauggcacccgcuauguccagaaaggagaauacagaacgaauccugaagacaucuaccccagcaacccuacugaugaugacgugagcagcggcuccuccagugaaaggagcagcacuucaggagguuacaucuuuuacaccuuuucuacuguacaccccaucccagacgaagacagucccuggaucaccgacagcacagacagaaucccugcuaccacuuugaugagcacuagugcuacagcaacugagacagcaaccaagaggcaagaaaccugggauugguuuucaugguuguuucuaccaucagagucaaagaaucaucuucacacaacaacacaaauggcugguacgucuucaaauaccaucucagcaggcugggagccaaaugaagaaaaugaagaugaaagagacagacaccucaguuuuucuggaucaggcauugaugaugaugaagauuuuaucuccagcaccauuucaaccacaccacgggcuuuugaccacacaaaacagaaccaggacuggacccaguggaacccaagccauucaaauccggaagugcuacuucagacaaccacaaggaugacugauguagacagaaauggcaccacugcuuaugaaggaaacuggaacccagaagcacacccuccccucauucaccaugagcaucaugaggaagaagagaccccacauucuacaagcacaauccaggcaacuccuaguaguacaacggaagaaacagcuacccagaaggaacagugguuuggcaacagauggcaugagggauaucgccaaacacccaaagaagacucccauucgacaacagggacagcugcagccucagcucauaccagccauccaaugcaaggaaggacaacaccaagcccagaggacaguuccuggacugauuucuucaacccaaucucacaccccaugggacgaggucaucaagcaggaagaaggauggauauggacuccagucauaguauaacgcuucagccuacugcaaauccaaacacagguuugguggaagauuuggacaggacaggaccucuuucaaugacaacgcagcagaguaauucucagagcuucucuacaucacaugaaggcuuggaagaagauaaagaccauccaacaacuucuacucugacaucaagcagagaccaagacacauuccaccccaguggggggucccauaccacucauggaucugaaucagauggacacucacaugggagucaagaagguggagcaaacacaaccucugguccuauaaggacaccccaaauuccagaauggcugaucaucuuggcaucccucuuggccuuggcuuugauucuugcaguuugcauugcagucaacagucgaagaaggugugggcagaagaaaaagcuagugaucaacaguggcaauggagcuguggaggacagaaagccaaguggacucaacggagaggccagcaagucucaggaaauggugcauuuggugaacaaggagucgucagaaacuccagaccaguuuaugacagcugaugagacaaggaaccugcagaauguggacaugaagauugggguguaacaccuacaccauuaucuuggaaagaaacaaccguuggaaacauaaccauuacagggagcugggacacuuaacagaugcaaugugcuacugauuguuucauugcgaaucuuuuuuagcauaaaauuuucuacucuuuuuguuuuuuguguuuuguucuuuaaagucagguccaauuuguaaaaacagcauugcuuucugaaauuagggcccaauuaauaaucagcaagaauuugaucguuccaguucccacuuggaggccuuucaucccucgggugugcuauggauggcuucuaacaaaaacuacacauauguauuccugaucgccaaccuuucccccaccagcuaaggacauuucccaggguuaauagggccuggucccugggaggaaauuugaauggguccauuuugcccuuccauagccuaaucccugggcauugcuuuccacugagguuggggguugggguguacuaguuacacaucuucaacagacccccucuagaaauuuuucagaugcuucugggagacacccaaagggugaagcuauuuaucuguaguaaacuauuuaucuguguuuuugaaauauuaaacccuggaucaguccuuugaucaguauaauuuuuuaaaguuacuuugucagaggcacaaaaggguuuaaacugauucauaauaaauaucuguacuucuucgaucuucaccuuuugugcugugauucuucaguuucuaaaccagcacugucugggucccuacaauguaucaggaagagcugagaaugguaaggagacucuucuaagucuucaucucagagacccugaguucccacucagacccacucagccaaaucucauggaagaccaaggagggcagcacuguuuuuguuuuuuguuuuuuguuuuuuuuuuuugacacuguccaaagguuuuccauccuguccuggaaucagaguuggaagcugaggagcuucagccucuuuuaugguuuaauggccaccuguucucuccugugaaaggcuuugcaaagucacauuaaguuugcaugaccuguuaucccuggggcccuauuucauagaggcuggcccuauuagugauuuccaaaaacaauauggaagugccuuuugaugucuuacaauaagagaagaagccaauggaaaugaaagagauuggcaaaggggaaggaugaugccauguagauccuguuugacauuuuuauggcuguauuuguaaacuuaaacacaccagugucuguucuugaugcaguugcuauuuaggaugaguuaagugccuggggagucccucaaaagguuaaagggauucccaucauuggaaucuuaucaccagauaggcaaguuuaugaccaaacaagagaguacuggcuuuauccucuaaccucauauuuucucccacuuggcaaguccuuuguggcauuuauucaucagucaggguguccgauugguccuagaacuuccaaaggcugcuugucauagaagccauugcaucuauaaagcaacggcuccuguuaaaugguaucuccuuucugaggcuccuacuaaaagucauuuguuaccuaaacuuaugugcuuaacaggcaaugcuucucagaccacaaagcagaaagaagaagaaaagcuccugacuaaaucagggcugggcuuagacagaguugaucuguagaauaucuuuaaaggagagaugucaacuuucugcacuauucccagccucugcuccucccugucuacccucuccccucccucucucccuccacuucaccccacaaucuugaaaaacuuccuuucucuucugugaacaucauuggccagauccauuuucaguggucuggauuucuuuuuauuuucuuuucaacuugaaagaaacuggacauuaggccacuauguguuguuacugccacuaguguucaagugccucuuguuuucccagagauuuccugggucugccagaggcccagacaggcucacucaagcucuuuaacugaaaagcaacaagccacuccaggacaagguucaaaaugguuacaacagccucuaccugucgccccagggagaaagggguagugauacaagucucauagccagagaugguuuuccacuccuucuagauauucccaaaaagaggcugagacaggagguuauuuucaauuuuauuuuggaauuaaauacuuuuuucccuuuauuacuguuguagucccucacuuggauauaccucuguuuucacgauagaaauaagggaggucuagagcuucuauuccuuggccauugucaacggagagcuggccaagucuucacaaacccuugcaacauugccugaaguuuauggaauaagauguauucucacucccuugaucucaagggcguaacucuggaagcacagcuugacuacacgucauuuuuaccaaugauuuucaggugaccugggcuaagucauuuaaacugggucuuuauaaaaguaaaaggccaacauuuaauuauuuugcaaagcaaccuaagagcuaaagauguaauuuuucuugcaauuguaaaucuuuugugucuccugaagacuucccuuaaaauuagcucugagugaaaaaucaaaagagacaaaagacaucuucgaauccauauuucaagccugguagaauuggcuuuucuagcagaaccuuuccaaaaguuuuauauugagauucauaacaacaccaagaauugauuuuguagccaacauucauucaauacuguuauaucagaggaguaggagagaggaaacauuugacuuaucuggaaaagcaaaauguacuuaagaauaagaauaacaugguccauucaccuuuauguuauagauaugucuuuguguaaaucauuuguuuugaguuuucaaagaauagcccauuguucauucuugugcuguacaaugaccacuguuauuguuacuuugacuuuucagagcacacccuuccucugguuuuuguauauuuauugauggaucaauaauaaugaggaaagcaugauauguauauugcugaguugaaagcacuuauuggaaaauauuaaaaggcuaacauuaaaagacuaaaggaaac

SEQ ID NO: 120 - RNA, 5729 nt, Homo sapiens
  source 1..5729: /mol_type=“unassigned RNA” /note=“Nucleotide sequence encoding homo sapiens CD44 isoform CD44v2-10” /organism=“Homo sapiens”
gagaagaaagccagugcgucucugggcgcaggggccaguggggcucggaggcacaggcaccccgcgacacuccagguuccccgacccacgucccuggcagccccgauuauuuacagccucagcagagcacggggcgggggcagaggggcccgcccgggagggcugcuacuucuuaaaaccucugcgggcugcuuagucacagccccccuugcuuggguguguccuucgcucgcucccucccuccgucuuaggucacuguuuucaaccucgaauaaaaacugcagccaacuuccgaggcagccucauugcccagcggaccccagccucugccagguucgguccgccauccucgucccguccuccgccggccccugccccgcgcccagggauccuccagcuccuuucgcccgcgcccuccguucgcuccggacaccauggacaaguuuugguggcacgcagccuggggacucugccucgugccgcugagccuggcgcagaucgauuugaauauaaccugccgcuuugcagguguauuccacguggagaaaaauggucgcuacagcaucucucggacggaggccgcugaccucugcaaggcuuucaauagcaccuugcccacaauggcccagauggagaaagcucugagcaucggauuugagaccugcagguauggguucauagaagggcacguggugauuccccggauccaccccaacuccaucugugcagcaaacaacacagggguguacauccucacauccaacaccucccaguaugacacauauugcuucaaugcuucagcuccaccugaagaagauuguacaucagucacagaccugcccaaugccuuugauggaccaauuaccauaacuauuguuaaccgugauggcacccgcuauguccagaaaggagaauacagaacgaauccugaagacaucuaccccagcaacccuacugaugaugacgugagcagcggcuccuccagugaaaggagcagcacuucaggagguuacaucuuuuacaccuuuucuacuguacaccccaucccagacgaagacagucccuggaucaccgacagcacagacagaaucccugcuaccacuuugaugagcacuagugcuacagcaacugagacagcaaccaagaggcaagaaaccugggauugguuuucaugguuguuucuaccaucagagucaaagaaucaucuucacacaacaacacaaauggcugguacgucuucaaauaccaucucagcaggcugggagccaaaugaagaaaaugaagaugaaagagacagacaccucaguuuuucuggaucaggcauugaugaugaugaagauuuuaucuccagcaccauuucaaccacaccacgggcuuuugaccacacaaaacagaaccaggacuggacccaguggaacccaagccauucaaauccggaagugcuacuucagacaaccacaaggaugacugauguagacagaaauggcaccacugcuuaugaaggaaacuggaacccagaagcacacccuccccucauucaccaugagcaucaugaggaagaagagaccccacauucuacaagcacaauccaggcaacuccuaguaguacaacggaagaaacagcuacccagaaggaacagugguuuggcaacagauggcaugagggauaucgccaaacacccaaagaagacucccauucgacaacagggacagcugcagccucagcucauaccagccauccaaugcaaggaaggacaacaccaagcccagaggacaguuccuggacugauuucuucaacccaaucucacaccccaugggacgaggucaucaagcaggaagaaggauggauauggacuccagucauaguauaacgcuucagccuacugcaaauccaaacacagguuugguggaagauuuggacaggacaggaccucuuucaaugacaacgcagcagaguaauucucagagcuucucuacaucacaugaaggcuuggaagaagauaaagaccauccaacaacuucuacucugacaucaagcaauaggaaugaugucacagguggaagaagagacccaaaucauucugaaggcucaacuacuuuacuggaagguuauaccucucauuacccacacacgaaggaaagcaggaccuucaucccagugaccucagcuaagacuggguccuuuggaguuacugcaguuacuguuggagauuccaacucuaaugucaaucguuccuuaucaggagaccaagacacauuccaccccaguggggggucccauaccacucauggaucugaaucagauggacacucacaugggagucaagaagguggagcaaacacaaccucugguccuauaaggacaccccaaauuccagaauggcugaucaucuuggcaucccucuuggccuuggcuuugauucuugcaguuugcauugcagucaacagucgaagaaggugugggcagaagaaaaagcuagugaucaacaguggcaauggagcuguggaggacagaaagccaaguggacucaacggagaggccagcaagucucaggaaauggugcauuuggugaacaaggagucgucagaaacuccagaccaguuuaugacagcugaugagacaaggaaccugcagaauguggacaugaagauugggguguaacaccuacaccauuaucuuggaaagaaacaaccguuggaaacauaaccauuacagggagcugggacacuuaacagaugcaaugugcuacugauuguuucauugcgaaucuuuuuuagcauaaaauuuucuacucuuuuuguuuuuuguguuuuguucuuuaaagucagguccaauuuguaaaaacagcauugcuuucugaaauuagggcccaauuaauaaucagcaagaauuugaucguuccaguucccacuuggaggccuuucaucccucgggugugcuauggauggcuucuaacaaaaacuacacauauguauuccugaucgccaaccuuucccccaccagcuaaggacauuucccaggguuaauagggccuggucccugggaggaaauuugaauggguccauuuugcccuuccauagccuaaucccugggcauugcuuuccacugagguuggggguugggguguacuaguuacacaucuucaacagacccccucuagaaauuuuucagaugcuucugggagacacccaaagggugaagcuauuuaucuguaguaaacuauuuaucuguguuuuugaaauauuaaacccuggaucaguccuuugaucaguauaauuuuuuaaaguuacuuugucagaggcacaaaaggguuuaaacugauucauaauaaauaucuguacuucuucgaucuucaccuuuugugcugugauucuucaguuucuaaaccagcacugucugggucccuacaauguaucaggaagagcugagaaugguaaggagacucuucuaagucuucaucucagagacccugaguucccacucagacccacucagccaaaucucauggaagaccaaggagggcagcacuguuuuuguuuuuuguuuuuuguuuuuuuuuuuugacacuguccaaagguuuuccauccuguccuggaaucagaguuggaagcugaggagcuucagccucuuuuaugguuuaauggccaccuguucucuccugugaaaggcuuugcaaagucacauuaaguuugcaugaccuguuaucccuggggcccuauuucauagaggcuggcccuauuagugauuuccaaaaacaauauggaagugccuuuugaugucuuacaauaagagaagaagccaauggaaaugaaagagauuggcaaaggggaaggaugaugccauguagauccuguuugacauuuuuauggcuguauuuguaaacuuaaacacaccagugucuguucuugaugcaguugcuauuuaggaugaguuaagugccuggggagucccucaaaagguuaaagggauucccaucauuggaaucuuaucaccagauaggcaaguuuaugaccaaacaagagaguacuggcuuuauccucuaaccucauauuuucucccacuuggcaaguccuuuguggcauuuauucaucagucaggguguccgauugguccuagaacuuccaaaggcugcuugucauagaagccauugcaucuauaaagcaacggcuccuguuaaaugguaucuccuuucugaggcuccuacuaaaagucauuuguuaccuaaacuuaugugcuuaacaggcaaugcuucucagaccacaaagcagaaagaagaagaaaagcuccugacuaaaucagggcugggcuuagacagaguugaucuguagaauaucuuuaaaggagagaugucaacuuucugcacuauucccagccucugcuccucccugucuacccucuccccucccucucucccuccacuucaccccacaaucuugaaaaacuuccuuucucuucugugaacaucauuggccagauccauuuucaguggucuggauuucuuuuuauuuucuuuucaacuugaaagaaacuggacauuaggccacuauguguuguuacugccacuaguguucaagugccucuuguuuucccagagauuuccugggucugccagaggcccagacaggcucacucaagcucuuuaacugaaaagcaacaagccacuccaggacaagguucaaaaugguuacaacagccucuaccugucgccccagggagaaagggguagugauacaagucucauagccagagaugguuuuccacuccuucuagauauucccaaaaagaggcugagacaggagguuauuuucaauuuuauuuuggaauuaaauacuuuuuucccuuuauuacuguuguagucccucacuuggauauaccucuguuuucacgauagaaauaagggaggucuagagcuucuauuccuuggccauugucaacggagagcuggccaagucuucacaaacccuugcaacauugccugaaguuuauggaauaagauguauucucacucccuugaucucaagggcguaacucuggaagcacagcuugacuacacgucauuuuuaccaaugauuuucaggugaccugggcuaagucauuuaaacugggucuuuauaaaaguaaaaggccaacauuuaauuauuuugcaaagcaaccuaagagcuaaagauguaauuuuucuugcaauuguaaaucuuuugugucuccugaagacuucccuuaaaauuagcucugagugaaaaaucaaaagagacaaaagacaucuucgaauccauauuucaagccugguagaauuggcuuuucuagcagaaccuuuccaaaaguuuuauauugagauucauaacaacaccaagaauugauuuuguagccaacauucauucaauacuguuauaucagaggaguaggagagaggaaacauuugacuuaucuggaaaagcaaaauguacuuaagaauaagaauaacaugguccauucaccuuuauguuauagauaugucuuuguguaaaucauuuguuuugaguuuucaaagaauagcccauuguucauucuugugcuguacaaugaccacuguuauuguuacuuugacuuuucagagcacacccuuccucugguuuuuguauauuuauugauggaucaauaauaaugaggaaagcaugauauguauauugcugaguugaaagcacuuauuggaaaauauuaaaaggcuaacauuaaaagacuaaaggaaac

SEQ ID NO: 121 - RNA, 5600 nt, Homo sapiens
  source 1..5600: /mol_type=“unassigned RNA” /note=“Nucleotide sequence encoding homo sapiens CD44 isoform CD44v2-10” /organism=“Homo sapiens”
gagaagaaagccagugcgucucugggcgcaggggccaguggggcucggaggcacaggcaccccgcgacacuccagguuccccgacccacgucccuggcagccccgauuauuuacagccucagcagagcacggggcgggggcagaggggcccgcccgggagggcugcuacuucuuaaaaccucugcgggcugcuuagucacagccccccuugcuuggguguguccuucgcucgcucccucccuccgucuuaggucacuguuuucaaccucgaauaaaaacugcagccaacuuccgaggcagccucauugcccagcggaccccagccucugccagguucgguccgccauccucgucccguccuccgccggccccugccccgcgcccagggauccuccagcuccuuucgcccgcgcccuccguucgcuccggacaccauggacaaguuuugguggcacgcagccuggggacucugccucgugccgcugagccuggcgcagaucgauuugaauauaaccugccgcuuugcagguguauuccacguggagaaaaauggucgcuacagcaucucucggacggaggccgcugaccucugcaaggcuuucaauagcaccuugcccacaauggcccagauggagaaagcucugagcaucggauuugagaccugcagguauggguucauagaagggcacguggugauuccccggauccaccccaacuccaucugugcagcaaacaacacagggguguacauccucacauccaacaccucccaguaugacacauauugcuucaaugcuucagcuccaccugaagaagauuguacaucagucacagaccugcccaaugccuuugauggaccaauuaccauaacuauuguuaaccgugauggcacccgcuauguccagaaaggagaauacagaacgaauccugaagacaucuaccccagcaacccuacugaugaugacgugagcagcggcuccuccagugaaaggagcagcacuucaggagguuacaucuuuuacaccuuuucuacuguacaccccaucccagacgaagacagucccuggaucaccgacagcacagacagaaucccugcuaccacuuugaugagcacuagugcuacagcaacugagacagcaaccaagaggcaagaaaccugggauugguuuucaugguuguuucuaccaucagagucaaagaaucaucuucacacaacaacacaaauggcugguacgucuucaaauaccaucucagcaggcugggagccaaaugaagaaaaugaagaugaaagagacagacaccucaguuuuucuggaucaggcauugaugaugaugaagauuuuaucuccagcaccauuucaaccacaccacgggcuuuugaccacacaaaacagaaccaggacuggacccaguggaacccaagccauucaaauccggaagugcuacuucagacaaccacaaggaugacugauguagacagaaauggcaccacugcuuaugaaggaaacuggaacccagaagcacacccuccccucauucaccaugagcaucaugaggaagaagagaccccacauucuacaagcacaacagccucagcucauaccagccauccaaugcaaggaaggacaacaccaagcccagaggacaguuccuggacugauuucuucaacccaaucucacaccccaugggacgaggucaucaagcaggaagaaggauggauauggacuccagucauaguauaacgcuucagccuacugcaaauccaaacacagguuugguggaagauuuggacaggacaggaccucuuucaaugacaacgcagcagaguaauucucagagcuucucuacaucacaugaaggcuuggaagaagauaaagaccauccaacaacuucuacucugacaucaagcaauaggaaugaugucacagguggaagaagagacccaaaucauucugaaggcucaacuacuuuacuggaagguuauaccucucauuacccacacacgaaggaaagcaggaccuucaucccagugaccucagcuaagacuggguccuuuggaguuacugcaguuacuguuggagauuccaacucuaaugucaaucguuccuuaucaggagaccaagacacauuccaccccaguggggggucccauaccacucauggaucugaaucagauggacacucacaugggagucaagaagguggagcaaacacaaccucugguccuauaaggacaccccaaauuccagaauggcugaucaucuuggcaucccucuuggccuuggcuuugauucuugcaguuugcauugcagucaacagucgaagaaggugugggcagaagaaaaagcuagugaucaacaguggcaauggagcuguggaggacagaaagccaaguggacucaacggagaggccagcaagucucaggaaauggugcauuuggugaacaaggagucgucagaaacuccagaccaguuuaugacagcugaugagacaaggaaccugcagaauguggacaugaagauugggguguaacaccuacaccauuaucuuggaaagaaacaaccguuggaaacauaaccauuacagggagcugggacacuuaacagaugcaaugugcuacugauuguuucauugcgaaucuuuuuuagcauaaaauuuucuacucuuuuuguuuuuuguguuuuguucuuuaaagucagguccaauuuguaaaaacagcauugcuuucugaaauuagggcccaauuaauaaucagcaagaauuugaucguuccaguucccacuuggaggccuuucaucccucgggugugcuauggauggcuucuaacaaaaacuacacauauguauuccugaucgccaaccuuucccccaccagcuaaggacauuucccaggguuaauagggccuggucccugggaggaaauuugaauggguccauuuugcccuuccauagccuaaucccugggcauugcuuuccacugagguuggggguugggguguacuaguuacacaucuucaacagacccccucuagaaauuuuucagaugcuucugggagacacccaaagggugaagcuauuuaucuguaguaaacuauuuaucuguguuuuugaaauauuaaacccuggaucaguccuuugaucaguauaauuuuuuaaaguuacuuugucagaggcacaaaaggguuuaaacugauucauaauaaauaucuguacuucuucgaucuucaccuuuugugcugugauucuucaguuucuaaaccagcacugucugggucccuacaauguaucaggaagagcugagaaugguaaggagacucuucuaagucuucaucucagagacccugaguucccacucagacccacucagccaaaucucauggaagaccaaggagggcagcacuguuuuuguuuuuuguuuuuuguuuuuuuuuuuugacacuguccaaagguuuuccauccuguccuggaaucagaguuggaagcugaggagcuucagccucuuuuaugguuuaauggccaccuguucucuccugugaaaggcuuugcaaagucacauuaaguuugcaugaccuguuaucccuggggcccuauuucauagaggcuggcccuauuagugauuuccaaaaacaauauggaagugccuuuugaugucuuacaauaagagaagaagccaauggaaaugaaagagauuggcaaaggggaaggaugaugccauguagauccuguuugacauuuuuauggcuguauuuguaaacuuaaacacaccagugucuguucuugaugcaguugcuauuuaggaugaguuaagugccuggggagucccucaaaagguuaaagggauucccaucauuggaaucuuaucaccagauaggcaaguuuaugaccaaacaagagaguacuggcuuuauccucuaaccucauauuuucucccacuuggcaaguccuuuguggcauuuauucaucagucaggguguccgauugguccuagaacuuccaaaggcugcuugucauagaagccauugcaucuauaaagcaacggcuccuguuaaaugguaucuccuuucugaggcuccuacuaaaagucauuuguuaccuaaacuuaugugcuuaacaggcaaugcuucucagaccacaaagcagaaagaagaagaaaagcuccugacuaaaucagggcugggcuuagacagaguugaucuguagaauaucuuuaaaggagagaugucaacuuucugcacuauucccagccucugcuccucccugucuacccucuccccucccucucucccuccacuucaccccacaaucuugaaaaacuuccuuucucuucugugaacaucauuggccagauccauuuucaguggucuggauuucuuuuuauuuucuuuucaacuugaaagaaacuggacauuaggccacuauguguuguuacugccacuaguguucaagugccucuuguuuucccagagauuuccugggucugccagaggcccagacaggcucacucaagcucuuuaacugaaaagcaacaagccacuccaggacaagguucaaaaugguuacaacagccucuaccugucgccccagggagaaagggguagugauacaagucucauagccagagaugguuuuccacuccuucuagauauucccaaaaagaggcugagacaggagguuauuuucaauuuuauuuuggaauuaaauacuuuuuucccuuuauuacuguuguagucccucacuuggauauaccucuguuuucacgauagaaauaagggaggucuagagcuucuauuccuuggccauugucaacggagagcuggccaagucuucacaaacccuugcaacauugccugaaguuuauggaauaagauguauucucacucccuugaucucaagggcguaacucuggaagcacagcuugacuacacgucauuuuuaccaaugauuuucaggugaccugggcuaagucauuuaaacugggucuuuauaaaaguaaaaggccaacauuuaauuauuuugcaaagcaaccuaagagcuaaagauguaauuuuucuugcaauuguaaaucuuuugugucuccugaagacuucccuuaaaauuagcucugagugaaaaaucaaaagagacaaaagacaucuucgaauccauauuucaagccugguagaauuggcuuuucuagcagaaccuuuccaaaaguuuuauauugagauucauaacaacaccaagaauugauuuuguagccaacauucauucaauacuguuauaucagaggaguaggagagaggaaacauuugacuuaucuggaaaagcaaaauguacuuaagaauaagaauaacaugguccauucaccuuuauguuauagauaugucuuuguguaaaucauuuguuuugaguuuucaaagaauagcccauuguucauucuugugcuguacaaugaccacuguuauuguuacuuugacuuuucagagcacacccuuccucugguuuuuguauauuuauugauggaucaauaauaaugaggaaagcaugauauguauauugcugaguugaaagcacuuauuggaaaauauuaaaaggcuaacauuaaaagacuaaaggaaac

SEQ ID NO: 122 - RNA, 5705 nt, Homo sapiens
  source 1..5705: /mol_type=“unassigned RNA” /note=“Nucleotide sequence encoding homo sapiens CD44 isoform CD44v2-10” /organism=“Homo sapiens”
gagaagaaagccagugcgucucugggcgcaggggccaguggggcucggaggcacaggcaccccgcgacacuccagguuccccgacccacgucccuggcagccccgauuauuuacagccucagcagagcacggggcgggggcagaggggcccgcccgggagggcugcuacuucuuaaaaccucugcgggcugcuuagucacagccccccuugcuuggguguguccuucgcucgcucccucccuccgucuuaggucacuguuuucaaccucgaauaaaaacugcagccaacuuccgaggcagccucauugcccagcggaccccagccucugccagguucgguccgccauccucgucccguccuccgccggccccugccccgcgcccagggauccuccagcuccuuucgcccgcgcccuccguucgcuccggacaccauggacaaguuuugguggcacgcagccuggggacucugccucgugccgcugagccuggcgcagaucgauuugaauauaaccugccgcuuugcagguguauuccacguggagaaaaauggucgcuacagcaucucucggacggaggccgcugaccucugcaaggcuuucaauagcaccuugcccacaauggcccagauggagaaagcucugagcaucggauuugagaccugcagguauggguucauagaagggcacguggugauuccccggauccaccccaacuccaucugugcagcaaacaacacagggguguacauccucacauccaacaccucccaguaugacacauauugcuucaaugcuucagcuccaccugaagaagauuguacaucagucacagaccugcccaaugccuuugauggaccaauuaccauaacuauuguuaaccgugauggcacccgcuauguccagaaaggagaauacagaacgaauccugaagacaucuaccccagcaacccuacugaugaugacgugagcagcggcuccuccagugaaaggagcagcacuucaggagguuacaucuuuuacaccuuuucuacuguacaccccaucccagacgaagacagucccuggaucaccgacagcacagacagaaucccugcuaccacuuugaugagcacuagugcuacagcaacugagacagcaaccaagaggcaagaaaccugggauugguuuucaugguuguuucuaccaucagagucaaagaaucaucuucacacaacaacacaaauggcugcaggcugggagccaaaugaagaaaaugaagaugaaagagacagacaccucaguuuuucuggaucaggcauugaugaugaugaagauuuuaucuccagcaccauuucaaccacaccacgggcuuuugaccacacaaaacagaaccaggacuggacccaguggaacccaagccauucaaauccggaagugcuacuucagacaaccacaaggaugacugauguagacagaaauggcaccacugcuuaugaaggaaacuggaacccagaagcacacccuccccucauucaccaugagcaucaugaggaagaagagaccccacauucuacaagcacaauccaggcaacuccuaguaguacaacggaagaaacagcuacccagaaggaacagugguuuggcaacagauggcaugagggauaucgccaaacacccaaagaagacucccauucgacaacagggacagcugcagccucagcucauaccagccauccaaugcaaggaaggacaacaccaagcccagaggacaguuccuggacugauuucuucaacccaaucucacaccccaugggacgaggucaucaagcaggaagaaggauggauauggacuccagucauaguauaacgcuucagccuacugcaaauccaaacacagguuugguggaagauuuggacaggacaggaccucuuucaaugacaacgcagcagaguaauucucagagcuucucuacaucacaugaaggcuuggaagaagauaaagaccauccaacaacuucuacucugacaucaagcaauaggaaugaugucacagguggaagaagagacccaaaucauucugaaggcucaacuacuuuacuggaagguuauaccucucauuacccacacacgaaggaaagcaggaccuucaucccagugaccucagcuaagacuggguccuuuggaguuacugcaguuacuguuggagauuccaacucuaaugucaaucguuccuuaucaggagaccaagacacauuccaccccaguggggggucccauaccacucauggaucugaaucagauggacacucacaugggagucaagaagguggagcaaacacaaccucugguccuauaaggacaccccaaauuccagaauggcugaucaucuuggcaucccucuuggccuuggcuuugauucuugcaguuugcauugcagucaacagucgaagaaggugugggcagaagaaaaagcuagugaucaacaguggcaauggagcuguggaggacagaaagccaaguggacucaacggagaggccagcaagucucaggaaauggugcauuuggugaacaaggagucgucagaaacuccagaccaguuuaugacagcugaugagacaaggaaccugcagaauguggacaugaagauugggguguaacaccuacaccauuaucuuggaaagaaacaaccguuggaaacauaaccauuacagggagcugggacacuuaacagaugcaaugugcuacugauuguuucauugcgaaucuuuuuuagcauaaaauuuucuacucuuuuuguuuuuuguguuuuguucuuuaaagucagguccaauuuguaaaaacagcauugcuuucugaaauuagggcccaauuaauaaucagcaagaauuugaucguuccaguucccacuuggaggccuuucaucccucgggugugcuauggauggcuucuaacaaaaacuacacauauguauuccugaucgccaaccuuucccccaccagcuaaggacauuucccaggguuaauagggccuggucccugggaggaaauuugaauggguccauuuugcccuuccauagccuaaucccugggcauugcuuuccacugagguuggggguugggguguacuaguuacacaucuucaacagacccccucuagaaauuuuucagaugcuucugggagacacccaaagggugaagcuauuuaucuguaguaaacuauuuaucuguguuuuugaaauauuaaacccuggaucaguccuuugaucaguauaauuuuuuaaaguuacuuugucagaggcacaaaaggguuuaaacugauucauaauaaauaucuguacuucuucgaucuucaccuuuugugcugugauucuucaguuucuaaaccagcacugucugggucccuacaauguaucaggaagagcugagaaugguaaggagacucuucuaagucuucaucucagagacccugaguucccacucagacccacucagccaaaucucauggaagaccaaggagggcagcacuguuuuuguuuuuuguuuuuuguuuuuuuuuuuugacacuguccaaagguuuuccauccuguccuggaaucagaguuggaagcugaggagcuucagccucuuuuaugguuuaauggccaccuguucucuccugugaaaggcuuugcaaagucacauuaaguuugcaugaccuguuaucccuggggcccuauuucauagaggcuggcccuauuagugauuuccaaaaacaauauggaagugccuuuugaugucuuacaauaagagaagaagccaauggaaaugaaagagauuggcaaaggggaaggaugaugccauguagauccuguuugacauuuuuauggcuguauuuguaaacuuaaacacaccagugucuguucuugaugcaguugcuauuuaggaugaguuaagugccuggggagucccucaaaagguuaaagggauucccaucauuggaaucuuaucaccagauaggcaaguuuaugaccaaacaagagaguacuggcuuuauccucuaaccucauauuuucucccacuuggcaaguccuuuguggcauuuauucaucagucaggguguccgauugguccuagaacuuccaaaggcugcuugucauagaagccauugcaucuauaaagcaacggcuccuguuaaaugguaucuccuuucugaggcuccuacuaaaagucauuuguuaccuaaacuuaugugcuuaacaggcaaugcuucucagaccacaaagcagaaagaagaagaaaagcuccugacuaaaucagggcugggcuuagacagaguugaucuguagaauaucuuuaaaggagagaugucaacuuucugcacuauucccagccucugcuccucccugucuacccucuccccucccucucucccuccacuucaccccacaaucuugaaaaacuuccuuucucuucugugaacaucauuggccagauccauuuucaguggucuggauuucuuuuuauuuucuuuucaacuugaaagaaacuggacauuaggccacuauguguuguuacugccacuaguguucaagugccucuuguuuucccagagauuuccugggucugccagaggcccagacaggcucacucaagcucuuuaacugaaaagcaacaagccacuccaggacaagguucaaaaugguuacaacagccucuaccugucgccccagggagaaagggguagugauacaagucucauagccagagaugguuuuccacuccuucuagauauucccaaaaagaggcugagacaggagguuauuuucaauuuuauuuuggaauuaaauacuuuuuucccuuuauuacuguuguagucccucacuuggauauaccucuguuuucacgauagaaauaagggaggucuagagcuucuauuccuuggccauugucaacggagagcuggccaagucuucacaaacccuugcaacauugccugaaguuuauggaauaagauguauucucacucccuugaucucaagggcguaacucuggaagcacagcuugacuacacgucauuuuuaccaaugauuuucaggugaccugggcuaagucauuuaaacugggucuuuauaaaaguaaaaggccaacauuuaauuauuuugcaaagcaaccuaagagcuaaagauguaauuuuucuugcaauuguaaaucuuuugugucuccugaagacuucccuuaaaauuagcucugagugaaaaaucaaaagagacaaaagacaucuucgaauccauauuucaagccugguagaauuggcuuuucuagcagaaccuuuccaaaaguuuuauauugagauucauaacaacaccaagaauugauuuuguagccaacauucauucaauacuguuauaucagaggaguaggagagaggaaacauuugacuuaucuggaaaagcaaaauguacuuaagaauaagaauaacaugguccauucaccuuuauguuauagauaugucuuuguguaaaucauuuguuuugaguuuucaaagaauagcccauuguucauucuugugcuguacaaugaccacuguuauuguuacuuugacuuuucagagcacacccuuccucugguuuuuguauauuuauugauggaucaauaauaaugaggaaagcaugauauguauauugcugaguugaaagcacuuauuggaaaauauuaaaaggcuaacauuaaaagacuaaaggaaac

SEQ ID NO: 123 - RNA, 5639 nt, Homo sapiens
  source 1..5639: /mol_type=“unassigned RNA” /note=“Nucleotide sequence encoding homo sapiens CD44 isoform CD44v2-v10” /organism=“Homo sapiens”
gagaagaaagccagugcgucucugggcgcaggggccaguggggcucggaggcacaggcaccccgcgacacuccagguuccccgacccacgucccuggcagccccgauuauuuacagccucagcagagcacggggcgggggcagaggggcccgcccgggagggcugcuacuucuuaaaaccucugcgggcugcuuagucacagccccccuugcuuggguguguccuucgcucgcucccucccuccgucuuaggucacuguuuucaaccucgaauaaaaacugcagccaacuuccgaggcagccucauugcccagcggaccccagccucugccagguucgguccgccauccucgucccguccuccgccggccccugccccgcgcccagggauccuccagcuccuuucgcccgcgcccuccguucgcuccggacaccauggacaaguuuugguggcacgcagccuggggacucugccucgugccgcugagccuggcgcagaucgauuugaauauaaccugccgcuuugcagguguauuccacguggagaaaaauggucgcuacagcaucucucggacggaggccgcugaccucugcaaggcuuucaauagcaccuugcccacaauggcccagauggagaaagcucugagcaucggauuugagaccugcagguauggguucauagaagggcacguggugauuccccggauccaccccaacuccaucugugcagcaaacaacacagggguguacauccucacauccaacaccucccaguaugacacauauugcuucaaugcuucagcuccaccugaagaagauuguacaucagucacagaccugcccaaugccuuugauggaccaauuaccauaacuauuguuaaccgugauggcacccgcuauguccagaaaggagaauacagaacgaauccugaagacaucuaccccagcaacccuacugaugaugacgugagcagcggcuccuccagugaaaggagcagcacuucaggagguuacaucuuuuacaccuuuucuacuguacaccccaucccagacgaagacagucccuggaucaccgacagcacagacagaaucccugcuaccacuuugaugagcacuagugcuacagcaacugagacagcaaccaagaggcaagaaaccugggauugguuuucaugguuguuucuaccaucagagucaaagaaucaucuucacacaacaacacaaauggcugguacgucuucaaauaccaucucagcaggcugggagccaaaugaagaaaaugaagaugaaagagacagacaccucaguuuuucuggaucaggcauugaugaugaugaagauuuuaucuccagcaccauuucaaccacaccacgggcuuuugaccacacaaaacagaaccaggacuggacccaguggaacccaagccauucaaauccggaagugcuacuucagacaaccacaaggaugacugauguagacagaaauggcaccacugcuuaugaaggaaacuggaacccagaagcacacccuccccucauucaccaugagcaucaugaggaagaagagaccccacauucuacaagcacaauccaggcaacuccuaguaguacaacggaagaaacagcuacccagaaggaacagugguuuggcaacagauggcaugagggauaucgccaaacacccaaagaagacucccauucgacaacagggacagcugcagccucagcucauaccagccauccaaugcaaggaaggacaacaccaagcccagaggacaguuccuggacugauuucuucaacccaaucucacaccccaugggacgaggucaucaagcaggaagaaggauggauauggacuccagucauaguauaacgcuucagccuacugcaaauccaaacacagguuugguggaagauuuggacaggacaggaccucuuucaaugacaacgcauaggaaugaugucacagguggaagaagagacccaaaucauucugaaggcucaacuacuuuacuggaagguuauaccucucauuacccacacacgaaggaaagcaggaccuucaucccagugaccucagcuaagacuggguccuuuggaguuacugcaguuacuguuggagauuccaacucuaaugucaaucguuccuuaucaggagaccaagacacauuccaccccaguggggggucccauaccacucauggaucugaaucagauggacacucacaugggagucaagaagguggagcaaacacaaccucugguccuauaaggacaccccaaauuccagaauggcugaucaucuuggcaucccucuuggccuuggcuuugauucuugcaguuugcauugcagucaacagucgaagaaggugugggcagaagaaaaagcuagugaucaacaguggcaauggagcuguggaggacagaaagccaaguggacucaacggagaggccagcaagucucaggaaauggugcauuuggugaacaaggagucgucagaaacuccagaccaguuuaugacagcugaugagacaaggaaccugcagaauguggacaugaagauugggguguaacaccuacaccauuaucuuggaaagaaacaaccguuggaaacauaaccauuacagggagcugggacacuuaacagaugcaaugugcuacugauuguuucauugcgaaucuuuuuuagcauaaaauuuucuacucuuuuuguuuuuuguguuuuguucuuuaaagucagguccaauuuguaaaaacagcauugcuuucugaaauuagggcccaauuaauaaucagcaagaauuugaucguuccaguucccacuuggaggccuuucaucccucgggugugcuauggauggcuucuaacaaaaacuacacauauguauuccugaucgccaaccuuucccccaccagcuaaggacauuucccaggguuaauagggccuggucccugggaggaaauuugaauggguccauuuugcccuuccauagccuaaucccugggcauugcuuuccacugagguuggggguugggguguacuaguuacacaucuucaacagacccccucuagaaauuuuucagaugcuucugggagacacccaaagggugaagcuauuuaucuguaguaaacuauuuaucuguguuuuugaaauauuaaacccuggaucaguccuuugaucaguauaauuuuuuaaaguuacuuugucagaggcacaaaaggguuuaaacugauucauaauaaauaucuguacuucuucgaucuucaccuuuugugcugugauucuucaguuucuaaaccagcacugucugggucccuacaauguaucaggaagagcugagaaugguaaggagacucuucuaagucuucaucucagagacccugaguucccacucagacccacucagccaaaucucauggaagaccaaggagggcagcacuguuuuuguuuuuuguuuuuuguuuuuuuuuuuugacacuguccaaagguuuuccauccuguccuggaaucagaguuggaagcugaggagcuucagccucuuuuaugguuuaauggccaccuguucucuccugugaaaggcuuugcaaagucacauuaaguuugcaugaccuguuaucccuggggcccuauuucauagaggcuggcccuauuagugauuuccaaaaacaauauggaagugccuuuugaugucuuacaauaagagaagaagccaauggaaaugaaagagauuggcaaaggggaaggaugaugccauguagauccuguuugacauuuuuauggcuguauuuguaaacuuaaacacaccagugucuguucuugaugcaguugcuauuuaggaugaguuaagugccuggggagucccucaaaagguuaaagggauucccaucauuggaaucuuaucaccagauaggcaaguuuaugaccaaacaagagaguacuggcuuuauccucuaaccucauauuuucucccacuuggcaaguccuuuguggcauuuauucaucagucaggguguccgauugguccuagaacuuccaaaggcugcuugucauagaagccauugcaucuauaaagcaacggcuccuguuaaaugguaucuccuuucugaggcuccuacuaaaagucauuuguuaccuaaacuuaugugcuuaacaggcaaugcuucucagaccacaaagcagaaagaagaagaaaagcuccugacuaaaucagggcugggcuuagacagaguugaucuguagaauaucuuuaaaggagagaugucaacuuucugcacuauucccagccucugcuccucccugucuacccucuccccucccucucucccuccacuucaccccacaaucuugaaaaacuuccuuucucuucugugaacaucauuggccagauccauuuucaguggucuggauuucuuuuuauuuucuuuucaacuugaaagaaacuggacauuaggccacuauguguuguuacugccacuaguguucaagugccucuuguuuucccagagauuuccugggucugccagaggcccagacaggcucacucaagcucuuuaacugaaaagcaacaagccacuccaggacaagguucaaaaugguuacaacagccucuaccugucgccccagggagaaagggguagugauacaagucucauagccagagaugguuuuccacuccuucuagauauucccaaaaagaggcugagacaggagguuauuuucaauuuuauuuuggaauuaaauacuuuuuucccuuuauuacuguuguagucccucacuuggauauaccucuguuuucacgauagaaauaagggaggucuagagcuucuauuccuuggccauugucaacggagagcuggccaagucuucacaaacccuugcaacauugccugaaguuuauggaauaagauguauucucacucccuugaucucaagggcguaacucuggaagcacagcuugacuacacgucauuuuuaccaaugauuuucaggugaccugggcuaagucauuuaaacugggucuuuauaaaaguaaaaggccaacauuuaauuauuuugcaaagcaaccuaagagcuaaagauguaauuuuucuugcaauuguaaaucuuuugugucuccugaagacuucccuuaaaauuagcucugagugaaaaaucaaaagagacaaaagacaucuucgaauccauauuucaagccugguagaauuggcuuuucuagcagaaccuuuccaaaaguuuuauauugagauucauaacaacaccaagaauugauuuuguagccaacauucauucaauacuguuauaucagaggaguaggagagaggaaacauuugacuuaucuggaaaagcaaaauguacuuaagaauaagaauaacaugguccauucaccuuuauguuauagauaugucuuuguguaaaucauuuguuuugaguuuucaaagaauagcccauuguucauucuugugcuguacaaugaccacuguuauuguuacuuugacuuuucagagcacacccuuccucugguuuuuguauauuuauugauggaucaauaauaaugaggaaagcaugauauguauauugcugaguugaaagcacuuauuggaaaauauuaaaaggcuaacauuaaaagacuaaaggaaac

SEQ ID NO: 124 - RNA, 2856 nt, Homo sapiens
  source 1..2856: /mol_type=“unassigned RNA” /note=“Nucleotide sequence encoding homo sapiens CD44 isoform CD44v2-v10” /organism=“Homo sapiens”
auuugaauauaaccugccgcuuugcagguguauuccacguggagaaaaauggucgcuacagcaucucucggacggaggccgcugaccucugcaaggcuuucaauagcaccuugcccacaauggcccagauggagaaagcucugagcaucggauuugagaccugcagguauggguucauagaagggcacguggugauuccccggauccaccccaacuccaucugugcagcaaacaacacagggguguacauccucacauccaacaccucccaguaugacacauauugcuucaaugcuucagcuccaccugaagaagauuguacaucagucacagaccugcccaaugccuuugauggaccaauuaccauaacuauuguuaaccgugauggcacccgcuauguccagaaaggagaauacagaacgaauccugaagacaucuaccccagcaacccuacugaugaugacgugagcagcggcuccuccagugaaaggagcagcacuucaggagguuacaucuuuuacaccuuuucuacuguacaccccaucccagacgaagacagucccuggaucaccgacagcacagacagaaucccugcuaccacuuugaugagcacuagugcuacagcaacugagacagcaaccaagaggcaagaaaccugggauugguuuucaugguuguuucuaccaucagagucaaagaaucaucuucacacaacaacacaaauggcugguacgucuucaaauaccaucucagcaggcugggagccaaaugaagaaaaugaagaugaaagagacagacaccucaguuuuucuggaucaggcauugaugaugaugaagauuuuaucuccagcaccauuucaaccacaccacgggcuuuugaccacacaaaacagaaccaggacuggacccaguggaacccaagccauucaaauccggaagugcuacuucagacaaccacaaggaugacugauguagacagaaauggcaccacugcuuaugaaggaaacuggaacccagaagcacacccuccccucauucaccaugagcaucaugaggaagaagagaccccacauucuacaagcacaauccaggcaacuccuaguaguacaacggaagaaacagcuacccagaaggaacagugguuuggcaacagauggcaugagggauaucgccaaacacccaaagaagacucccauucgacaacagggacagcugcagccucagcucauaccagccauccaaugcaaggaaggacaacaccaagcccagaggacaguuccuggacugauuucuucaacccaaucucacaccccaugggacgaggucaucaagcaggaagaaggauggauauggacuccagucauaguauaacgcuucagccuacugcaaauccaaacacagguuugguggaagauuuggacaggacaggaccucuuucaaugacaacgcagcagaguaauucucagagcuucucuacaucacaugaaggcuuggaagaagauaaagaccauccaacaacuucuacucugacaucaagcaauaggaaugaugucacagguggaagaagagacccaaaucauucugaaggcucaacuacuuuacuggaagguuauaccucucauuacccacacacgaaggaaagcaggaccuucaucccagugaccucagcuaagacuggguccuuuggaguuacugcaguuacuguuggagauuccaacucuaaugucaaucguuccuuaucaggagaccaagacacauuccaccccaguggggggucccauaccacucauggaucugaaucagauggacacucacaugggagucaagaagguggagcaaacacaaccucugguccuauaaggacaccccaaauuccagaauggcugaucaucuuggcaucccucuuggccuuggcuuugauucuugcaguuugcauugcagucaacagucgaagaaggugugggcagaagaaaaagcuagugaucaacaguggcaauggagcuguggaggacagaaagccaaguggacucaacggagaggccagcaagucucaggaaauggugcauuuggugaacaaggagucgucagaaacuccagaccaguuuaugacagcugaugagacaaggaaccugcagaauguggacaugaagauugggguguaacaccuacaccauuaucuuggaaagaaacaaccguuggaaacauaaccauuacagggagcugggacacuuaacagaugcaaugugcuacugauuguuucauugcgaaucuuuuuuagcauaaaauuuucuacucuuuuuguuuuuuguguuuuguucuuuaaagucagguccaauuuguaaaaacagcauugcuuucugaaauuagggcccaauuaauaaucagcaagaauuugaucguuccaguucccacuuggaggccuuucaucccucgggugugcuauggauggcuucuaacaaaaacuacacauauguauuccugaucgccaaccuuucccccaccagcuaaggacauuucccaggguuaauagggccuggucccugggaggaaauuugaauggguccauuuugcccuuccauagccuaaucccugggcauugcuuuccacugagguuggggguugggguguacuaguuacacaucuucaacagacccccucuagaaauuuuucagaugcuucugggagacacccaaagggugaagcuauuuaucuguaguaaacuauuuaucuguguuuuugaaauauuaaacccuggaucaguccuuugaucaguauaauuuuuuaaaguuacuuugucagaggcacaaaaggguuuaaacugauucauaauaaauaucuguacuucuucgaucuuca

SEQ ID NO: 125 - RNA, 5636 nt, Homo sapiens
  source 1..5636: /mol_type=“unassigned RNA” /note=“Nucleotide sequence encoding homo sapiens CD44 isoform CD44v2-v10” /organism=“Homo sapiens”
gagaagaaagccagugcgucucugggcgcaggggccaguggggcucggaggcacaggcaccccgcgacacuccagguuccccgacccacgucccuggcagccccgauuauuuacagccucagcagagcacggggcgggggcagaggggcccgcccgggagggcugcuacuucuuaaaaccucugcgggcugcuuagucacagccccccuugcuuggguguguccuucgcucgcucccucccuccgucuuaggucacuguuuucaaccucgaauaaaaacugcagccaacuuccgaggcagccucauugcccagcggaccccagccucugccagguucgguccgccauccucgucccguccuccgccggccccugccccgcgcccagggauccuccagcuccuuucgcccgcgcccuccguucgcuccggacaccauggacaaguuuugguggcacgcagccuggggacucugccucgugccgcugagccuggcgcagaucgauuugaauauaaccugccgcuuugcagguguauuccacguggagaaaaauggucgcuacagcaucucucggacggaggccgcugaccucugcaaggcuuucaauagcaccuugcccacaauggcccagauggagaaagcucugagcaucggauuugagaccugcagguauggguucauagaagggcacguggugauuccccggauccaccccaacuccaucugugcagcaaacaacacagggguguacauccucacauccaacaccucccaguaugacacauauugcuucaaugcuucagcuccaccugaagaagauuguacaucagucacagaccugcccaaugccuuugauggaccaauuaccauaacuauuguuaaccgugauggcacccgcuauguccagaaaggagaauacagaacgaauccugaagacaucuaccccagcaacccuacugaugaugacgugagcagcggcuccuccagugaaaggagcagcacuucaggagcuuugaugagcacuagugcuacagcaacugagacagcaaccaagaggcaagaaaccugggauugguuuucaugguuguuucuaccaucagagucaaagaaucaucuucacacaacaacacaaauggcugguacgucuucaaauaccaucucagcaggcugggagccaaaugaagaaaaugaagaugaaagagacagacaccucaguuuuucuggaucaggcauugaugaugaugaagauuuuaucuccagcaccauuucaaccacaccacgggcuuuugaccacacaaaacagaaccaggacuggacccaguggaacccaagccauucaaauccggaagugcuacuucagacaaccacaaggaugacugauguagacagaaauggcaccacugcuuaugaaggaaacuggaacccagaagcacacccuccccucauucaccaugagcaucaugaggaagaagagaccccacauucuacaagcacaauccaggcaacuccuaguaguacaacggaagaaacagcuacccagaaggaacagugguuuggcaacagauggcaugagggauaucgccaaacacccaaagaagacucccauucgacaacagggacagcugcagccucagcucauaccagccauccaaugcaaggaaggacaacaccaagcccagaggacaguuccuggacugauuucuucaacccaaucucacaccccaugggacgaggucaucaagcaggaagaaggauggauauggacuccagucauaguauaacgcuucagccuacugcaaauccaaacacagguuugguggaagauuuggacaggacaggaccucuuucaaugacaacgcagcagaguaauucucagagcuucucuacaucacaugaaggcuuggaagaagauaaagaccauccaacaacuucuacucugacaucaagcaauaggaaugaugucacagguggaagaagagacccaaaucauucugaaggcucaacuacuuuacuggaagguuauaccucucauuacccacacacgaaggaaagcaggaccuucaucccagugaccucagcuaagacuggguccuuuggaguuacugcaguuacuguuggagauuccaacucuaaugucaaucguuccuuaucaggagaccaagacacauuccaccccaguggggggucccauaccacucauggaucugaaucagauggacacucacaugggagucaagaagguggagcaaacacaaccucugguccuauaaggacaccccaaauuccagaauggcugaucaucuuggcaucccucuuggccuuggcuuugauucuugcaguuugcauugcagucaacagucgaagaaggugugggcagaagaaaaagcuagugaucaacaguggcaauggagcuguggaggacagaaagccaaguggacucaacggagaggccagcaagucucaggaaauggugcauuuggugaacaaggagucgucagaaacuccagaccaguuuaugacagcugaugagacaaggaaccugcagaauguggacaugaagauugggguguaacaccuacaccauuaucuuggaaagaaacaaccguuggaaacauaaccauuacagggagcugggacacuuaacagaugcaaugugcuacugauuguuucauugcgaaucuuuuuuagcauaaaauuuucuacucuuuuuguuuuuuguguuuuguucuuuaaagucagguccaauuuguaaaaacagcauugcuuucugaaauuagggcccaauuaauaaucagcaagaauuugaucguuccaguucccacuuggaggccuuucaucccucgggugugcuauggauggcuucuaacaaaaacuacacauauguauuccugaucgccaaccuuucccccaccagcuaaggacauuucccaggguuaauagggccuggucccugggaggaaauuugaauggguccauuuugcccuuccauagccuaaucccugggcauugcuuuccacugagguuggggguugggguguacuaguuacacaucuucaacagacccccucuagaaauuuuucagaugcuucugggagacacccaaagggugaagcuauuuaucuguaguaaacuauuuaucuguguuuuugaaauauuaaacccuggaucaguccuuugaucaguauaauuuuuuaaaguuacuuugucagaggcacaaaaggguuuaaacugauucauaauaaauaucuguacuucuucgaucuucaccuuuugugcugugauucuucaguuucuaaaccagcacugucugggucccuacaauguaucaggaagagcugagaaugguaaggagacucuucuaagucuucaucucagagacccugaguucccacucagacccacucagccaaaucucauggaagaccaaggagggcagcacuguuuuuguuuuuuguuuuuuguuuuuuuuuuuugacacuguccaaagguuuuccauccuguccuggaaucagaguuggaagcugaggagcuucagccucuuuuaugguuuaauggccaccuguucucuccugugaaaggcuuugcaaagucacauuaaguuugcaugaccuguuaucccuggggcccuauuucauagaggcuggcccuauuagugauuuccaaaaacaauauggaagugccuuuugaugucuuacaauaagagaagaagccaauggaaaugaaagagauuggcaaaggggaaggaugaugccauguagauccuguuugacauuuuuauggcuguauuuguaaacuuaaacacaccagugucuguucuugaugcaguugcuauuuaggaugaguuaagugccuggggagucccucaaaagguuaaagggauucccaucauuggaaucuuaucaccagauaggcaaguuuaugaccaaacaagagaguacuggcuuuauccucuaaccucauauuuucucccacuuggcaaguccuuuguggcauuuauucaucagucaggguguccgauugguccuagaacuuccaaaggcugcuugucauagaagccauugcaucuauaaagcaacggcuccuguuaaaugguaucuccuuucugaggcuccuacuaaaagucauuuguuaccuaaacuuaugugcuuaacaggcaaugcuucucagaccacaaagcagaaagaagaagaaaagcuccugacuaaaucagggcugggcuuagacagaguugaucuguagaauaucuuuaaaggagagaugucaacuuucugcacuauucccagccucugcuccucccugucuacccucuccccucccucucucccuccacuucaccccacaaucuugaaaaacuuccuuucucuucugugaacaucauuggccagauccauuuucaguggucuggauuucuuuuuauuuucuuuucaacuugaaagaaacuggacauuaggccacuauguguuguuacugccacuaguguucaagugccucuuguuuucccagagauuuccugggucugccagaggcccagacaggcucacucaagcucuuuaacugaaaagcaacaagccacuccaggacaagguucaaaaugguuacaacagccucuaccugucgccccagggagaaagggguagugauacaagucucauagccagagaugguuuuccacuccuucuagauauucccaaaaagaggcugagacaggagguuauuuucaauuuuauuuuggaauuaaauacuuuuuucccuuuauuacuguuguagucccucacuuggauauaccucuguuuucacgauagaaauaagggaggucuagagcuucuauuccuuggccauugucaacggagagcuggccaagucuucacaaacccuugcaacauugccugaaguuuauggaauaagauguauucucacucccuugaucucaagggcguaacucuggaagcacagcuugacuacacgucauuuuuaccaaugauuuucaggugaccugggcuaagucauuuaaacugggucuuuauaaaaguaaaaggccaacauuuaauuauuuugcaaagcaaccuaagagcuaaagauguaauuuuucuugcaauuguaaaucuuuugugucuccugaagacuucccuuaaaauuagcucugagugaaaaaucaaaagagacaaaagacaucuucgaauccauauuucaagccugguagaauuggcuuuucuagcagaaccuuuccaaaaguuuuauauugagauucauaacaacaccaagaauugauuuuguagccaacauucauucaauacuguuauaucagaggaguaggagagaggaaacauuugacuuaucuggaaaagcaaaauguacuuaagaauaagaauaacaugguccauucaccuuuauguuauagauaugucuuuguguaaaucauuuguuuugaguuuucaaagaauagcccauuguucauucuugugcuguacaaugaccacuguuauuguuacuuugacuuuucagagcacacccuuccucugguuuuuguauauuuauugauggaucaauaauaaugaggaaagcaugauauguauauugcugaguugaaagcacuuauuggaaaauauuaaaaggcuaacauuaaaagacuaaaggaaac

SEQ ID NO: 126 - RNA, 5600 nt, Homo sapiens
  source 1..5600: /mol_type=“unassigned RNA” /note=“Nucleotide sequence homo sapiens CD44 isoform CD44v3-10” /organism=“Homo sapiens”
gagaagaaagccagugcgucucugggcgcaggggccaguggggcucggaggcacaggcaccccgcgacacuccagguuccccgacccacgucccuggcagccccgauuauuuacagccucagcagagcacggggcgggggcagaggggcccgcccgggagggcugcuacuucuuaaaaccucugcgggcugcuuagucacagccccccuugcuuggguguguccuucgcucgcucccucccuccgucuuaggucacuguuuucaaccucgaauaaaaacugcagccaacuuccgaggcagccucauugcccagcggaccccagccucugccagguucgguccgccauccucgucccguccuccgccggccccugccccgcgcccagggauccuccagcuccuuucgcccgcgcccuccguucgcuccggacaccauggacaaguuuugguggcacgcagccuggggacucugccucgugccgcugagccuggcgcagaucgauuugaauauaaccugccgcuuugcagguguauuccacguggagaaaaauggucgcuacagcaucucucggacggaggccgcugaccucugcaaggcuuucaauagcaccuugcccacaauggcccagauggagaaagcucugagcaucggauuugagaccugcagguauggguucauagaagggcacguggugauuccccggauccaccccaacuccaucugugcagcaaacaacacagggguguacauccucacauccaacaccucccaguaugacacauauugcuucaaugcuucagcuccaccugaagaagauuguacaucagucacagaccugcccaaugccuuugauggaccaauuaccauaacuauuguuaaccgugauggcacccgcuauguccagaaaggagaauacagaacgaauccugaagacaucuaccccagcaacccuacugaugaugacgugagcagcggcuccuccagugaaaggagcagcacuucaggagguuacaucuuuuacaccuuuucuacuguacaccccaucccagacgaagacagucccuggaucaccgacagcacagacagaaucccugcuaccaguacgucuucaaauaccaucucagcaggcugggagccaaaugaagaaaaugaagaugaaagagacagacaccucaguuuuucuggaucaggcauugaugaugaugaagauuuuaucuccagcaccauuucaaccacaccacgggcuuuugaccacacaaaacagaaccaggacuggacccaguggaacccaagccauucaaauccggaagugcuacuucagacaaccacaaggaugacugauguagacagaaauggcaccacugcuuaugaaggaaacuggaacccagaagcacacccuccccucauucaccaugagcaucaugaggaagaagagaccccacauucuacaagcacaauccaggcaacuccuaguaguacaacggaagaaacagcuacccagaaggaacagugguuuggcaacagauggcaugagggauaucgccaaacacccaaagaagacucccauucgacaacagggacagcugcagccucagcucauaccagccauccaaugcaaggaaggacaacaccaagcccagaggacaguuccuggacugauuucuucaacccaaucucacaccccaugggacgaggucaucaagcaggaagaaggauggauauggacuccagucauaguauaacgcuucagccuacugcaaauccaaacacagguuugguggaagauuuggacaggacaggaccucuuucaaugacaacgcagcagaguaauucucagagcuucucuacaucacaugaaggcuuggaagaagauaaagaccauccaacaacuucuacucugacaucaagcaauaggaaugaugucacagguggaagaagagacccaaaucauucugaaggcucaacuacuuuacuggaagguuauaccucucauuacccacacacgaaggaaagcaggaccuucaucccagugaccucagcuaagacuggguccuuuggaguuacugcaguuacuguuggagauuccaacucuaaugucaaucguuccuuaucaggagaccaagacacauuccaccccaguggggggucccauaccacucauggaucugaaucagauggacacucacaugggagucaagaagguggagcaaacacaaccucugguccuauaaggacaccccaaauuccagaauggcugaucaucuuggcaucccucuuggccuuggcuuugauucuugcaguuugcauugcagucaacagucgaagaaggugugggcagaagaaaaagcuagugaucaacaguggcaauggagcuguggaggacagaaagccaaguggacucaacggagaggccagcaagucucaggaaauggugcauuuggugaacaaggagucgucagaaacuccagaccaguuuaugacagcugaugagacaaggaaccugcagaauguggacaugaagauugggguguaacaccuacaccauuaucuuggaaagaaacaaccguuggaaacauaaccauuacagggagcugggacacuuaacagaugcaaugugcuacugauuguuucauugcgaaucuuuuuuagcauaaaauuuucuacucuuuuuguuuuuuguguuuuguucuuuaaagucagguccaauuuguaaaaacagcauugcuuucugaaauuagggcccaauuaauaaucagcaagaauuugaucguuccaguucccacuuggaggccuuucaucccucgggugugcuauggauggcuucuaacaaaaacuacacauauguauuccugaucgccaaccuuucccccaccagcuaaggacauuucccaggguuaauagggccuggucccugggaggaaauuugaauggguccauuuugcccuuccauagccuaaucccugggcauugcuuuccacugagguuggggguugggguguacuaguuacacaucuucaacagacccccucuagaaauuuuucagaugcuucugggagacacccaaagggugaagcuauuuaucuguaguaaacuauuuaucuguguuuuugaaauauuaaacccuggaucaguccuuugaucaguauaauuuuuuaaaguuacuuugucagaggcacaaaaggguuuaaacugauucauaauaaauaucuguacuucuucgaucuucaccuuuugugcugugauucuucaguuucuaaaccagcacugucugggucccuacaauguaucaggaagagcugagaaugguaaggagacucuucuaagucuucaucucagagacccugaguucccacucagacccacucagccaaaucucauggaagaccaaggagggcagcacuguuuuuguuuuuuguuuuuuguuuuuuuuuuuugacacuguccaaagguuuuccauccuguccuggaaucagaguuggaagcugaggagcuucagccucuuuuaugguuuaauggccaccuguucucuccugugaaaggcuuugcaaagucacauuaaguuugcaugaccuguuaucccuggggcccuauuucauagaggcuggcccuauuagugauuuccaaaaacaauauggaagugccuuuugaugucuuacaauaagagaagaagccaauggaaaugaaagagauuggcaaaggggaaggaugaugccauguagauccuguuugacauuuuuauggcuguauuuguaaacuuaaacacaccagugucuguucuugaugcaguugcuauuuaggaugaguuaagugccuggggagucccucaaaagguuaaagggauucccaucauuggaaucuuaucaccagauaggcaaguuuaugaccaaacaagagaguacuggcuuuauccucuaaccucauauuuucucccacuuggcaaguccuuuguggcauuuauucaucagucaggguguccgauugguccuagaacuuccaaaggcugcuugucauagaagccauugcaucuauaaagcaacggcuccuguuaaaugguaucuccuuucugaggcuccuacuaaaagucauuuguuaccuaaacuuaugugcuuaacaggcaaugcuucucagaccacaaagcagaaagaagaagaaaagcuccugacuaaaucagggcugggcuuagacagaguugaucuguagaauaucuuuaaaggagagaugucaacuuucugcacuauucccagccucugcuccucccugucuacccucuccccucccucucucccuccacuucaccccacaaucuugaaaaacuuccuuucucuucugugaacaucauuggccagauccauuuucaguggucuggauuucuuuuuauuuucuuuucaacuugaaagaaacuggacauuaggccacuauguguuguuacugccacuaguguucaagugccucuuguuuucccagagauuuccugggucugccagaggcccagacaggcucacucaagcucuuuaacugaaaagcaacaagccacuccaggacaagguucaaaaugguuacaacagccucuaccugucgccccagggagaaagggguagugauacaagucucauagccagagaugguuuuccacuccuucuagauauucccaaaaagaggcugagacaggagguuauuuucaauuuuauuuuggaauuaaauacuuuuuucccuuuauuacuguuguagucccucacuuggauauaccucuguuuucacgauagaaauaagggaggucuagagcuucuauuccuuggccauugucaacggagagcuggccaagucuucacaaacccuugcaacauugccugaaguuuauggaauaagauguauucucacucccuugaucucaagggcguaacucuggaagcacagcuugacuacacgucauuuuuaccaaugauuuucaggugaccugggcuaagucauuuaaacugggucuuuauaaaaguaaaaggccaacauuuaauuauuuugcaaagcaaccuaagagcuaaagauguaauuuuucuugcaauuguaaaucuuuugugucuccugaagacuucccuuaaaauuagcucugagugaaaaaucaaaagagacaaaagacaucuucgaauccauauuucaagccugguagaauuggcuuuucuagcagaaccuuuccaaaaguuuuauauugagauucauaacaacaccaagaauugauuuuguagccaacauucauucaauacuguuauaucagaggaguaggagagaggaaacauuugacuuaucuggaaaagcaaaauguacuuaagaauaagaauaacaugguccauucaccuuuauguuauagauaugucuuuguguaaaucauuuguuuugaguuuucaaagaauagcccauuguucauucuugugcuguacaaugaccacuguuauuguuacuuugacuuuucagagcacacccuuccucugguuuuuguauauuuauugauggaucaauaauaaugaggaaagcaugauauguauauugcugaguugaaagcacuuauuggaaaauauuaaaaggcuaacauuaaaagacuaaaggaaac

SEQ ID NO: 127 - RNA, 2069 nt, Homo sapiens
  source 1..2069: /mol_type=“unassigned RNA” /note=“Nucleotide sequence encoding homo sapiens CD44 isoform CD44v3-10” /organism=“Homo sapiens”
auuugaauauaaccugccgcuuugcagguguauuccacguggagaaaaauggucgcuacagcaucucucggacggaggccgcugaccucugcaaggcuuucaauagcaccuugcccacaauggcccagauggagaaagcucugagcaucggauuugagaccugcagguauggguucauagaagggcacguggugauuccccggauccaccccaacuccaucugugcagcaaacaacacagggguguacauccucacauccaacaccucccaguaugacacauauugcuucaaugcuucagcuccaccugaagaagauuguacaucagucacagaccugcccaaugccuuugauggaccaauuaccauaacuauuguuaaccgugauggcacccgcuauguccagaaaggagaauacagaacgaauccugaagacaucuaccccagcaacccuacugaugaugacgugagcagcggcuccuccagugaaaggagcagcacuucaggagguuacaucuuuuacaccuuuucuacuguacaccccaucccagacgaagacagucccuggaucaccgacagcacagacagaaucccugcuaccaguacgucuucaaauaccaucucagcaggcugggagccaaaugaagaaaaugaagaugaaagagacagacaccucaguuuuucuggaucaggcauugaugaugaugaagauuuuaucuccagcaccauuucaaccacaccacgggcuuuugaccacacaaaacagaaccaggacuggacccaguggaacccaagccauucaaauccggaagugcuacuucagacaaccacaaggaugacugauguagacagaaauggcaccacugcuuaugaaggaaacuggaacccagaagcacacccuccccucauucaccaugagcaucaugaggaagaagagaccccacauucuacaagcacaauccaggcaacuccuaguaguacaacggaagaaacagcuacccagaaggaacagugguuuggcaacagauggcaugagggauaucgccaaacacccaaagaagacucccauucgacaacagggacagcugcagccucagcucauaccagccauccaaugcaaggaaggacaacaccaagcccagaggacaguuccuggacugauuucuucaacccaaucucacaccccaugggacgaggucaucaagcaggaagaaggauggauauggacuccagucauaguauaacgcuucagccuacugcaaauccaaacacagguuugguggaagauuuggacaggacaggaccucuuucaaugacaacgcagcagaguaauucucagagcuucucuacaucacaugaaggcuuggaagaagauaaagaccauccaacaacuucuacucugacaucaagcaauaggaaugaugucacagguggaagaagagacccaaaucauucugaaggcucaacuacuuuacuggaagguuauaccucucauuacccacacacgaaggaaagcaggaccuucaucccagugaccucagcuaagacuggguccuuuggaguuacugcaguuacuguuggagauuccaacucuaaugucaaucguuccuuaucaggagaccaagacacauuccaccccaguggggggucccauaccacucauggaucugaaucagauggacacucacaugggagucaagaagguggagcaaacacaaccucugguccuauaaggacaccccaaauuccagaauggcugaucaucuuggcaucccucuuggccuuggcuuugauucuugcaguuugcauugcagucaacagucgaagaaggugugggcagaagaaaaagcuagugaucaacaguggcaauggagcuguggaggacagaaagccaaguggacucaacggagaggccagcaagucucaggaaauggugcauuuggugaacaaggagucgucagaaacuccagaccaguuuaugacagcugaugagacaaggaaccugcagaauguggacaugaagauugggguguaacaccuacaccauuaucuuggaaagaaacaaccguug

SEQ ID NO: 128 - RNA, 4982 nt, Homo sapiens
  source 1..4982: /mol_type=“unassigned RNA” /note=“Nucleotide sequence encoding homo sapiens CD44 isoform CD44v8-10” /organism=“Homo sapiens”
gagaagaaagccagugcgucucugggcgcaggggccaguggggcucggaggcacaggcaccccgcgacacuccagguuccccgacccacgucccuggcagccccgauuauuuacagccucagcagagcacggggcgggggcagaggggcccgcccgggagggcugcuacuucuuaaaaccucugcgggcugcuuagucacagccccccuugcuuggguguguccuucgcucgcucccucccuccgucuuaggucacuguuuucaaccucgaauaaaaacugcagccaacuuccgaggcagccucauugcccagcggaccccagccucugccagguucgguccgccauccucgucccguccuccgccggccccugccccgcgcccagggauccuccagcuccuuucgcccgcgcccuccguucgcuccggacaccauggacaaguuuugguggcacgcagccuggggacucugccucgugccgcugagccuggcgcagaucgauuugaauauaaccugccgcuuugcagguguauuccacguggagaaaaauggucgcuacagcaucucucggacggaggccgcugaccucugcaaggcuuucaauagcaccuugcccacaauggcccagauggagaaagcucugagcaucggauuugagaccugcagguauggguucauagaagggcacguggugauuccccggauccaccccaacuccaucugugcagcaaacaacacagggguguacauccucacauccaacaccucccaguaugacacauauugcuucaaugcuucagcuccaccugaagaagauuguacaucagucacagaccugcccaaugccuuugauggaccaauuaccauaacuauuguuaaccgugauggcacccgcuauguccagaaaggagaauacagaacgaauccugaagacaucuaccccagcaacccuacugaugaugacgugagcagcggcuccuccagugaaaggagcagcacuucaggagguuacaucuuuuacaccuuuucuacuguacaccccaucccagacgaagacagucccuggaucaccgacagcacagacagaaucccugcuaccaauauggacuccagucauaguauaacgcuucagccuacugcaaauccaaacacagguuugguggaagauuuggacaggacaggaccucuuucaaugacaacgcagcagaguaauucucagagcuucucuacaucacaugaaggcuuggaagaagauaaagaccauccaacaacuucuacucugacaucaagcaauaggaaugaugucacagguggaagaagagacccaaaucauucugaaggcucaacuacuuuacuggaagguuauaccucucauuacccacacacgaaggaaagcaggaccuucaucccagugaccucagcuaagacuggguccuuuggaguuacugcaguuacuguuggagauuccaacucuaaugucaaucguuccuuaucaggagaccaagacacauuccaccccaguggggggucccauaccacucauggaucugaaucagauggacacucacaugggagucaagaagguggagcaaacacaaccucugguccuauaaggacaccccaaauuccagaauggcugaucaucuuggcaucccucuuggccuuggcuuugauucuugcaguuugcauugcagucaacagucgaagaaggugugggcagaagaaaaagcuagugaucaacaguggcaauggagcuguggaggacagaaagccaaguggacucaacggagaggccagcaagucucaggaaauggugcauuuggugaacaaggagucgucagaaacuccagaccaguuuaugacagcugaugagacaaggaaccugcagaauguggacaugaagauugggguguaacaccuacaccauuaucuuggaaagaaacaaccguuggaaacauaaccauuacagggagcugggacacuuaacagaugcaaugugcuacugauuguuucauugcgaaucuuuuuuagcauaaaauuuucuacucuuuuuguuuuuuguguuuuguucuuuaaagucagguccaauuuguaaaaacagcauugcuuucugaaauuagggcccaauuaauaaucagcaagaauuugaucguuccaguucccacuuggaggccuuucaucccucgggugugcuauggauggcuucuaacaaaaacuacacauauguauuccugaucgccaaccuuucccccaccagcuaaggacauuucccaggguuaauagggccuggucccugggaggaaauuugaauggguccauuuugcccuuccauagccuaaucccugggcauugcuuuccacugagguuggggguugggguguacuaguuacacaucuucaacagacccccucuagaaauuuuucagaugcuucugggagacacccaaagggugaagcuauuuaucuguaguaaacuauuuaucuguguuuuugaaauauuaaacccuggaucaguccuuugaucaguauaauuuuuuaaaguuacuuugucagaggcacaaaaggguuuaaacugauucauaauaaauaucuguacuucuucgaucuucaccuuuugugcugugauucuucaguuucuaaaccagcacugucugggucccuacaauguaucaggaagagcugagaaugguaaggagacucuucuaagucuucaucucagagacccugaguucccacucagacccacucagccaaaucucauggaagaccaaggagggcagcacuguuuuuguuuuuuguuuuuuguuuuuuuuuuuugacacuguccaaagguuuuccauccuguccuggaaucagaguuggaagcugaggagcuucagccucuuuuaugguuuaauggccaccuguucucuccugugaaaggcuuugcaaagucacauuaaguuugcaugaccuguuaucccuggggcccuauuucauagaggcuggcccuauuagugauuuccaaaaacaauauggaagugccuuuugaugucuuacaauaagagaagaagccaauggaaaugaaagagauuggcaaaggggaaggaugaugccauguagauccuguuugacauuuuuauggcuguauuuguaaacuuaaacacaccagugucuguucuugaugcaguugcuauuuaggaugaguuaagugccuggggagucccucaaaagguuaaagggauucccaucauuggaaucuuaucaccagauaggcaaguuuaugaccaaacaagagaguacuggcuuuauccucuaaccucauauuuucucccacuuggcaaguccuuuguggcauuuauucaucagucaggguguccgauugguccuagaacuuccaaaggcugcuugucauagaagccauugcaucuauaaagcaacggcuccuguuaaaugguaucuccuuucugaggcuccuacuaaaagucauuuguuaccuaaacuuaugugcuuaacaggcaaugcuucucagaccacaaagcagaaagaagaagaaaagcuccugacuaaaucagggcugggcuuagacagaguugaucuguagaauaucuuuaaaggagagaugucaacuuucugcacuauucccagccucugcuccucccugucuacccucuccccucccucucucccuccacuucaccccacaaucuugaaaaacuuccuuucucuucugugaacaucauuggccagauccauuuucaguggucuggauuucuuuuuauuuucuuuucaacuugaaagaaacuggacauuaggccacuauguguuguuacugccacuaguguucaagugccucuuguuuucccagagauuuccugggucugccagaggcccagacaggcucacucaagcucuuuaacugaaaagcaacaagccacuccaggacaagguucaaaaugguuacaacagccucuaccugucgccccagggagaaagggguagugauacaagucucauagccagagaugguuuuccacuccuucuagauauucccaaaaagaggcugagacaggagguuauuuucaauuuuauuuuggaauuaaauacuuuuuucccuuuauuacuguuguagucccucacuuggauauaccucuguuuucacgauagaaauaagggaggucuagagcuucuauuccuuggccauugucaacggagagcuggccaagucuucacaaacccuugcaacauugccugaaguuuauggaauaagauguauucucacucccuugaucucaagggcguaacucuggaagcacagcuugacuacacgucauuuuuaccaaugauuuucaggugaccugggcuaagucauuuaaacugggucuuuauaaaaguaaaaggccaacauuuaauuauuuugcaaagcaaccuaagagcuaaagauguaauuuuucuugcaauuguaaaucuuuugugucuccugaagacuucccuuaaaauuagcucugagugaaaaaucaaaagagacaaaagacaucuucgaauccauauuucaagccugguagaauuggcuuuucuagcagaaccuuuccaaaaguuuuauauugagauucauaacaacaccaagaauugauuuuguagccaacauucauucaauacuguuauaucagaggaguaggagagaggaaacauuugacuuaucuggaaaagcaaaauguacuuaagaauaagaauaacaugguccauucaccuuuauguuauagauaugucuuuguguaaaucauuuguuuugaguuuucaaagaauagcccauuguucauucuugugcuguacaaugaccacuguuauuguuacuuugacuuuucagagcacacccuuccucugguuuuuguauauuuauugauggaucaauaauaaugaggaaagcaugauauguauauugcugaguugaaagcacuuauuggaaaauauuaaaaggcuaacauuaaaagacuaaaggaaac

SEQ ID NO: 129 - RNA, 2103 nt, Homo sapiens
  source 1..2103: /mol_type=“unassigned RNA” /note=“Nucleotide sequence encoding homo sapiens CD44 isoform CD44v8-v10” /organism=“Homo sapiens”
auuugaauauaaccugccgcuuugcagguguauuccacguggagaaaaauggucgcuacagcaucucucggacggaggccgcugaccucugcaaggcuuucaauagcaccuugcccacaauggcccagauggagaaagcucugagcaucggauuugagaccugcagguauggguucauagaagggcacguggugauuccccggauccaccccaacuccaucugugcagcaaacaacacagggguguacauccucacauccaacaccucccaguaugacacauauugcuucaaugcuucagcuccaccugaagaagauuguacaucagucacagaccugcccaaugccuuugauggaccaauuaccauaacuauuguuaaccgugauggcacccgcuauguccagaaaggagaauacagaacgaauccugaagacaucuaccccagcaacccuacugaugaugacgugagcagcggcuccuccagugaaaggagcagcacuucaggagguuacaucuuuuacaccuuuucuacuguacaccccaucccagacgaagacagucccuggaucaccgacagcacagacagaaucccugcuaccaauauggacuccagucauaguauaacgcuucagccuacugcaaauccaaacacagguuugguggaagauuuggacaggacaggaccucuuucaaugacaacgcagcagaguaauucucagagcuucucuacaucacaugaaggcuuggaagaagauaaagaccauccaacaacuucuacucugacaucaagcaauaggaaugaugucacagguggaagaagagacccaaaucauucugaaggcucaacuacuuuacuggaagguuauaccucucauuacccacacacgaaggaaagcaggaccuucaucccagugaccucagcuaagacuggguccuuuggaguuacugcaguuacuguuggagauuccaacucuaaugucaaucguuccuuaucaggagaccaagacacauuccaccccaguggggggucccauaccacucauggaucugaaucagauggacacucacaugggagucaagaagguggagcaaacacaaccucugguccuauaaggacaccccaaauuccagaauggcugaucaucuuggcaucccucuuggccuuggcuuugauucuugcaguuugcauugcagucaacagucgaagaaggugugggcagaagaaaaagcuagugaucaacaguggcaauggagcuguggaggacagaaagccaaguggacucaacggagaggccagcaagucucaggaaauggugcauuuggugaacaaggagucgucagaaacuccagaccaguuuaugacagcugaugagacaaggaaccugcagaauguggacaugaagauugggguguaacaccuacaccauuaucuuggaaagaaacaaccguuggaaacauaaccauuacagggagcugggacacuuaacagaugcaaugugcuacugauuguuucauugcgaaucuuuuuuagcauaaaauuuucuacucuuuuuguuuuuuguguuuuguucuuuaaagucagguccaauuuguaaaaacagcauugcuuucugaaauuagggcccaauuaauaaucagcaagaauuugaucguuccaguucccacuuggaggccuuucaucccucgggugugcuauggauggcuucuaacaaaaacuacacauauguauuccugaucgccaaccuuucccccaccagcuaaggacauuucccaggguuaauagggccuggucccugggaggaaauuugaauggguccauuuugcccuuccauagccuaaucccugggcauugcuuuccacugagguuggggguugggguguacuaguuacacaucuucaacagacccccucuagaaauuuuucagaugcuucugggagacacccaaagggugaagcuauuuaucuguaguaaacuauuuaucuguguuuuugaaauauuaaacccuggaucaguccuuugaucaguauaauuuuuuaaaguuacuuugucagaggcacaaaaggguuuaaacugauucauaauaaauaucuguacuucuucga

SEQ ID NO: 130 - RNA, 4718 nt, Homo sapiens
  source 1..4718: /mol_type=“unassigned RNA” /note=“Nucleotide sequence encoding homo sapiens CD44 isoform CD44v6” /organism=“Homo sapiens”
gagaagaaagccagugcgucucugggcgcaggggccaguggggcucggaggcacaggcaccccgcgacacuccagguuccccgacccacgucccuggcagccccgauuauuuacagccucagcagagcacggggcgggggcagaggggcccgcccgggagggcugcuacuucuuaaaaccucugcgggcugcuuagucacagccccccuugcuuggguguguccuucgcucgcucccucccuccgucuuaggucacuguuuucaaccucgaauaaaaacugcagccaacuuccgaggcagccucauugcccagcggaccccagccucugccagguucgguccgccauccucgucccguccuccgccggccccugccccgcgcccagggauccuccagcuccuuucgcccgcgcccuccguucgcuccggacaccauggacaaguuuugguggcacgcagccuggggacucugccucgugccgcugagccuggcgcagaucgauuugaauauaaccugccgcuuugcagguguauuccacguggagaaaaauggucgcuacagcaucucucggacggaggccgcugaccucugcaaggcuuucaauagcaccuugcccacaauggcccagauggagaaagcucugagcaucggauuugagaccugcagguauggguucauagaagggcacguggugauuccccggauccaccccaacuccaucugugcagcaaacaacacagggguguacauccucacauccaacaccucccaguaugacacauauugcuucaaugcuucagcuccaccugaagaagauuguacaucagucacagaccugcccaaugccuuugauggaccaauuaccauaacuauuguuaaccgugauggcacccgcuauguccagaaaggagaauacagaacgaauccugaagacaucuaccccagcaacccuacugaugaugacgugagcagcggcuccuccagugaaaggagcagcacuucaggagguuacaucuuuuacaccuuuucuacuguacaccccaucccagacgaagacagucccuggaucaccgacagcacagacagaaucccugcuaccacagccucagcucauaccagccauccaaugcaaggaaggacaacaccaagcccagaggacaguuccuggacugauuucuucaacccaaucucacaccccaugggacgaggucaucaagcaggaagaaggaugggagaccaagacacauuccaccccaguggggggucccauaccacucauggaucugaaucagauggacacucacaugggagucaagaagguggagcaaacacaaccucugguccuauaaggacaccccaaauuccagaauggcugaucaucuuggcaucccucuuggccuuggcuuugauucuugcaguuugcauugcagucaacagucgaagaaggugugggcagaagaaaaagcuagugaucaacaguggcaauggagcuguggaggacagaaagccaaguggacucaacggagaggccagcaagucucaggaaauggugcauuuggugaacaaggagucgucagaaacuccagaccaguuuaugacagcugaugagacaaggaaccugcagaauguggacaugaagauugggguguaacaccuacaccauuaucuuggaaagaaacaaccguuggaaacauaaccauuacagggagcugggacacuuaacagaugcaaugugcuacugauuguuucauugcgaaucuuuuuuagcauaaaauuuucuacucuuuuuguuuuuuguguuuuguucuuuaaagucagguccaauuuguaaaaacagcauugcuuucugaaauuagggcccaauuaauaaucagcaagaauuugaucguuccaguucccacuuggaggccuuucaucccucgggugugcuauggauggcuucuaacaaaaacuacacauauguauuccugaucgccaaccuuucccccaccagcuaaggacauuucccaggguuaauagggccuggucccugggaggaaauuugaauggguccauuuugcccuuccauagccuaaucccugggcauugcuuuccacugagguuggggguugggguguacuaguuacacaucuucaacagacccccucuagaaauuuuucagaugcuucugggagacacccaaagggugaagcuauuuaucuguaguaaacuauuuaucuguguuuuugaaauauuaaacccuggaucaguccuuugaucaguauaauuuuuuaaaguuacuuugucagaggcacaaaaggguuuaaacugauucauaauaaauaucuguacuucuucgaucuucaccuuuugugcugugauucuucaguuucuaaaccagcacugucugggucccuacaauguaucaggaagagcugagaaugguaaggagacucuucuaagucuucaucucagagacccugaguucccacucagacccacucagccaaaucucauggaagaccaaggagggcagcacuguuuuuguuuuuuguuuuuuguuuuuuuuuuuugacacuguccaaagguuuuccauccuguccuggaaucagaguuggaagcugaggagcuucagccucuuuuaugguuuaauggccaccuguucucuccugugaaaggcuuugcaaagucacauuaaguuugcaugaccuguuaucccuggggcccuauuucauagaggcuggcccuauuagugauuuccaaaaacaauauggaagugccuuuugaugucuuacaauaagagaagaagccaauggaaaugaaagagauuggcaaaggggaaggaugaugccauguagauccuguuugacauuuuuauggcuguauuuguaaacuuaaacacaccagugucuguucuugaugcaguugcuauuuaggaugaguuaagugccuggggagucccucaaaagguuaaagggauucccaucauuggaaucuuaucaccagauaggcaaguuuaugaccaaacaagagaguacuggcuuuauccucuaaccucauauuuucucccacuuggcaaguccuuuguggcauuuauucaucagucaggguguccgauugguccuagaacuuccaaaggcugcuugucauagaagccauugcaucuauaaagcaacggcuccuguuaaaugguaucuccuuucugaggcuccuacuaaaagucauuuguuaccuaaacuuaugugcuuaacaggcaaugcuucucagaccacaaagcagaaagaagaagaaaagcuccugacuaaaucagggcugggcuuagacagaguugaucuguagaauaucuuuaaaggagagaugucaacuuucugcacuauucccagccucugcuccucccugucuacccucuccccucccucucucccuccacuucaccccacaaucuugaaaaacuuccuuucucuucugugaacaucauuggccagauccauuuucaguggucuggauuucuuuuuauuuucuuuucaacuugaaagaaacuggacauuaggccacuauguguuguuacugccacuaguguucaagugccucuuguuuucccagagauuuccugggucugccagaggcccagacaggcucacucaagcucuuuaacugaaaagcaacaagccacuccaggacaagguucaaaaugguuacaacagccucuaccugucgccccagggagaaagggguagugauacaagucucauagccagagaugguuuuccacuccuucuagauauucccaaaaagaggcugagacaggagguuauuuucaauuuuauuuuggaauuaaauacuuuuuucccuuuauuacuguuguagucccucacuuggauauaccucuguuuucacgauagaaauaagggaggucuagagcuucuauuccuuggccauugucaacggagagcuggccaagucuucacaaacccuugcaacauugccugaaguuuauggaauaagauguauucucacucccuugaucucaagggcguaacucuggaagcacagcuugacuacacgucauuuuuaccaaugauuuucaggugaccugggcuaagucauuuaaacugggucuuuauaaaaguaaaaggccaacauuuaauuauuuugcaaagcaaccuaagagcuaaagauguaauuuuucuugcaauuguaaaucuuuugugucuccugaagacuucccuuaaaauuagcucugagugaaaaaucaaaagagacaaaagacaucuucgaauccauauuucaagccugguagaauuggcuuuucuagcagaaccuuuccaaaaguuuuauauugagauucauaacaacaccaagaauugauuuuguagccaacauucauucaauacuguuauaucagaggaguaggagagaggaaacauuugacuuaucuggaaaagcaaaauguacuuaagaauaagaauaacaugguccauucaccuuuauguuauagauaugucuuuguguaaaucauuuguuuugaguuuucaaagaauagcccauuguucauucuugugcuguacaaugaccacuguuauuguuacuuugacuuuucagagcacacccuuccucugguuuuuguauauuuauugauggaucaauaauaaugaggaaagcaugauauguauauugcugaguugaaagcacuuauuggaaaauauuaaaaggcuaacauuaaaagacuaaaggaaac

SEQ ID NO: 131 - RNA, 4688 nt, Homo sapiens
  source 1..4688: /mol_type=“unassigned RNA” /note=“Nucleotide sequence encoding homo sapiens CD44 isoform CD44v8” /organism=“Homo sapiens”
gagaagaaagccagugcgucucugggcgcaggggccaguggggcucggaggcacaggcaccccgcgacacuccagguuccccgacccacgucccuggcagccccgauuauuuacagccucagcagagcacggggcgggggcagaggggcccgcccgggagggcugcuacuucuuaaaaccucugcgggcugcuuagucacagccccccuugcuuggguguguccuucgcucgcucccucccuccgucuuaggucacuguuuucaaccucgaauaaaaacugcagccaacuuccgaggcagccucauugcccagcggaccccagccucugccagguucgguccgccauccucgucccguccuccgccggccccugccccgcgcccagggauccuccagcuccuuucgcccgcgcccuccguucgcuccggacaccauggacaaguuuugguggcacgcagccuggggacucugccucgugccgcugagccuggcgcagaucgauuugaauauaaccugccgcuuugcagguguauuccacguggagaaaaauggucgcuacagcaucucucggacggaggccgcugaccucugcaaggcuuucaauagcaccuugcccacaauggcccagauggagaaagcucugagcaucggauuugagaccugcagguauggguucauagaagggcacguggugauuccccggauccaccccaacuccaucugugcagcaaacaacacagggguguacauccucacauccaacaccucccaguaugacacauauugcuucaaugcuucagcuccaccugaagaagauuguacaucagucacagaccugcccaaugccuuugauggaccaauuaccauaacuauuguuaaccgugauggcacccgcuauguccagaaaggagaauacagaacgaauccugaagacaucuaccccagcaacccuacugaugaugacgugagcagcggcuccuccagugaaaggagcagcacuucaggagguuacaucuuuuacaccuuuucuacuguacaccccaucccagacgaagacagucccuggaucaccgacagcacagacagaaucccugcuaccaauauggacuccagucauaguauaacgcuucagccuacugcaaauccaaacacagguuugguggaagauuuggacaggacaggaccucuuucaaugacaacgcgagaccaagacacauuccaccccaguggggggucccauaccacucauggaucugaaucagauggacacucacaugggagucaagaagguggagcaaacacaaccucugguccuauaaggacaccccaaauuccagaauggcugaucaucuuggcaucccucuuggccuuggcuuugauucuugcaguuugcauugcagucaacagucgaagaaggugugggcagaagaaaaagcuagugaucaacaguggcaauggagcuguggaggacagaaagccaaguggacucaacggagaggccagcaagucucaggaaauggugcauuuggugaacaaggagucgucagaaacuccagaccaguuuaugacagcugaugagacaaggaaccugcagaauguggacaugaagauugggguguaacaccuacaccauuaucuuggaaagaaacaaccguuggaaacauaaccauuacagggagcugggacacuuaacagaugcaaugugcuacugauuguuucauugcgaaucuuuuuuagcauaaaauuuucuacucuuuuuguuuuuuguguuuuguucuuuaaagucagguccaauuuguaaaaacagcauugcuuucugaaauuagggcccaauuaauaaucagcaagaauuugaucguuccaguucccacuuggaggccuuucaucccucgggugugcuauggauggcuucuaacaaaaacuacacauauguauuccugaucgccaaccuuucccccaccagcuaaggacauuucccaggguuaauagggccuggucccugggaggaaauuugaauggguccauuuugcccuuccauagccuaaucccugggcauugcuuuccacugagguuggggguugggguguacuaguuacacaucuucaacagacccccucuagaaauuuuucagaugcuucugggagacacccaaagggugaagcuauuuaucuguaguaaacuauuuaucuguguuuuugaaauauuaaacccuggaucaguccuuugaucaguauaauuuuuuaaaguuacuuugucagaggcacaaaaggguuuaaacugauucauaauaaauaucuguacuucuucgaucuucaccuuuugugcugugauucuucaguuucuaaaccagcacugucugggucccuacaauguaucaggaagagcugagaaugguaaggagacucuucuaagucuucaucucagagacccugaguucccacucagacccacucagccaaaucucauggaagaccaaggagggcagcacuguuuuuguuuuuuguuuuuuguuuuuuuuuuuugacacuguccaaagguuuuccauccuguccuggaaucagaguuggaagcugaggagcuucagccucuuuuaugguuuaauggccaccuguucucuccugugaaaggcuuugcaaagucacauuaaguuugcaugaccuguuaucccuggggcccuauuucauagaggcuggcccuauuagugauuuccaaaaacaauauggaagugccuuuugaugucuuacaauaagagaagaagccaauggaaaugaaagagauuggcaaaggggaaggaugaugccauguagauccuguuugacauuuuuauggcuguauuuguaaacuuaaacacaccagugucuguucuugaugcaguugcuauuuaggaugaguuaagugccuggggagucccucaaaagguuaaagggauucccaucauuggaaucuuaucaccagauaggcaaguuuaugaccaaacaagagaguacuggcuuuauccucuaaccucauauuuucucccacuuggcaaguccuuuguggcauuuauucaucagucaggguguccgauugguccuagaacuuccaaaggcugcuugucauagaagccauugcaucuauaaagcaacggcuccuguuaaaugguaucuccuuucugaggcuccuacuaaaagucauuuguuaccuaaacuuaugugcuuaacaggcaaugcuucucagaccacaaagcagaaagaagaagaaaagcuccugacuaaaucagggcugggcuuagacagaguugaucuguagaauaucuuuaaaggagagaugucaacuuucugcacuauucccagccucugcuccucccugucuacccucuccccucccucucucccuccacuucaccccacaaucuugaaaaacuuccuuucucuucugugaacaucauuggccagauccauuuucaguggucuggauuucuuuuuauuuucuuuucaacuugaaagaaacuggacauuaggccacuauguguuguuacugccacuaguguucaagugccucuuguuuucccagagauuuccugggucugccagaggcccagacaggcucacucaagcucuuuaacugaaaagcaacaagccacuccaggacaagguucaaaaugguuacaacagccucuaccugucgccccagggagaaagggguagugauacaagucucauagccagagaugguuuuccacuccuucuagauauucccaaaaagaggcugagacaggagguuauuuucaauuuuauuuuggaauuaaauacuuuuuucccuuuauuacuguuguagucccucacuuggauauaccucuguuuucacgauagaaauaagggaggucuagagcuucuauuccuuggccauugucaacggagagcuggccaagucuucacaaacccuugcaacauugccugaaguuuauggaauaagauguauucucacucccuugaucucaagggcguaacucuggaagcacagcuugacuacacgucauuuuuaccaaugauuuucaggugaccugggcuaagucauuuaaacugggucuuuauaaaaguaaaaggccaacauuuaauuauuuugcaaagcaaccuaagagcuaaagauguaauuuuucuugcaauuguaaaucuuuugugucuccugaagacuucccuuaaaauuagcucugagugaaaaaucaaaagagacaaaagacaucuucgaauccauauuucaagccugguagaauuggcuuuucuagcagaaccuuuccaaaaguuuuauauugagauucauaacaacaccaagaauugauuuuguagccaacauucauucaauacuguuauaucagaggaguaggagagaggaaacauuugacuuaucuggaaaagcaaaauguacuuaagaauaagaauaacaugguccauucaccuuuauguuauagauaugucuuuguguaaaucauuuguuuugaguuuucaaagaauagcccauuguucauucuugugcuguacaaugaccacuguuauuguuacuuugacuuuucagagcacacccuuccucugguuuuuguauauuuauugauggaucaauaauaaugaggaaagcaugauauguauauugcugaguugaaagcacuuauuggaaaauauuaaaaggcuaacauuaaaagacuaaaggaaac

SEQ ID NO: 132 - RNA, 4790 nt, Homo sapiens
  source 1..4790: /mol_type=“unassigned RNA” /note=“Nucleotide sequence encoding homo sapiens CD44 isoform CD44v10” /organism=“Homo sapiens”
gagaagaaagccagugcgucucugggcgcaggggccaguggggcucggaggcacaggcaccccgcgacacuccagguuccccgacccacgucccuggcagccccgauuauuuacagccucagcagagcacggggcgggggcagaggggcccgcccgggagggcugcuacuucuuaaaaccucugcgggcugcuuagucacagccccccuugcuuggguguguccuucgcucgcucccucccuccgucuuaggucacuguuuucaaccucgaauaaaaacugcagccaacuuccgaggcagccucauugcccagcggaccccagccucugccagguucgguccgccauccucgucccguccuccgccggccccugccccgcgcccagggauccuccagcuccuuucgcccgcgcccuccguucgcuccggacaccauggacaaguuuugguggcacgcagccuggggacucugccucgugccgcugagccuggcgcagaucgauuugaauauaaccugccgcuuugcagguguauuccacguggagaaaaauggucgcuacagcaucucucggacggaggccgcugaccucugcaaggcuuucaauagcaccuugcccacaauggcccagauggagaaagcucugagcaucggauuugagaccugcagguauggguucauagaagggcacguggugauuccccggauccaccccaacuccaucugugcagcaaacaacacagggguguacauccucacauccaacaccucccaguaugacacauauugcuucaaugcuucagcuccaccugaagaagauuguacaucagucacagaccugcccaaugccuuugauggaccaauuaccauaacuauuguuaaccgugauggcacccgcuauguccagaaaggagaauacagaacgaauccugaagacaucuaccccagcaacccuacugaugaugacgugagcagcggcuccuccagugaaaggagcagcacuucaggagguuacaucuuuuacaccuuuucuacuguacaccccaucccagacgaagacagucccuggaucaccgacagcacagacagaaucccugcuaccaauaggaaugaugucacagguggaagaagagacccaaaucauucugaaggcucaacuacuuuacuggaagguuauaccucucauuacccacacacgaaggaaagcaggaccuucaucccagugaccucagcuaagacuggguccuuuggaguuacugcaguuacuguuggagauuccaacucuaaugucaaucguuccuuaucaggagaccaagacacauuccaccccaguggggggucccauaccacucauggaucugaaucagauggacacucacaugggagucaagaagguggagcaaacacaaccucugguccuauaaggacaccccaaauuccagaauggcugaucaucuuggcaucccucuuggccuuggcuuugauucuugcaguuugcauugcagucaacagucgaagaaggugugggcagaagaaaaagcuagugaucaacaguggcaauggagcuguggaggacagaaagccaaguggacucaacggagaggccagcaagucucaggaaauggugcauuuggugaacaaggagucgucagaaacuccagaccaguuuaugacagcugaugagacaaggaaccugcagaauguggacaugaagauugggguguaacaccuacaccauuaucuuggaaagaaacaaccguuggaaacauaaccauuacagggagcugggacacuuaacagaugcaaugugcuacugauuguuucauugcgaaucuuuuuuagcauaaaauuuucuacucuuuuuguuuuuuguguuuuguucuuuaaagucagguccaauuuguaaaaacagcauugcuuucugaaauuagggcccaauuaauaaucagcaagaauuugaucguuccaguucccacuuggaggccuuucaucccucgggugugcuauggauggcuucuaacaaaaacuacacauauguauuccugaucgccaaccuuucccccaccagcuaaggacauuucccaggguuaauagggccuggucccugggaggaaauuugaauggguccauuuugcccuuccauagccuaaucccugggcauugcuuuccacugagguuggggguugggguguacuaguuacacaucuucaacagacccccucuagaaauuuuucagaugcuucugggagacacccaaagggugaagcuauuuaucuguaguaaacuauuuaucuguguuuuugaaauauuaaacccuggaucaguccuuugaucaguauaauuuuuuaaaguuacuuugucagaggcacaaaaggguuuaaacugauucauaauaaauaucuguacuucuucgaucuucaccuuuugugcugugauucuucaguuucuaaaccagcacugucugggucccuacaauguaucaggaagagcugagaaugguaaggagacucuucuaagucuucaucucagagacccugaguucccacucagacccacucagccaaaucucauggaagaccaaggagggcagcacuguuuuuguuuuuuguuuuuuguuuuuuuuuuuugacacuguccaaagguuuuccauccuguccuggaaucagaguuggaagcugaggagcuucagccucuuuuaugguuuaauggccaccuguucucuccugugaaaggcuuugcaaagucacauuaaguuugcaugaccuguuaucccuggggcccuauuucauagaggcuggcccuauuagugauuuccaaaaacaauauggaagugccuuuugaugucuuacaauaagagaagaagccaauggaaaugaaagagauuggcaaaggggaaggaugaugccauguagauccuguuugacauuuuuauggcuguauuuguaaacuuaaacacaccagugucuguucuugaugcaguugcuauuuaggaugaguuaagugccuggggagucccucaaaagguuaaagggauucccaucauuggaaucuuaucaccagauaggcaaguuuaugaccaaacaagagaguacuggcuuuauccucuaaccucauauuuucucccacuuggcaaguccuuuguggcauuuauucaucagucaggguguccgauugguccuagaacuuccaaaggcugcuugucauagaagccauugcaucuauaaagcaacggcuccuguuaaaugguaucuccuuucugaggcuccuacuaaaagucauuuguuaccuaaacuuaugugcuuaacaggcaaugcuucucagaccacaaagcagaaagaagaagaaaagcuccugacuaaaucagggcugggcuuagacagaguugaucuguagaauaucuuuaaaggagagaugucaacuuucugcacuauucccagccucugcuccucccugucuacccucuccccucccucucucccuccacuucaccccacaaucuugaaaaacuuccuuucucuucugugaacaucauuggccagauccauuuucaguggucuggauuucuuuuuauuuucuuuucaacuugaaagaaacuggacauuaggccacuauguguuguuacugccacuaguguucaagugccucuuguuuucccagagauuuccugggucugccagaggcccagacaggcucacucaagcucuuuaacugaaaagcaacaagccacuccaggacaagguucaaaaugguuacaacagccucuaccugucgccccagggagaaagggguagugauacaagucucauagccagagaugguuuuccacuccuucuagauauucccaaaaagaggcugagacaggagguuauuuucaauuuuauuuuggaauuaaauacuuuuuucccuuuauuacuguuguagucccucacuuggauauaccucuguuuucacgauagaaauaagggaggucuagagcuucuauuccuuggccauugucaacggagagcuggccaagucuucacaaacccuugcaacauugccugaaguuuauggaauaagauguauucucacucccuugaucucaagggcguaacucuggaagcacagcuugacuacacgucauuuuuaccaaugauuuucaggugaccugggcuaagucauuuaaacugggucuuuauaaaaguaaaaggccaacauuuaauuauuuugcaaagcaaccuaagagcuaaagauguaauuuuucuugcaauuguaaaucuuuugugucuccugaagacuucccuuaaaauuagcucugagugaaaaaucaaaagagacaaaagacaucuucgaauccauauuucaagccugguagaauuggcuuuucuagcagaaccuuuccaaaaguuuuauauugagauucauaacaacaccaagaauugauuuuguagccaacauucauucaauacuguuauaucagaggaguaggagagaggaaacauuugacuuaucuggaaaagcaaaauguacuuaagaauaagaauaacaugguccauucaccuuuauguuauagauaugucuuuguguaaaucauuuguuuugaguuuucaaagaauagcccauuguucauucuugugcuguacaaugaccacuguuauuguuacuuugacuuuucagagcacacccuuccucugguuuuuguauauuuauugauggaucaauaauaaugaggaaagcaugauauguauauugcugaguugaaagcacuuauuggaaaauauuaaaaggcuaacauuaaaagacuaaaggaaac

SEQ ID NO: 133 - RNA, 501 nt, Homo sapiens
  source 1..501: /mol_type=“unassigned RNA” /note=“Nucleotide sequence encoding exon C1 of homo sapiens CD44” /organism=“Homo sapiens”
gagaagaaagccagugcgucucugggcgcaggggccaguggggcucggaggcacaggcaccccgcgacacuccagguuccccgacccacgucccuggcagccccgauuauuuacagccucagcagagcacggggcgggggcagaggggcccgcccgggagggcugcuacuucuuaaaaccucugcgggcugcuuagucacagccccccuugcuuggguguguccuucgcucgcucccucccuccgucuuaggucacuguuuucaaccucgaauaaaaacugcagccaacuuccgaggcagccucauugcccagcggaccccagccucugccagguucgguccgccauccucgucccguccuccgccggccccugccccgcgcccagggauccuccagcuccuuucgcccgcgcccuccguucgcuccggacaccauggacaaguuuugguggcacgcagccuggggacucugccucgugccgcugagccuggcgcagaucg

SEQ ID NO: 134 - RNA, 166 nt, Homo sapiens
  source 1..166: /mol_type=“unassigned RNA” /note=“Nucleotide sequence encoding exon C2 of homo sapiens CD44 ” /organism=“Homo sapiens”
auuugaauauaaccugccgcuuugcagguguauuccacguggagaaaaauggucgcuacagcaucucucggacggaggccgcugaccucugcaaggcuuucaauagcaccuugcccacaauggcccagauggagaaagcucugagcaucggauuugagaccugcag

SEQ ID NO: 135 - RNA, 134 nt, Homo sapiens
  source 1..134: /mol_type=“unassigned RNA” /note=“Nucleotide sequence encoding exon C3 of homo sapiens CD44” /organism=“Homo sapiens”
guauggguucauagaagggcacguggugauuccccggauccaccccaacuccaucugugcagcaaacaacacagggguguacauccucacauccaacaccucccaguaugacacauauugcuucaaugcuucag

SEQ ID NO: 136 - RNA, 69 nt, Homo sapiens
  source 1..69: /mol_type=“unassigned RNA” /note=“Nucleotide sequence encoding exon C4 of homo sapiens CD44 ” /organism=“Homo sapiens”
cuccaccugaagaagauuguacaucagucacagaccugcccaaugccuuugauggaccaauuaccauaa

SEQ ID NO: 137 - RNA, 231 nt, Homo sapiens
  source 1..231: /mol_type=“unassigned RNA” /note=“Nucleotide sequence encoding exon C5 of homo sapiens CD44 ” /organism=“Homo sapiens”
cuauuguuaaccgugauggcacccgcuauguccagaaaggagaauacagaacgaauccugaagacaucuaccccagcaacccuacugaugaugacgugagcagcggcuccuccagugaaaggagcagcacuucaggagguuacaucuuuuacaccuuuucuacuguacaccccaucccagacgaagacagucccuggaucaccgacagcacagacagaaucccugcuacca

SEQ ID NO: 138 - RNA, 129 nt, Homo sapiens
  source 1..129: /mol_type=“unassigned RNA” /note=“Nucleotide sequence encoding exon V2 of homo sapiens CD44 ” /organism=“Homo sapiens”
cuuugaugagcacuagugcuacagcaacugagacagcaaccaagaggcaagaaaccugggauugguuuucaugguuguuucuaccaucagagucaaagaaucaucuucacacaacaacacaaauggcug

SEQ ID NO: 139 - RNA, 126 nt, Homo sapiens
  source 1..126: /mol_type=“unassigned RNA” /note=“Nucleotide sequence encoding exon V3 of homo sapiens CD44 ” /organism=“Homo sapiens”
guacgucuucaaauaccaucucagcaggcugggagccaaaugaagaaaaugaagaugaaagagacagacaccucaguuuuucuggaucaggcauugaugaugaugaagauuuuaucuccagcacca

SEQ ID NO: 140 - RNA, 114 nt, Homo sapiens
  source 1..114: /mol_type=“unassigned RNA” /note=“Nucleotide sequence encoding exon V4 of homo sapiens CD44 ” /organism=“Homo sapiens”
uuucaaccacaccacgggcuuuugaccacacaaaacagaaccaggacuggacccaguggaacccaagccauucaaauccggaagugcuacuucagacaaccacaaggaugacug

SEQ ID NO: 141 - RNA, 117 nt, Homo sapiens
  source 1..117: /mol_type=“unassigned RNA” /note=“Nucleotide sequence encoding exon V5 of homo sapiens CD44 ” /organism=“Homo sapiens”
auguagacagaaauggcaccacugcuuaugaaggaaacuggaacccagaagcacacccuccccucauucaccaugagcaucaugaggaagaagagaccccacauucuacaagcacaa

SEQ ID NO: 142 - RNA, 129 nt, Homo sapiens
  source 1..129: /mol_type=“unassigned RNA” /note=“Nucleotide sequence encoding exon V6 of homo sapiens CD44 ” /organism=“Homo sapiens”
uccaggcaacuccuaguaguacaacggaagaaacagcuacccagaaggaacagugguuuggcaacagauggcaugagggauaucgccaaacacccaaagaagacucccauucgacaacagggacagcug

SEQ ID NO: 143 - RNA, 132 nt, Homo sapiens
  source 1..132: /mol_type=“unassigned RNA” /note=“Nucleotide sequence encoding exon V7 of homo sapiens CD44 ” /organism=“Homo sapiens”
cagccucagcucauaccagccauccaaugcaaggaaggacaacaccaagcccagaggacaguuccuggacugauuucuucaacccaaucucacaccccaugggacgaggucaucaagcaggaagaaggaugg

SEQ ID NO: 144 - RNA, 102 nt, Homo sapiens
  source 1..102: /mol_type=“unassigned RNA” /note=“Nucleotide sequence encoding exon V8 of homo sapiens CD44 ” /organism=“Homo sapiens”
auauggacuccagucauaguauaacgcuucagccuacugcaaauccaaacacagguuugguggaagauuuggacaggacaggaccucuuucaaugacaacgc

SEQ ID NO: 145 - RNA, 90 nt, Homo sapiens
  source 1..90: /mol_type=“unassigned RNA” /note=“Nucleotide sequence encoding exon V9 of homo sapiens CD44 ” /organism=“Homo sapiens”
agcagaguaauucucagagcuucucuacaucacaugaaggcuuggaagaagauaaagaccauccaacaacuucuacucugacaucaagca

SEQ ID NO: 146 - RNA, 204 nt, Homo sapiens
  source 1..204: /mol_type=“unassigned RNA” /note=“Nucleotide sequence encoding exon V10 of homo sapiens CD44 ” /organism=“Homo sapiens”
auaggaaugaugucacagguggaagaagagacccaaaucauucugaaggcucaacuacuuuacuggaagguuauaccucucauuacccacacacgaaggaaagcaggaccuucaucccagugaccucagcuaagacuggguccuuuggaguuacugcaguuacuguuggagauuccaacucuaaugucaaucguuccuuaucag

SEQ ID NO: 147 - RNA, 63 nt, Homo sapiens
  source 1..63: /mol_type=“unassigned RNA” /note=“Nucleotide sequence encoding exon C6 of homo sapiens CD44 ” /organism=“Homo sapiens”
gagaccaagacacauuccaccccaguggggggucccauaccacucauggaucugaaucagaug

SEQ ID NO: 148 - RNA, 72 nt, Homo sapiens
  source 1..72: /mol_type=“unassigned RNA” /note=“Nucleotide sequence encoding exon C7 of homo sapiens CD44 ” /organism=“Homo sapiens”
gacacucacaugggagucaagaagguggagcaaacacaaccucugguccuauaaggacaccccaaauuccag

SEQ ID NO: 149 - RNA, 79 nt, Homo sapiens
  source 1..79: /mol_type=“unassigned RNA” /note=“Nucleotide sequence encoding exon C8 of homo sapiens CD44 ” /organism=“Homo sapiens”
aauggcugaucaucuuggcaucccucuuggccuuggcuuugauucuugcaguuugcauugcagucaacagucgaagaag

SEQ ID NO: 150 - RNA, 3271 nt, Homo sapiens
  source 1..3271: /mol_type=“unassigned RNA” /note=“Nucleotide sequence encoding exon C9 of homo sapiens CD44 ” /organism=“Homo sapiens”
gugugggcagaagaaaaagcuagugaucaacaguggcaauggagcuguggaggacagaaagccaaguggacucaacggagaggccagcaagucucaggaaauggugcauuuggugaacaaggagucgucagaaacuccagaccaguuuaugacagcugaugagacaaggaaccugcagaauguggacaugaagauugggguguaacaccuacaccauuaucuuggaaagaaacaaccguuggaaacauaaccauuacagggagcugggacacuuaacagaugcaaugugcuacugauuguuucauugcgaaucuuuuuuagcauaaaauuuucuacucuuuuuguuuuuuguguuuuguucuuuaaagucagguccaauuuguaaaaacagcauugcuuucugaaauuagggcccaauuaauaaucagcaagaauuugaucguuccaguucccacuuggaggccuuucaucccucgggugugcuauggauggcuucuaacaaaaacuacacauauguauuccugaucgccaaccuuucccccaccagcuaaggacauuucccaggguuaauagggccuggucccugggaggaaauuugaauggguccauuuugcccuuccauagccuaaucccugggcauugcuuuccacugagguuggggguugggguguacuaguuacacaucuucaacagacccccucuagaaauuuuucagaugcuucugggagacacccaaagggugaagcuauuuaucuguaguaaacuauuuaucuguguuuuugaaauauuaaacccuggaucaguccuuugaucaguauaauuuuuuaaaguuacuuugucagaggcacaaaaggguuuaaacugauucauaauaaauaucuguacuucuucgaucuucaccuuuugugcugugauucuucaguuucuaaaccagcacugucugggucccuacaauguaucaggaagagcugagaaugguaaggagacucuucuaagucuucaucucagagacccugaguucccacucagacccacucagccaaaucucauggaagaccaaggagggcagcacuguuuuuguuuuuuguuuuuuguuuuuuuuuuuugacacuguccaaagguuuuccauccuguccuggaaucagaguuggaagcugaggagcuucagccucuuuuaugguuuaauggccaccuguucucuccugugaaaggcuuugcaaagucacauuaaguuugcaugaccuguuaucccuggggcccuauuucauagaggcuggcccuauuagugauuuccaaaaacaauauggaagugccuuuugaugucuuacaauaagagaagaagccaauggaaaugaaagagauuggcaaaggggaaggaugaugccauguagauccuguuugacauuuuuauggcuguauuuguaaacuuaaacacaccagugucuguucuugaugcaguugcuauuuaggaugaguuaagugccuggggagucccucaaaagguuaaagggauucccaucauuggaaucuuaucaccagauaggcaaguuuaugaccaaacaagagaguacuggcuuuauccucuaaccucauauuuucucccacuuggcaaguccuuuguggcauuuauucaucagucaggguguccgauugguccuagaacuuccaaaggcugcuugucauagaagccauugcaucuauaaagcaacggcuccuguuaaaugguaucuccuuucugaggcuccuacuaaaagucauuuguuaccuaaacuuaugugcuuaacaggcaaugcuucucagaccacaaagcagaaagaagaagaaaagcuccugacuaaaucagggcugggcuuagacagaguugaucuguagaauaucuuuaaaggagagaugucaacuuucugcacuauucccagccucugcuccucccugucuacccucuccccucccucucucccuccacuucaccccacaaucuugaaaaacuuccuuucucuucugugaacaucauuggccagauccauuuucaguggucuggauuucuuuuuauuuucuuuucaacuugaaagaaacuggacauuaggccacuauguguuguuacugccacuaguguucaagugccucuuguuuucccagagauuuccugggucugccagaggcccagacaggcucacucaagcucuuuaacugaaaagcaacaagccacuccaggacaagguucaaaaugguuacaacagccucuaccugucgccccagggagaaagggguagugauacaagucucauagccagagaugguuuuccacuccuucuagauauucccaaaaagaggcugagacaggagguuauuuucaauuuuauuuuggaauuaaauacuuuuuucccuuuauuacuguuguagucccucacuuggauauaccucuguuuucacgauagaaauaagggaggucuagagcuucuauuccuuggccauugucaacggagagcuggccaagucuucacaaacccuugcaacauugccugaaguuuauggaauaagauguauucucacucccuugaucucaagggcguaacucuggaagcacagcuugacuacacgucauuuuuaccaaugauuuucaggugaccugggcuaagucauuuaaacugggucuuuauaaaaguaaaaggccaacauuuaauuauuuugcaaagcaaccuaagagcuaaagauguaauuuuucuugcaauuguaaaucuuuugugucuccugaagacuucccuuaaaauuagcucugagugaaaaaucaaaagagacaaaagacaucuucgaauccauauuucaagccugguagaauuggcuuuucuagcagaaccuuuccaaaaguuuuauauugagauucauaacaacaccaagaauugauuuuguagccaacauucauucaauacuguuauaucagaggaguaggagagaggaaacauuugacuuaucuggaaaagcaaaauguacuuaagaauaagaauaacaugguccauucaccuuuauguuauagauaugucuuuguguaaaucauuuguuuugaguuuucaaagaauagcccauuguucauucuugugcuguacaaugaccacuguuauuguuacuuugacuuuucagagcacacccuuccucugguuuuuguauauuuauugauggaucaauaauaaugaggaaagcaugauauguauauugcugaguugaaagcacuuauuggaaaauauuaaaaggcuaacauuaaaagacuaaaggaaac

SEQ ID NO: 151 - RNA, 2087 nt, Homo sapiens
  source 1..2087: /mol_type=“unassigned RNA” /note=“Nucleotide sequence encoding acid synthetase 1 (HAS1)” /organism=“Homo sapiens”
cggagagaagagagagcccggccagacccacugcgaugagacagcaggacgcgcccaagcccacuccugcagccugccgcugcuccggccuggcccggagggugcugaccaucgccuucgcccugcucauccugggccucaugaccugggccuacgccgccggggugccgcuggccuccgaucgcuacggccuccuggccuucggccucuacggggccuuccuuucagcgcaccugguggcgcagagccucuucgcguaccuggagcaccggcggguggcggcggcggcgcgggggccgcuggaugcagccaccgcgcgcaguguggcgcugaccaucuccgccuaccaggaggaccccgcguaccugcgccagugccuggcguccgcccgcgcccugcuguacccgcgcgcgcggcugcgcguccucauggugguggauggcaaccgcgccgaggaccucuacauggucgacauguuccgcgaggucuucgcugacgaggaccccgccacguacgugugggacggcaacuaccaccagcccugggaacccgcggcggcgggcgcggugggcgccggagccuaucgggagguggaggcggaggauccugggcggcuggcaguggaggcgcuggugaggacucgcaggugcgugugcguggcgcagcgcuggggcggcaagcgcgaggucauguacacagccuucaaggcgcucggagauucgguggacuacgugcaggucugugacucggacacaagguuggaccccauggcacugcuggagcucgugcggguacuggacgaggacccccggguaggggcuguugguggggacgugcggauccuuaacccucuggacuccugggucagcuuccuaagcagccugcgauacuggguagccuucaauguggagcgggcuugucagagcuacuuccacuguguauccugcaucagcgguccucuaggccuauauaggaauaaccucuugcagcaguuucuugaggccugguacaaccagaaguuccuggguacccacuguacuuuuggggaugaccggcaccucaccaaccgcaugcucagcauggguuaugcuaccaaguacaccuccaggucccgcugcuacucagagacgcccucguccuuccugcgguggcugagccagcagacacgcugguccaagucguacuuccgugaguggcuguacaacgcgcucugguggcaccggcaccaugcguggaugaccuacgaggcgguggucuccggccuguuccccuucuucguggcggccacugugcugcgucuguucuacgcgggccgcccuugggcgcugcugugggugcugcugugcgugcagggcguggcacuggccaaggcggccuucgcggccuggcugcggggcugccugcgcauggugcuucugucgcucuacgcgccccucuacauguguggccuccugccugccaaguuccuggcgcuagucaccaugaaccagaguggcuggggcaccucgggccggcggaagcuggccgcuaacuacgucccucugcugccccuggcgcucugggcgcugcugcugcuugggggccugguccgcagcguagcacacgaggccagggccgacuggagcggcccuucccgcgcagccgaggccuaccacuuggccgcgggggccggcgccuacgugggcuacuggguggccauguugacgcuguacugggugggcgugcggaggcuuugccggcggcggaccgggggcuaccgcguccaggugugaguccagccacgcggaugccgccucaagggucuucaggggaggccagaggagagcugcugggccccgagccacgaacuugcugggugguucucugggccucaguuucccuccucugcaaaacgagggggucagcccaagauucuucagucuggacuauauugggacugggacuucugggucuccagggaggguauuuauuggucagggugugggaucugaggaguggagggaaaggguccugcuuucuccucguucuuauuuaaucuccauuucuacugugugaucaggauguaauaaagaauuuuauuuauuuuc

SEQ ID NO: 152 - RNA, 4190 nt, Homo sapiens
  source 1..4190: /mol_type=“unassigned RNA” /note=“Nucleotide sequence encoding hyaluronic acid synthetase 2 (HAS2) ” /organism=“Homo sapiens”
gaagucaagacgucuggaaagaauuacccaguccuggcuucgagcagcccauugaaccagagacuugaaacagccccagccaaagacuuuucucccaauucugcgcuuccuggguucugcugagucuuccacaggcuuuuuuuuuuuuuuuuuuuuuuuuuaagacgaaaaagagauuuucuguuaucgggggcagaaagacugaagcccaaaaaaaaaaaaaaaaaaaaaagaaaagaaaagaaaaaagaaaaguuaauuuauuuuuaaagcauaauuuuuuuaagaauuagacugaagugcaacggaaacauaaagagaauauuagugaaauuauuuuuuaaaguggggaagaaucaaacauuuaagacuccccuauccuuuuuaaauguuguuuuuaaauuucuuauuuuuuuuggccggucgucucaaauucaucugaucucuuauuaccucaauuuuggaaacugcccgccaccgacccuccgggaccacacagacaggcugaggacgacuuuaugaccaagagcugaacaagaugcauugugagagguuucuauguauccugagaauaauuggaaccacacucuuuggagucucucuccuccuuggaaucacagcugcuuauauuguuggcuaccaguuuauccaaacggauaauuacuauuucucuuuuggacuguauggugccuuuuuggcaucacaccucaucauccaaagccuguuugccuuuuuggagcaccgaaaaaugaaaaaaucccuagaaacccccauaaaguugaacaaaacaguugcccuuugcaucgcugccuaucaagaagauccagacuacuuaaggaaauguuugcaaucugugaaaaggcuaaccuacccugggauuaaaguugucauggucauagaugggaacucagaagaugaccuuuacaugauggacaucuucagugaagucaugggcagagacaaaucagccacuuauaucuggaagaacaacuuccacgaaaagggucccggugagacagaugagucacauaaagaaagcucgcaacacguaacgcaauuggucuuguccaacaaaaguaucugcaucaugcaaaaaugggguggaaaaagagaagucauguacacagccuucagagcacugggacgaaguguggauuauguacagguuugugauucagacacuaugcuugacccagccucaucuguggagaugguaaaaguuuuagaagaagaucccaugguuggagguguugggggagauguccagauuuuaaacaaguacgauuccuggaucucauuccucagcaguguaagauauuggauggcuuuuaauauagaaagggccugucagucuuauuuuggguguguucagugcauuaguggaccucugggaauguacagaaacuccuuguugcaugaguuuguggaagauugguacaaucaagaauuuaugggcaaccaauguagcuuuggugaugacaggcaucucacgaaccgggugcugagccugggcuaugcaacaaaauacacagcucgaucuaagugccuuacugaaacaccuauagaauaucucagauggcuaaaccagcagacccguuggagcaaguccuacuuccgagaauggcuguacaaugcaaugugguuucacaaacaucacuuguggaugaccuacgaagcgauuaucacuggauucuuuccuuucuuucucauugccacaguaauccagcucuucuaccgggguaaaauuuggaacauucuccucuucuuguuaacuguccagcuaguaggucucauaaaaucaucuuuugccagcugccuuagaggaaauaucgucauggucuucaugucucucuacucaguguuauacaugucgaguuuacuucccgccaagauguuugcaauugcaacaauaaacaaagcuggguggggcacaucaggaaggaaaaccauuguuguuaauuucauaggacucauuccaguaucaguuugguuuacaauccuccuggguggugugauuuucaccauuuauaaggagucuaaaaggccauuuucagaauccaaacagacaguucuaauuguuggaacguugcucuaugcaugcuauugggucaugcuuuugacgcuguauguaguucucaucaauaaguguggcaggcggaagaagggacaacaauaugacauggugcuugauguaugaucuuccauguuuugacguuugcagucacacacaacaccuuaguuccucuaggggcuguacaguauuguggcaucagauaaugccaccaaaggagacauaucacugcugcugggacuugaacaaagacauuuauauggguuuauuuucauucugccaaaguaaaacaauacaucaacaagaagaaacucagauuuaaccuguuauuucuaugaaaaugggaugaauucuuuguuuaugcacuuuuuccuuacugugcauccgccugaaaguguuuugcccuauauaccucacuagccaugcuuuauguggguuaucauggaagaaaaggauuuuggaaacucaaggaaaaguucuuucaaccuauacaaccuaacuuauggacuguuuugauagaugauaauuuuuuuuuuuuaggaaggauuuucuuuuuaacuuuaccaaaugaaaugccaaaggaaguuuuaaaggccguuggcugugcuguauuuugauauaauuguacuguguuuuuaaauuuuguaugccaaucuuaaagacaaauuuugcauauucucuauuuuacuuuucugccaaaauaaaccuguucuuccuuuuuuaaaauaaaauaaguucuuaaaaaauuuauacuuaaaaaauccugcccaaaaugugaagcuugguugacugauguucaugauagaaagaauaaaauguuucucucucucuaccuuuuaaaauugaauaguuuauuucugugaaagaaguauuuaaacuuucaauauuuuaacuuuuuguuuuuauuucuuuuagaaaaggccaauauaccuaucacacuuuggaaguaaaaauacacacuuucguguguaccuaaaaaaaaaaucguugaaaaucaaggccaaagguagugcaauuuuuucauuaagauuuaaaaaaaagggaaugauagucuuugaaagaaaacaguaggcauccagcacuggacaaaacauggguaucaaagaugaauaaucuuuggagauucuggcaguguuuucccagaacaagucaaguggaaaguggagaaauuaucuguauaauuuuggacacauacaaugcaguuuaucaaagguuuuguucuguggccugaauuuacuggguccuaccuacacauugaacauguuuugccugucuuuuuuuuuuuuuucaacuugccaguucacuuuacauguuaguauaauguuuacacgggugaguuggauaaauuauaaaacauauaaauucaaaauuggcagauagaaucaccaaguaucuauccucuuuuacuuucaaaugaggaauuuuguuuuucugaauuacacagaucaucacuuccuauuuccuguucuggaccuguauaaaaaugucuacacaguagaagugacaucaagguuuaauaaguauaucaaugauuggcacauauaaaaauuguugaaccacauacucugaacuuggcuaauuuaguuacugcaaggccuccauuauccaguuuuauuuuuuacacgauugaccuugccuuguagcuggugcuguguagaccuguguugaaaacacaaucggaauauaugaauaauugaauaaacagcauuauggugaggcagagacacauggagaaguguuaaaaaaaaaaugggcuuccugccuuucugccucuuuuuuaugcagucaucuauguuacaucuauccugccuaagaaaaagcugcacauccuaccuucagaguacaaaaagguacaucugagacucaagacucucacugauuggagagcuuguggaaaacaaaacacaccaugccaauaaaugagaugaaaacuugaguuugccuuuuuaacuauuuauguucuaaguuaagcuuugauaacauucaaaugucaaauucucucauucuuauaaaaaguugaauuaauugccuguauuuauuuuagcaauuauucaauguauuuccaguauaggauguauaguauaauuaauuuuuuguaaauaaaauauuuuugauaaga

SEQ ID NO: 153 - RNA, 4220 nt, Homo sapiens
  source 1..4220: /mol_type=“unassigned RNA” /note=“Nucleotide sequence encoding hyaluronic acid synthetase 3 (HAS3) ” /organism=“Homo sapiens”
gagugcguucgcggcugcuuugaccuggugggcgccgccuccggcacugcaccgaggcggggcgccagcgcccagguugcugggcuggccuuggcgcccccuuccccuacccagagcgcaggcaggcaggggggucccgccgcgccccuucagcauaugccggugcagcugacgacagcccugcguguggugggcaccagccuguuugcccuggcagugcuggguggcauccuggcagccuaugugacgggcuaccaguucauccacacggaaaagcacuaccuguccuucggccuguacggcgccauccugggccugcaccugcucauucagagccuuuuugccuuccuggagcaccggcgcaugcgacgugccggccaggcccugaagcugcccuccccgcggcggggcucgguggcacugugcauugccgcauaccaggaggacccugacuacuugcgcaagugccugcgcucggcccagcgcaucuccuucccugaccucaagguggucauggugguggauggcaaccgccaggaggacgccuacaugcuggacaucuuccacgaggugcugggcggcaccgagcaggccggcuucuuuguguggcgcagcaacuuccaugaggcaggcgagggugagacggaggccagccugcaggagggcauggaccgugugcgggauguggugcgggccagcaccuucucgugcaucaugcagaaguggggaggcaagcgcgaggucauguacacggccuucaaggcccucggcgauucgguggacuacauccaggugugcgacucugacacugugcuggauccagccugcaccaucgagaugcuucgaguccuggaggaggauccccaaguagggggagucgggggagauguccagauccucaacaaguacgacucauggauuuccuuccugagcagcgugcgguacuggauggccuucaacguggagcgggccugccaguccuacuuuggcugugugcaguguauuagugggcccuugggcauguaccgcaacagccuccuccagcaguuccuggaggacugguaccaucagaaguuccuaggcagcaagugcagcuucggggaugaccggcaccucaccaaccgaguccugagccuuggcuaccgaacuaaguauaccgcgcgcuccaagugccucacagagacccccacuaaguaccuccgguggcucaaccagcaaacccgcuggagcaagucuuacuuccgggaguggcucuacaacucucugugguuccauaagcaccaccucuggaugaccuacgagucaguggucacggguuucuuccccuucuuccucauugccacgguuauacagcuuuucuaccggggccgcaucuggaacauucuccucuuccugcugacggugcagcuggugggcauuaucaaggccaccuacgccugcuuccuucggggcaaugcagagaugaucuucaugucccucuacucccuccucuauauguccagccuucugccggccaagaucuuugccauugcuaccaucaacaaaucuggcuggggcaccucuggccgaaaaaccauuguggugaacuucauuggccucauuccuguguccaucuggguggcaguucuccugggagggcuggccuacacagcuuauugccaggaccuguucagugagacagagcuagccuuccuugucucuggggcuauacuguauggcugcuacuggguggcccuccucaugcuauaucuggccaucaucgcccggcgaugugggaagaagccggagcaguacagcuuggcuuuugcugaggugugacauggcccccaagcagagcggguaaagugcaauggguaagggagggaaggggaauggaagagaaaagacagggugggagggaggagggagugcuguguuuuagucucuuaaugguccaaaggacaaaucuaaaaugcaaagaacggugauguaguauggccugacagcucuguuuagaggaggcaacacugaucccccagaugcagggcugcaggggauucuguguuuucagacugccugucugcuugcaucugcacauaggcaguagccuccuccugggcuccagagggcacucagaaguugugcuaaaccaaguuaagucccauucaguggcaacuugugauagguaccugagugacggcaaccugcggaaggagguucucccagcccaucugaacacaaccagagguggcaggagaauuucuacugagcgagcugggccgguuaguguaugucacccccaccccaccccuaaguagucaucaaugcaauaagauugcgccugagauacaaggcccagaagccugaucuuugggcaucagaaaacaggguccaggaauggugcuuuaugugagauaccccacuccacaucaacauuccagggaugagccaaaccagcagggaguuagcacugaacugcuuuuaaaagugcacauuaaaaaggaaaguuugccaggaggaacaaagagauugugguggugcuaaaggaggccauaagcuacacagaggccuuggguguuccaccuggaaacugcucagacgucuagauggguucuuagcuugucugugaucucugcuggggagauaaaaagauuaagccccaacauguucagaaaagaagugaagucuuggguauuuuaaccuguauacucuugaauuccucucaaauucagcucugaucugaggcuaagacacacuccccacuucacuuucuucaaagccacauuuuuugagguaucacugcagucaccucuucuacccucaucaucauagguaagguuuucaagguggcaauuggggcggagccccggcucuuauagaagcuucagcaggaggcaagcguguucucagcacauaugggaacuaugaggagccucugaucaaauuggcuacaaucuuggagcugcuuggacggauuccuuggcagccggguuagcaugugugacuuucaggcuacuguucuugacaaucaucuccaauggaaagcuuuucaguguucccaaagugaacucucaaauccaaaaugguuaucuuugagaccauccauucuccucaguggcuucuccagggaauucuuacagccaaguugugacagucacugcauuugccugcuucuuuccagaaaccaaacuaggagaugaaacugguuccuacauccuaagguucuugcuuucucucucaugccuccugaggcuguuuuuggcuguuuucccucugcugcuuuuggggaaugaggggaagccauuuuccagugacuugcaauccaggcuguucucagcguuuugaguuuaaaaccugggauccugacuaagccuuugacuuaaggguugcuugcuugcccuccaaauguccuuucucagaggggccagcuaacccgugcagaaccagcacuaagguggacagcagacaagagggcaagccucuaguguaccaagugcuuccuacaaagacgcaaggugugcucugagccacagaugggcaaacccuggugcuuuccuucaucucccacgaacucaaggguuuuccagguguagcuaacaguugccacaucacacagaccucgaguuucugguaagacugcugguugacaucagacccaacccaugaaggcuggaaggcagcaggcauuugcuaaggcagcugauccaggcaaucguucugcuggccaagaaguuaaacuauuuugagcauuagaauggaggaaauccggucagccaagugcagaguucagacuucgcuaagggcuuguuuuucuucagcauuuacuugaagauuaauguaggaugacaggcucuccuggcuguccuaccaucagcucugccuugcacuguggucgucaacuuuccucaaaucaaaaacaggcagguacagguagugggcucacaacguuugaccucgacugguuuuucuaaguuauuuuguacauuuuucagcagcaaaaccaaacugggucuucagcuuuauccccguuucuugcaagggaagagccuuuauacaauuggacgcauuuugguuuuuccucauugagaauucaaauccucuuuuguauuguuucuacaauaauuuguaaacauauuuauuuuuaccugcuuuuuuuuuuuuuuuuaauuuucaggucaaguuuuuuauacugcacuuauuugucaaaauaaagauucucacauaugccgguuaucucgaaa